(19) United States
(12) Patent Application Publication
(10) Pub. No.: US 2024/0270866 A1
(43) Pub. Date: Aug. 15, 2024

(54) Anti-SIRP-Alpha Antibodies and Methods of Use Thereof

(71) Applicant: Alector LLC, South San Francisco, CA (US)

(72) Inventors: Andrew Pincetic, San Francisco, CA (US); Arnon Rosenthal, Woodside, CA (US)

(73) Assignee: Alector LLC, South San Francisco, CA (US)

(21) Appl. No.: 18/423,604

(22) Filed: Jan. 26, 2024

Related U.S. Application Data
(63) Continuation of application No. PCT/US2022/074255, filed on Jul. 28, 2022.
(60) Provisional application No. 63/227,649, filed on Jul. 30, 2021.

(51) Int. Cl. C07K 16/28; A61K 39/00; A61P 25/28; G01N 33/68

(52) U.S. Cl. CPC C07K 16/2896; A61P 25/28; G01N 33/6854; A61K 2039/505; C07K 2317/24; C07K 2317/31; C07K 2317/56; C07K 2317/92

(57) ABSTRACT

The present disclosure provides antibodies and antibody fragments that bind to human SIRPA. In some embodiments, the antibodies are SIRPA agonists. The antibodies and antibody fragments disclosed herein may be used for the treatment and/or diagnosis of diseases, disorders, and conditions associated with reduced SIRPA activity.

### CROSS-REFERENCE TO RELATED APPLICATIONS

This application is a continuation of International Application No. PCT/US2022/074255, filed on Jul. 28, 2022, which claims the benefit of priority to U.S. Provisional Application No. 63/227,649, filed Jul. 30, 2021, the contents of both of which are incorporated herein by reference for all purposes.

### SEQUENCE LISTING

The present application is being filed with a Sequence Listing which has been submitted electronically in XML format and is hereby incorporated by reference in its entirety. The Sequence Listing is provided as a file entitled “01209-0013-00PCT_ST26.xml”, created on Jul. 21, 2022, and is 524,000 bytes in size.

### FIELD

The present disclosure relates to anti-SIRPα antibodies and uses (e.g., therapeutic uses) of such antibodies.

### BACKGROUND

An appropriate immune response triggered upon challenge requires specific signals for immune activation and the return to homeostasis to provide survival advantages to the host. Professional phagocytes, cells such as macrophages, serve a pivotal role to satisfy this broad mandate by interpreting local environmental cues to orchestrate pro-inflammatory responses or resolve inflammation. However, despite numerous homeostatic control mechanisms, excessive, misdirected, or chronic immune activation form the basis of inflammatory disorders, often with an autoimmune etiology. A need exists for the development of anti-inflammatory therapies that can attenuate these chronic inflammatory conditions with the goal of restoring normal tissue homeostasis.

Phagocyte activity is modulated by the expression of inhibitory receptors, principal among these is signal regulatory protein α (SIRPA). This receptor belongs to the SIRP family of transmembrane receptors, which are primarily expressed within the myeloid cell lineage (including MΦ, DC, granulocytes, etc.) and are characterized by an extracellular region containing 2 membrane-proximal IgC domains and a distal IgV domain. Unique among this family, SIRPA contains an intracellular, cytoplasmic immunoreceptor tyrosine-based inhibitory motif (ITIM). Upon receptor cross-linking, tyrosine-phosphorylated ITIM sites recruit and activate Src homology region 2 domain-containing phosphatases-1/2 (SHP-1/2) to negatively regulate cellular functions, such as phagocytosis or inflammatory cytokine release. CD47 serves as the principal ligand for SIRPA, and its broad expression in most cell types, including endothelial/epithelial cells, leukocytes, and erythrocytes, suggests that it mediates a “don't-eat-me” signal to protect healthy cells from phagocyte-dependent clearance. In support of this view, several studies show that adoptive transfer of red blood cells or leukocytes from CD47-knockout mice into wild-type recipients results in rapid clearance of CD47-deficient cells. Conversely, positional genetic analysis of multiple strains of immune-compromised mice receiving human hematopoietic cells identified the SIRPA allele in NOD mice as the causal factor for successful engraftment in xenotransplantation models. Subsequent studies demonstrated that the allelic variant of SIRPA expressed in NOD mice retained the ability to bind human CD47 expressed on human hematopoietic stem cells, and thus, suppress macrophage-dependent graft rejection.

Regulated expression of SIRPA and CD47 establishes a homeostatic control mechanism to modulate phagocytic cell activity. For example, apoptotic cells downregulate expression of CD47 to facilitate engulfment by resident macrophages while viable cells remain unharmed. Likewise, inflammatory stimuli, such as LPS, decrease SIRPA expression in MP and DC to potentiate their activation during inflammation. However, dysregulation of SIRPA or CD47 expression contributes to inflammatory disorders. For example, hemophagocytic lymphohistiocytosis (HLH) is a syndrome with excessive immune activation characterized by deregulated engulfment of hematopoietic stem cells (HSC) by bone marrow macrophages. Consequently, more than 80% of HLH patients present with cytopenia. Cellular analysis revealed that CD47 is significantly downregulated in the CD34+CD38− HSC fraction from HLH patients during disease relapse rendering these cells prone to macrophage-mediated clearance. In another example, transcriptomic and proteomic profiles from pathological multiple sclerosis (MS) brain lesions obtained from deceased patients revealed reduced expression of CD47 at the mRNA and protein level. A study investigating the role of CD47 in the pathology of a mouse model of MS observed that administering an anti-CD47 blocking antibody at the peak of paralysis exacerbated neuroinflammation and disease severity suggesting that CD47 serves to modulate inflammation through SIRPA.

Apart from human patient data establishing a correlation between CD47 expression levels and inflammation, animal models of autoimmunity suggest that drug interventions that stimulate the SIRPA pathway ameliorate disease. For example, in trinitrobenzene sulfonic acid (TNBS)-induced colitis, which is a mouse model of intestinal inflammation driven by pro-inflammatory SIRPA+CD103− dendritic cells, administration of recombinant CD47-Fc fusion protein prior to TNBS injections protects from colonic inflammation as determined by body weight, intestinal tissue damage, and serum cytokine levels. Similar observations have been reported in collagen antibody-induced arthritis, a mouse model of rheumatoid arthritis, in which prophylactic treatment with CD47-Fc reduced disease incidence and total disease scores. Thus, the SIRPA-CD47 axis may serve as the basis for the development of novel therapeutic strategies to alleviate inflammatory disorders.

Microglial SIRPA is downregulated in Alzheimer's disease pathology and has been shown to play a key role in regulating synaptic remodeling in the central nervous system. (Ding et al, 2021, Nature Communications, 12:2030; Lehrman et al, 2018, Neuron, 100:120-134). Such studies demonstrated that CD47-SIRPA signaling prevents excess microglial phagocytosis. In particular, the “don't eat me” signals associated with the SIRPA-CD47 axis are required to prevent excess pruning and aberrant microglial engulfment during postnatal development, suggesting that CD47 protects certain synaptic populations from targeting by microglial cells. Additionally, loss of microglial SIRPA results in increased synaptic loss mediated, at least in part, by microglial engulfment, and enhanced cognitive impairment (See, e.g., Ding et al, 2021, Nature Communications, 12:2030).

Anti-SIRPA antibodies have been previously described in, e.g., International Patent Application Publication Nos: WO2018/057669, WO 2018/026600, WO 2017/178653, WO2017/068164, WO2016/063233, WO2016/205042, WO2015/138600, WO2013/0956352, WO2009/091547, WO2009/131453, and WO2009/046541.

Accordingly, there is a need for therapeutic anti-SIRPA therapies to treat diseases, disorders, and conditions associated with reduced SIRPA activity.

All references cited herein, including patent applications and publications, are hereby incorporated by reference in their entirety.

### SUMMARY

The present disclosure relates to antibodies that specifically bind to SIRPA. In some embodiments, the antibodies are SIRPA agonists. The disclosure includes multiple embodiments, including, but not limited to, the following embodiments.

Embodiment 1 is an isolated antibody that specifically binds to human SIRPA, wherein the antibody comprises a heavy chain variable region (VH) and a light chain variable region (VL), wherein the heavy chain variable region (VH) comprises:
a. an HVR-H1 comprising an amino acid sequence chosen from any one of SEQ ID NOs: 102-122, 318-328, and 346-349;
b. an HVR-H2 comprising an amino acid sequence chosen from any one of SEQ ID NOs: 123-146 and 329-345; and
c. an HVR-H3 comprising an amino acid sequence chosen from any one of SEQ ID NOs: 147-201, 350-360, and 371-386.

Embodiment 2 is the antibody of embodiment 1, wherein the light chain variable region (VL) comprises:
a. an HVR-L1 comprising an amino acid sequence chosen from any one of SEQ ID NOs: 7-28;
b. an HVR-L2 comprising an amino acid sequence chosen from any one of SEQ ID NOs: 29-40; and
c. an HVR-L3 comprising an amino acid sequence chosen from any one of SEQ ID NOs: 41-101.

Embodiment 3 is the antibody of embodiment 1 or 2, wherein the antibody comprises a VH that is at least 90%, at least 95%, at least 97%, or at least 99% identical to an amino acid sequence chosen from any one of SEQ ID NOs: 202-256, 361-370, and 387-412.

Embodiment 4 is the antibody of any one of embodiments 1-3, wherein the antibody comprises a VL that is at least 90%, at least 95%, at least 97%, or at least 99% identical to an amino acid sequence chosen from any one of SEQ ID NOs: 257-317, 261, 264, 269, 275, 312, and 413.

Embodiment 5 is the antibody of any one of embodiments 1-4, wherein the VH comprises 1, 2, 3, 4, 5, 6, 7, 8, 9, or 10 amino acid substitutions, insertions, and/or deletions compared to an amino acid sequence chosen from any one of SEQ ID NOs: 202-256, 361-370, and 387-412.

Embodiment 6 is the antibody of any one of embodiments 1-5, wherein the VL comprises 1, 2, 3, 4, 5, 6, 7, 8, 9, or 10 amino acid substitutions, insertions, and/or deletions compared to an amino acid sequence chosen from any one of SEQ ID NOs: 257-317, 261, 264, 269, 275, 312, and 413.

Embodiment 7 is the antibody of any one of embodiments 1-6, wherein the antibody comprises a VH comprising an amino acid sequence chosen from any one of SEQ ID NOs: 202-256, 361-370, and 387-412.

Embodiment 8 is the antibody of any one of embodiments 1-7, wherein the antibody comprises a VL comprising an amino acid sequence chosen from any one of SEQ ID NOs: 257-317, 261, 264, 269, 275, 312, and 413.

Embodiment 9 is an isolated antibody that specifically binds to human SIRPA, wherein the antibody comprises a VH comprising HVR-H1, HVR-H2, and HVR-H3 and a VL comprising HVR-L1, HVR-L2, and HVR-L3 of any one of antibodies SA-1, SA-2, SA-3, SA-4, SA-5, SA-6, SA-7, SA-8, SA-9, SA-10, SA-11, SA-12, SA-13, SA-14, SA-15, SA-16, SA-17, SA-18, SA-19, SA-20, SA-21, SA-22, SA-23, SA-24, SA-25, SA-26, SA-27, SA-28, SA-29, SA-30, SA-31, SA-32, SA-33, SA-34, SA-35, SA-36, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56, SA-57, SA-58, SA-59, SA-60, SA-61, SA-62, SA-5-57, SA-5-58, SA-5-59, SA-5-61, SA-8-62, SA-8-64, SA-8-66, SA-8-67, SA-13-68, SA-13-69, SA-13-71, SA-19-72, SA-19-73, SA-19-74, SA-19-75, SA-19-76, SA-19-77, SA-19-78, SA-19-79, SA-19-80, SA-19-81, SA-19-82, SA-19-83, SA-19-84, SA-56-85, SA-56-86, SA-56-87, SA-56-88, SA-56-89, SA-56-90, SA-56-91, SA-56-92, SA-56-93, SA-56-94, SA-56-95, or SA-56-96 (as shown in Tables 3 and 7).

Embodiment 10 is the isolated antibody of embodiment 9, wherein the antibody comprises a VH and/or a VL at least 90%, at least 95%, at least 97%, or at least 99% identical to those of any one of antibodies: SA-1, SA-2, SA-3, SA-4, SA-5, SA-6, SA-7, SA-8, SA-9, SA-10, SA-11, SA-12, SA-13, SA-14, SA-15, SA-16, SA-17, SA-18, SA-19, SA-20, SA-21, SA-22, SA-23, SA-24, SA-25, SA-26, SA-27, SA-28, SA-29, SA-30, SA-31, SA-32, SA-33, SA-34, SA-35, SA-36, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56, SA-57, SA-58, SA-59, SA-60, SA-61, SA-62, SA-5-57, SA-5-58, SA-5-59, SA-5-61, SA-8-62, SA-8-64, SA-8-66, SA-8-67, SA-13-68, SA-13-69, SA-13-71, SA-19-72, SA-19-73, SA-19-74, SA-19-75, SA-19-76, SA-19-77, SA-19-78, SA-19-79, SA-19-80, SA-19-81, SA-19-82, SA-19-83, SA-19-84, SA-56-85, SA-56-86, SA-56-87, SA-56-88, SA-56-89, SA-56-90, SA-56-91, SA-56-92, SA-56-93, SA-56-94, SA-56-95, or SA-56-96 (as shown in Tables 3 and 7).

Embodiment 11 is the isolated antibody of embodiment 9 or embodiment 10, wherein the antibody comprises the VH and/or the VL of any one of antibodies: SA-1, SA-2, SA-3, SA-4, SA-5, SA-6, SA-7, SA-8, SA-9, SA-10, SA-11, SA-12, SA-13, SA-14, SA-15, SA-16, SA-17, SA-18, SA-19, SA-20, SA-21, SA-22, SA-23, SA-24, SA-25, SA-26, SA-27, SA-28, SA-29, SA-30, SA-31, SA-32, SA-33, SA-34, SA-35, SA-36, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56, SA-57, SA-58, SA-59, SA-60, SA-61, SA-62, SA-5-57, SA-5-58, SA-5-59, SA-5-61, SA-8-62, SA-8-64, SA-8-66, SA-8-67, SA-13-68, SA-13-69, SA-13-71, SA-19-72, SA-19-73, SA-19-74, SA-19-75, SA-19-76, SA-19-77, SA-19-78, SA-19-79, SA-19-80, SA-19-81, SA-19-82, SA-19-83, SA-19-84, SA-56-85, SA-56-86, SA-56-87, SA-56-88, SA-56-89, SA-56-90, SA-56-91, SA-56-92, SA-56-93, SA-56-94, SA-56-95, or SA-56-96 (as shown in Tables 3 and 7).

Embodiment 12 is an isolated antibody that specifically binds to human SIRPA, wherein the antibody comprises:
a. a VH comprising an HVR-H1 comprising the amino acid sequence of SEQ ID NO: 102, an HVR-H2 comprising the amino acid sequence of SEQ ID NO: 123, and an HVR-H3 comprising the amino acid sequence of SEQ ID NO: 147; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 41;
b. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 148; and a VL comprising an HVR-L1 comprising the amino acid sequence of SEQ ID NO: 8, an HVR-L2 comprising the amino acid sequence of SEQ ID NO: 30, and an HVR-L3 comprising the amino acid sequence of SEQ ID NO: 42;
c. a VH comprising an HVR-H1 comprising the amino acid sequence of SEQ ID NO: 104, an HVR-H2 comprising the amino acid sequence of SEQ ID NO: 125, and an HVR-H3 comprising the amino acid sequence of SEQ ID NO: 149; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 43
d. a VH comprising an HVR-H1 comprising the amino acid sequence of SEQ ID NO: 105, an HVR-H2 comprising the amino acid sequence of SEQ ID NO: 125, and an HVR-H3 comprising the amino acid sequence of SEQ ID NO: 150; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: +4;
e. a VH comprising an HVR-H1 comprising the amino acid sequence of SEQ ID NO: 106, an HVR-H2 comprising the amino acid sequence of SEQ ID NO: 125, and an HVR-H3 comprising the amino acid sequence of SEQ ID NO: 151; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
f. a VH comprising an HVR-H1 comprising the amino acid sequence of SEQ ID NO: 107, an HVR-H2 comprising the amino acid sequence of SEQ ID NO: 126, and an HVR-H3 comprising the amino acid sequence of SEQ ID NO: 152; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 46;
g. a VH comprising an HVR-H1 comprising the amino acid sequence of SEQ ID NO: 103, an HVR-H2 comprising the amino acid sequence of SEQ ID NO: 127, and an HVR-H3 comprising the amino acid sequence of SEQ ID NO: 153; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 47;
h. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
i. a VH comprising an HVR-H1 comprising the amino acid sequence of SEQ ID NO: 106, an HVR-H2 comprising the amino acid sequence of SEQ ID NO: 128, and an HVR-H3 comprising the amino acid sequence of SEQ ID NO: 155; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 49;
j. a VH comprising an HVR-H1 comprising the amino acid sequence of SEQ ID NO: 107, an HVR-H2 comprising the amino acid sequence of SEQ ID NO: 126, and an HVR-H3 comprising the amino acid sequence of SEQ ID NO: 156; and a VL comprising an HVR-L1 comprising the amino acid sequence of SEQ ID NO: 13, an HVR-L2 comprising the amino acid sequence of SEQ ID NO: 34, and an HVR-L3 comprising the amino acid sequence of SEQ ID NO: 50;
k. a VH comprising an HVR-H1 comprising the amino acid sequence of SEQ ID NO: 102, an HVR-H2 comprising the amino acid sequence of SEQ ID NO: 123, and an HVR-H3 comprising the amino acid sequence of SEQ ID NO: 157; and a VL comprising an HVR-L1 comprising the amino acid sequence of SEQ ID NO: 13, an HVR-L2 comprising the amino acid sequence of SEQ ID NO: 35, and an HVR-L3 comprising the amino acid sequence of SEQ ID NO: 51;
l. a VH comprising an HVR-H1 comprising the amino acid sequence of SEQ ID NO: 108, an HVR-H2 comprising the amino acid sequence of SEQ ID NO: 129, and an HVR-H3 comprising the amino acid sequence of SEQ ID NO: 158; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 52;
m. a VH comprising an HVR-H1 comprising the amino acid sequence of SEQ ID NO: 106, an HVR-H2 comprising the amino acid sequence of SEQ ID NO: 125, and an HVR-H3 comprising the amino acid sequence of SEQ ID NO: 159; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
n. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 160; and a VL comprising an HVR-L1 comprising the amino acid sequence of SEQ ID NO: 15, an HVR-L2 comprising the amino acid sequence of SEQ ID NO: 33, and an HVR-L3 comprising the amino acid sequence of SEQ ID NO: 54;
o. a VH comprising an HVR-H1 comprising the amino acid sequence of SEQ ID NO: 110, an HVR-H2 comprising the amino acid sequence of SEQ ID NO: 131, and an HVR-H3 comprising the amino acid sequence of SEQ ID NO: 161; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 55;
p. a VH comprising an HVR-H1 comprising the amino acid sequence of SEQ ID NO: 111, an HVR-H2 comprising the amino acid sequence of SEQ ID NO: 132, and an HVR-H3 comprising the amino acid sequence of SEQ ID NO: 162; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 56;
q. a VH comprising an HVR-H1 comprising the amino acid sequence of SEQ ID NO: 110, an HVR-H2 comprising the amino acid sequence of SEQ ID NO: 133, and an HVR-H3 comprising the amino acid sequence of SEQ ID NO: 163; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 57;
r. a VH comprising an HVR-H1 comprising the amino acid sequence of SEQ ID NO: 112, an HVR-H2 comprising the amino acid sequence of SEQ ID NO: 134, and an HVR-H3 comprising the amino acid sequence of SEQ ID NO: 164; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 58;
s. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 165; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
t. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 60;
u. a VH comprising an HVR-H1 comprising the amino acid sequence of SEQ ID NO: 113, an HVR-H2 comprising the amino acid sequence of SEQ ID NO: 135, and an HVR-H3 comprising the amino acid sequence of SEQ ID NO: 166; and a VL comprising an HVR-L1 comprising the amino acid sequence of SEQ ID NO: 8, an HVR-L2 comprising the amino acid sequence of SEQ ID NO: 30, and an HVR-L3 comprising the amino acid sequence of SEQ ID NO: 61;
v. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 167; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 62;
w. a VH comprising an HVR-H1 comprising the amino acid sequence of SEQ ID NO: 114, an HVR-H2 comprising the amino acid sequence of SEQ ID NO: 136, and an HVR-H3 comprising the amino acid sequence of SEQ ID NO: 168; and a VL comprising an HVR-L1 comprising the amino acid sequence of SEQ ID NO: 17, an HVR-L2 comprising the amino acid sequence of SEQ ID NO: 37, and an HVR-L3 comprising the amino acid sequence of SEQ ID NO: 63;
x. a VH comprising an HVR-H1 comprising the amino acid sequence of SEQ ID NO: 103, an HVR-H2 comprising the amino acid sequence of SEQ ID NO: 137, and an HVR-H3 comprising the amino acid sequence of SEQ ID NO: 169; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 64;
y. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 170; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 65;
z. a VH comprising an HVR-H1 comprising the amino acid sequence of SEQ ID NO: 111, an HVR-H2 comprising the amino acid sequence of SEQ ID NO: 132, and an HVR-H3 comprising the amino acid sequence of SEQ ID NO: 171; and a VL comprising an HVR-L1 comprising the amino acid sequence of SEQ ID NO: 18, an HVR-L2 comprising the amino acid sequence of SEQ ID NO: 32, and an HVR-L3 comprising the amino acid sequence of SEQ ID NO: 66;
aa. a VH comprising an HVR-H1 comprising the amino acid sequence of SEQ ID NO: 115, an HVR-H2 comprising the amino acid sequence of SEQ ID NO: 139, and an HVR-H3 comprising the amino acid sequence of SEQ ID NO: 172; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 67;
bb. a VH comprising an HVR-H1 comprising the amino acid sequence of SEQ ID NO: 116, an HVR-H2 comprising the amino acid sequence of SEQ ID NO: 140, and an HVR-H3 comprising the amino acid sequence of SEQ ID NO: 173; and a VL comprising an HVR-L1 comprising the amino acid sequence of SEQ ID NO: 19, an HVR-L2 comprising the amino acid sequence of SEQ ID NO: 38, and an HVR-L3 comprising the amino acid sequence of SEQ ID NO: 68;
cc. a VH comprising an HVR-H1 comprising the amino acid sequence of SEQ ID NO: 116, an HVR-H2 comprising the amino acid sequence of SEQ ID NO: 140, and an HVR-H3 comprising the amino acid sequence of SEQ ID NO: 173; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 69;
dd. a VH comprising an HVR-H1 comprising the amino acid sequence of SEQ ID NO: 103, an HVR-H2 comprising the amino acid sequence of SEQ ID NO: 137, and an HVR-H3 comprising the amino acid sequence of SEQ ID NO: 174; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 70;
ee. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 170; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 71;
ff. a VH comprising an HVR-H1 comprising the amino acid sequence of SEQ ID NO: 117, an HVR-H2 comprising the amino acid sequence of SEQ ID NO: 137, and an HVR-H3 comprising the amino acid sequence of SEQ ID NO: 175; and a VL comprising an HVR-L1 comprising the amino acid sequence of SEQ ID NO: 21, an HVR-L2 comprising the amino acid sequence of SEQ ID NO: 36, and an HVR-L3 comprising the amino acid sequence of SEQ ID NO: 72;
gg. a VH comprising an HVR-H1 comprising the amino acid sequence of SEQ ID NO: 110, an HVR-H2 comprising the amino acid sequence of SEQ ID NO: 133, and an HVR-H3 comprising the amino acid sequence of SEQ ID NO: 176; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 73;
hh. a VH comprising an HVR-H1 comprising the amino acid sequence of SEQ ID NO: 118, an HVR-H2 comprising the amino acid sequence of SEQ ID NO: 141, and an HVR-H3 comprising the amino acid sequence of SEQ ID NO: 177; and a VL comprising an HVR-L1 comprising the amino acid sequence of SEQ ID NO: 13, an HVR-L2 comprising the amino acid sequence of SEQ ID NO: 35, and an HVR-L3 comprising the amino acid sequence of SEQ ID NO: 74;
ii. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 178; and a VL comprising an HVR-L1 comprising the amino acid sequence of SEQ ID NO: 13, an HVR-L2 comprising the amino acid sequence of SEQ ID NO: 39, and an HVR-L3 comprising the amino acid sequence of SEQ ID NO: 75;
jj. a VH comprising an HVR-H1 comprising the amino acid sequence of SEQ ID NO: 113, an HVR-H2 comprising the amino acid sequence of SEQ ID NO: 135, and an HVR-H3 comprising the amino acid sequence of SEQ ID NO: 179; and a VL comprising an HVR-L1 comprising the amino acid sequence of SEQ ID NO: 22, an HVR-L2 comprising the amino acid sequence of SEQ ID NO: 31, and an HVR-L3 comprising the amino acid sequence of SEQ ID NO: 76;
kk. a VH comprising an HVR-H1 comprising the amino acid sequence of SEQ ID NO: 116, an HVR-H2 comprising the amino acid sequence of SEQ ID NO: 139, and an HVR-H3 comprising the amino acid sequence of SEQ ID NO: 180; and a VL comprising an HVR-L1 comprising the amino acid sequence of SEQ ID NO: 23, an HVR-L2 comprising the amino acid sequence of SEQ ID NO: 38, and an HVR-L3 comprising the amino acid sequence of SEQ ID NO: 77;
ll. a VH comprising an HVR-H1 comprising the amino acid sequence of SEQ ID NO: 116, an HVR-H2 comprising the amino acid sequence of SEQ ID NO: 139, and an HVR-H3 comprising the amino acid sequence of SEQ ID NO: 180; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 78;
mm. a VH comprising an HVR-H1 comprising the amino acid sequence of SEQ ID NO: 120, an HVR-H2 comprising the amino acid sequence of SEQ ID NO: 129, and an HVR-H3 comprising the amino acid sequence of SEQ ID NO: 181; and a VL comprising an HVR-L1 comprising the amino acid sequence of SEQ ID NO: 24, an HVR-L2 comprising the amino acid sequence of SEQ ID NO: 33, and an HVR-L3 comprising the amino acid sequence of SEQ ID NO: 79;
nn. a VH comprising an HVR-H1 comprising the amino acid sequence of SEQ ID NO: 119, an HVR-H2 comprising the amino acid sequence of SEQ ID NO: 142, and an HVR-H3 comprising the amino acid sequence of SEQ ID NO: 182; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 80;
oo. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 183; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 81;
pp. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 183; and a VL comprising an HVR-L1 comprising the amino acid sequence of SEQ ID NO: 25, an HVR-L2 comprising the amino acid sequence of SEQ ID NO: 36, and an HVR-L3 comprising the amino acid sequence of SEQ ID NO: 82;
qq. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 184; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 83;
rr. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 185; and a VL comprising an HVR-L1 comprising the amino acid sequence of SEQ ID NO: 26 an HVR-L2 comprising the amino acid sequence of SEQ ID NO: 32, and an HVR-L3 comprising the amino acid sequence of SEQ ID NO: 84;
ss. a VH comprising an HVR-H1 comprising the amino acid sequence of SEQ ID NO: 114, an HVR-H2 comprising the amino acid sequence of SEQ ID NO: 141, and an HVR-H3 comprising the amino acid sequence of SEQ ID NO: 186; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 85;
tt. a VH comprising an HVR-H1 comprising the amino acid sequence of SEQ ID NO: 104, an HVR-H2 comprising the amino acid sequence of SEQ ID NO: 125, and an HVR-H3 comprising the amino acid sequence of SEQ ID NO: 187; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 86;
uu. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 188; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 87;
vv. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 189; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 88;
ww. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 190; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 89;
xx. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 191; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 90;
yy. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 192; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 91;
zz. a VH comprising an HVR-H1 comprising the amino acid sequence of SEQ ID NO: 118, an HVR-H2 comprising the amino acid sequence of SEQ ID NO: 141, and an HVR-H3 comprising the amino acid sequence of SEQ ID NO: 193; and a VL comprising an HVR-L1 comprising the amino acid sequence of SEQ ID NO: 20, an HVR-L2 comprising the amino acid sequence of SEQ ID NO: 40, and an HVR-L3 comprising the amino acid sequence of SEQ ID NO: 92;
aaa. a VH comprising an HVR-H1 comprising the amino acid sequence of SEQ ID NO: 116, an HVR-H2 comprising the amino acid sequence of SEQ ID NO: 145, and an HVR-H3 comprising the amino acid sequence of SEQ ID NO: 194; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 93;
bbb. a VH comprising an HVR-H1 comprising the amino acid sequence of SEQ ID NO: 115, an HVR-H2 comprising the amino acid sequence of SEQ ID NO: 139, and an HVR-H3 comprising the amino acid sequence of SEQ ID NO: 195; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 94;
ccc. a VH comprising an HVR-H1 comprising the amino acid sequence of SEQ ID NO: 112, an HVR-H2 comprising the amino acid sequence of SEQ ID NO: 146, and an HVR-H3 comprising the amino acid sequence of SEQ ID NO: 196; and a VL comprising an HVR-L1 comprising the amino acid sequence of SEQ ID NO: 27, an HVR-L2 comprising the amino acid sequence of SEQ ID NO: 35, and an HVR-L3 comprising the amino acid sequence of SEQ ID NO: 95;
ddd. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
eee. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 198; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 97;
fff. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 199; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 98;
ggg. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 200; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 99;
hhh. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 201; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 100;
iii. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 178; and a VL comprising an HVR-L1 comprising the amino acid sequence of SEQ ID NO: 13, an HVR-L2 comprising the amino acid sequence of SEQ ID NO: 39, and an HVR-L3 comprising the amino acid sequence of SEQ ID NO: 75;
jjj. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 201; and a VL comprising an HVR-L1 comprising the amino acid sequence of SEQ ID NO: 28, an HVR-L2 comprising the amino acid sequence of SEQ ID NO: 33, and an HVR-L3 comprising the amino acid sequence of SEQ ID NO: 101;
kkk. a VH comprising an HVR-H1 comprising the amino acid sequence of SEQ ID NO: 318, an HVR-H2 comprising the amino acid sequence of SEQ ID NO: 329, and an HVR-H3 comprising the amino acid sequence of SEQ ID NO: 350; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
lll. a VH comprising an HVR-H1 comprising the amino acid sequence of SEQ ID NO: 318, an HVR-H2 comprising the amino acid sequence of SEQ ID NO: 330, and an HVR-H3 comprising the amino acid sequence of SEQ ID NO: 351; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
mmm. a VH comprising an HVR-H1 comprising the amino acid sequence of SEQ ID NO: 319, an HVR-H2 comprising the amino acid sequence of SEQ ID NO: 331, and an HVR-H3 comprising the amino acid sequence of SEQ ID NO: 352; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
nnn. a VH comprising an HVR-H1 comprising the amino acid sequence of SEQ ID NO: 318, an HVR-H2 comprising the amino acid sequence of SEQ ID NO: 125, and an HVR-H3 comprising the amino acid sequence of SEQ ID NO: 353; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
ooo. a VH comprising an HVR-H1 comprising the amino acid sequence of SEQ ID NO: 320, an HVR-H2 comprising the amino acid sequence of SEQ ID NO: 332, and an HVR-H3 comprising the amino acid sequence of SEQ ID NO: 371; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
ppp. a VH comprising an HVR-H1 comprising the amino acid sequence of SEQ ID NO: 320, an HVR-H2 comprising the amino acid sequence of SEQ ID NO: 332, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
qqq. a VH comprising an HVR-H1 comprising the amino acid sequence of SEQ ID NO: 103, an HVR-H2 comprising the amino acid sequence of SEQ ID NO: 333, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 45;
rrr. a VH comprising an HVR-H1 comprising the amino acid sequence of SEQ ID NO: 320, an HVR-H2 comprising the amino acid sequence of SEQ ID NO: 334, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
sss. a VH comprising an HVR-H1 comprising the amino acid sequence of SEQ ID NO: 321, an HVR-H2 comprising the amino acid sequence of SEQ ID NO: 125, and an HVR-H3 comprising the amino acid sequence of SEQ ID NO: 372; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
ttt. a VH comprising an HVR-H1 comprising the amino acid sequence of SEQ ID NO: 321, an HVR-H2 comprising the amino acid sequence of SEQ ID NO: 335, and an HVR-H3 comprising the amino acid sequence of SEQ ID NO: 373; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
uuu. a VH comprising an HVR-H1 comprising the amino acid sequence of SEQ ID NO: 322, an HVR-H2 comprising the amino acid sequence of SEQ ID NO: 125, and an HVR-H3 comprising the amino acid sequence of SEQ ID NO: 159; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
vvv. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 354; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
www. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 355; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
xxx. a VH comprising an HVR-H1 comprising the amino acid sequence of SEQ ID NO: 346, an HVR-H2 comprising the amino acid sequence of SEQ ID NO: 336, and an HVR-H3 comprising the amino acid sequence of SEQ ID NO: 356; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
yyy. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 357; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
zzz. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 374; and a VL comprising an HVR-L1 comprising the amino acid sequence of SEQ ID NO: 33, an HVR-L2 comprising the amino acid sequence of SEQ ID NO: and an HVR-L3 comprising the amino acid sequence of SEQ ID NO: 59;
aaaa. a VH comprising an HVR-H1 comprising the amino acid sequence of SEQ ID NO: 347, an HVR-H2 comprising the amino acid sequence of SEQ ID NO: 338, and an HVR-H3 comprising the amino acid sequence of SEQ ID NO: 165; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
bbbb. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 375; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
cccc. a VH comprising an HVR-H1 comprising the amino acid sequence of SEQ ID NO: 103, an HVR-H2 comprising the amino acid sequence of SEQ ID NO: 339, and an HVR-H3 comprising the amino acid sequence of SEQ ID NO: 376; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
dddd. a VH comprising an HVR-H1 comprising the amino acid sequence of SEQ ID NO: 103, an HVR-H2 comprising the amino acid sequence of SEQ ID NO: 336, and an HVR-H3 comprising the amino acid sequence of SEQ ID NO: 377; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
eeee. a VH comprising an HVR-H1 comprising the amino acid sequence of SEQ ID NO: 348, an HVR-H2 comprising the amino acid sequence of SEQ ID NO: 337, and an HVR-H3 comprising the amino acid sequence of SEQ ID NO: 378; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
ffff. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 379; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
gggg. a VH comprising an HVR-H1 comprising the amino acid sequence of SEQ ID NO: 349, an HVR-H2 comprising the amino acid sequence of SEQ ID NO: 340, and an HVR-H3 comprising the amino acid sequence of SEQ ID NO: 380; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
hhhh. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 381; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
iiii. a VH comprising an HVR-H1 comprising the amino acid sequence of SEQ ID NO: 324, an HVR-H2 comprising the amino acid sequence of SEQ ID NO: 341, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
jjjj. a VH comprising an HVR-H1 comprising the amino acid sequence of SEQ ID NO: 325, an HVR-H2 comprising the amino acid sequence of SEQ ID NO: 342, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
kkkk. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 382; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
llll. a VH comprising an HVR-H1 comprising the amino acid sequence of SEQ ID NO: 325, an HVR-H2 comprising the amino acid sequence of SEQ ID NO: 343, and an HVR-H3 comprising the amino acid sequence of SEQ ID NO: 383; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
mmmm. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
nnnn. a VH comprising an HVR-H1 comprising the amino acid sequence of SEQ ID NO: 326, an HVR-H2 comprising the amino acid sequence of SEQ ID NO: 341, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
oooo. a VH comprising an HVR-H1 comprising the amino acid sequence of SEQ ID NO: 327, an HVR-H2 comprising the amino acid sequence of SEQ ID NO: 341, and an HVR-H3 comprising the amino acid sequence of SEQ ID NO: 384; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
pppp. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 385; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
qqqq. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 386; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
rrrr. a VH comprising an HVR-H1 comprising the amino acid sequence of SEQ ID NO: 325, an HVR-H2 comprising the amino acid sequence of SEQ ID NO: 341 and an HVR-H3 comprising the amino acid sequence of SEQ ID NO: 358; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
ssss. a VH comprising an HVR-H1 comprising the amino acid sequence of SEQ ID NO: 327, an HVR-H2 comprising the amino acid sequence of SEQ ID NO: 344, and an HVR-H3 comprising the amino acid sequence of SEQ ID NO: 359; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96; or
tttt. a VH comprising an HVR-H1 comprising the amino acid sequence of SEQ ID NO: 328, an HVR-H2 comprising the amino acid sequence of SEQ ID NO: 345, and an HVR-H3 comprising the amino acid sequence of SEQ ID NO: 360; and a VL comprising an HVR-L1 comprising the amino acid sequence of SEQ ID NO:12, an HVR-L2 comprising the amino acid sequence of SEQ ID NO: 32, and an HVR-L3 comprising the amino acid sequence of SEQ ID NO: 96.

Embodiment 13 is the antibody of embodiment 12, wherein the antibody comprises:
a. the HVRs of embodiment 12.a. and further comprises a VH that is at least 90%, at least 95%, at least 97%, or at least 99% identical to the amino acid sequence of SEQ ID NO: 202;
b. the HVRs of embodiment 12.b. and further comprises a VH that is at least 90%, at least 95%, at least 97%, or at least 99% identical to the amino acid sequence of SEQ ID NO: 203;
c. the HVRs of embodiment 12.c. and further comprises a VH that is at least 90%, at least 95%, at least 97%, or at least 99% identical to the amino acid sequence of SEQ ID NO: 204
d. the HVRs of embodiment 12.d. and further comprises a VH that is at least 90%, at least 95%, at least 97%, or at least 99% identical to the amino acid sequence of SEQ ID NO: 205;
e. the HVRs of embodiment 12.e. and further comprises a VH that is at least 90%, at least 95%, at least 97%, or at least 99% identical to the amino acid sequence of SEQ ID NO: 206;
f. the HVRs of embodiment 12.f. and further comprises a VH that is at least 90%, at least 95%, at least 97%, or at least 99% identical to the amino acid sequence of SEQ ID NO: 207;
g. the HVRs of embodiment 12.g. and further comprises a VH that is at least 90%, at least 95%, at least 97%, or at least 99% identical to the amino acid sequence of SEQ ID NO: 208;
h. the HVRs of embodiment 12.h. and further comprises a VH that is at least 90%, at least 95%, at least 97%, or at least 99% identical to the amino acid sequence of SEQ ID NO: 209;
i. the HVRs of embodiment 12.i. and further comprises a VH that is at least 90%, at least 95%, at least 97%, or at least 99% identical to the amino acid sequence of SEQ ID NO: 210;
j. the HVRs of embodiment 12.j. and further comprises a VH that is at least 90%, at least 95%, at least 97%, or at least 99% identical to the amino acid sequence of SEQ ID NO: 211;
k. the HVRs of embodiment 12.k. and further comprises a VH that is at least 90%, at least 95%, at least 97%, or at least 99% identical to the amino acid sequence of SEQ ID NO: 212;
l. the HVRs of embodiment 12.1. and further comprises a VH that is at least 90%, at least 95%, at least 97%, or at least 99% identical to the amino acid sequence of SEQ ID NO: 213;
m. the HVRs of embodiment 12.m. and further comprises a VH that is at least 90%, at least 95%, at least 97%, or at least 99% identical to the amino acid sequence of SEQ ID NO: 214;
n. the HVRs of embodiment 12.n. and further comprises a VH that is at least 90%, at least 95%, at least 97%, or at least 99% identical to the amino acid sequence of SEQ ID NO: 215;
o. the HVRs of embodiment 12.0. and further comprises a VH that is at least 90%, at least 95%, at least 97%, or at least 99% identical to the amino acid sequence of SEQ ID NO: 216;
p. the HVRs of embodiment 12.p. and further comprises a VH that is at least 90%, at least 95%, at least 97%, or at least 99% identical to the amino acid sequence of SEQ ID NO: 217;
q. the HVRs of embodiment 12.q. and further comprises a VH that is at least 90%, at least 95%, at least 97%, or at least 99% identical to the amino acid sequence of SEQ ID NO: 218;
r. the HVRs of embodiment 12.r. and further comprises a VH that is at least 90%, at least 95%, at least 97%, or at least 99% identical to the amino acid sequence of SEQ ID NO: 219;
s. the HVRs of embodiment 12.s. and further comprises a VH that is at least 90%, at least 95%, at least 97%, or at least 99% identical to the amino acid sequence of SEQ ID NO: 220;
t. the HVRs of embodiment 12.t. and further comprises a VH that is at least 90%, at least 95%, at least 97%, or at least 99% identical to the amino acid sequence of SEQ ID NO: 209;
u. the HVRs of embodiment 12.u. and further comprises a VH that is at least 90%, at least 95%, at least 97%, or at least 99% identical to the amino acid sequence of SEQ ID NO: 221;
v. the HVRs of embodiment 12.v. and further comprises a VH that is at least 90%, at least 95%, at least 97%, or at least 99% identical to the amino acid sequence of SEQ ID NO: 222;
w. the HVRs of embodiment 12.w. and further comprises a VH that is at least 90%, at least 95%, at least 97%, or at least 99% identical to the amino acid sequence of SEQ ID NO: 223;
x. the HVRs of embodiment 12.x. and further comprises a VH that is at least 90%, at least 95%, at least 97%, or at least 99% identical to the amino acid sequence of SEQ ID NO: 224;
y. the HVRs of embodiment 12.y. and further comprises a VH that is at least 90%, at least 95%, at least 97%, or at least 99% identical to the amino acid sequence of SEQ ID NO: 225;
z. the HVRs of embodiment 12.z. and further comprises a VH that is at least 90%, at least 95%, at least 97%, or at least 99% identical to the amino acid sequence of SEQ ID NO: 226;
aa. the HVRs of embodiment 12.aa. and further comprises a VH that is at least 90%, at least 95%, at least 97%, or at least 99% identical to the amino acid sequence of SEQ ID NO: 227;
bb. the HVRs of embodiment 12.bb. and further comprises a VH that is at least 90%, at least 95%, at least 97%, or at least 99% identical to the amino acid sequence of SEQ ID NO: 228;
cc. the HVRs of embodiment 12.cc. and further comprises a VH that is at least 90%, at least 95%, at least 97%, or at least 99% identical to the amino acid sequence of SEQ ID NO: 228;
dd. the HVRs of embodiment 12.dd. and further comprises a VH that is at least 90%, at least 95%, at least 97%, or at least 99% identical to the amino acid sequence of SEQ ID NO: 229;
ee. the HVRs of embodiment 12.ee. and further comprises a VH that is at least 90%, at least 95%, at least 97%, or at least 99% identical to the amino acid sequence of SEQ ID NO: 225;
ff. the HVRs of embodiment 12.ff. and further comprises a VH that is at least 90%, at least 95%, at least 97%, or at least 99% identical to the amino acid sequence of SEQ ID NO: 230;
gg. the HVRs of embodiment 12.gg. and further comprises a VH that is at least 90%, at least 95%, at least 97%, or at least 99% identical to the amino acid sequence of SEQ ID NO: 231;
hh. the HVRs of embodiment 12.hh. and further comprises a VH that is at least 90%, at least 95%, at least 97%, or at least 99% identical to the amino acid sequence of SEQ ID NO: 232;
ii. the HVRs of embodiment 12.ii. and further comprises a VH that is at least 90%, at least 95%, at least 97%, or at least 99% identical to the amino acid sequence of SEQ ID NO: 233;
jj. the HVRs of embodiment 12.jj. and further comprises a VH that is at least 90%, at least 95%, at least 97%, or at least 99% identical to the amino acid sequence of SEQ ID NO: 234;
kk. the HVRs of embodiment 12.kk. and further comprises a VH that is at least 90%, at least 95%, at least 97%, or at least 99% identical to the amino acid sequence of SEQ ID NO: 235;
ll. the HVRs of embodiment 12.ll. and further comprises a VH that is at least 90%, at least 95%, at least 97%, or at least 99% identical to the amino acid sequence of SEQ ID NO: 235;
mm. the HVRs of embodiment 12.mm. and further comprises a VH that is at least 90%, at least 95%, at least 97%, or at least 99% identical to the amino acid sequence of SEQ ID NO: 236;
nn. the HVRs of embodiment 12.nn. and further comprises a VH that is at least 90%, at least 95%, at least 97%, or at least 99% identical to the amino acid sequence of SEQ ID NO: 237;
oo. the HVRs of embodiment 12.oo. and further comprises a VH that is at least 90%, at least 95%, at least 97%, or at least 99% identical to the amino acid sequence of SEQ ID NO: 238;
pp. the HVRs of embodiment 12.pp. and further comprises a VH that is at least 90%, at least 95%, at least 97%, or at least 99% identical to the amino acid sequence of SEQ ID NO: 238;
qq. the HVRs of embodiment 12.qq. and further comprises a VH that is at least 90%, at least 95%, at least 97%, or at least 99% identical to the amino acid sequence of SEQ ID NO: 239;
rr. the HVRs of embodiment 12.rr. and further comprises a VH that is at least 90%, at least 95%, at least 97%, or at least 99% identical to the amino acid sequence of SEQ ID NO: 240;
ss. the HVRs of embodiment 12.ss. and further comprises a VH that is at least 90%, at least 95%, at least 97%, or at least 99% identical to the amino acid sequence of SEQ ID NO: 241;
tt. the HVRs of embodiment 12.tt. and further comprises a VH that is at least 90%, at least 95%, at least 97%, or at least 99% identical to the amino acid sequence of SEQ ID NO: 242;
uu. the HVRs of embodiment 12.uu. and further comprises a VH that is at least 90%, at least 95%, at least 97%, or at least 99% identical to the amino acid sequence of SEQ ID NO: 243;
vv. the HVRs of embodiment 12.vv. and further comprises a VH that is at least 90%, at least 95%, at least 97%, or at least 99% identical to the amino acid sequence of SEQ ID NO: 244;
ww. the HVRs of embodiment 12.ww. and further comprises a VH that is at least 90%, at least 95%, at least 97%, or at least 99% identical to the amino acid sequence of SEQ ID NO: 245;
xx. the HVRs of embodiment 12.xx. and further comprises a VH that is at least 90%, at least 95%, at least 97%, or at least 99% identical to the amino acid sequence of SEQ ID NO: 246;
yy. the HVRs of embodiment 12.yy. and further comprises a VH that is at least 90%, at least 95%, at least 97%, or at least 99% identical to the amino acid sequence of SEQ ID NO: 247;
zz. the HVRs of embodiment 12.zz. and further comprises a VH that is at least 90%, at least 95%, at least 97%, or at least 99% identical to the amino acid sequence of SEQ ID NO: 248;
aaa. the HVRs of embodiment 12.aaa. and further comprises a VH that is at least 90%, at least 95%, at least 97%, or at least 99% identical to the amino acid sequence of SEQ ID NO: 249;
bbb. the HVRs of embodiment 12.bbb. and further comprises a VH that is at least 90%, at least 95%, at least 97%, or at least 99% identical to the amino acid sequence of SEQ ID NO: 250;
ccc. the HVRs of embodiment 12.ccc. and further comprises a VH that is at least 90%, at least 95%, at least 97%, or at least 99% identical to the amino acid sequence of SEQ ID NO: 251;
ddd. the HVRs of embodiment 12.ddd. and further comprises a VH that is at least 90%, at least 95%, at least 97%, or at least 99% identical to the amino acid sequence of SEQ ID NO: 252;
eee. the HVRs of embodiment 12.eee. and further comprises a VH that is at least 90%, at least 95%, at least 97%, or at least 99% identical to the amino acid sequence of SEQ ID NO: 253;
fff. the HVRs of embodiment 12.fff. and further comprises a VH that is at least 90%, at least 95%, at least 97%, or at least 99% identical to the amino acid sequence of SEQ ID NO: 254;
ggg. the HVRs of embodiment 12.ggg. and further comprises a VH that is at least 90%, at least 95%, at least 97%, or at least 99% identical to the amino acid sequence of SEQ ID NO: 255;
hhh. the HVRs of embodiment 12.hhh. and further comprises a VH that is at least 90%, at least 95%, at least 97%, or at least 99% identical to the amino acid sequence of SEQ ID NO: 256;
iii. the HVRs of embodiment 12.iii. and further comprises a VH that is at least 90%, at least 95%, at least 97%, or at least 99% identical to the amino acid sequence of SEQ ID NO: 233;
jjj. the HVRs of embodiment 12.jjj. and further comprises a VH that is at least 90%, at least 95%, at least 97%, or at least 99% identical to the amino acid sequence of SEQ ID NO: 256;
kkk. the HVRs of embodiment 12.kkk. and further comprises a VH that is at least 90%, at least 95%, at least 97%, or at least 99% identical to the amino acid sequence of SEQ ID NO: 361;
lll. the HVRs of embodiment 12.lll. and further comprises a VH that is at least 90%, at least 95%, at least 97%, or at least 99% identical to the amino acid sequence of SEQ ID NO: 362;
mmm. the HVRs of embodiment 12.mmm. and further comprises a VH that is at least 90%, at least 95%, at least 97%, or at least 99% identical to the amino acid sequence of SEQ ID NO: 363;
nnn. the HVRs of embodiment 12.nnn. and further comprises a VH that is at least 90%, at least 95%, at least 97%, or at least 99% identical to the amino acid sequence of SEQ ID NO: 364;
ooo. the HVRs of embodiment 12.ooo. and further comprises a VH that is at least 90%, at least 95%, at least 97%, or at least 99% identical to the amino acid sequence of SEQ ID NO: 365;
ppp. the HVRs of embodiment 12.ppp. and further comprises a VH that is at least 90%, at least 95%, at least 97%, or at least 99% identical to the amino acid sequence of SEQ ID NO: 366;
qqq. the HVRs of embodiment 12.qqq. and further comprises a VH that is at least 90%, at least 95%, at least 97%, or at least 99% identical to the amino acid sequence of SEQ ID NO: 367;
rrr. the HVRs of embodiment 12.rrr. and further comprises a VH that is at least 90%, at least 95%, at least 97%, or at least 99% identical to the amino acid sequence of SEQ ID NO: 368;
sss. the HVRs of embodiment 12.sss. and further comprises a VH that is at least 90%, at least 95%, at least 97%, or at least 99% identical to the amino acid sequence of SEQ ID NO: 369;
ttt. the HVRs of embodiment 12.ttt. and further comprises a VH that is at least 90%, at least 95%, at least 97%, or at least 99% identical to the amino acid sequence of SEQ ID NO: 370;
uuu. the HVRs of embodiment 12.uuu. and further comprises a VH that is at least 90%, at least 95%, at least 97%, or at least 99% identical to the amino acid sequence of SEQ ID NO: 387;
vvv. the HVRs of embodiment 12.vvv. and further comprises a VH that is at least 90%, at least 95%, at least 97%, or at least 99% identical to the amino acid sequence of SEQ ID NO: 388;
www. the HVRs of embodiment 12.www. and further comprises a VH that is at least 90%, at least 95%, at least 97%, or at least 99% identical to the amino acid sequence of SEQ ID NO: 389;
xxx. the HVRs of embodiment 12.xxx. and further comprises a VH that is at least 90%, at least 95%, at least 97%, or at least 99% identical to the amino acid sequence of SEQ ID NO: 390;
yyy. the HVRs of embodiment 12.yyy. and further comprises a VH that is at least 90%, at least 95%, at least 97%, or at least 99% identical to the amino acid sequence of SEQ ID NO: 391;
zzz. the HVRs of embodiment 12.zzz. and further comprises a VH that is at least 90%, at least 95%, at least 97%, or at least 99% identical to the amino acid sequence of SEQ ID NO: 392;
aaaa. the HVRs of embodiment 12.aaaa. and further comprises a VH that is at least 90%, at least 95%, at least 97%, or at least 99% identical to the amino acid sequence of SEQ ID NO: 393;
bbbb. the HVRs of embodiment 12.bbbb. and further comprises a VH that is at least 90%, at least 95%, at least 97%, or at least 99% identical to the amino acid sequence of SEQ ID NO: 394;
cccc. the HVRs of embodiment 12.cccc. and further comprises a VH that is at least 90%, at least 95%, at least 97%, or at least 99% identical to the amino acid sequence of SEQ ID NO: 395;
dddd. the HVRs of embodiment 12.dddd. and further comprises a VH that is at least 90%, at least 95%, at least 97%, or at least 99% identical to the amino acid sequence of SEQ ID NO: 396;
eeee. the HVRs of embodiment 12.eeee. and further comprises a VH that is at least 90%, at least 95%, at least 97%, or at least 99% identical to the amino acid sequence of SEQ ID NO: 398;
ffff. the HVRs of embodiment 12.ffff. and further comprises a VH that is at least 90%, at least 95%, at least 97%, or at least 99% identical to the amino acid sequence of SEQ ID NO: 399;
gggg. the HVRs of embodiment 12.gggg. and further comprises a VH that is at least 90%, at least 95%, at least 97%, or at least 99% identical to the amino acid sequence of SEQ ID NO: 400;
hhhh. the HVRs of embodiment 9.hhhh. and further comprises a VH that is at least 90%, at least 95%, at least 97%, or at least 99% identical to the amino acid sequence of SEQ ID NO: 401;
iiii. the HVRs of embodiment 12.iiii. and further comprises a VH that is at least 90%, at least 95%, at least 97%, or at least 99% identical to the amino acid sequence of SEQ ID NO: 402;
jjjj. the HVRs of embodiment 12.jjjj. and further comprises a VH that is at least 90%, at least 95%, at least 97%, or at least 99% identical to the amino acid sequence of SEQ ID NO: 403;
kkkk. the HVRs of embodiment 12.kkkk. and further comprises a VH that is at least 90%, at least 95%, at least 97%, or at least 99% identical to the amino acid sequence of SEQ ID NO: 404;
llll. the HVRs of embodiment 12.llll. and further comprises a VH that is at least 90%, at least 95%, at least 97%, or at least 99% identical to the amino acid sequence of SEQ ID NO: 405;
mmmm. the HVRs of embodiment 12.mmmm. and further comprises a VH that is at least 90%, at least 95%, at least 97%, or at least 99% identical to the amino acid sequence of SEQ ID NO: 406;
nnnn. the HVRs of embodiment 12.nnnn. and further comprises a VH that is at least 90%, at least 95%, at least 97%, or at least 99% identical to the amino acid sequence of SEQ ID NO: 407;
oooo. the HVRs of embodiment 12.oooo. and further comprises a VH that is at least 90%, at least 95%, at least 97%, or at least 99% identical to the amino acid sequence of SEQ ID NO: 408;
pppp. the HVRs of embodiment 12.pppp. and further comprises a VH that is at least 90%, at least 95%, at least 97%, or at least 99% identical to the amino acid sequence of SEQ ID NO: 409;
qqqq. the HVRs of embodiment 12.qqqq. and further comprises a VH that is at least 90%, at least 95%, at least 97%, or at least 99% identical to the amino acid sequence of SEQ ID NO: 410;
rrrr. the HVRs of embodiment 12.rrrr. and further comprises a VH that is at least 90%, at least 95%, at least 97%, or at least 99% identical to the amino acid sequence of SEQ ID NO: 411;
ssss. the HVRs of embodiment 12.ssss. and further comprises a VH that is at least 90%, at least 95%, at least 97%, or at least 99% identical to the amino acid sequence of SEQ ID NO: 397; or
tttt. the HVRs of embodiment 12.tttt. and further comprises a VH that is at least 90%, at least 95%, at least 97%, or at least 99% identical to the amino acid sequence of SEQ ID NO: 412.

Embodiment 14 is the antibody of embodiment 12 or 13, wherein the antibody comprises:
a. the HVRs of embodiment 12.a. and further comprises a VL that is at least 90%, at least 95%, at least 97%, or at least 99% identical to the amino acid sequence of SEQ ID NO: 257;
b. the HVRs of embodiment 12.b. and further comprises a VL that is at least 90%, at least 95%, at least 97%, or at least 99% identical to the amino acid sequence of SEQ ID NO: 258;
c. the HVRs of embodiment 12.c. and further comprises a VL that is at least 90%, at least 95%, at least 97%, or at least 99% identical to the amino acid sequence of SEQ ID NO: 259;
d. the HVRs of embodiment 12.d. and further comprises a VL that is at least 90%, at least 95%, at least 97%, or at least 99% identical to the amino acid sequence of SEQ ID NO: 260;
e. the HVRs of embodiment 12.e. and further comprises a VL that is at least 90%, at least 95%, at least 97%, or at least 99% identical to the amino acid sequence of SEQ ID NO: 261;
f. the HVRs of embodiment 12.f. and further comprises a VL that is at least 90%, at least 95%, at least 97%, or at least 99% identical to the amino acid sequence of SEQ ID NO: 262;
g. the HVRs of embodiment 12.g. and further comprises a VL that is at least 90%, at least 95%, at least 97%, or at least 99% identical to the amino acid sequence of SEQ ID NO: 263;
h. the HVRs of embodiment 12.h. and further comprises a VL that is at least 90%, at least 95%, at least 97%, or at least 99% identical to the amino acid sequence of SEQ ID NO: 264;
i. the HVRs of embodiment 12.i. and further comprises a VL that is at least 90%, at least 95%, at least 97%, or at least 99% identical to the amino acid sequence of SEQ ID NO: 265;
j. the HVRs of embodiment 12.j. and further comprises a VL that is at least 90%, at least 95%, at least 97%, or at least 99% identical to the amino acid sequence of SEQ ID NO: 266;
k. the HVRs of embodiment 12.k. and further comprises a VL that is at least 90%, at least 95%, at least 97%, or at least 99% identical to the amino acid sequence of SEQ ID NO: 267;
l. the HVRs of embodiment 12.l. and further comprises a VL that is at least 90%, at least 95%, at least 97%, or at least 99% identical to the amino acid sequence of SEQ ID NO: 268;
m. the HVRs of embodiment 12.m. and further comprises a VL that is at least 90%, at least 95%, at least 97%, or at least 99% identical to the amino acid sequence of SEQ ID NO: 269;
n. the HVRs of embodiment 12.n. and further comprises a VL that is at least 90%, at least 95%, at least 97%, or at least 99% identical to the amino acid sequence of SEQ ID NO: 270;
o. the HVRs of embodiment 12.o. and further comprises a VL that is at least 90%, at least 95%, at least 97%, or at least 99% identical to the amino acid sequence of SEQ ID NO: 271;
p. the HVRs of embodiment 12.p. and further comprises a VL that is at least 90%, at least 95%, at least 97%, or at least 99% identical to the amino acid sequence of SEQ ID NO: 272;
q. the HVRs of embodiment 12.q. and further comprises a VL that is at least 90%, at least 95%, at least 97%, or at least 99% identical to the amino acid sequence of SEQ ID NO: 273;
r. the HVRs of embodiment 12.r. and further comprises a VL that is at least 90%, at least 95%, at least 97%, or at least 99% identical to the amino acid sequence of SEQ ID NO: 274;
s. the HVRs of embodiment 12.s. and further comprises a VL that is at least 90%, at least 95%, at least 97%, or at least 99% identical to the amino acid sequence of SEQ ID NO: 275;
t. the HVRs of embodiment 12.t. and further comprises a VL that is at least 90%, at least 95%, at least 97%, or at least 99% identical to the amino acid sequence of SEQ ID NO: 276;
u. the HVRs of embodiment 12.u. and further comprises a VL that is at least 90%, at least 95%, at least 97%, or at least 99% identical to the amino acid sequence of SEQ ID NO: 277;
v. the HVRs of embodiment 12.v. and further comprises a VL that is at least 90%, at least 95%, at least 97%, or at least 99% identical to the amino acid sequence of SEQ ID NO: 278;
w. the HVRs of embodiment 12.w. and further comprises a VL that is at least 90%, at least 95%, at least 97%, or at least 99% identical to the amino acid sequence of SEQ ID NO: 279;
x. the HVRs of embodiment 12.x. and further comprises a VL that is at least 90%, at least 95%, at least 97%, or at least 99% identical to the amino acid sequence of SEQ ID NO: 280;
y. the HVRs of embodiment 12.y. and further comprises a VL that is at least 90%, at least 95%, at least 97%, or at least 99% identical to the amino acid sequence of SEQ ID NO: 281;
z. the HVRs of embodiment 12.z. and further comprises a VL that is at least 90%, at least 95%, at least 97%, or at least 99% identical to the amino acid sequence of SEQ ID NO: 282;
aa. the HVRs of embodiment 12.ss. and further comprises a VL that is at least 90%, at least 95%, at least 97%, or at least 99% identical to the amino acid sequence of SEQ ID NO: 283;
bb. the HVRs of embodiment 12.bb. and further comprises a VL that is at least 90%, at least 95%, at least 97%, or at least 99% identical to the amino acid sequence of SEQ ID NO: 284;
Cc. the HVRs of embodiment 12.cc. and further comprises a VL that is at least 90%, at least 95%, at least 97%, or at least 99% identical to the amino acid sequence of SEQ ID NO: 285;
dd. the HVRs of embodiment 12.dd. and further comprises a VL that is at least 90%, at least 95%, at least 97%, or at least 99% identical to the amino acid sequence of SEQ ID NO: 286;
ee. the HVRs of embodiment 12.ee. and further comprises a VL that is at least 90%, at least 95%, at least 97%, or at least 99% identical to the amino acid sequence of SEQ ID NO: 287;
ff. the HVRs of embodiment 12.ff. and further comprises a VL that is at least 90%, at least 95%, at least 97%, or at least 99% identical to the amino acid sequence of SEQ ID NO: 288;
gg. the HVRs of embodiment 12.gg. and further comprises a VL that is at least 90%, at least 95%, at least 97%, or at least 99% identical to the amino acid sequence of SEQ ID NO: 289;
hh. the HVRs of embodiment 12.hh. and further comprises a VL that is at least 90%, at least 95%, at least 97%, or at least 99% identical to the amino acid sequence of SEQ ID NO: 290;
ii. the HVRs of embodiment 12.ii. and further comprises a VL that is at least 90%, at least 95%, at least 97%, or at least 99% identical to the amino acid sequence of SEQ ID NO: 291;
jj. the HVRs of embodiment 12.jj. and further comprises a VL that is at least 90%, at least 95%, at least 97%, or at least 99% identical to the amino acid sequence of SEQ ID NO: 292;
kk. the HVRs of embodiment 12.kk. and further comprises a VL that is at least 90%, at least 95%, at least 97%, or at least 99% identical to the amino acid sequence of SEQ ID NO: 293;
ll. the HVRs of embodiment 12.ll. and further comprises a VL that is at least 90%, at least 95%, at least 97%, or at least 99% identical to the amino acid sequence of SEQ ID NO: 294;
mm. the HVRs of embodiment 12.mm. and further comprises a VL that is at least 90%, at least 95%, at least 97%, or at least 99% identical to the amino acid sequence of SEQ ID NO: 295;
nn. the HVRs of embodiment 12.nn. and further comprises a VL that is at least 90%, at least 95%, at least 97%, or at least 99% identical to the amino acid sequence of SEQ ID NO: 296;
oo. the HVRs of embodiment 12.oo. and further comprises a VL that is at least 90%, at least 95%, at least 97%, or at least 99% identical to the amino acid sequence of SEQ ID NO: 297;
pp. the HVRs of embodiment 12.pp. and further comprises a VL that is at least 90%, at least 95%, at least 97%, or at least 99% identical to the amino acid sequence of SEQ ID NO: 298;
qq. the HVRs of embodiment 12.qq. and further comprises a VL that is at least 90%, at least 95%, at least 97%, or at least 99% identical to the amino acid sequence of SEQ ID NO: 299;
rr. the HVRs of embodiment 12.rr. and further comprises a VL that is at least 90%, at least 95%, at least 97%, or at least 99% identical to the amino acid sequence of SEQ ID NO: 300;
ss. the HVRs of embodiment 12.ss. and further comprises a VL that is at least 90%, at least 95%, at least 97%, or at least 99% identical to the amino acid sequence of SEQ ID NO: 301;
tt. the HVRs of embodiment 12.tt. and further comprises a VL that is at least 90%, at least 95%, at least 97%, or at least 99% identical to the amino acid sequence of SEQ ID NO: 302;
uu. the HVRs of embodiment 12.uu. and further comprises a VL that is at least 90%, at least 95%, at least 97%, or at least 99% identical to the amino acid sequence of SEQ ID NO: 303;
vv. the HVRs of embodiment 12.vv. and further comprises a VL that is at least 90%, at least 95%, at least 97%, or at least 99% identical to the amino acid sequence of SEQ ID NO: 304;
ww. the HVRs of embodiment 12.ww. and further comprises a VL that is at least 90%, at least 95%, at least 97%, or at least 99% identical to the amino acid sequence of SEQ ID NO: 305;
xx. the HVRs of embodiment 12.xx. and further comprises a VL that is at least 90%, at least 95%, at least 97%, or at least 99% identical to the amino acid sequence of SEQ ID NO: 306;
yy. the HVRs of embodiment 12.yy. and further comprises a VL that is at least 90%, at least 95%, at least 97%, or at least 99% identical to the amino acid sequence of SEQ ID NO: 307;
zz. the HVRs of embodiment 12.zz. and further comprises a VL that is at least 90%, at least 95%, at least 97%, or at least 99% identical to the amino acid sequence of SEQ ID NO: 308;
aaa. the HVRs of embodiment 12.aaa. and further comprises a VL that is at least 90%, at least 95%, at least 97%, or at least 99% identical to the amino acid sequence of SEQ ID NO: 309;
bbb. the HVRs of embodiment 12.bbb. and further comprises a VL that is at least 90%, at least 95%, at least 97%, or at least 99% identical to the amino acid sequence of SEQ ID NO: 310;
ccc. the HVRs of embodiment 12.ccc. and further comprises a VL that is at least 90%, at least 95%, at least 97%, or at least 99% identical to the amino acid sequence of SEQ ID NO: 311;
ddd. the HVRs of embodiment 12.ddd. and further comprises a VL that is at least 90%, at least 95%, at least 97%, or at least 99% identical to the amino acid sequence of SEQ ID NO: 312;
eee. the HVRs of embodiment 12.eee. and further comprises a VL that is at least 90%, at least 95%, at least 97%, or at least 99% identical to the amino acid sequence of SEQ ID NO: 313;
fff. the HVRs of embodiment 12.fff. and further comprises a VL that is at least 90%, at least 95%, at least 97%, or at least 99% identical to the amino acid sequence of SEQ ID NO: 314;
ggg. the HVRs of embodiment 12.ggg. and further comprises a VL that is at least 90%, at least 95%, at least 97%, or at least 99% identical to the amino acid sequence of SEQ ID NO: 315;
hhh. the HVRs of embodiment 12.hhh. and further comprises a VL that is at least 90%, at least 95%, at least 97%, or at least 99% identical to the amino acid sequence of SEQ ID NO: 316;
iii. the HVRs of embodiment 12.iii. and further comprises a VL that is at least 90%, at least 95%, at least 97%, or at least 99% identical to the amino acid sequence of SEQ ID NO: 291;
jjj. the HVRs of embodiment 12.jjj. and further comprises a VL that is at least 90%, at least 95%, at least 97%, or at least 99% identical to the amino acid sequence of SEQ ID NO: 317;
kkk. the HVRs of embodiment 12.kkk. and further comprises a VL that is at least 90%, at least 95%, at least 97%, or at least 99% identical to the amino acid sequence of SEQ ID NO: 261;
lll. the HVRs of embodiment 12.lll. and further comprises a VL that is at least 90%, at least 95%, at least 97%, or at least 99% identical to the amino acid sequence of SEQ ID NO: 261;
mmm. the HVRs of embodiment 12.mmm. and further comprises a VL that is at least 90%, at least 95%, at least 97%, or at least 99% identical to the amino acid sequence of SEQ ID NO: 261;
nnn. the HVRs of embodiment 12.nnn. and further comprises a VL that is at least 90%, at least 95%, at least 97%, or at least 99% identical to the amino acid sequence of SEQ ID NO: 261;
ooo. the HVRs of embodiment 12.ooo. and further comprises a VL that is at least 90%, at least 95%, at least 97%, or at least 99% identical to the amino acid sequence of SEQ ID NO: 264;
ppp. the HVRs of embodiment 12.ppp. and further comprises a VL that is at least 90%, at least 95%, at least 97%, or at least 99% identical to the amino acid sequence of SEQ ID NO: 264;
qqq. the HVRs of embodiment 12.qqq. and further comprises a VL that is at least 90%, at least 95%, at least 97%, or at least 99% identical to the amino acid sequence of SEQ ID NO: 264;
rrr. the HVRs of embodiment 12.rrr. and further comprises a VL that is at least 90%, at least 95%, at least 97%, or at least 99% identical to the amino acid sequence of SEQ ID NO: 264;
sss. the HVRs of embodiment 12.sss. and further comprises a VL that is at least 90%, at least 95%, at least 97%, or at least 99% identical to the amino acid sequence of SEQ ID NO: 269;
ttt. the HVRs of embodiment 12.ttt. and further comprises a VL that is at least 90%, at least 95%, at least 97%, or at least 99% identical to the amino acid sequence of SEQ ID NO: 269;
uuu. the HVRs of embodiment 12.uuu. and further comprises a VL that is at least 90%, at least 95%, at least 97%, or at least 99% identical to the amino acid sequence of SEQ ID NO: 269;
vvv. the HVRs of embodiment 12.vvv. and further comprises a VL that is at least 90%, at least 95%, at least 97%, or at least 99% identical to the amino acid sequence of SEQ ID NO: 275;
www. the HVRs of embodiment 12.www. and further comprises a VL that is at least 90%, at least 95%, at least 97%, or at least 99% identical to the amino acid sequence of SEQ ID NO: 275;
xxx. the HVRs of embodiment 12.xxx. and further comprises a VL that is at least 90%, at least 95%, at least 97%, or at least 99% identical to the amino acid sequence of SEQ ID NO: 275;
yyy. the HVRs of embodiment 12.yyy. and further comprises a VL that is at least 90%, at least 95%, at least 97%, or at least 99% identical to the amino acid sequence of SEQ ID NO: 275;
zzz. the HVRs of embodiment 12.zzz. and further comprises a VL that is at least 90%, at least 95%, at least 97%, or at least 99% identical to the amino acid sequence of SEQ ID NO: 275;
aaaa. the HVRs of embodiment 12.aaaa. and further comprises a VL that is at least 90%, at least 95%, at least 97%, or at least 99% identical to the amino acid sequence of SEQ ID NO: 275;
bbbb. the HVRs of embodiment 12.bbbb. and further comprises a VL that is at least 90%, at least 95%, at least 97%, or at least 99% identical to the amino acid sequence of SEQ ID NO: 275;
cccc. the HVRs of embodiment 12.cccc. and further comprises a VL that is at least 90%, at least 95%, at least 97%, or at least 99% identical to the amino acid sequence of SEQ ID NO: 275;
dddd. the HVRs of embodiment 12.dddd. and further comprises a VL that is at least 90%, at least 95%, at least 97%, or at least 99% identical to the amino acid sequence of SEQ ID NO: 275;
eeee. the HVRs of embodiment 12.eeee. and further comprises a VL that is at least 90%, at least 95%, at least 97%, or at least 99% identical to the amino acid sequence of SEQ ID NO: 264;
ffff. the HVRs of embodiment 12.ffff. and further comprises a VL that is at least 90%, at least 95%, at least 97%, or at least 99% identical to the amino acid sequence of SEQ ID NO: 275;
gggg. the HVRs of embodiment 12.gggg. and further comprises a VL that is at least 90%, at least 95%, at least 97%, or at least 99% identical to the amino acid sequence of SEQ ID NO: 275;
hhhh. the HVRs of embodiment 12.hhhh. and further comprises a VL that is at least 90%, at least 95%, at least 97%, or at least 99% identical to the amino acid sequence of SEQ ID NO: 275;
iiii. the HVRs of embodiment 12.iiii. and further comprises a VL that is at least 90%, at least 95%, at least 97%, or at least 99% identical to the amino acid sequence of SEQ ID NO: 416;
jjjj. the HVRs of embodiment 12.jjjj. and further comprises a VL that is at least 90%, at least 95%, at least 97%, or at least 99% identical to the amino acid sequence of SEQ ID NO: 312;
kkkk. the HVRs of embodiment 12.kkkk. and further comprises a VL that is at least 90%, at least 95%, at least 97%, or at least 99% identical to the amino acid sequence of SEQ ID NO: 312;
llll. the HVRs of embodiment 12.llll. and further comprises a VL that is at least 90%, at least 95%, at least 97%, or at least 99% identical to the amino acid sequence of SEQ ID NO: 312;
mmmm. the HVRs of embodiment 12.mmmm. and further comprises a VL that is at least 90%, at least 95%, at least 97%, or at least 99% identical to the amino acid sequence of SEQ ID NO: 312;
nnnn. the HVRs of embodiment 12.nnnn. and further comprises a VL that is at least 90%, at least 95%, at least 97%, or at least 99% identical to the amino acid sequence of SEQ ID NO: 312;
oooo. the HVRs of embodiment 12.oooo. and further comprises a VL that is at least 90%, at least 95%, at least 97%, or at least 99% identical to the amino acid sequence of SEQ ID NO: 312;
pppp. the HVRs of embodiment 12.pppp. and further comprises a VL that is at least 90%, at least 95%, at least 97%, or at least 99% identical to the amino acid sequence of SEQ ID NO: 312;
qqqq. the HVRs of embodiment 12.qqqq. and further comprises a VL that is at least 90%, at least 95%, at least 97%, or at least 99% identical to the amino acid sequence of SEQ ID NO: 312;
rrrr. the HVRs of embodiment 12.rrrr. and further comprises a VL that is at least 90%, at least 95%, at least 97%, or at least 99% identical to the amino acid sequence of SEQ ID NO: 312;
ssss. the HVRs of embodiment 12.ssss. and further comprises a VL that is at least 90%, at least 95%, at least 97%, or at least 99% identical to the amino acid sequence of SEQ ID NO: 312; or
tttt. the HVRs of embodiment 12.tttt. and further comprises a VL that is at least 90%, at least 95%, at least 97%, or at least 99% identical to the amino acid sequence of SEQ ID NO: 312.

Embodiment 15 is the antibody of any one of embodiments 12-14, wherein the antibody comprises:
a. the HVRs of embodiment 12.a. and further comprises a VH comprising the amino acid sequence of SEQ ID NO: 202;
b. the HVRs of embodiment 12.b. and further comprises a VH comprising the amino acid sequence of SEQ ID NO: 203;
c. the HVRs of embodiment 12.c. and further comprises a VH comprising the amino acid sequence of SEQ ID NO: 204;
d. the HVRs of embodiment 12.d. and further comprises a VH comprising the amino acid sequence of SEQ ID NO: 205;
e. the HVRs of embodiment 12.e. and further comprises a VH comprising the amino acid sequence of SEQ ID NO: 206;
f. the HVRs of embodiment 12.f. and further comprises a VH comprising the amino acid sequence of SEQ ID NO: 207;
g. the HVRs of embodiment 12.g. and further comprises a VH comprising the amino acid sequence of SEQ ID NO: 208;
h. the HVRs of embodiment 12.h. and further comprises a VH comprising the amino acid sequence of SEQ ID NO: 209;
i. the HVRs of embodiment 12.i. and further comprises a VH comprising the amino acid sequence of SEQ ID NO: 210;
j. the HVRs of embodiment 12.j. and further comprises a VH comprising the amino acid sequence of SEQ ID NO: 211;
k. the HVRs of embodiment 12.k. and further comprises a VH comprising the amino acid sequence of SEQ ID NO: 212;
l. the HVRs of embodiment 12.l. and further comprises a VH comprising the amino acid sequence of SEQ ID NO: 213;
m. the HVRs of embodiment 12.m. and further comprises a VH comprising the amino acid sequence of SEQ ID NO: 214;
n. the HVRs of embodiment 12.n. and further comprises a VH comprising the amino acid sequence of SEQ ID NO: 215;
o. the HVRs of embodiment 12.o. and further comprises a VH comprising the amino acid sequence of SEQ ID NO: 216;
p. the HVRs of embodiment 12.p. and further comprises a VH comprising the amino acid sequence of SEQ ID NO: 217;
q. the HVRs of embodiment 12.q. and further comprises a VH comprising the amino acid sequence of SEQ ID NO: 218;
r. the HVRs of embodiment 12.r. and further comprises a VH comprising the amino acid sequence of SEQ ID NO: 219;
s. the HVRs of embodiment 12.s. and further comprises a VH comprising the amino acid sequence of SEQ ID NO: 220;
t. the HVRs of embodiment 12.t. and further comprises a VH comprising the amino acid sequence of SEQ ID NO: 209;
u. the HVRs of embodiment 12.u. and further comprises a VH comprising the amino acid sequence of SEQ ID NO: 221;
v. the HVRs of embodiment 12.v. and further comprises a VH comprising the amino acid sequence of SEQ ID NO: 222;
w. the HVRs of embodiment 12.w. and further comprises a VH comprising the amino acid sequence of SEQ ID NO: 223;
x. the HVRs of embodiment 12.x. and further comprises a VH comprising the amino acid sequence of SEQ ID NO: 224;
y. the HVRs of embodiment 12.y. and further comprises a VH comprising the amino acid sequence of SEQ ID NO: 225;
z. the HVRs of embodiment 12.z. and further comprises a VH comprising the amino acid sequence of SEQ ID NO: 226;
aa. the HVRs of embodiment 12.aa. and further comprises a VH comprising the amino acid sequence of SEQ ID NO: 227;
bb. the HVRs of embodiment 12.bb. and further comprises a VH comprising the amino acid sequence of SEQ ID NO: 228;
cc. the HVRs of embodiment 12.cc. and further comprises a VH comprising the amino acid sequence of SEQ ID NO: 228;
dd. the HVRs of embodiment 12.dd. and further comprises a VH comprising the amino acid sequence of SEQ ID NO: 229;
ee. the HVRs of embodiment 12.ee. and further comprises a VH comprising the amino acid sequence of SEQ ID NO: 225;
ff. the HVRs of embodiment 12.ff. and further comprises a VH comprising the amino acid sequence of SEQ ID NO: 230;
gg. the HVRs of embodiment 12.gg. and further comprises a VH comprising the amino acid sequence of SEQ ID NO: 231;
hh. the HVRs of embodiment 12.hh. and further comprises a VH comprising the amino acid sequence of SEQ ID NO: 232;
ii. the HVRs of embodiment 12.ii. and further comprises a VH comprising the amino acid sequence of SEQ ID NO: 233;
jj. the HVRs of embodiment 12.jj. and further comprises a VH comprising the amino acid sequence of SEQ ID NO: 234;
kk. the HVRs of embodiment 12.kk. and further comprises a VH comprising the amino acid sequence of SEQ ID NO: 235;
ll. the HVRs of embodiment 12.ll. and further comprises a VH comprising the amino acid sequence of SEQ ID NO: 235;
mm. the HVRs of embodiment 12.mm. and further comprises a VH comprising the amino acid sequence of SEQ ID NO: 236;
nn. the HVRs of embodiment 12.nn. and further comprises a VH comprising the amino acid sequence of SEQ ID NO: 237;
oo. the HVRs of embodiment 12.oo. and further comprises a VH comprising the amino acid sequence of SEQ ID NO: 238;
pp. the HVRs of embodiment 12.pp. and further comprises a VH comprising the amino acid sequence of SEQ ID NO: 238;
qq. the HVRs of embodiment 12.qq. and further comprises a VH comprising the amino acid sequence of SEQ ID NO: 239;
rr. the HVRs of embodiment 12.rr. and further comprises a VH comprising the amino acid sequence of SEQ ID NO: 240;
ss. the HVRs of embodiment 12.ss. and further comprises a VH comprising the amino acid sequence of SEQ ID NO: 241;
tt. the HVRs of embodiment 12.tt. and further comprises a VH comprising the amino acid sequence of SEQ ID NO: 242;
uu. the HVRs of embodiment 12.uu. and further comprises a VH comprising the amino acid sequence of SEQ ID NO: 243;
vv. the HVRs of embodiment 12.vv. and further comprises a VH comprising the amino acid sequence of SEQ ID NO: 244;
ww. the HVRs of embodiment 12.ww. and further comprises a VH comprising the amino acid sequence of SEQ ID NO: 245;
xx. the HVRs of embodiment 12.xx. and further comprises a VH comprising the amino acid sequence of SEQ ID NO: 246;
yy. the HVRs of embodiment 12.yy. and further comprises a VH comprising the amino acid sequence of SEQ ID NO: 247;
zz. the HVRs of embodiment 12.zz. and further comprises a VH comprising the amino acid sequence of SEQ ID NO: 248;
aaa. the HVRs of embodiment 12.aaa. and further comprises a VH comprising the amino acid sequence of SEQ ID NO: 249;
bbb. the HVRs of embodiment 12.bbb. and further comprises a VH comprising the amino acid sequence of SEQ ID NO: 250;
ccc. the HVRs of embodiment 12.ccc. and further comprises a VH comprising the amino acid sequence of SEQ ID NO: 251;
ddd. the HVRs of embodiment 12.ddd. and further comprises a VH comprising the amino acid sequence of SEQ ID NO: 252;
eee. the HVRs of embodiment 12.eee. and further comprises a VH comprising the amino acid sequence of SEQ ID NO: 253;
fff. the HVRs of embodiment 12.fff. and further comprises a VH comprising the amino acid sequence of SEQ ID NO: 254;
ggg. the HVRs of embodiment 12.ggg. and further comprises a VH comprising the amino acid sequence of SEQ ID NO: 255;
hhh. the HVRs of embodiment 12.hhh. and further comprises a VH comprising the amino acid sequence of SEQ ID NO: 256;
iii. the HVRs of embodiment 12.iii. and further comprises a VH comprising the amino acid sequence of SEQ ID NO: 233;
jjj. the HVRs of embodiment 12.jjj. and further comprises a VH comprising the amino acid sequence of SEQ ID NO: 256;
kkk. the HVRs of embodiment 12.kkk. and further comprises a VH comprising the amino acid sequence of SEQ ID NO: 361;
lll. the HVRs of embodiment 12.lll. and further comprises a VH comprising the amino acid sequence of SEQ ID NO: 362;
mmm. the HVRs of embodiment 12.mmm. and further comprises a VH comprising the amino acid sequence of SEQ ID NO: 363;
nnn. the HVRs of embodiment 12.nnn. and further comprises a VH comprising the amino acid sequence of SEQ ID NO: 364;
ooo. the HVRs of embodiment 12.ooo. and further comprises a VH comprising the amino acid sequence of SEQ ID NO: 365;
ppp. the HVRs of embodiment 12.ppp. and further comprises a VH comprising the amino acid sequence of SEQ ID NO: 366;
qqq. the HVRs of embodiment 12.qqq. and further comprises a VH comprising the amino acid sequence of SEQ ID NO: 367;
rrr. the HVRs of embodiment 12.rrr. and further comprises a VH comprising the amino acid sequence of SEQ ID NO: 368;
sss. the HVRs of embodiment 12.sss. and further comprises a VH comprising the amino acid sequence of SEQ ID NO: 369;
ttt. the HVRs of embodiment 12.ttt. and further comprises a VH comprising the amino acid sequence of SEQ ID NO: 370;
uuu. the HVRs of embodiment 12.uuu. and further comprises a VH comprising the amino acid sequence of SEQ ID NO: 387;
vvv. the HVRs of embodiment 12.vvv. and further comprises a VH comprising the amino acid sequence of SEQ ID NO: 388;
www. the HVRs of embodiment 12.www. and further comprises a VH comprising the amino acid sequence of SEQ ID NO: 389;
xxx. the HVRs of embodiment 12.xxx. and further comprises a VH comprising the amino acid sequence of SEQ ID NO: 390;
yyy. the HVRs of embodiment 12.yyy. and further comprises a VH comprising the amino acid sequence of SEQ ID NO: 391;
zzz. the HVRs of embodiment 12.zzz. and further comprises a VH comprising the amino acid sequence of SEQ ID NO: 392;
aaaa. the HVRs of embodiment 12.aaaa. and further comprises a VH comprising the amino acid sequence of SEQ ID NO: 393;
bbbb. the HVRs of embodiment 12.bbbb. and further comprises a VH comprising the amino acid sequence of SEQ ID NO: 394;
cccc. the HVRs of embodiment 12.cccc. and further comprises a VH comprising the amino acid sequence of SEQ ID NO: 395;
dddd. the HVRs of embodiment 12.dddd. and further comprises a VH comprising the amino acid sequence of SEQ ID NO: 396;
eeee. the HVRs of embodiment 12.eeee. and further comprises a VH comprising the amino acid sequence of SEQ ID NO: 398;
ffff. the HVRs of embodiment 12.ffff. and further comprises a VH comprising the amino acid sequence of SEQ ID NO: 399;
gggg. the HVRs of embodiment 12.gggg. and further comprises a VH comprising the amino acid sequence of SEQ ID NO: 400;
hhhh. the HVRs of embodiment 9.hhhh. and further comprises a VH comprising the amino acid sequence of SEQ ID NO: 401;
iiii. the HVRs of embodiment 12.iiii. and further comprises a VH comprising the amino acid sequence of SEQ ID NO: 402;
jjjj. the HVRs of embodiment 12.jjjj. and further comprises a VH comprising the amino acid sequence of SEQ ID NO: 403;
kkkk. the HVRs of embodiment 12.kkkk. and further comprises a VH comprising the amino acid sequence of SEQ ID NO: 404;
llll. the HVRs of embodiment 12.llll. and further comprises a VH comprising the amino acid sequence of SEQ ID NO: 405;
mmmm. the HVRs of embodiment 12.mmmm. and further comprises a VH comprising the amino acid sequence of SEQ ID NO: 406;
nnnn. the HVRs of embodiment 12.nnnn. and further comprises a VH comprising the amino acid sequence of SEQ ID NO: 407;
oooo. the HVRs of embodiment 12.oooo. and further comprises a VH comprising the amino acid sequence of SEQ ID NO: 408;
pppp. the HVRs of embodiment 12.pppp. and further comprises a VH comprising the amino acid sequence of SEQ ID NO: 409;
qqqq. the HVRs of embodiment 12.qqqq. and further comprises a VH comprising the amino acid sequence of SEQ ID NO: 410;
rrrr. the HVRs of embodiment 12.rrrr. and further comprises a VH comprising the amino acid sequence of SEQ ID NO: 411;
ssss. the HVRs of embodiment 12.ssss. and further comprises a VH comprising the amino acid sequence of SEQ ID NO: 397; or
tttt. the HVRs of embodiment 12.tttt. and further comprises a VH comprising the amino acid sequence of SEQ ID NO: 412.

Embodiment 16 is the antibody of any one of embodiments 12-15, wherein the antibody comprises:
a. the HVRs of embodiment 12.a. and further comprises a VL comprising the amino acid sequence of SEQ ID NO: 257;
b. the HVRs of embodiment 12.b. and further comprises a VL comprising the amino acid sequence of SEQ ID NO: 258;
c. the HVRs of embodiment 12.c. and further comprises a VL comprising the amino acid sequence of SEQ ID NO: 259
d. the HVRs of embodiment 12.d. and further comprises a VL comprising the amino acid sequence of SEQ ID NO: 260;
e. the HVRs of embodiment 12.e. and further comprises a VL comprising the amino acid sequence of SEQ ID NO: 261;
f. the HVRs of embodiment 12.f. and further comprises a VL comprising the amino acid sequence of SEQ ID NO: 262;
g. the HVRs of embodiment 12.g. and further comprises a VL comprising the amino acid sequence of SEQ ID NO: 263;
h. the HVRs of embodiment 12.h. and further comprises a VL comprising the amino acid sequence of SEQ ID NO: 264;
i. the HVRs of embodiment 12.i. and further comprises a VL comprising the amino acid sequence of SEQ ID NO: 265;
j. the HVRs of embodiment 12.j. and further comprises a VL comprising the amino acid sequence of SEQ ID NO: 266;
k. the HVRs of embodiment 12.k. and further comprises a VL comprising the amino acid sequence of SEQ ID NO: 267;
l. the HVRs of embodiment 12.l. and further comprises a VL comprising the amino acid sequence of SEQ ID NO: 268;
m. the HVRs of embodiment 12.m. and further comprises a VL comprising the amino acid sequence of SEQ ID NO: 269;
n. the HVRs of embodiment 12.n. and further comprises a VL comprising the amino acid sequence of SEQ ID NO: 270;
o. the HVRs of embodiment 12.o. and further comprises a VL comprising the amino acid sequence of SEQ ID NO: 271;
p. the HVRs of embodiment 12.p. and further comprises a VL comprising the amino acid sequence of SEQ ID NO: 272;
q. the HVRs of embodiment 12.q. and further comprises a VL comprising the amino acid sequence of SEQ ID NO: 273;
r. the HVRs of embodiment 12.r. and further comprises a VL comprising the amino acid sequence of SEQ ID NO: 274;
s. the HVRs of embodiment 12.s. and further comprises a VL comprising the amino acid sequence of SEQ ID NO: 275;
t. the HVRs of embodiment 12.t. and further comprises a VL comprising the amino acid sequence of SEQ ID NO: 276;
u. the HVRs of embodiment 12.u. and further comprises a VL comprising the amino acid sequence of SEQ ID NO: 277;
v. the HVRs of embodiment 12.v. and further comprises a VL comprising the amino acid sequence of SEQ ID NO: 278;
w. the HVRs of embodiment 12.w. and further comprises a VL comprising the amino acid sequence of SEQ ID NO: 279;
x. the HVRs of embodiment 12.x. and further comprises a VL comprising the amino acid sequence of SEQ ID NO: 280;
y. the HVRs of embodiment 12.y. and further comprises a VL comprising the amino acid sequence of SEQ ID NO: 281;
z. the HVRs of embodiment 12.z. and further comprises a VL comprising the amino acid sequence of SEQ ID NO: 282;
aa. the HVRs of embodiment 12.ss. and further comprises a VL comprising the amino acid sequence of SEQ ID NO: 283;
bb. the HVRs of embodiment 12.bb. and further comprises a VL comprising the amino acid sequence of SEQ ID NO: 284;
cc. the HVRs of embodiment 12.cc. and further comprises a VL comprising the amino acid sequence of SEQ ID NO: 285;
dd. the HVRs of embodiment 12.dd. and further comprises a VL comprising the amino acid sequence of SEQ ID NO: 286;
ee. the HVRs of embodiment 12.ee. and further comprises a VL comprising the amino acid sequence of SEQ ID NO: 287;
ff. the HVRs of embodiment 12.ff. and further comprises a VL comprising the amino acid sequence of SEQ ID NO: 288;
gg. the HVRs of embodiment 12.gg. and further comprises a VL comprising the amino acid sequence of SEQ ID NO: 289;
hh. the HVRs of embodiment 12.hh. and further comprises a VL comprising the amino acid sequence of SEQ ID NO: 290;
ii. the HVRs of embodiment 12.ii. and further comprises a VL comprising the amino acid sequence of SEQ ID NO: 291;
jj. the HVRs of embodiment 12.jj. and further comprises a VL comprising the amino acid sequence of SEQ ID NO: 292;
kk. the HVRs of embodiment 12.kk. and further comprises a VL comprising the amino acid sequence of SEQ ID NO: 293;
ll. the HVRs of embodiment 12.ll. and further comprises a VL comprising the amino acid sequence of SEQ ID NO: 294;
mm. the HVRs of embodiment 12.mm. and further comprises a VL comprising the amino acid sequence of SEQ ID NO: 295;
nn. the HVRs of embodiment 12.nn. and further comprises a VL comprising the amino acid sequence of SEQ ID NO: 296;
oo. the HVRs of embodiment 12.oo. and further comprises a VL comprising the amino acid sequence of SEQ ID NO: 297;
pp. the HVRs of embodiment 12.pp. and further comprises a VL comprising the amino acid sequence of SEQ ID NO: 298;
qq. the HVRs of embodiment 12.qq. and further comprises a VL comprising the amino acid sequence of SEQ ID NO: 299;
rr. the HVRs of embodiment 12.rr. and further comprises a VL comprising the amino acid sequence of SEQ ID NO: 300;
ss. the HVRs of embodiment 12.ss. and further comprises a VL comprising the amino acid sequence of SEQ ID NO: 301;
tt. the HVRs of embodiment 12.tt. and further comprises a VL comprising the amino acid sequence of SEQ ID NO: 302;
uu. the HVRs of embodiment 12.uu. and further comprises a VL comprising the amino acid sequence of SEQ ID NO: 303;
vv. the HVRs of embodiment 12.vv. and further comprises a VL comprising the amino acid sequence of SEQ ID NO: 304;
ww. the HVRs of embodiment 12.ww. and further comprises a VL comprising the amino acid sequence of SEQ ID NO: 305;
xx. the HVRs of embodiment 12.xx. and further comprises a VL comprising the amino acid sequence of SEQ ID NO: 306;
yy. the HVRs of embodiment 12.yy. and further comprises a VL comprising the amino acid sequence of SEQ ID NO: 307;
zz. the HVRs of embodiment 12.zz. and further comprises a VL comprising the amino acid sequence of SEQ ID NO: 308;
aaa. the HVRs of embodiment 12.aaa. and further comprises a VL comprising the amino acid sequence of SEQ ID NO: 309;
bbb. the HVRs of embodiment 12.bbb. and further comprises a VL comprising the amino acid sequence of SEQ ID NO: 310;
ccc. the HVRs of embodiment 12.ccc. and further comprises a VL comprising the amino acid sequence of SEQ ID NO: 311;
ddd. the HVRs of embodiment 12.ddd. and further comprises a VL comprising the amino acid sequence of SEQ ID NO: 312;
eee. the HVRs of embodiment 12.eee. and further comprises a VL comprising the amino acid sequence of SEQ ID NO: 313;
fff. the HVRs of embodiment 12.fff. and further comprises a VL comprising the amino acid sequence of SEQ ID NO: 314;
ggg. the HVRs of embodiment 12.ggg. and further comprises a VL comprising the amino acid sequence of SEQ ID NO: 315;
hhh. the HVRs of embodiment 12.hhh. and further comprises a VL comprising the amino acid sequence of SEQ ID NO: 316;
iii. the HVRs of embodiment 12.iii. and further comprises a VL comprising the amino acid sequence of SEQ ID NO: 291;
jjj. the HVRs of embodiment 12.jjj. and further comprises a VL comprising the amino acid sequence of SEQ ID NO: 317;
kkk. the HVRs of embodiment 12.kkk. and further comprises a VL comprising the amino acid sequence of SEQ ID NO: 261;
lll. the HVRs of embodiment 12.lll. and further comprises a VL comprising the amino acid sequence of SEQ ID NO: 261;
mmm. the HVRs of embodiment 12.mmm. and further comprises a VL comprising the amino acid sequence of SEQ ID NO: 261;
nnn. the HVRs of embodiment 12.nnn. and further comprises a VL comprising the amino acid sequence of SEQ ID NO: 261;
ooo. the HVRs of embodiment 12.ooo. and further comprises a VL comprising the amino acid sequence of SEQ ID NO: 264;
ppp. the HVRs of embodiment 12.ppp. and further comprises a VL comprising the amino acid sequence of SEQ ID NO: 264;
qqq. the HVRs of embodiment 12.qqq. and further comprises a VL comprising the amino acid sequence of SEQ ID NO: 264;
rrr. the HVRs of embodiment 12.rrr. and further comprises a VL comprising the amino acid sequence of SEQ ID NO: 264;
sss. the HVRs of embodiment 12.sss. and further comprises a VL comprising the amino acid sequence of SEQ ID NO: 269;
ttt. the HVRs of embodiment 12.ttt. and further comprises a VL comprising the amino acid sequence of SEQ ID NO: 269;
uuu. the HVRs of embodiment 12.uuu. and further comprises a VL comprising the amino acid sequence of SEQ ID NO: 269;
vvv. the HVRs of embodiment 12.vvv. and further comprises a VL comprising the amino acid sequence of SEQ ID NO: 275;
www. the HVRs of embodiment 12.www. and further comprises a VL comprising the amino acid sequence of SEQ ID NO: 275;
xxx. the HVRs of embodiment 12.xxx. and further comprises a VL comprising the amino acid sequence of SEQ ID NO: 275;
yyy. the HVRs of embodiment 12.yyy. and further comprises a VL comprising the amino acid sequence of SEQ ID NO: 275;
zzz. the HVRs of embodiment 12.zzz. and further comprises a VL comprising the amino acid sequence of SEQ ID NO: 275;
aaaa. the HVRs of embodiment 12.aaaa. and further comprises a VL comprising the amino acid sequence of SEQ ID NO: 275;
bbbb. the HVRs of embodiment 12.bbbb. and further comprises a VL comprising the amino acid sequence of SEQ ID NO: 275;
cccc. the HVRs of embodiment 12.cccc. and further comprises a VL comprising the amino acid sequence of SEQ ID NO: 275;
dddd. the HVRs of embodiment 12.dddd. and further comprises a VL comprising the amino acid sequence of SEQ ID NO: 275;
eeee. the HVRs of embodiment 12.eeee. and further comprises a VL comprising the amino acid sequence of SEQ ID NO: 264;
ffff. the HVRs of embodiment 12.ffff. and further comprises a VL comprising the amino acid sequence of SEQ ID NO: 275;
ggg. the HVRs of embodiment 12.gggg. and further comprises a VL comprising the amino acid sequence of SEQ ID NO: 275;
hhhh. the HVRs of embodiment 12.hhhh. and further comprises a VL comprising the amino acid sequence of SEQ ID NO: 275;
iiii. the HVRs of embodiment 12.iiii. and further comprises a VL comprising the amino acid sequence of SEQ ID NO: 416;
jjjj. the HVRs of embodiment 12.jjjj. and further comprises a VL comprising the amino acid sequence of SEQ ID NO: 312;
kkkk. the HVRs of embodiment 12.kkkk. and further comprises a VL comprising the amino acid sequence of SEQ ID NO: 312;
llll. the HVRs of embodiment 12.llll. and further comprises a VL comprising the amino acid sequence of SEQ ID NO: 312;
mmmm. the HVRs of embodiment 12.mmmm. and further comprises a VL comprising the amino acid sequence of SEQ ID NO: 312;
nnnn. the HVRs of embodiment 12.nnnn. and further comprises a VL comprising the amino acid sequence of SEQ ID NO: 312;
oooo. the HVRs of embodiment 12.oooo. and further comprises a VL comprising the amino acid sequence of SEQ ID NO: 312;
pppp. the HVRs of embodiment 12.pppp. and further comprises a VL comprising the amino acid sequence of SEQ ID NO: 312;
qqqq. the HVRs of embodiment 12.qqqq. and further comprises a VL comprising the amino acid sequence of SEQ ID NO: 312;
rrrr. the HVRs of embodiment 12.rrrr. and further comprises a VL comprising the amino acid sequence of SEQ ID NO: 312;
ssss. the HVRs of embodiment 12.ssss. and further comprises a VL comprising the amino acid sequence of SEQ ID NO: 312; or
tttt. the HVRs of embodiment 12.tttt. and further comprises a VL comprising the amino acid sequence of SEQ ID NO: 312.

Embodiment 17 is the antibody of any one of embodiments 9-13, wherein the antibody comprises:
a. a VH comprising the amino acid sequence of SEQ ID NO: 202 and a VL comprising the amino acid sequence of SEQ ID NO: 257;
b. a VH comprising the amino acid sequence of SEQ ID NO: 203 and a VL comprising the amino acid sequence of SEQ ID NO: 258;
c. a VH comprising the amino acid sequence of SEQ ID NO: 204 and a VL comprising the amino acid sequence of SEQ ID NO: 259;
d. a VH comprising the amino acid sequence of SEQ ID NO: 205 and a VL comprising the amino acid sequence of SEQ ID NO: 260;
e. a VH comprising the amino acid sequence of SEQ ID NO: 206 and a VL comprising the amino acid sequence of SEQ ID NO: 261;
f. a VH comprising the amino acid sequence of SEQ ID NO: 207 and a VL comprising the amino acid sequence of SEQ ID NO: 262;
g. a VH comprising the amino acid sequence of SEQ ID NO: 208 and a VL comprising the amino acid sequence of SEQ ID NO: 263;
h. a VH comprising the amino acid sequence of SEQ ID NO: 209 and a VL comprising the amino acid sequence of SEQ ID NO: 264;
i. a VH comprising the amino acid sequence of SEQ ID NO: 210 and a VL comprising the amino acid sequence of SEQ ID NO: 265;
j. a VH comprising the amino acid sequence of SEQ ID NO: 211 and a VL comprising the amino acid sequence of SEQ ID NO: 266;
k. a VH comprising the amino acid sequence of SEQ ID NO: 212 and a VL comprising the amino acid sequence of SEQ ID NO: 267;
l. a VH comprising the amino acid sequence of SEQ ID NO: 213 and a VL comprising the amino acid sequence of SEQ ID NO: 268;
m. a VH comprising the amino acid sequence of SEQ ID NO: 214 and a VL comprising the amino acid sequence of SEQ ID NO: 269;
n. a VH comprising the amino acid sequence of SEQ ID NO: 215 and a VL comprising the amino acid sequence of SEQ ID NO: 270;
o. a VH comprising the amino acid sequence of SEQ ID NO: 216 and a VL comprising the amino acid sequence of SEQ ID NO: 271;
p. a VH comprising the amino acid sequence of SEQ ID NO: 217 and a VL comprising the amino acid sequence of SEQ ID NO: 272;
q. a VH comprising the amino acid sequence of SEQ ID NO: 218 and a VL comprising the amino acid sequence of SEQ ID NO: 273;
r. a VH comprising the amino acid sequence of SEQ ID NO: 219 and a VL comprising the amino acid sequence of SEQ ID NO: 274;
s. a VH comprising the amino acid sequence of SEQ ID NO: 220 and a VL comprising the amino acid sequence of SEQ ID NO: 275;
t. a VH comprising the amino acid sequence of SEQ ID NO: 209 and a VL comprising the amino acid sequence of SEQ ID NO: 276;
u. a VH comprising the amino acid sequence of SEQ ID NO: 221 and a VL comprising the amino acid sequence of SEQ ID NO: 277;
v. a VH comprising the amino acid sequence of SEQ ID NO: 222 and a VL comprising the amino acid sequence of SEQ ID NO: 278;
w. a VH comprising the amino acid sequence of SEQ ID NO: 223 and a VL comprising the amino acid sequence of SEQ ID NO: 279;
x. a VH comprising the amino acid sequence of SEQ ID NO: 224 and a VL comprising the amino acid sequence of SEQ ID NO: 280;
y. a VH comprising the amino acid sequence of SEQ ID NO: 225 and a VL comprising the amino acid sequence of SEQ ID NO: 281;
z. a VH comprising the amino acid sequence of SEQ ID NO: 226 and a VL comprising the amino acid sequence of SEQ ID NO: 282;
aa. a VH comprising the amino acid sequence of SEQ ID NO: 227 and a VL comprising the amino acid sequence of SEQ ID NO: 283;
bb. a VH comprising the amino acid sequence of SEQ ID NO: 228 and a VL comprising the amino acid sequence of SEQ ID NO: 284;
cc. a VH comprising the amino acid sequence of SEQ ID NO: 228 and a VL comprising the amino acid sequence of SEQ ID NO: 285;
dd. a VH comprising the amino acid sequence of SEQ ID NO: 229 and a VL comprising the amino acid sequence of SEQ ID NO: 286;
ee. a VH comprising the amino acid sequence of SEQ ID NO: 225 and a VL comprising the amino acid sequence of SEQ ID NO: 287;
ff. a VH comprising the amino acid sequence of SEQ ID NO: 230 and a VL comprising the amino acid sequence of SEQ ID NO: 288;
gg. a VH comprising the amino acid sequence of SEQ ID NO: 231 and a VL comprising the amino acid sequence of SEQ ID NO: 289;
hh. a VH comprising the amino acid sequence of SEQ ID NO: 232 and a VL comprising the amino acid sequence of SEQ ID NO: 290;
ii. a VH comprising the amino acid sequence of SEQ ID NO: 233 and a VL comprising the amino acid sequence of SEQ ID NO: 291;
jj. a VH comprising the amino acid sequence of SEQ ID NO: 234 and a VL comprising the amino acid sequence of SEQ ID NO: 292;
kk. a VH comprising the amino acid sequence of SEQ ID NO: 235 and a VL comprising the amino acid sequence of SEQ ID NO: 293;
ll. a VH comprising the amino acid sequence of SEQ ID NO: 235 and a VL comprising the amino acid sequence of SEQ ID NO: 294;
mm. a VH comprising the amino acid sequence of SEQ ID NO: 236 and a VL comprising the amino acid sequence of SEQ ID NO: 295;
nn. a VH comprising the amino acid sequence of SEQ ID NO: 237 and a VL comprising the amino acid sequence of SEQ ID NO: 296;
oo. a VH comprising the amino acid sequence of SEQ ID NO: 238 and a VL comprising the amino acid sequence of SEQ ID NO: 297;
pp. a VH comprising the amino acid sequence of SEQ ID NO: 238 and a VL comprising the amino acid sequence of SEQ ID NO: 298;
qq. a VH comprising the amino acid sequence of SEQ ID NO: 239 and a VL comprising the amino acid sequence of SEQ ID NO: 299;
rr. a VH comprising the amino acid sequence of SEQ ID NO: 240 and a VL comprising the amino acid sequence of SEQ ID NO: 300;
ss. a VH comprising the amino acid sequence of SEQ ID NO: 241 and a VL comprising the amino acid sequence of SEQ ID NO: 301;
tt. a VH comprising the amino acid sequence of SEQ ID NO: 242 and a VL comprising the amino acid sequence of SEQ ID NO: 302;
uu. a VH comprising the amino acid sequence of SEQ ID NO: 243 and a VL comprising the amino acid sequence of SEQ ID NO: 303;
vv. a VH comprising the amino acid sequence of SEQ ID NO: 244 and a VL comprising the amino acid sequence of SEQ ID NO: 304;
ww. a VH comprising the amino acid sequence of SEQ ID NO: 245 and a VL comprising the amino acid sequence of SEQ ID NO: 305;
xx. a VH comprising the amino acid sequence of SEQ ID NO: 246 and a VL comprising the amino acid sequence of SEQ ID NO: 306;
yy. a VH comprising the amino acid sequence of SEQ ID NO: 247 and a VL comprising the amino acid sequence of SEQ ID NO: 307;
zz. a VH comprising the amino acid sequence of SEQ ID NO: 248 and a VL comprising the amino acid sequence of SEQ ID NO: 308;
aaa. a VH comprising the amino acid sequence of SEQ ID NO: 249 and a VL comprising the amino acid sequence of SEQ ID NO: 309;
bbb. a VH comprising the amino acid sequence of SEQ ID NO: 250 and a VL comprising the amino acid sequence of SEQ ID NO: 310;
ccc. a VH comprising the amino acid sequence of SEQ ID NO: 251 and a VL comprising the amino acid sequence of SEQ ID NO: 311;
ddd. a VH comprising the amino acid sequence of SEQ ID NO: 252 and a VL comprising the amino acid sequence of SEQ ID NO: 312;
eee. a VH comprising the amino acid sequence of SEQ ID NO: 253 and a VL comprising the amino acid sequence of SEQ ID NO: 313;
fff. a VH comprising the amino acid sequence of SEQ ID NO: 254 and a VL comprising the amino acid sequence of SEQ ID NO: 314;
ggg. a VH comprising the amino acid sequence of SEQ ID NO: 255 and a VL comprising the amino acid sequence of SEQ ID NO: 315;
hhh. a VH comprising the amino acid sequence of SEQ ID NO: 256 and a VL comprising the amino acid sequence of SEQ ID NO: 316;
iii. a VH comprising the amino acid sequence of SEQ ID NO: 233 and a VL comprising the amino acid sequence of SEQ ID NO: 291;
jjj. a VH comprising the amino acid sequence of SEQ ID NO: 256 and a VL comprising the amino acid sequence of SEQ ID NO: 317;
kkk. a VH comprising the amino acid sequence of SEQ ID NO: 361 and a VL comprising the amino acid sequence of SEQ ID NO: 261;
lll. a VH comprising the amino acid sequence of SEQ ID NO: 362 and a VL comprising the amino acid sequence of SEQ ID NO: 261;
mmm. a VH comprising the amino acid sequence of SEQ ID NO: 363 and a VL comprising the amino acid sequence of SEQ ID NO: 261;
nnn. a VH comprising the amino acid sequence of SEQ ID NO: 364 and a VL comprising the amino acid sequence of SEQ ID NO: 261;
ooo. a VH comprising the amino acid sequence of SEQ ID NO: 365 and a VL comprising the amino acid sequence of SEQ ID NO: 264;
ppp. a VH comprising the amino acid sequence of SEQ ID NO: 366 and a VL comprising the amino acid sequence of SEQ ID NO: 264;
qqq. a VH comprising the amino acid sequence of SEQ ID NO: 367 and a VL comprising the amino acid sequence of SEQ ID NO: 264;
rrr. a VH comprising the amino acid sequence of SEQ ID NO: 368 and a VL comprising the amino acid sequence of SEQ ID NO: 264;
sss. a VH comprising the amino acid sequence of SEQ ID NO: 369 and a VL comprising the amino acid sequence of SEQ ID NO: 269;
ttt. a VH comprising the amino acid sequence of SEQ ID NO: 370 and a VL comprising the amino acid sequence of SEQ ID NO: 269;
uuu. a VH comprising the amino acid sequence of SEQ ID NO: 387 and a VL comprising the amino acid sequence of SEQ ID NO: 269;
vvv. a VH comprising the amino acid sequence of SEQ ID NO: 388 and a VL comprising the amino acid sequence of SEQ ID NO: 275;
www. a VH comprising the amino acid sequence of SEQ ID NO: 389 and a VL comprising the amino acid sequence of SEQ ID NO: 275;
xxx. a VH comprising the amino acid sequence of SEQ ID NO: 390 and a VL comprising the amino acid sequence of SEQ ID NO: 275;
yyy. a VH comprising the amino acid sequence of SEQ ID NO: 391 and a VL comprising the amino acid sequence of SEQ ID NO: 275;
zzz. a VH comprising the amino acid sequence of SEQ ID NO: 392 and a VL comprising the amino acid sequence of SEQ ID NO: 275;
aaaa. a VH comprising the amino acid sequence of SEQ ID NO: 393 and a VL comprising the amino acid sequence of SEQ ID NO: 275;
bbbb. a VH comprising the amino acid sequence of SEQ ID NO: 394 and a VL comprising the amino acid sequence of SEQ ID NO: 275;
cccc. a VH comprising the amino acid sequence of SEQ ID NO: 395 and a VL comprising the amino acid sequence of SEQ ID NO: 275;
dddd. a VH comprising the amino acid sequence of SEQ ID NO: 396 and a VL comprising the amino acid sequence of SEQ ID NO: 275;
eeee. a VH comprising the amino acid sequence of SEQ ID NO: 398 and a VL comprising the amino acid sequence of SEQ ID NO: 264;
ffff. a VH comprising the amino acid sequence of SEQ ID NO: 399 and a VL comprising the amino acid sequence of SEQ ID NO: 275;
gggg. a VH comprising the amino acid sequence of SEQ ID NO: 400 and a VL comprising the amino acid sequence of SEQ ID NO: 275;
hhhh. a VH comprising the amino acid sequence of SEQ ID NO: 401 and a VL comprising the amino acid sequence of SEQ ID NO: 275;
iiii. a VH comprising the amino acid sequence of SEQ ID NO: 402 and a VL comprising the amino acid sequence of SEQ ID NO: 413;
jjjj. a VH comprising the amino acid sequence of SEQ ID NO: 403 and a VL comprising the amino acid sequence of SEQ ID NO: 312;
kkkk. a VH comprising the amino acid sequence of SEQ ID NO: 404 and a VL comprising the amino acid sequence of SEQ ID NO: 312;
llll. a VH comprising the amino acid sequence of SEQ ID NO: 405 and a VL comprising the amino acid sequence of SEQ ID NO: 312;
mmmm. a VH comprising the amino acid sequence of SEQ ID NO: 406 and a VL comprising the amino acid sequence of SEQ ID NO: 312;
nnnn. a VH comprising the amino acid sequence of SEQ ID NO: 407 and a VL comprising the amino acid sequence of SEQ ID NO: 312;
oooo. a VH comprising the amino acid sequence of SEQ ID NO: 408 and a VL comprising the amino acid sequence of SEQ ID NO: 312;
pppp. a VH comprising the amino acid sequence of SEQ ID NO: 409 and a VL comprising the amino acid sequence of SEQ ID NO: 312;
qqqq. a VH comprising the amino acid sequence of SEQ ID NO: 410 and a VL comprising the amino acid sequence of SEQ ID NO: 312;
rrrr. a VH comprising the amino acid sequence of SEQ ID NO: 411 and a VL comprising the amino acid sequence of SEQ ID NO: 312;
ssss. a VH comprising the amino acid sequence of SEQ ID NO: 397 and a VL comprising the amino acid sequence of SEQ ID NO: 312;
tttt. a VH comprising the amino acid sequence of SEQ ID NO: 412 and a VL comprising the amino acid sequence of SEQ ID NO: 312.

Embodiment 18 is an isolated antibody that specifically binds to human SIRPA, wherein the antibody comprises:
a. a VH and VL, wherein the VH comprises the HVR-H1, HVR-H2, and HVR-H3 of the antibody SA-1 (as shown in Table 3) and the VL comprises the HVR-L1, HVR-L2, and HVR-L3 of the antibody SA-1 (as shown in Table 2);
b. a VH and VL, wherein the VH comprises the HVR-H1, HVR-H2, and HVR-H3 of the antibody SA-2 (as shown in Table 3) and the VL comprises the HVR-L1, HVR-L2, and HVR-L3 of the antibody SA-2 (as shown in Table 2);
c. a VH and VL, wherein the VH comprises the HVR-H1, HVR-H2, and HVR-H3 of the antibody SA-3 (as shown in Table 3) and the VL comprises the HVR-L1, HVR-L2, and HVR-L3 of the antibody SA-3 (as shown in Table 2);
d. a VH and VL, wherein the VH comprises the HVR-H1, HVR-H2, and HVR-H3 of the antibody SA-4 (as shown in Table 3) and the VL comprises the HVR-L1, HVR-L2, and HVR-L3 of the antibody SA-4 (as shown in Table 2);
e. a VH and VL, wherein the VH comprises the HVR-H1, HVR-H2, and HVR-H3 of the antibody SA-5 (as shown in Table 3) and the VL comprises the HVR-L1, HVR-L2, and HVR-L3 of the antibody SA-5 (as shown in Table 2);
f. a VH and VL, wherein the VH comprises the HVR-H1, HVR-H2, and HVR-H3 of the antibody SA-6 (as shown in Table 3) and the VL comprises the HVR-L1, HVR-L2, and HVR-L3 of the antibody SA-6 (as shown in Table 2);
g. a VH and VL, wherein the VH comprises the HVR-H1, HVR-H2, and HVR-H3 of the antibody SA-7 (as shown in Table 3) and the VL comprises the HVR-L1, HVR-L2, and HVR-L3 of the antibody SA-7 (as shown in Table 2);
h. a VH and VL, wherein the VH comprises the HVR-H1, HVR-H2, and HVR-H3 of the antibody SA-8 (as shown in Table 3) and the VL comprises the HVR-L1, HVR-L2, and HVR-L3 of the antibody SA-8 (as shown in Table 2);
i. a VH and VL, wherein the VH comprises the HVR-H1, HVR-H2, and HVR-H3 of the antibody SA-9 (as shown in Table 3) and the VL comprises the HVR-L1, HVR-L2, and HVR-L3 of the antibody SA-9 (as shown in Table 2);
j. a VH and VL, wherein the VH comprises the HVR-H1, HVR-H2, and HVR-H3 of the antibody SA-10 (as shown in Table 3) and the VL comprises the HVR-L1, HVR-L2, and HVR-L3 of the antibody SA-10 (as shown in Table 2);
k. a VH and VL, wherein the VH comprises the HVR-H1, HVR-H2, and HVR-H3 of the antibody SA-11 (as shown in Table 3) and the VL comprises the HVR-L1, HVR-L2, and HVR-L3 of the antibody SA-11 (as shown in Table 2);
l. a VH and VL, wherein the VH comprises the HVR-H1, HVR-H2, and HVR-H3 of the antibody SA-12 (as shown in Table 3) and the VL comprises the HVR-L1, HVR-L2, and HVR-L3 of the antibody SA-12 (as shown in Table 2);
m. a VH and VL, wherein the VH comprises the HVR-H1, HVR-H2, and HVR-H3 of the antibody SA-13 (as shown in Table 3) and the VL comprises the HVR-L1, HVR-L2, and HVR-L3 of the antibody SA-13 (as shown in Table 2);
n. a VH and VL, wherein the VH comprises the HVR-H1, HVR-H2, and HVR-H3 of the antibody SA-14 (as shown in Table 3) and the VL comprises the HVR-L1, HVR-L2, and HVR-L3 of the antibody SA-14 (as shown in Table 2);
o. a VH and VL, wherein the VH comprises the HVR-H1, HVR-H2, and HVR-H3 of the antibody SA-15 (as shown in Table 3) and the VL comprises the HVR-L1, HVR-L2, and HVR-L3 of the antibody SA-15 (as shown in Table 2);
p. a VH and VL, wherein the VH comprises the HVR-H1, HVR-H2, and HVR-H3 of the antibody SA-16 (as shown in Table 3) and the VL comprises the HVR-L1, HVR-L2, and HVR-L3 of the antibody SA-16 (as shown in Table 2);
q. a VH and VL, wherein the VH comprises the HVR-H1, HVR-H2, and HVR-H3 of the antibody SA-17 (as shown in Table 3) and the VL comprises the HVR-L1, HVR-L2, and HVR-L3 of the antibody SA-17 (as shown in Table 2);
r. a VH and VL, wherein the VH comprises the HVR-H1, HVR-H2, and HVR-H3 of the antibody SA-18 (as shown in Table 3) and the VL comprises the HVR-L1, HVR-L2, and HVR-L3 of the antibody SA-18 (as shown in Table 2);
s. a VH and VL, wherein the VH comprises the HVR-H1, HVR-H2, and HVR-H3 of the antibody SA-19 (as shown in Table 3) and the VL comprises the HVR-L1, HVR-L2, and HVR-L3 of the antibody SA-19 (as shown in Table 2);
t. a VH and VL, wherein the VH comprises the HVR-H1, HVR-H2, and HVR-H3 of the antibody SA-20 (as shown in Table 3) and the VL comprises the HVR-L1, HVR-L2, and HVR-L3 of the antibody SA-20 (as shown in Table 2);
u. a VH and VL, wherein the VH comprises the HVR-H1, HVR-H2, and HVR-H3 of the antibody SA-21 (as shown in Table 3) and the VL comprises the HVR-L1, HVR-L2, and HVR-L3 of the antibody SA-21 (as shown in Table 2);
v. a VH and VL, wherein the VH comprises the HVR-H1, HVR-H2, and HVR-H3 of the antibody SA-22 (as shown in Table 3) and the VL comprises the HVR-L1, HVR-L2, and HVR-L3 of the antibody SA-22 (as shown in Table 2);
w. a VH and VL, wherein the VH comprises the HVR-H1, HVR-H2, and HVR-H3 of the antibody SA-23 (as shown in Table 3) and the VL comprises the HVR-L1, HVR-L2, and HVR-L3 of the antibody SA-23 (as shown in Table 2);
x. a VH and VL, wherein the VH comprises the HVR-H1, HVR-H2, and HVR-H3 of the antibody SA-24 (as shown in Table 3) and the VL comprises the HVR-L1, HVR-L2, and HVR-L3 of the antibody SA-24 (as shown in Table 2);
y. a VH and VL, wherein the VH comprises the HVR-H1, HVR-H2, and HVR-H3 of the antibody SA-25 (as shown in Table 3) and the VL comprises the HVR-L1, HVR-L2, and HVR-L3 of the antibody SA-25 (as shown in Table 2);
z. a VH and VL, wherein the VH comprises the HVR-H1, HVR-H2, and HVR-H3 of the antibody SA-26 (as shown in Table 3) and the VL comprises the HVR-L1, HVR-L2, and HVR-L3 of the antibody SA-26 (as shown in Table 2);
aa. a VH and VL, wherein the VH comprises the HVR-H1, HVR-H2, and HVR-H3 of the antibody SA-27 (as shown in Table 3) and the VL comprises the HVR-L1, HVR-L2, and HVR-L3 of the antibody SA-27 (as shown in Table 2);
bb. a VH and VL, wherein the VH comprises the HVR-H1, HVR-H2, and HVR-H3 of the antibody SA-28 (as shown in Table 3) and the VL comprises the HVR-L1, HVR-L2, and HVR-L3 of the antibody SA-28 (as shown in Table 2);
cc. a VH and VL, wherein the VH comprises the HVR-H1, HVR-H2, and HVR-H3 of the antibody SA-29 (as shown in Table 3) and the VL comprises the HVR-L1, HVR-L2, and HVR-L3 of the antibody SA-29 (as shown in Table 2);
dd. a VH and VL, wherein the VH comprises the HVR-H1, HVR-H2, and HVR-H3 of the antibody SA-30 (as shown in Table 3) and the VL comprises the HVR-L1, HVR-L2, and HVR-L3 of the antibody SA-30 (as shown in Table 2);
ee. a VH and VL, wherein the VH comprises the HVR-H1, HVR-H2, and HVR-H3 of the antibody SA-31 (as shown in Table 3) and the VL comprises the HVR-L1, HVR-L2, and HVR-L3 of the antibody SA-31 (as shown in Table 2);
ff. a VH and VL, wherein the VH comprises the HVR-H1, HVR-H2, and HVR-H3 of the antibody SA-32 (as shown in Table 3) and the VL comprises the HVR-L1, HVR-L2, and HVR-L3 of the antibody SA-32 (as shown in Table 2);
gg. a VH and VL, wherein the VH comprises the HVR-H1, HVR-H2, and HVR-H3 of the antibody SA-33 (as shown in Table 3) and the VL comprises the HVR-L1, HVR-L2, and HVR-L3 of the antibody SA-33 (as shown in Table 2);
hh. a VH and VL, wherein the VH comprises the HVR-H1, HVR-H2, and HVR-H3 of the antibody SA-34 (as shown in Table 3) and the VL comprises the HVR-L1, HVR-L2, and HVR-L3 of the antibody SA-34 (as shown in Table 2);
ii. a VH and VL, wherein the VH comprises the HVR-H1, HVR-H2, and HVR-H3 of the antibody SA-35 (as shown in Table 3) and the VL comprises the HVR-L1, HVR-L2, and HVR-L3 of the antibody SA-35 (as shown in Table 2);
jj. a VH and VL, wherein the VH comprises the HVR-H1, HVR-H2, and HVR-H3 of the antibody SA-3+(as shown in Table 3) and the VL comprises the HVR-L1, HVR-L2, and HVR-L3 of the antibody SA-36 (as shown in Table 2);
kk. a VH and VL, wherein the VH comprises the HVR-H1, HVR-H2, and HVR-H3 of the antibody SA-37 (as shown in Table 3) and the VL comprises the HVR-L1, HVR-L2, and HVR-L3 of the antibody SA-37 (as shown in Table 2);
ll. a VH and VL, wherein the VH comprises the HVR-H1, HVR-H2, and HVR-H3 of the antibody SA-38 (as shown in Table 3) and the VL comprises the HVR-L1, HVR-L2, and HVR-L3 of the antibody SA-38 (as shown in Table 2);
mm. a VH and VL, wherein the VH comprises the HVR-H1, HVR-H2, and HVR-H3 of the antibody SA-39 (as shown in Table 3) and the VL comprises the HVR-L1, HVR-L2, and HVR-L3 of the antibody SA-39 (as shown in Table 2);
nn. a VH and VL, wherein the VH comprises the HVR-H1, HVR-H2, and HVR-H3 of the antibody SA-40 (as shown in Table 3) and the VL comprises the HVR-L1, HVR-L2, and HVR-L3 of the antibody SA-40 (as shown in Table 2);
oo. a VH and VL, wherein the VH comprises the HVR-H1, HVR-H2, and HVR-H3 of the antibody SA-41 (as shown in Table 3) and the VL comprises the HVR-L1, HVR-L2, and HVR-L3 of the antibody SA-41 (as shown in Table 2);
pp. a VH and VL, wherein the VH comprises the HVR-H1, HVR-H2, and HVR-H3 of the antibody SA-42 (as shown in Table 3) and the VL comprises the HVR-L1, HVR-L2, and HVR-L3 of the antibody SA-42 (as shown in Table 2);
qq. a VH and VL, wherein the VH comprises the HVR-H1, HVR-H2, and HVR-H3 of the antibody SA-43 (as shown in Table 3) and the VL comprises the HVR-L1, HVR-L2, and HVR-L3 of the antibody SA-43 (as shown in Table 2);
rr. a VH and VL, wherein the VH comprises the HVR-H1, HVR-H2, and HVR-H3 of the antibody SA-44 (as shown in Table 3) and the VL comprises the HVR-L1, HVR-L2, and HVR-L3 of the antibody SA-44 (as shown in Table 2);
ss. a VH and VL, wherein the VH comprises the HVR-H1, HVR-H2, and HVR-H3 of the antibody SA-45 (as shown in Table 3) and the VL comprises the HVR-L1, HVR-L2, and HVR-L3 of the antibody SA-45 (as shown in Table 2);
tt. a VH and VL, wherein the VH comprises the HVR-H1, HVR-H2, and HVR-H3 of the antibody SA-46 (as shown in Table 3) and the VL comprises the HVR-L1, HVR-L2, and HVR-L3 of the antibody SA-46 (as shown in Table 2);
uu. a VH and VL, wherein the VH comprises the HVR-H1, HVR-H2, and HVR-H3 of the antibody SA-47 (as shown in Table 3) and the VL comprises the HVR-L1, HVR-L2, and HVR-L3 of the antibody SA-47 (as shown in Table 2);
vv. a VH and VL, wherein the VH comprises the HVR-H1, HVR-H2, and HVR-H3 of the antibody SA-48 (as shown in Table 3) and the VL comprises the HVR-L1, HVR-L2, and HVR-L3 of the antibody SA-48 (as shown in Table 2);
ww. a VH and VL, wherein the VH comprises the HVR-H1, HVR-H2, and HVR-H3 of the antibody SA-49 (as shown in Table 3) and the VL comprises the HVR-L1, HVR-L2, and HVR-L3 of the antibody SA-49 (as shown in Table 2);
xx. a VH and VL, wherein the VH comprises the HVR-H1, HVR-H2, and HVR-H3 of the antibody SA-50 (as shown in Table 3) and the VL comprises the HVR-L1, HVR-L2, and HVR-L3 of the antibody SA-50 (as shown in Table 2);
yy. a VH and VL, wherein the VH comprises the HVR-H1, HVR-H2, and HVR-H3 of the antibody SA-51 (as shown in Table 3) and the VL comprises the HVR-L1, HVR-L2, and HVR-L3 of the antibody SA-51 (as shown in Table 2);
zz. a VH and VL, wherein the VH comprises the HVR-H1, HVR-H2, and HVR-H3 of the antibody SA-52 (as shown in Table 3) and the VL comprises the HVR-L1, HVR-L2, and HVR-L3 of the antibody SA-52 (as shown in Table 2);
aaa. a VH and VL, wherein the VH comprises the HVR-H1, HVR-H2, and HVR-H3 of the antibody SA-53 (as shown in Table 3) and the VL comprises the HVR-L1, HVR-L2, and HVR-L3 of the antibody SA-53 (as shown in Table 2);
bbb. a VH and VL, wherein the VH comprises the HVR-H1, HVR-H2, and HVR-H3 of the antibody SA-54 (as shown in Table 3) and the VL comprises the HVR-L1, HVR-L2, and HVR-L3 of the antibody SA-54 (as shown in Table 2);
ccc. a VH and VL, wherein the VH comprises the HVR-H1, HVR-H2, and HVR-H3 of the antibody SA-55 (as shown in Table 3) and the VL comprises the HVR-L1, HVR-L2, and HVR-L3 of the antibody SA-55 (as shown in Table 2);
ddd. a VH and VL, wherein the VH comprises the HVR-H1, HVR-H2, and HVR-H3 of the antibody SA-56 (as shown in Table 3) and the VL comprises the HVR-L1, HVR-L2, and HVR-L3 of the antibody SA-56 (as shown in Table 2);
eee. a VH and VL, wherein the VH comprises the HVR-H1, HVR-H2, and HVR-H3 of the antibody SA-57 (as shown in Table 3) and the VL comprises the HVR-L1, HVR-L2, and HVR-L3 of the antibody SA-57 (as shown in Table 2);
fff. a VH and VL, wherein the VH comprises the HVR-H1, HVR-H2, and HVR-H3 of the antibody SA-58 (as shown in Table 3) and the VL comprises the HVR-L1, HVR-L2, and HVR-L3 of the antibody SA-58 (as shown in Table 2);
ggg. a VH and VL, wherein the VH comprises the HVR-H1, HVR-H2, and HVR-H3 of the antibody SA-59 (as shown in Table 3) and the VL comprises the HVR-L1, HVR-L2, and HVR-L3 of the antibody SA-59 (as shown in Table 2);
hhh. a VH and VL, wherein the VH comprises the HVR-H1, HVR-H2, and HVR-H3 of the antibody SA-60 (as shown in Table 3) and the VL comprises the HVR-L1, HVR-L2, and HVR-L3 of the antibody SA-60 (as shown in Table 2);
iii. a VH and VL, wherein the VH comprises the HVR-H1, HVR-H2, and HVR-H3 of the antibody SA-61 (as shown in Table 3) and the VL comprises the HVR-L1, HVR-L2, and HVR-L3 of the antibody SA-61 (as shown in Table 2);
jjj. a VH and VL, wherein the VH comprises the HVR-H1, HVR-H2, and HVR-H3 of the antibody SA-62 (as shown in Table 3) and the VL comprises the HVR-L1, HVR-L2, and HVR-L3 of the antibody SA-62 (as shown in Table 2);
kkk. a VH and VL, wherein the VH comprises the HVR-H1, HVR-H2, and HVR-H3 of the antibody SA-5-57 (as shown in Table 7) and the VL comprises the HVR-L1, HVR-L2, and HVR-L3 of the antibody SA-5-57 (as shown in Table 6);
lll. a VH and VL, wherein the VH comprises the HVR-H1, HVR-H2, and HVR-H3 of the antibody SA-5-58 (as shown in Table 7) and the VL comprises the HVR-L1, HVR-L2, and HVR-L3 of the antibody SA-5-58 (as shown in Table 6);
mmm. a VH and VL, wherein the VH comprises the HVR-H1, HVR-H2, and HVR-H3 of the antibody SA-5-59 (as shown in Table 7) and the VL comprises the HVR-L1, HVR-L2, and HVR-L3 of the antibody SA-5-59 (as shown in Table 6);
nnn. a VH and VL, wherein the VH comprises the HVR-H1, HVR-H2, and HVR-H3 of the antibody SA-5-61 (as shown in Table 7) and the VL comprises the HVR-L1, HVR-L2, and HVR-L3 of the antibody SA-5-61 (as shown in Table 6);
ooo. a VH and VL, wherein the VH comprises the HVR-H1, HVR-H2, and HVR-H3 of the antibody SA-8-62 (as shown in Table 7) and the VL comprises the HVR-L1, HVR-L2, and HVR-L3 of the antibody SA-8-62 (as shown in Table 6);
ppp. a VH and VL, wherein the VH comprises the HVR-H1, HVR-H2, and HVR-H3 of the antibody SA-8-64 (as shown in Table 7) and the VL comprises the HVR-L1, HVR-L2, and HVR-L3 of the antibody SA-8-64 (as shown in Table 6);
qqq. a VH and VL, wherein the VH comprises the HVR-H1, HVR-H2, and HVR-H3 of the antibody SA-8-66 (as shown in Table 7) and the VL comprises the HVR-L1, HVR-L2, and HVR-L3 of the antibody SA-8-66 (as shown in Table 6);
rrr. a VH and VL, wherein the VH comprises the HVR-H1, HVR-H2, and HVR-H3 of the antibody SA-8-67 (as shown in Table 7) and the VL comprises the HVR-L1, HVR-L2, and HVR-L3 of the antibody SA-8-67 (as shown in Table 6);
sss. a VH and VL, wherein the VH comprises the HVR-H1, HVR-H2, and HVR-H3 of the antibody SA-13-68 (as shown in Table 7) and the VL comprises the HVR-L1, HVR-L2, and HVR-L3 of the antibody SA-13-68 (as shown in Table 6);
ttt. a VH and VL, wherein the VH comprises the HVR-H1, HVR-H2, and HVR-H3 of the antibody SA-13-69 (as shown in Table 7) and the VL comprises the HVR-L1, HVR-L2, and HVR-L3 of the antibody SA-13-69 (as shown in Table 6);
uuu. a VH and VL, wherein the VH comprises the HVR-H1, HVR-H2, and HVR-H3 of the antibody SA-13-71 (as shown in Table 7) and the VL comprises the HVR-L1, HVR-L2, and HVR-L3 of the antibody SA-13-71 (as shown in Table 6);
vvv. a VH and VL, wherein the VH comprises the HVR-H1, HVR-H2, and HVR-H3 of the antibody SA-19-72 (as shown in Table 7) and the VL comprises the HVR-L1, HVR-L2, and HVR-L3 of the antibody SA-19-72 (as shown in Table 6);
www. a VH and VL, wherein the VH comprises the HVR-H1, HVR-H2, and HVR-H3 of the antibody SA-19-73 (as shown in Table 7) and the VL comprises the HVR-L1, HVR-L2, and HVR-L3 of the antibody SA-19-73 (as shown in Table 6);
xxx. a VH and VL, wherein the VH comprises the HVR-H1, HVR-H2, and HVR-H3 of the antibody SA-19-74 (as shown in Table 7) and the VL comprises the HVR-L1, HVR-L2, and HVR-L3 of the antibody SA-19-74 (as shown in Table 6);
yyy. a VH and VL, wherein the VH comprises the HVR-H1, HVR-H2, and HVR-H3 of the antibody SA-19-75 (as shown in Table 7) and the VL comprises the HVR-L1, HVR-L2, and HVR-L3 of the antibody SA-19-75 (as shown in Table 6);
zzz. a VH and VL, wherein the VH comprises the HVR-H1, HVR-H2, and HVR-H3 of the antibody SA-19-76 (as shown in Table 7) and the VL comprises the HVR-L1, HVR-L2, and HVR-L3 of the antibody SA-19-76 (as shown in Table 6);
aaaa. a VH and VL, wherein the VH comprises the HVR-H1, HVR-H2, and HVR-H3 of the antibody SA-19-77 (as shown in Table 7) and the VL comprises the HVR-L1, HVR-L2, and HVR-L3 of the antibody SA-19-77 (as shown in Table 6);
bbbb. a VH and VL, wherein the VH comprises the HVR-H1, HVR-H2, and HVR-H3 of the antibody SA-19-78 (as shown in Table 7) and the VL comprises the HVR-L1, HVR-L2, and HVR-L3 of the antibody SA-19-78 (as shown in Table 6);
cccc. a VH and VL, wherein the VH comprises the HVR-H1, HVR-H2, and HVR-H3 of the antibody SA-19-79 (as shown in Table 7) and the VL comprises the HVR-L1, HVR-L2, and HVR-L3 of the antibody SA-19-79 (as shown in Table 6);
dddd. a VH and VL, wherein the VH comprises the HVR-H1, HVR-H2, and HVR-H3 of the antibody SA-19-80 (as shown in Table 7) and the VL comprises the HVR-L1, HVR-L2, and HVR-L3 of the antibody SA-19-80 (as shown in Table 6);
eeee. a VH and VL, wherein the VH comprises the HVR-H1, HVR-H2, and HVR-H3 of the antibody SA-19-81 (as shown in Table 7) and the VL comprises the HVR-L1, HVR-L2, and HVR-L3 of the antibody SA-19-81 (as shown in Table 6);
ffff. a VH and VL, wherein the VH comprises the HVR-H1, HVR-H2, and HVR-H3 of the antibody SA-19-82 (as shown in Table 7) and the VL comprises the HVR-L1, HVR-L2, and HVR-L3 of the antibody SA-19-82 (as shown in Table 6);
gggg. a VH and VL, wherein the VH comprises the HVR-H1, HVR-H2, and HVR-H3 of the antibody SA-19-83 (as shown in Table 7) and the VL comprises the HVR-L1, HVR-L2, and HVR-L3 of the antibody SA-19-83 (as shown in Table 6);
hhhh. a VH and VL, wherein the VH comprises the HVR-H1, HVR-H2, and HVR-H3 of the antibody SA-19-84 (as shown in Table 7) and the VL comprises the HVR-L1, HVR-L2, and HVR-L3 of the antibody SA-19-84 (as shown in Table 6);
iiii. a VH and VL, wherein the VH comprises the HVR-H1, HVR-H2, and HVR-H3 of the antibody SA-56-85 (as shown in Table 7) and the VL comprises the HVR-L1, HVR-L2, and HVR-L3 of the antibody SA-56-85 (as shown in Table 6);
jjjj. a VH and VL, wherein the VH comprises the HVR-H1, HVR-H2, and HVR-H3 of the antibody SA-56-86 (as shown in Table 7) and the VL comprises the HVR-L1, HVR-L2, and HVR-L3 of the antibody SA-56-86 (as shown in Table 6);
kkkk. a VH and VL, wherein the VH comprises the HVR-H1, HVR-H2, and HVR-H3 of the antibody SA-56-87 (as shown in Table 7) and the VL comprises the HVR-L1, HVR-L2, and HVR-L3 of the antibody SA-56-87 (as shown in Table 6);
llll. a VH and VL, wherein the VH comprises the HVR-H1, HVR-H2, and HVR-H3 of the antibody SA-56-88 (as shown in Table 7) and the VL comprises the HVR-L1, HVR-L2, and HVR-L3 of the antibody SA-56-88 (as shown in Table 6);
mmmm. a VH and VL, wherein the VH comprises the HVR-H1, HVR-H2, and HVR-H3 of the antibody SA-56-89 (as shown in Table 7) and the VL comprises the HVR-L1, HVR-L2, and HVR-L3 of the antibody SA-56-89 (as shown in Table 6);
nnnn. a VH and VL, wherein the VH comprises the HVR-H1, HVR-H2, and HVR-H3 of the antibody SA-56-90 (as shown in Table 7) and the VL comprises the HVR-L1, HVR-L2, and HVR-L3 of the antibody SA-56-90 (as shown in Table 6);
oooo. a VH and VL, wherein the VH comprises the HVR-H1, HVR-H2, and HVR-H3 of the antibody SA-56-91 (as shown in Table 7) and the VL comprises the HVR-L1, HVR-L2, and HVR-L3 of the antibody SA-56-91 (as shown in Table 6);
pppp. a VH and VL, wherein the VH comprises the HVR-H1, HVR-H2, and HVR-H3 of the antibody SA-56-92 (as shown in Table 7) and the VL comprises the HVR-L1, HVR-L2, and HVR-L3 of the antibody SA-56-92 (as shown in Table 6);
qqqq. a VH and VL, wherein the VH comprises the HVR-H1, HVR-H2, and HVR-H3 of the antibody SA-56-93 (as shown in Table 7) and the VL comprises the HVR-L1, HVR-L2, and HVR-L3 of the antibody SA-56-93 (as shown in Table 6);
rrrr. a VH and VL, wherein the VH comprises the HVR-H1, HVR-H2, and HVR-H3 of the antibody SA-56-94 (as shown in Table 7) and the VL comprises the HVR-L1, HVR-L2, and HVR-L3 of the antibody SA-56-94 (as shown in Table 6);
ssss. a VH and VL, wherein the VH comprises the HVR-H1, HVR-H2, and HVR-H3 of the antibody SA-56-95 (as shown in Table 7) and the VL comprises the HVR-L1, HVR-L2, and HVR-L3 of the antibody SA-56-95 (as shown in Table 6); or
tttt. a VH and VL, wherein the VH comprises the HVR-H1, HVR-H2, and HVR-H3 of the antibody SA-56-96 (as shown in Table 7) and the VL comprises the HVR-L1, HVR-L2, and HVR-L3 of the antibody SA-56-96 (as shown in Table 6).

Embodiment 19 is the antibody of any one of embodiments 1-17, wherein the antibody has one or more of the following properties:
a. the antibody increases SIRPA activity, SIRPA signaling, CD47-induced SIRPA signaling, or any combination thereof, in macrophages, dendritic cells, and/or microglial cells;
b. the antibody decreases phagocytic activity by phagocytic cells, decreases dendritic cell cytokine release (e.g., release of TNFalpha), suppresses synapse elimination in microglia-neuron co-cultures, suppresses synapse elimination in mouse models, or any combination thereof;
c. the antibody has an affinity (KD) to human SIRPA of less than 1 μM, less than 100 nM, less than 10 nM, less than 1 nM, less than 0.1 nM, less than 0.01 nM, or less than 0.001 nM;
d. the antibody has an affinity (KD) to human SIRPAv1 (SEQ ID NO: 1) of 1 nM to 50 nM, 0.3 nM to 2 nM, or 2 nM to 24 nM;
e. the antibody has higher affinity to human SIRPA than to mouse SIRPA;
f. the antibody does not bind to mouse SIRPA;
g. the antibody has higher affinity to human SIRPA than to human SIRPB;
h. the antibody does not bind to human SIRPB;
i. the antibody binds to one or more amino acids in human SIRPA v1 (SEQ ID NO: 1) chosen from D40, R54, and W68;
j. the antibody has reduced binding affinity to a human SIRPAv1 (SEQ ID NO: 1) in comprising one or more of the following substitutions: D40A, R54A, W68A, compared to wild-type SIRPAv1 consisting of SEQ ID NO: 1.

Embodiment 20 is the antibody of any one of embodiments 1-19, wherein the antibody is a monoclonal antibody.

Embodiment 21 is the antibody of any one of embodiments 1-20, wherein the antibody is a humanized antibody.

Embodiment 22 is the antibody of any one of embodiments 1-21, wherein the antibody is an antigen binding fragment, such as an Fab, Fab′, Fab′-SH, F(ab′)₂, Fv, or scFv fragment.

Embodiment 23 is the antibody of any one of embodiments 1-22, wherein the antibody is a bispecific or multispecific antibody.

Embodiment 24 is the antibody of any one of embodiments 1-23, wherein the antibody is of the IgG class, the IgM class, or the IgA class.

Embodiment 25 is the antibody of embodiment 24, wherein the antibody is of the IgG class and is of a human IgG1, IgG2, IgG3, or IgG4 isotype or of a mouse IgG1 or IgG2 isotype.

Embodiment 26 is the antibody of any one of embodiments 1-25, wherein the antibody binds to an inhibitory Fc receptor.

Embodiment 27 is the antibody of embodiment 26, wherein the inhibitory Fc receptor is inhibitory Fc-gamma receptor IIB (FcgRIIB).

Embodiment 28 is the antibody of embodiment 27, wherein the antibody decreases cellular levels of FcgRIIB.

Embodiment 29 is the antibody of any one of embodiments 1-28, wherein the anti-SIRPA antibody has a human or mouse IgG1 isotype and comprises one or more amino acid substitutions in the Fc region at an amino acid residue selected from the group consisting of: N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, N325S, L328F, T394D, and any combination thereof, wherein the numbering of the residues is according to EU numbering, or comprises an amino acid deletion in the Fc region at a position corresponding to glycine 236.

Embodiment 30 is the antibody of any one of embodiments 1-29, wherein the antibody comprises one or more amino acid substitutions in the Fc region at a residue position selected from the group consisting of: C127S, L234A, L234F, L235A, L235E, S267E, K322A, N325S, L328F, A330S, P331S, E345R, E430G, S440Y, and any combination thereof, wherein the numbering of the amino acid residues is according to EU or Kabat numbering.

Embodiment 31 is a pharmaceutical composition comprising the anti-SIRPA antibody of any one of embodiments 1-30 and a pharmaceutically acceptable carrier.

Embodiment 32 is an isolated nucleic acid comprising a nucleic acid sequence encoding the anti-SIRPA antibody of any one of embodiments 1-30.

Embodiment 33 is an isolated vector comprising the nucleic acid of embodiment 32.

Embodiment 34 is an isolated host cell comprising the nucleic acid of embodiment 32 or the vector of embodiment 33.

Embodiment 35 is a method of producing an antibody that binds to human SIRPA, comprising culturing the cell of embodiment 34 so that the antibody is produced.

Embodiment 36 is the method of embodiment 35, further comprising recovering the antibody produced by the cell.

Embodiment 37 is a method of treating a disease or disorder associated with inflammation, transplant rejection, autoimmunity, or cognitive impairment, the method comprising administering to an individual in need thereof a therapeutically effective amount of an anti-SIRPA antibody of any one of embodiments 1-30, thereby treating the disease or disorder.

Embodiment 38 is the method of embodiment 37, wherein the disease or disorder is chosen from inflammation, intestinal inflammation, intestinal inflammation associated with colitis, rheumatoid arthritis, organ/graft transplant rejection, multiple sclerosis, synaptic pruning in neurons, synaptic loss in neurons, synaptic pruning by microglia, and cognitive impairment.

Embodiment 39 is a method of detecting the presence of SIRPA in a sample or an individual, the method comprising an anti-SIRPA antibody of any one of embodiments 1-30.

Embodiment 40 is the method of embodiment 39, further comprising quantification of antigen-bound anti-SIRPA antibody.

### BRIEF DESCRIPTION OF THE DRAWINGS

FIG. 1A shows an amino acid sequence alignment between the two most common alleles of human SIRPA protein (v1 and v2) depicting the divergent residues within the ligand-binding domain. Accession numbers are NP542970 (SEQ ID NO: 1) and CAA71403 (SEQ ID NO: 2), respectively.

FIG. 1B shows an amino acid sequence alignment between the human SIRPA v1 protein (SEQ ID NO: 1) and the human SIRPb1 (SEQ ID NO: 4) protein, depicting the homology between the two proteins. Accession numbers are NP542970 and O00241, respectively.

FIG. 2 shows an amino acid sequence alignment between human SIRPA protein (SEQ ID NO: 1) and mouse SIRPA (SEQ ID NO: 3) protein, depicting the homology between the two proteins. Accession numbers are NP542970 and Q6P618, respectively.

FIG. 3 shows the relative MFI values of anti-SIRPA antibodies of the present disclosure binding to Chinese hamster ovary cell line (CHO) overexpressing human SIRPA compared to CHO cells overexpressing mouse SIRPA. Results are expressed as fold over background. The background level is set to 1 on y-axis.

FIG. 4A shows induction of human SIRPA-dependent luciferase expression in a cell-based reporter assay. BWZ/NFAT-luciferase reporter cells (BWZ) were engineered to stably express human SIRPA-DAP12 chimera (BWZ-HuSIRPA). Cells were stimulated overnight with full-length anti-SIRPA antibodies or human IgG1 isotype control adsorbed onto 96-well plate at 10 mg/mL.

FIG. 4B shows induction of human SIRPb1-dependent luciferase expression in a cell-based reporter assay. BWZ/NFAT-luciferase reporter cells (BWZ) were engineered to stably co-express human SIRPb1 and DAP12 (BWZ-HuSIRPb1). Cells were stimulated overnight with full-length anti-SIRPA antibodies or human IgG1 isotype control adsorbed onto 96-well plate at 10 mg/mL.

FIG. 5A shows epitope binning of anti-SIRPA antibodies against the CD47-blocking anti-SIRPA antibody clone SE7C2 (Santa Cruz Biotechnology). Results are presented as relative MFI values of PE-conjugated SE7C2 binding to BWZ-HuSIRPA cells pre-incubated with 10 mg/mL of test antibodies or human IgG1 isotype control. Unlabeled cells establish the background signal set to 1 on the y-axis.

FIG. 5B shows the relative MFI values of recombinant soluble CD47 binding to BWZ-HuSIRPA cells in the presence of potential ligand blocking anti-SIRPA antibodies or human IgG1 isotype control. Recombinant CD47 contains a C-terminal HIS-tag and surface bound protein is detected with a PE-conjugated anti-HIS tag antibody. Results are depicted as fold over background by dividing MFI values of samples incubated with HuCD47 and antibodies by the MFI value of cells stained with anti-HIS tag PE in the absence of HuCD47.

FIG. 5C shows the relative MFI values of recombinant soluble CD47 binding to BWZ-HuSIRPA cells in the presence of ligand enhancing anti-SIRPA antibodies SA-13 and SA-56, or human IgG1 isotype control.

FIG. 5D shows luminescence values of BWZ-human SIRPA reporter cells incubated in the presence or absence of plate-bound CD47 and the indicated test antibodies. Results are presented as fold over background with radiance values from unstimulated reported cells set to 1 on the y-axis.

FIG. 6A shows luminescence values from BWZ-huSIRPA reporter cells co-cultured with Raji B cells in the presence of anti-SIRPA antibodies or isotype control. Results are presented as fold-over background with radiance values from isotype control-treated reporter cells mixed with Raji cells set to 1 on the y-axis. Reporter cells were treated with bin 2 anti-SIRPA antibodies SA-56 and SA-13, and for comparison, with bin 1 anti-SIRPA antibodies 12D6 and 1H11.

FIG. 6B shows luminescence values from BWZ-huSIRPb1 reporter cells co-cultured with Raji B cells in the presence of anti-SIRPA antibodies or isotype control. Bin 2 anti-SIRPA antibodies, SA-56 and SA-13, are SIRPA-specific, but bin 1 anti-SIRPA antibodies 12D6 and 1H11 are SIRPA/b1 cross-reactive.

FIG. 7A shows induction of human SIRPA-dependent luciferase expression in a cell-based reporter assay by affinity matured anti-SIRPA antibodies derived from parental anti-SIRPA antibodies SA-5 and SA-8.

FIG. 7B shows induction of human SIRPb1-dependent luciferase expression in a cell-based reporter assay by affinity matured anti-SIRPA antibodies derived from parental anti-SIRPA antibodies SA-5 and SA-8.

FIG. 7C shows induction of human SIRPA-dependent luciferase expression in a cell-based reporter assay by affinity matured anti-SIRPA antibodies derived from parental anti-SIRPA antibody SA-56.

FIG. 7D shows induction of human SIRPb1-dependent luciferase expression in a cell-based reporter assay by affinity matured anti-SIRPA antibodies derived from parental anti-SIRPA antibodies SA-19 and SA-56. BWZ-huSIRPA and BWZ-huSIRPb1 cells were stimulated overnight with full-length anti-SIRPA antibodies or human IgG1 isotype control adsorbed onto 96-well plate at 10 mg/mL. AM4-5 is an anti-SIRPA/b1 cross-reactive antibody that serves as a positive control.

FIG. 8A shows luminescence values of BWZ-huSIRPA reporter cells incubated in the presence of plate-bound CD47 and affinity matured anti-SIRPA antibodies derived from parental anti-SIRPA antibodies SA-5 and SA-8.

FIG. 8B shows luminescence values of BWZ-huSIRPA reporter cells incubated in the presence of plate-bound CD47 and affinity matured anti-SIRPA antibodies derived from parental anti-SIRPA antibody SA-19.

FIG. 8C and FIG. 8D show luminescence values of BWZ-huSIRPA reporter cells incubated in the presence of plate-bound CD47 and affinity matured anti-SIRPA antibodies derived from parent anti-SIRPA antibody SA-56. Results are presented as fold over background with radiance values from unstimulated reporter cells set to 1 on the y-axis. AM4-5 is an anti-SIRPA/b1 cross-reactive antibody that blocks CD47 binding to SIRPA and serves as a positive control.

FIG. 9 shows luminescence values from BWZ-huSIRPA reporter cells co-cultured with Raji B cells in the presence of affinity matured anti-SIRPA antibodies or isotype control. Results are presented as fold-over background with radiance values from isotype control-treated reporter cells mixed with Raji cells set to 1 on the y-axis. Reporter cells were treated with bin 2 anti-SIRPA antibodies derived from parent anti-SIRPA antibody SA-56 (SA-85=SA-56-85; SA-89=SA-56-89; and SA-94=SA-56-94).

FIG. 10A illustrates the critical residues for antigen binding by anti-SIRPA antibodies. Primary critical residues for anti-SIRPA antibodies SA-56-90 and SA-56-94 (A) binding to antigen (shown in black spheres) were identified as those that were negative for experimental antibody binding but positive for the control antibody. Critical residues are shown on a Phyre2-derived model for SIRPA, based on a human SIRPA crystal structure (PDB ID #2WNG; Hatherley et al., 2009).

FIG. 10B aligns the epitope of anti-SIRPA antibodies SA-56-90 and SA-56-94 (black spheres), with the CD47 binding site (white spheres) on the crystal structure of the D1 domain of SIRPA (PDB ID #2UV3).

FIG. 11 shows the phagocytosis of Raji B cells by human macrophages. Monocyte-derived macrophages were treated overnight with either isotype control or affinity matured anti-SIRPA antibodies, SA-56-90 and SA-56-94, at 5 mg/mL. Raji cells labeled with pHrodo were added to macrophages and incubated at 37 C for 2 hours. Phagocytosis was measured by counting percent of CD14+/pHrodo+ macrophage population with the value of isotype-treated macrophages set to 1 on the y-axis.

FIG. 12 shows the release of TNFa by LPS-stimulated human dendritic cells in the presence of anti-SIRPA antibody SA-56-90. Monocyte-derived dendritic cells were seeded onto wells coated with either isotype control, anti-SIRPA antibody (SA-56-90), or anti-SIRPb1 antibody (SB-1) and stimulated with 0.5 ng/mL LPS overnight at 37 C.

### DETAILED DESCRIPTION

The present disclosure relates to anti-SIRPA antibodies (e.g., monoclonal antibodies); methods of making and using such antibodies; pharmaceutical compositions comprising such antibodies; nucleic acids encoding such antibodies; and host cells comprising nucleic acids encoding such antibodies.

The present disclosure provides anti-SIRPA antibodies that specifically recognize human SIRPA. Anti-SIRPA antibodies of the present disclosure recognize a unique epitope on the D1 domain of human SIRPA that borders the CD47-binding site. As a result of binding this epitope, anti-SIRPA antibodies provided in this disclosure enhance the interaction between human SIRPA and human CD47, thereby stimulating the inhibitory SIRPA signaling pathway in myeloid cells. Such agonistic anti-SIRPA antibodies of the present disclosure are useful for treating, preventing, or reducing risk of a disease or pathology associated with low or reduced SIRPA expression, activity, or signaling.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### I. DEFINITIONS

The terms “SIRPa” or “SIRPa polypeptide” or “SIRPA” or “SIRPA polypeptide” are used interchangeably herein refer herein to any native SIRPA from any vertebrate source, including mammals such as primates (e.g., humans and cynos) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed SIRPA as well as any form of SIRPA that results from processing in the cell. In some embodiments, the SIRPA is human SIRPA. In some embodiments, the amino acid sequence of an exemplary SIRPA is Uniprot Accession No. P78324 as of 25 Apr. 2018. In some embodiments, the amino acid sequence of an exemplary human SIRPA v1 is SEQ ID NO: 1. In some embodiments, the amino acid sequence of an exemplary human SIRPA v2 is GenBank CAA71403.

The terms “anti-SIRPA antibody,” an “antibody that binds to SIRPA,” and “antibody that specifically binds SIRPA” refer to an antibody that is capable of binding SIRPA with sufficient affinity such that the antibody is useful as a diagnostic and/or therapeutic agent in targeting SIRPA. In one embodiment, the extent of binding of an anti-SIRPA antibody to an unrelated, non-SIRPA polypeptide is less than about 10% of the binding of the antibody to SIRPA as measured, e.g., by a radioimmunoassay (RIA). In certain embodiments, an antibody that binds to MerTK has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-MerTK antibody binds to an epitope of MerTK that is conserved among MerTK from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4th ed. (W.B. Saunders Co., 2000).

The “variable region” or “variable domain” of an antibody, such as an anti-MerTK antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-MerTK antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-SIRPA antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-SIRPA antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-SIRPA antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-SIRPA antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-MerTK antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-SIRPA antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-MerTK antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions. HVRs for particular antibodies herein may also be provided in the tables of this disclosure.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino acid modification” at a specified position, e.g., of an anti-SIRPA antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FOR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., SIRPA or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a SIRPA polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “agonist” antibody or an “activating” antibody is an antibody that induces (e.g., increases) one or more activities or functions of the antigen after the antibody binds the antigen.

An “isolated” antibody, such as an isolated anti-SIRPA antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-SIRPA antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “rector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### II. ANTI-SIRPA ANTIBODIES

Provided herein are anti-SIRPA antibodies. Antibodies provided herein are useful, e.g., for the diagnosis or treatment of SIRPA-associated disorders.

SIRPA is a single-pass type I membrane protein. Within the amino acid sequence of human SIRPA (SEQ ID NO:1), an extracellular domain is located at amino acid residues 31-373; a transmembrane domain is located at amino acid residues 374-394; and an intracellular domain is located at amino acid residues 395-504. Human SIRPA comprises a single V-set and two C1-sets of Ig super family (IgSF) domains, referred to as the D1 domain, the D2 domain, and the D3 domain, respectively. The D1 domain comprises amino acid residues 32-137 of human SIRPA; the D2 domain comprises amino acid residues 148-247 of human SIRPA; and the D3 domain comprises amino acid residues 254-348 of human SIRPA. As one of skill in the art will appreciate, the beginning and ending residues of the domains of the present disclosure may vary depending upon the computer modeling program used or the method used for determining the domain.

In some embodiments, anti-SIRPA antibodies of the present disclosure may bind a conformational epitope. In some embodiments, anti-SIRPA antibodies of the present disclosure may bind a discontinuous SIRPA epitope. In some embodiments, the discontinuous SIRPA epitope comprises two or more peptides, three or more peptides, four or more peptides, five or more peptides, six or more peptides, seven or more peptides, eight or more peptides, nine or more peptides, or 10 or more peptides. In some embodiments, anti-SIRPA antibodies of the present disclosure may bind a SIRPA epitope comprising one or more peptides. As disclosed herein, SIRPA epitopes may comprise one or more peptides comprising five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues of the amino acid sequence of SEQ ID NO: 1, or five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues on a mammalian SIRPA protein corresponding to the amino acid sequence of SEQ ID NO: 1.

Evidence provided herein shows that anti-SIRPA antibodies of the present disclosure bind to the D1 domain of human SIRPAv1. Accordingly, in some embodiments, an anti-SIRPA antibody of the present disclosure binds to the D1 domain of human SIRPA. In some embodiments, an anti-SIRPA antibody of the present disclosure binds to the D1 domain of human SIRPAv1. In some embodiments, an anti-SIRPA antibody of the present disclosure binds to a region comprising amino acid residues 32-137 of SEQ ID NO:1. In some embodiments, an anti-SIRPA antibody of the present disclosure binds to amino acid residues within human SIRPA, wherein the human SIRPA amino acid residues involved in binding by an anti-SIRPA antibody of the present disclosure comprise amino acid residues D40, R54, and W 68 of SEQ ID NO:1.

Evidence provided herein shows that anti-SIRPA antibodies of the present disclosure increased the binding of CD47 to SIRPA. In some embodiments, an anti-SIRPA antibody of the present disclosure increases the binding of CD47 to SIRPA. In some embodiments, an anti-SIRPA antibody of the present disclosure increases the binding of CD47 to human SIRPA. In some embodiments, an anti-SIRPA antibody of the present disclosure increases the binding of CD47 to human SIRPAv1. In some embodiments, an anti-SIRPA antibody of the present disclosure binds to the D1 domain of SIRPA and increases the binding of CD47 to SIRPA. In some embodiments, an anti-SIRPA antibody of the present disclosure binds to a region comprising amino acid residues 32-137 of SEQ ID NO:1 and increases the binding of CD47 to SIRPA. In yet other embodiments, an anti-SIRPA antibody of the present disclosure binds to certain amino acid residues within SIRPA, wherein the SIRPA amino acid residues involved in binding by an anti-SIRPA antibody of the present disclosure comprise amino acid residues D40, R54, and W68 of SEQ ID NO:1, and further wherein the anti-SIRPA antibody increases the binding of CD47 to SIRPA.

In one aspect, the present disclosure provides isolated (e.g., monoclonal) antibodies that bind to an epitope within a SIRPA protein or polypeptide of the present disclosure. SIRPA proteins or polypeptides of the present disclosure include, without limitation, a mammalian SIRPA protein or polypeptide, human SIRPA protein or polypeptide, mouse (murine) SIRPA protein or polypeptide, and cynomolgus SIRPA protein or polypeptide. SIRPA proteins and polypeptides of the present disclosure include naturally occurring variants of SIRPA. In some embodiments, SIRPA proteins and polypeptides of the present disclosure are membrane bound. In some embodiments, SIRPA proteins and polypeptides of the present disclosure are a soluble extracellular domain of SIRPA.

### A. Competitively Inhibit Binding

In some embodiments, an anti-SIRPA antibody of the present disclosure binds to an epitope of human SIRPA that is the same or overlaps with the SIRPA epitope bound by at least one antibody selected from any of the antibodies provided herein.

In some embodiments, the anti-SIRPA antibody competes with another antibody for binding to SIRPA. Any suitable competition assay or SIRPA binding assay known in the art, such as BIAcore® analysis (surface plasmon resonance), ELISA assays, or flow cytometry, may be utilized to determine whether an anti-SIRPA antibody competes with one or more antibodies selected from SA-1, SA-2, SA-3, SA-4, SA-5 (including SA-5-57, SA-5-58, SA-5-59, and SA-5-61), SA-6, SA-7, SA-8 (including SA-8-62, SA-8-64, SA-8-66, and SA-8-67), SA-9, SA-10, SA-11, SA-12, SA-13 (including SA-13-6, SA-13-69, and SA-13-71), SA-14, SA-15, SA-16, SA-17, SA-18, SA-19 (including SA-19-72, SA-19-73, SA-19-74, SA-19-75, SA-19-76, SA-19-77, SA-19-78, SA-19-79, SA-19-80, SA-19-81, SA-19-82, SA-19-83, and SA-19-84), SA-20, SA-21, SA-22, SA-23, SA-24, SA-25, SA-26, SA-27, SA-28, SA-29, SA-30, SA-31, SA-32, SA-33, SA-34, SA-35, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56 (including SA-56-85, SA-56-86, SA-56-87, SA-56-88, SA-56-89, SA-56, 90, SA-56, 91, SA-56-92, SA-56-93, SA-56-94, SA-56-95, and SA-56-96), SA-57, SA-58, SA-59, SA-60, SA-61, and SA-62, and any combination thereof for binding to SIRPA. In an exemplary competition assay, immobilized SIRPA or cells expressing SIRPA on the cell surface are incubated in a solution comprising a first labeled antibody that binds to SIRPA (e.g., human or non-human primate) and a second unlabeled antibody that is being tested for its ability to compete with the first antibody for binding to SIRPA. The second antibody may be present in a hybridoma supernatant. As a control, immobilized SIRPA or cells expressing SIRPA is incubated in a solution comprising the first labeled antibody but not the second unlabeled antibody. After incubation under conditions permissive for binding of the first antibody to SIRPA, excess unbound antibody is removed, and the amount of label associated with immobilized SIRPA or cells expressing SIRPA is measured. If the amount of label associated with immobilized SIRPA or cells expressing SIRPA is substantially reduced in the test sample relative to the control sample, then that indicates that the second antibody is competing with the first antibody for binding to SIRPA. See, Harlow and Lane (1988) Antibodies: A Laboratory Manual ch. 14 (Cold Spring Harbor Laboratory, Cold Spring Harbor, NY).

In some embodiments, SIRPA is expressed in a cell. In some embodiments, SIRPA is expressed in phagocytic cells, including without limitation, macrophages and dendritic cells. In some embodiments, SIRPA is expressed in monocytes, natural killer cells, natural killer T cells, microglia, endothelial cells, and megakaryocytes.

Evidence provided herein demonstrates that anti-SIRPA antibodies of the present disclosure are effective at decreasing phagocytosis activity by phagocytic cells. In some embodiments, an anti-SIRPA antibody of the present disclosure decreases phagocytosis activity by phagocytic cells. In some embodiments, an anti-SIRPA antibody of the present disclosure decreases tumor cell phagocytosis by phagocytic cells. In some embodiments, the phagocytic cells are macrophages. In some embodiments, the phagocytic cells are dendritic cells.

The present disclosure shows that anti-SIRPA antibodies described herein are effective at increasing SIRPA activity and SIRPA signaling in cells. Evidence is also provided herein showing anti-SIRPA antibodies of the present disclosure are effective at increasing CD47-induced (or CD47-mediated) SIRPA signaling in cells. Accordingly, in some embodiments, an anti-SIRPA antibody of the present disclosure is effective at increasing SIRPA activity in a cell. In some embodiments, an anti-SIRPA antibody of the present disclosure is effective at increasing SIRPA signaling in a cell. In other embodiments, an anti-SIRPA antibody of the present disclosure increases CD47-induced SIRPA signaling (e.g., CD47-mediated SIRPA signaling) in a cell. In some embodiments, an anti-SIRPA antibody of the present disclosure increases SIRPA activity in macrophages, dendritic cells, and/or microglial cells. In some embodiments, and anti-SIRPA antibody of the present disclosure increases SIRPA signaling in macrophages, dendritic cells, and/or microglial cells. In some embodiments, an anti-SIRPA antibody of the present disclosure increases CD47-induced or CD47-mediated SIRPA signaling in macrophages, dendritic cells, and/or microglial cells.

In some embodiments, an anti-SIRPA antibody of the present disclosure increases synaptic density. In some embodiments, an anti-SIRPA antibody of the present disclosure increases synaptic density in the brain. In some embodiments, an anti-SIRPA antibody of the present disclosure increases synaptic density in neurons.

In some embodiments, an anti-SIRPA antibody of the present disclosure decreases synaptic elimination.

Synapsins are a family of neuron-specific phosphoproteins associated with the regulation of neurotransmitter release at synapses. Synapsins are thought to be involved in regulating the number of synaptic vesicles available for release via exocytosis. (See, e.g., Mirza and Zahid, 2018, Neurosci Bull, 34:349-358.) Provided herein are anti-SIRPA antibodies effective at increases synapsin levels. In some embodiments, an anti-SIRPA antibody of the present disclosure increases synapsin levels in neurons.

Postsynaptic density (PSD) is an electron-dense region localized at the postsynaptic sites of excitatory synapses. Postsynaptic density-95 (PSD-95) is one of the most abundant proteins of the PSD and plays a role in promoting synapse maturation and regulating synaptic strength and plasticity. Provided herein are anti-SIRPA antibodies effective at increasing PSD-95 levels in neurons. In some embodiments, an anti-SIRPA antibody of the present disclosure increases PSD-95 levels in neurons. In some embodiments, an anti-SIRPA antibody of the present disclosure increases synapsin levels and increases PSD-95 levels in neurons.

Evidence provided herein demonstrates that anti-SIRPA antibodies of the present disclosure are effective at decreasing cytokine release from dendritic cells. In particular, the present disclosure shows that anti-SIRPA antibodies of the present disclosure decrease TNFa release from dendritic cells. In some embodiments, anti-SIRPA antibodies of the present disclosure decrease cytokine release from dendritic cells. In some embodiments, anti-SIRPA antibodies of the present disclosure decrease TNFa release from dendritic cells. In some embodiments, anti-SIRPA antibodies of the present disclosure decrease TNFa release by about 42% compared to that observed in the absence of an anti-SIRPA antibody of the present disclosure.

### B. Exemplary Antibodies and Certain Other Antibody Embodiments

The present disclosure provides anti-SIRPA antibodies. In some embodiments, provided herein are anti-SIRPA antibodies comprising at least one, two, three, four, five, or six HVRs selected from: (a) HVR-L1 comprising an amino acid sequence selected from the group consisting of SEQ ID NOs:7-28; (b) HVR-L2 comprising an amino acid sequence selected from the group consisting of SEQ ID NOs:29-40; (c) HVR-L3 comprising an amino acid sequence selected from the group consisting of SEQ ID NOs:41-101; (d) HVR-H1 comprising an amino acid sequence selected from the group consisting of SEQ ID NOs:102-122; (e) HVR-H2 comprising an amino acid sequence selected from the group consisting of SEQ ID NOs:123-146; and (f) HVR-H3 comprising an amino acid sequence selected from the group consisting of SEQ ID NOs:147-201.

In some embodiments, provided herein are anti-SIRPA antibodies comprising at least one, at least two, or all three V_{L} HVR sequences selected from (a) HVR-L1 comprising an amino acid sequence selected from the group consisting of SEQ ID NOs:7-28; (b) HVR-L2 comprising an amino acid sequence selected from the group consisting of SEQ ID NOs:29-40; and (c) HVR-L3 comprising an amino acid sequence selected from the group consisting of SEQ ID NOs:41-101.

In some embodiments, provided herein are anti-SIRPA antibodies comprising at least one, at least two, or all three V_{H} HVR sequences selected from (a) HVR-H1 comprising an amino acid sequence selected from the group consisting of SEQ ID NOs:102-122; (b) HVR-H2 comprising an amino acid sequence selected from the group consisting of SEQ ID NOs:123-146; and (c) HVR-H3 comprising an amino acid sequence selected from the group consisting of SEQ ID NOs:147-201.

In some embodiments, provided herein are anti-SIRPA antibodies comprising (a) a V_{L} domain comprising at least one, at least two, or all three V_{L} HVR sequences selected from (i) HVR-L1 comprising an amino acid sequence selected from the group consisting of SEQ ID NOs:7-28, (ii) HVR-L2 comprising an amino acid sequence selected from the group consisting of SEQ ID NOs:29-40, and (iii) HVR-L3 comprising an amino acid sequence selected from the group consisting of SEQ ID NOs:41-101, and (b) a V_{H} domain comprising at least one, at least two, or all three V_{H} HVR sequences selected from (i) HVR-H1 comprising an amino acid sequence selected from the group consisting of SEQ ID NOs:102-122, (ii) HVR-H2 comprising an amino acid sequence selected from the group consisting of SEQ ID NOs:123-146 and (iii) HVR-H3 comprising an amino acid sequence selected from the group consisting of SEQ ID NOs:147-201.

In some embodiments, provided herein are anti-SIRPA antibodies comprising: (a) HVR-L1 comprising the amino acid sequence of SEQ ID NO:7; (b) HVR-L2 comprising the amino acid sequence of SEQ ID NO:29; (c) HVR-L3 comprising the amino acid sequence of SEQ ID NO:41; (d) HVR-H1 comprising the amino acid sequence of SEQ ID NO:102; (e) HVR-H2 comprising the amino acid sequence of SEQ ID NO:123; and (f) HVR-H3 comprising the amino acid sequence of SEQ ID NO:147; (a) HVR-L1 comprising the amino acid sequence of SEQ ID NO:8; (b) HVR-L2 comprising the amino acid sequence of SEQ ID NO:30; (c) HVR-L3 comprising the amino acid sequence of SEQ ID NO:42; (d) HVR-H1 comprising the amino acid sequence of SEQ ID NO:103; (e) HVR-H2 comprising the amino acid sequence of SEQ ID NO:124; and (f) HVR-H3 comprising the amino acid sequence of SEQ ID NO:148; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:43; (d) HVR-H1 comprising the amino acid sequence of SEQ ID NO:104; (e) HVR-H2 comprising the amino acid sequence of SEQ ID NO:125; and (f) HVR-H3 comprising the amino acid sequence of SEQ ID NO:149; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:44; (d) HVR-H1 comprising the amino acid sequence of SEQ ID NO:105; (e) HVR-H2 comprising the amino acid sequence of SEQ ID NO:125; and (f) HVR-H3 comprising the amino acid sequence of SEQ ID NO:150; (a) HVR-L1 comprising the amino acid sequence of SEQ ID NO:9; (b) HVR-L2 comprising the amino acid sequence of SEQ ID NO:32; (c) HVR-L3 comprising the amino acid sequence of SEQ ID NO:45; (d) HVR-H1 comprising the amino acid sequence of SEQ ID NO:106; (e) HVR-H2 comprising the amino acid sequence of SEQ ID NO:125; and (f) HVR-H3 comprising the amino acid sequence of SEQ ID NO:151; (a) HVR-L1 comprising the amino acid sequence of SEQ ID NO:7; (b) HVR-L2 comprising the amino acid sequence of SEQ ID NO:29; (c) HVR-L3 comprising the amino acid sequence of SEQ ID NO:46; (d) HVR-H1 comprising the amino acid sequence of SEQ ID NO:107; (e) HVR-H2 comprising the amino acid sequence of SEQ ID NO:126; and (f) HVR-H3 comprising the amino acid sequence of SEQ ID NO:152; (a) HVR-L1 comprising the amino acid sequence of SEQ ID NO:10; (b) HVR-L2 comprising the amino acid sequence of SEQ ID NO:30; (c) HVR-L3 comprising the amino acid sequence of SEQ ID NO:47; (d) HVR-H1 comprising the amino acid sequence of SEQ ID NO:103; (e) HVR-H2 comprising the amino acid sequence of SEQ ID NO:127; and (f) HVR-H3 comprising the amino acid sequence of SEQ ID NO:153; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:103; (e) HVR-H2 comprising the amino acid sequence of SEQ ID NO:124; and (f) HVR-H3 comprising the amino acid sequence of SEQ ID NO:154; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:49; (d) HVR-H1 comprising the amino acid sequence of SEQ ID NO:106; (e) HVR-H2 comprising the amino acid sequence of SEQ ID NO:128; and (f) HVR-H3 comprising the amino acid sequence of SEQ ID NO:155; (a) HVR-L1 comprising the amino acid sequence of SEQ ID NO:13; (b) HVR-L2 comprising the amino acid sequence of SEQ ID NO:34; (c) HVR-L3 comprising the amino acid sequence of SEQ ID NO:50; (d) HVR-H1 comprising the amino acid sequence of SEQ ID NO:107; (e) HVR-H2 comprising the amino acid sequence of SEQ ID NO:126; and (f) HVR-H3 comprising the amino acid sequence of SEQ ID NO:156; (a) HVR-L1 comprising the amino acid sequence of SEQ ID NO:13; (b) HVR-L2 comprising the amino acid sequence of SEQ ID NO:35; (c) HVR-L3 comprising the amino acid sequence of SEQ ID NO:51; (d) HVR-H1 comprising the amino acid sequence of SEQ ID NO:102; (e) HVR-H2 comprising the amino acid sequence of SEQ ID NO:123; and (f) HVR-H3 comprising the amino acid sequence of SEQ ID NO:157; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:52; (d) HVR-H1 comprising the amino acid sequence of SEQ ID NO:108; (e) HVR-H2 comprising the amino acid sequence of SEQ ID NO:129; and (f) HVR-H3 comprising the amino acid sequence of SEQ ID NO:158; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:53; (d) HVR-H1 comprising the amino acid sequence of SEQ ID NO:106; (e) HVR-H2 comprising the amino acid sequence of SEQ ID NO:125; and (f) HVR-H3 comprising the amino acid sequence of SEQ ID NO:159; (a) HVR-L1 comprising the amino acid sequence of SEQ ID NO:15; (b) HVR-L2 comprising the amino acid sequence of SEQ ID NO:33; (c) HVR-L3 comprising the amino acid sequence of SEQ ID NO:54; (d) HVR-H1 comprising the amino acid sequence of SEQ ID NO:109; (e) HVR-H2 comprising the amino acid sequence of SEQ ID NO:130; and (f) HVR-H3 comprising the amino acid sequence of SEQ ID NO:160; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:55; (d) HVR-H1 comprising the amino acid sequence of SEQ ID NO:110; (e) HVR-H2 comprising the amino acid sequence of SEQ ID NO:131; and (f) HVR-H3 comprising the amino acid sequence of SEQ ID NO:161; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:56; (d) HVR-H1 comprising the amino acid sequence of SEQ ID NO:111; (e) HVR-H2 comprising the amino acid sequence of SEQ ID NO:132; and (f) HVR-H3 comprising the amino acid sequence of SEQ ID NO:162; (a) HVR-L1 comprising the amino acid sequence of SEQ ID NO:16; (b) HVR-L2 comprising the amino acid sequence of SEQ ID NO:36; (c) HVR-L3 comprising the amino acid sequence of SEQ ID NO:57; (d) HVR-H1 comprising the amino acid sequence of SEQ ID NO:110; (e) HVR-H2 comprising the amino acid sequence of SEQ ID NO:133; and (f) HVR-H3 comprising the amino acid sequence of SEQ ID NO:163; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:58; (d) HVR-H1 comprising the amino acid sequence of SEQ ID NO:112; (e) HVR-H2 comprising the amino acid sequence of SEQ ID NO:134; and (f) HVR-H3 comprising the amino acid sequence of SEQ ID NO:164; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:124; and (f) HVR-H3 comprising the amino acid sequence of SEQ ID NO:165; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:60; (d) HVR-H1 comprising the amino acid sequence of SEQ ID NO:103; (e) HVR-H2 comprising the amino acid sequence of SEQ ID NO:124; and (f) HVR-H3 comprising the amino acid sequence of SEQ ID NO:154; (a) HVR-L1 comprising the amino acid sequence of SEQ ID NO:8; (b) HVR-L2 comprising the amino acid sequence of SEQ ID NO:30; (c) HVR-L3 comprising the amino acid sequence of SEQ ID NO:61; (d) HVR-H1 comprising the amino acid sequence of SEQ ID NO:113; (e) HVR-H2 comprising the amino acid sequence of SEQ ID NO:135; and (f) HVR-H3 comprising the amino acid sequence of SEQ ID NO:166; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:62; (d) HVR-H1 comprising the amino acid sequence of SEQ ID NO:109; (e) HVR-H2 comprising the amino acid sequence of SEQ ID NO:130; and (f) HVR-H3 comprising the amino acid sequence of SEQ ID NO:167; (a) HVR-L1 comprising the amino acid sequence of SEQ ID NO:17; (b) HVR-L2 comprising the amino acid sequence of SEQ ID NO:37; (c) HVR-L3 comprising the amino acid sequence of SEQ ID NO:63; (d) HVR-H1 comprising the amino acid sequence of SEQ ID NO:114; (e) HVR-H2 comprising the amino acid sequence of SEQ ID NO:136; and (f) HVR-H3 comprising the amino acid sequence of SEQ ID NO:168; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:64; (d) HVR-H1 comprising the amino acid sequence of SEQ ID NO:103; (e) HVR-H2 comprising the amino acid sequence of SEQ ID NO:137; and (f) HVR-H3 comprising the amino acid sequence of SEQ ID NO:169; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:65; (d) HVR-H1 comprising the amino acid sequence of SEQ ID NO:110; (e) HVR-H2 comprising the amino acid sequence of SEQ ID NO:138; and (f) HVR-H3 comprising the amino acid sequence of SEQ ID NO:170; (a) HVR-L1 comprising the amino acid sequence of SEQ ID NO:18; (b) HVR-L2 comprising the amino acid sequence of SEQ ID NO:32; (c) HVR-L3 comprising the amino acid sequence of SEQ ID NO:66; (d) HVR-H1 comprising the amino acid sequence of SEQ ID NO:111; (e) HVR-H2 comprising the amino acid sequence of SEQ ID NO:132; and (f) HVR-H3 comprising the amino acid sequence of SEQ ID NO:171; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:67; (d) HVR-H1 comprising the amino acid sequence of SEQ ID NO:115; (e) HVR-H2 comprising the amino acid sequence of SEQ ID NO:139; and (f) HVR-H3 comprising the amino acid sequence of SEQ ID NO:172; (a) HVR-L1 comprising the amino acid sequence of SEQ ID NO:19; (b) HVR-L2 comprising the amino acid sequence of SEQ ID NO:38; (c) HVR-L3 comprising the amino acid sequence of SEQ ID NO:68; (d) HVR-H1 comprising the amino acid sequence of SEQ ID NO:116; (e) HVR-H2 comprising the amino acid sequence of SEQ ID NO:140; and (f) HVR-H3 comprising the amino acid sequence of SEQ ID NO:173; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:69; (d) HVR-H1 comprising the amino acid sequence of SEQ ID NO:116; (e) HVR-H2 comprising the amino acid sequence of SEQ ID NO:140; and (f) HVR-H3 comprising the amino acid sequence of SEQ ID NO:173; (a) HVR-L1 comprising the amino acid sequence of SEQ ID NO:10; (b) HVR-L2 comprising the amino acid sequence of SEQ ID NO:30; (c) HVR-L3 comprising the amino acid sequence of SEQ ID NO:70; (d) HVR-H1 comprising the amino acid sequence of SEQ ID NO:103; (e) HVR-H2 comprising the amino acid sequence of SEQ ID NO:137; and (f) HVR-H3 comprising the amino acid sequence of SEQ ID NO:174; (a) HVR-L1 comprising the amino acid sequence of SEQ ID NO:20; (b) HVR-L2 comprising the amino acid sequence of SEQ ID NO:38; (c) HVR-L3 comprising the amino acid sequence of SEQ ID NO:71; (d) HVR-H1 comprising the amino acid sequence of SEQ ID NO:110; (e) HVR-H2 comprising the amino acid sequence of SEQ ID NO:138; and (f) HVR-H3 comprising the amino acid sequence of SEQ ID NO:170; (a) HVR-L1 comprising the amino acid sequence of SEQ ID NO:21; (b) HVR-L2 comprising the amino acid sequence of SEQ ID NO:36; (c) HVR-L3 comprising the amino acid sequence of SEQ ID NO:72; (d) HVR-H1 comprising the amino acid sequence of SEQ ID NO:117; (e) HVR-H2 comprising the amino acid sequence of SEQ ID NO:137; and (f) HVR-H3 comprising the amino acid sequence of SEQ ID NO:175; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:73; (d) HVR-H1 comprising the amino acid sequence of SEQ ID NO:110; (e) HVR-H2 comprising the amino acid sequence of SEQ ID NO:133; and (f) HVR-H3 comprising the amino acid sequence of SEQ ID NO:176; (a) HVR-L1 comprising the amino acid sequence of SEQ ID NO:13; (b) HVR-L2 comprising the amino acid sequence of SEQ ID NO:35; (c) HVR-L3 comprising the amino acid sequence of SEQ ID NO:74; (d) HVR-H1 comprising the amino acid sequence of SEQ ID NO:118; (e) HVR-H2 comprising the amino acid sequence of SEQ ID NO:141; and (f) HVR-H3 comprising the amino acid sequence of SEQ ID NO:177; (a) HVR-L1 comprising the amino acid sequence of SEQ ID NO:13; (b) HVR-L2 comprising the amino acid sequence of SEQ ID NO:39; (c) HVR-L3 comprising the amino acid sequence of SEQ ID NO:75; (d) HVR-H1 comprising the amino acid sequence of SEQ ID NO:119; (e) HVR-H2 comprising the amino acid sequence of SEQ ID NO:129; and (f) HVR-H3 comprising the amino acid sequence of SEQ ID NO:178; (a) HVR-L1 comprising the amino acid sequence of SEQ ID NO:22; (b) HVR-L2 comprising the amino acid sequence of SEQ ID NO:31; (c) HVR-L3 comprising the amino acid sequence of SEQ ID NO:76; (d) HVR-H1 comprising the amino acid sequence of SEQ ID NO:113; (e) HVR-H2 comprising the amino acid sequence of SEQ ID NO:135; and (f) HVR-H3 comprising the amino acid sequence of SEQ ID NO:179; (a) HVR-L1 comprising the amino acid sequence of SEQ ID NO:23; (b) HVR-L2 comprising the amino acid sequence of SEQ ID NO:38; (c) HVR-L3 comprising the amino acid sequence of SEQ ID NO:77; (d) HVR-H1 comprising the amino acid sequence of SEQ ID NO:116; (e) HVR-H2 comprising the amino acid sequence of SEQ ID NO:139; and (f) HVR-H3 comprising the amino acid sequence of SEQ ID NO:180; (a) HVR-L1 comprising the amino acid sequence of SEQ ID NO:16; (b) HVR-L2 comprising the amino acid sequence of SEQ ID NO:36; (c) HVR-L3 comprising the amino acid sequence of SEQ ID NO:78; (d) HVR-H1 comprising the amino acid sequence of SEQ ID NO:116; (e) HVR-H2 comprising the amino acid sequence of SEQ ID NO:139; and (f) HVR-H3 comprising the amino acid sequence of SEQ ID NO:180; (a) HVR-L1 comprising the amino acid sequence of SEQ ID NO:24; (b) HVR-L2 comprising the amino acid sequence of SEQ ID NO:33; (c) HVR-L3 comprising the amino acid sequence of SEQ ID NO:79; (d) HVR-H1 comprising the amino acid sequence of SEQ ID NO:120; (e) HVR-H2 comprising the amino acid sequence of SEQ ID NO:129; and (f) HVR-H3 comprising the amino acid sequence of SEQ ID NO:181; (a) HVR-L1 comprising the amino acid sequence of SEQ ID NO:20; (b) HVR-L2 comprising the amino acid sequence of SEQ ID NO:38; (c) HVR-L3 comprising the amino acid sequence of SEQ ID NO:80; (d) HVR-H1 comprising the amino acid sequence of SEQ ID NO:119; (e) HVR-H2 comprising the amino acid sequence of SEQ ID NO:142; and (f) HVR-H3 comprising the amino acid sequence of SEQ ID NO:182; (a) HVR-L1 comprising the amino acid sequence of SEQ ID NO:16; (b) HVR-L2 comprising the amino acid sequence of SEQ ID NO:36; (c) HVR-L3 comprising the amino acid sequence of SEQ ID NO:81; (d) HVR-H1 comprising the amino acid sequence of SEQ ID NO:121; (e) HVR-H2 comprising the amino acid sequence of SEQ ID NO:143; and (f) HVR-H3 comprising the amino acid sequence of SEQ ID NO:183; (a) HVR-L1 comprising the amino acid sequence of SEQ ID NO:25; (b) HVR-L2 comprising the amino acid sequence of SEQ ID NO:36; (c) HVR-L3 comprising the amino acid sequence of SEQ ID NO:82; (d) HVR-H1 comprising the amino acid sequence of SEQ ID NO:121; (e) HVR-H2 comprising the amino acid sequence of SEQ ID NO:143; and (f) HVR-H3 comprising the amino acid sequence of SEQ ID NO:183; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:83; (d) HVR-H1 comprising the amino acid sequence of SEQ ID NO:122; (e) HVR-H2 comprising the amino acid sequence of SEQ ID NO:139; and (f) HVR-H3 comprising the amino acid sequence of SEQ ID NO:184; (a) HVR-L1 comprising the amino acid sequence of SEQ ID NO:26; (b) HVR-L2 comprising the amino acid sequence of SEQ ID NO:32; (c) HVR-L3 comprising the amino acid sequence of SEQ ID NO:84; (d) HVR-H1 comprising the amino acid sequence of SEQ ID NO:109; (e) HVR-H2 comprising the amino acid sequence of SEQ ID NO:144; and (f) HVR-H3 comprising the amino acid sequence of SEQ ID NO:185; (a) HVR-L1 comprising the amino acid sequence of SEQ ID NO:20; (b) HVR-L2 comprising the amino acid sequence of SEQ ID NO:38; (c) HVR-L3 comprising the amino acid sequence of SEQ ID NO:85; (d) HVR-H1 comprising the amino acid sequence of SEQ ID NO:114; (e) HVR-H2 comprising the amino acid sequence of SEQ ID NO:141; and (f) HVR-H3 comprising the amino acid sequence of SEQ ID NO:186; (a) HVR-L1 comprising the amino acid sequence of SEQ ID NO:20; (b) HVR-L2 comprising the amino acid sequence of SEQ ID NO:38; (c) HVR-L3 comprising the amino acid sequence of SEQ ID NO: 86; (d) HVR-H1 comprising the amino acid sequence of SEQ ID NO:104; (e) HVR-H2 comprising the amino acid sequence of SEQ ID NO:125; and (f) HVR-H3 comprising the amino acid sequence of SEQ ID NO:187; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:87; (d) HVR-H1 comprising the amino acid sequence of SEQ ID NO:119; (e) HVR-H2 comprising the amino acid sequence of SEQ ID NO:129; and (f) HVR-H3 comprising the amino acid sequence of SEQ ID NO:188; (a) HVR-L1 comprising the amino acid sequence of SEQ ID NO:10; (b) HVR-L2 comprising the amino acid sequence of SEQ ID NO:30; (c) HVR-L3 comprising the amino acid sequence of SEQ ID NO:88; (d) HVR-H1 comprising the amino acid sequence of SEQ ID NO:103; (e) HVR-H2 comprising the amino acid sequence of SEQ ID NO:124; and (f) HVR-H3 comprising the amino acid sequence of SEQ ID NO:189; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:89; (d) HVR-H1 comprising the amino acid sequence of SEQ ID NO:109; (e) HVR-H2 comprising the amino acid sequence of SEQ ID NO:130; and (f) HVR-H3 comprising the amino acid sequence of SEQ ID NO:190; (a) HVR-L1 comprising the amino acid sequence of SEQ ID NO:10; (b) HVR-L2 comprising the amino acid sequence of SEQ ID NO:30; (c) HVR-L3 comprising the amino acid sequence of SEQ ID NO:90; (d) HVR-H1 comprising the amino acid sequence of SEQ ID NO:109; (e) HVR-H2 comprising the amino acid sequence of SEQ ID NO:144; and (f) HVR-H3 comprising the amino acid sequence of SEQ ID NO:191; (a) HVR-L1 comprising the amino acid sequence of SEQ ID NO:20; (b) HVR-L2 comprising the amino acid sequence of SEQ ID NO:38; (c) HVR-L3 comprising the amino acid sequence of SEQ ID NO:91; (d) HVR-H1 comprising the amino acid sequence of SEQ ID NO:110; (e) HVR-H2 comprising the amino acid sequence of SEQ ID NO:138; and (f) HVR-H3 comprising the amino acid sequence of SEQ ID NO:192; (a) HVR-L1 comprising the amino acid sequence of SEQ ID NO:20; (b) HVR-L2 comprising the amino acid sequence of SEQ ID NO:40; (c) HVR-L3 comprising the amino acid sequence of SEQ ID NO:92; (d) HVR-H1 comprising the amino acid sequence of SEQ ID NO:118; (e) HVR-H2 comprising the amino acid sequence of SEQ ID NO:141; and (f) HVR-H3 comprising the amino acid sequence of SEQ ID NO:193; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:93; (d) HVR-H1 comprising the amino acid sequence of SEQ ID NO:116; (e) HVR-H2 comprising the amino acid sequence of SEQ ID NO:145; and (f) HVR-H3 comprising the amino acid sequence of SEQ ID NO:194; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:94; (d) HVR-H1 comprising the amino acid sequence of SEQ ID NO:115; (e) HVR-H2 comprising the amino acid sequence of SEQ ID NO:139; and (f) HVR-H3 comprising the amino acid sequence of SEQ ID NO:195; (a) HVR-L1 comprising the amino acid sequence of SEQ ID NO:27; (b) HVR-L2 comprising the amino acid sequence of SEQ ID NO:35; (c) HVR-L3 comprising the amino acid sequence of SEQ ID NO:95; (d) HVR-H1 comprising the amino acid sequence of SEQ ID NO:112; (e) HVR-H2 comprising the amino acid sequence of SEQ ID NO:146; and (f) HVR-H3 comprising the amino acid sequence of SEQ ID NO:196; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:109; (e) HVR-H2 comprising the amino acid sequence of SEQ ID NO:130; and (f) HVR-H3 comprising the amino acid sequence of SEQ ID NO:197; (a) HVR-L1 comprising the amino acid sequence of SEQ ID NO:7; (b) HVR-L2 comprising the amino acid sequence of SEQ ID NO:31; (c) HVR-L3 comprising the amino acid sequence of SEQ ID NO:97; (d) HVR-H1 comprising the amino acid sequence of SEQ ID NO:122; (e) HVR-H2 comprising the amino acid sequence of SEQ ID NO:139; and (f) HVR-H3 comprising the amino acid sequence of SEQ ID NO:198; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:98; (d) HVR-H1 comprising the amino acid sequence of SEQ ID NO:122; (e) HVR-H2 comprising the amino acid sequence of SEQ ID NO:139; and (f) HVR-H3 comprising the amino acid sequence of SEQ ID NO:199; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:99; (d) HVR-H1 comprising the amino acid sequence of SEQ ID NO:121; (e) HVR-H2 comprising the amino acid sequence of SEQ ID NO:143; and (f) HVR-H3 comprising the amino acid sequence of SEQ ID NO:200; (a) HVR-L1 comprising the amino acid sequence of SEQ ID NO:7; (b) HVR-L2 comprising the amino acid sequence of SEQ ID NO:29; (c) HVR-L3 comprising the amino acid sequence of SEQ ID NO:100; (d) HVR-H1 comprising the amino acid sequence of SEQ ID NO:109; (e) HVR-H2 comprising the amino acid sequence of SEQ ID NO:144; and (f) HVR-H3 comprising the amino acid sequence of SEQ ID NO:201; (a) HVR-L1 comprising the amino acid sequence of SEQ ID NO:13; (b) HVR-L2 comprising the amino acid sequence of SEQ ID NO:39; (c) HVR-L3 comprising the amino acid sequence of SEQ ID NO:75; (d) HVR-H1 comprising the amino acid sequence of SEQ ID NO:119; (e) HVR-H2 comprising the amino acid sequence of SEQ ID NO:129; and (f) HVR-H3 comprising the amino acid sequence of SEQ ID NO:178; and (a) HVR-L1 comprising the amino acid sequence of SEQ ID NO:28; (b) HVR-L2 comprising the amino acid sequence of SEQ ID NO:33; (c) HVR-L3 comprising the amino acid sequence of SEQ ID NO:101; (d) HVR-H1 comprising the amino acid sequence of SEQ ID NO:109; (e) HVR-H2 comprising the amino acid sequence of SEQ ID NO:144; and (f) HVR-H3 comprising the amino acid sequence of SEQ ID NO:201.

In another aspect, an anti-SIRPA antibody comprises a light chain variable domain (V_{L}) sequence having at least 90%, 91%, 92%, 93%, 94%, 95%, 96%, 97%, 98%, 99%, or 100% sequence identity to an amino acid sequence selected from the group consisting of SEQ ID NOs:257-317. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs:257-317 contains substitutions (e.g., conservative substitutions), insertions, or deletions relative to the reference sequence, but an anti-SIRPA antibody comprising that sequence retains the ability to bind to SIRPA. In certain embodiments, a total of 1 to 10 amino acids have been substituted, inserted, and/or deleted in SEQ ID NO:257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, or 317. In certain embodiments, a total of 1 to 5 amino acids have been substituted, inserted and/or deleted in SEQ ID NO: 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, or 317. In certain embodiments, substitutions, insertions, or deletions occur in regions outside the HVRs (i.e., in the FRs). Optionally, the anti-SIRPA antibody comprises the V_{L} sequence of SEQ ID NO: 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, or 317, including post-translational modifications of that sequence. In a particular embodiment, the VL comprises one, two or three HVRs selected from: (a) HVR-L1 comprising an amino acid sequence selected from the group consisting of SEQ ID NOs:7-28, (b) HVR-L2 comprising an amino acid sequence selected from the group consisting of SEQ ID NOs:29-40, and (c) HVR-L3 comprising an amino acid sequence selected from the group consisting of SEQ ID NOs:41-101.

In another aspect, an anti-SIRPA antibody is provided, wherein the antibody comprises a heavy chain variable domain (V_{H}) having at least 90%, 91%, 92%, 93%, 94%, 95%, 96%, 97%, 98%, 99%, or 100% sequence identity to an amino acid sequence selected from the group consisting of SEQ ID NOs:202-256. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs:202-256, and contains substitutions (e.g., conservative substitutions), insertions, or deletions relative to the reference sequence, but an anti-SIRPA antibody comprising that sequence retains the ability to bind to SIRPA. In some embodiments, a total of 1 to 10 amino acids have been substituted, inserted and/or deleted in SEQ ID NO:202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, or 257. In certain embodiments, a total of 1 to 5 amino acids have been substituted, inserted and/or deleted in SEQ ID NO: 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, or 257. In certain embodiments, the substitutions, insertions, or deletions occur in regions outside the HVRs (i.e., in the FRs). Optionally, the anti-SIRPA antibody comprises the V_{H} sequence of SEQ ID NO: 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, or 257, including post-translational modifications of that sequence. In a particular embodiment, the VH comprises one, two or three HVRs selected from (a) HVR-H1 comprising an amino acid sequence selected from the group consisting of SEQ ID NOs:102-122, (b) HVR-H2 comprising an amino acid sequence selected from the group consisting of SEQ ID NOs:123-146, and (c) HVR-H3 comprising an amino acid sequence selected from the group consisting of SEQ ID NOs:147-201.

In some embodiments, an anti-SIRPA antibody is provided, wherein the antibody comprises a V_{L} as in any of the embodiments provided above, and a V_{H} as in any of the embodiments provided above. In some embodiments, provided herein are anti-SIRPA antibodies, wherein the antibody comprises a V_{L} as in any of the embodiments provided above, and a V_{H} as in any of the embodiments provided above. In one embodiment, the antibody comprises the V_{L} and V_{H} sequences in SEQ ID NOs:257-317 and SEQ ID NOs:202-256, respectively, including post-translational modifications of those sequences.

In some embodiments, provided herein are anti-SIRPA antibodies comprising a light chain variable domain (V_{L}) and a heavy chain variable domain (V_{H}), wherein the V_{L} and V_{H} are selected from the group consisting of: V_{L} comprising the amino acid sequence of SEQ ID NO:257 and V_{H} comprising the amino acid sequence of SEQ ID NO:202; V_{L} comprising the amino acid sequence of SEQ ID NO:258 and V_{H} comprising the amino acid sequence of SEQ ID NO:203; V_{L} comprising the amino acid sequence of SEQ ID NO:259 and V_{H} comprising the amino acid sequence of SEQ ID NO:204; V_{L} comprising the amino acid sequence of SEQ ID NO:260 and V_{H} comprising the amino acid sequence of SEQ ID NO:205; V_{L} comprising the amino acid sequence of SEQ ID NO:261 and V_{H} comprising the amino acid sequence of SEQ ID NO:206; V_{L} comprising the amino acid sequence of SEQ ID NO:262 and V_{H} comprising the amino acid sequence of SEQ ID NO:207; V_{L} comprising the amino acid sequence of SEQ ID NO:263 and V_{H} comprising the amino acid sequence of SEQ ID NO:208; V_{L} comprising the amino acid sequence of SEQ ID NO:264 and V_{H} comprising the amino acid sequence of SEQ ID NO:209; V_{L} comprising the amino acid sequence of SEQ ID NO:265 and V_{H} comprising the amino acid sequence of SEQ ID NO:210; V_{L} comprising the amino acid sequence of SEQ ID NO:266 and V_{H} comprising the amino acid sequence of SEQ ID NO:211; V_{L} comprising the amino acid sequence of SEQ ID NO:267 and V_{H} comprising the amino acid sequence of SEQ ID NO:212; V_{L} comprising the amino acid sequence of SEQ ID NO:268 and V_{H} comprising the amino acid sequence of SEQ ID NO:213; V_{L} comprising the amino acid sequence of SEQ ID NO:269 and V_{H} comprising the amino acid sequence of SEQ ID NO:214; V_{L} comprising the amino acid sequence of SEQ ID NO:270 and V_{H} comprising the amino acid sequence of SEQ ID NO:215; V_{L} comprising the amino acid sequence of SEQ ID NO:271 and V_{H} comprising the amino acid sequence of SEQ ID NO:216; V_{L} comprising the amino acid sequence of SEQ ID NO:272 and V_{H} comprising the amino acid sequence of SEQ ID NO:217; V_{L} comprising the amino acid sequence of SEQ ID NO:273 and V_{H} comprising the amino acid sequence of SEQ ID NO:218; V_{L} comprising the amino acid sequence of SEQ ID NO:274 and V_{H} comprising the amino acid sequence of SEQ ID NO:219; V_{L} comprising the amino acid sequence of SEQ ID NO:275 and V_{H} comprising the amino acid sequence of SEQ ID NO:220; V_{L} comprising the amino acid sequence of SEQ ID NO:276 and V_{H} comprising the amino acid sequence of SEQ ID NO:209; V_{L} comprising the amino acid sequence of SEQ ID NO:277 and V_{H} comprising the amino acid sequence of SEQ ID NO:221; V_{L} comprising the amino acid sequence of SEQ ID NO:278 and V_{H} comprising the amino acid sequence of SEQ ID NO:222; V_{L} comprising the amino acid sequence of SEQ ID NO:279 and V_{H} comprising the amino acid sequence of SEQ ID NO:223; V_{L} comprising the amino acid sequence of SEQ ID NO:280 and V_{H} comprising the amino acid sequence of SEQ ID NO:224; V_{L} comprising the amino acid sequence of SEQ ID NO:281 and V_{H} comprising the amino acid sequence of SEQ ID NO:225; V_{L} comprising the amino acid sequence of SEQ ID NO:282 and V_{H} comprising the amino acid sequence of SEQ ID NO:226; V_{L} comprising the amino acid sequence of SEQ ID NO:283 and V_{H} comprising the amino acid sequence of SEQ ID NO:227; V_{L} comprising the amino acid sequence of SEQ ID NO:284 and V_{H} comprising the amino acid sequence of SEQ ID NO:228; V_{L} comprising the amino acid sequence of SEQ ID NO:285 and V_{H} comprising the amino acid sequence of SEQ ID NO:228; V_{L} comprising the amino acid sequence of SEQ ID NO:286 and V_{H} comprising the amino acid sequence of SEQ ID NO:229; V_{L} comprising the amino acid sequence of SEQ ID NO:287 and V_{H} comprising the amino acid sequence of SEQ ID NO:225; V_{L} comprising the amino acid sequence of SEQ ID NO:288 and V_{H} comprising the amino acid sequence of SEQ ID NO:230; V_{L} comprising the amino acid sequence of SEQ ID NO:289 and V_{H} comprising the amino acid sequence of SEQ ID NO:231; V_{L} comprising the amino acid sequence of SEQ ID NO:290 and V_{H} comprising the amino acid sequence of SEQ ID NO:232; V_{L} comprising the amino acid sequence of SEQ ID NO:291 and V_{H} comprising the amino acid sequence of SEQ ID NO:233; V_{L} comprising the amino acid sequence of SEQ ID NO:292 and V_{H} comprising the amino acid sequence of SEQ ID NO:234; V_{L} comprising the amino acid sequence of SEQ ID NO:293 and V_{H} comprising the amino acid sequence of SEQ ID NO:235; V_{L} comprising the amino acid sequence of SEQ ID NO:294 and V_{H} comprising the amino acid sequence of SEQ ID NO:235; V_{L} comprising the amino acid sequence of SEQ ID NO:295 and V_{H} comprising the amino acid sequence of SEQ ID NO:236; V_{L} comprising the amino acid sequence of SEQ ID NO:296 and V_{H} comprising the amino acid sequence of SEQ ID NO:237; V_{L} comprising the amino acid sequence of SEQ ID NO:297 and V_{H} comprising the amino acid sequence of SEQ ID NO:238; V_{L} comprising the amino acid sequence of SEQ ID NO:298 and V_{H} comprising the amino acid sequence of SEQ ID NO:238; V_{L} comprising the amino acid sequence of SEQ ID NO:299 and V_{H} comprising the amino acid sequence of SEQ ID NO:239; V_{L} comprising the amino acid sequence of SEQ ID NO:300 and V_{H} comprising the amino acid sequence of SEQ ID NO:240; V_{L} comprising the amino acid sequence of SEQ ID NO:301 and V_{H} comprising the amino acid sequence of SEQ ID NO:241; V_{L} comprising the amino acid sequence of SEQ ID NO:302 and V_{H} comprising the amino acid sequence of SEQ ID NO:242; V_{L} comprising the amino acid sequence of SEQ ID NO:303 and V_{H} comprising the amino acid sequence of SEQ ID NO:243; V_{L} comprising the amino acid sequence of SEQ ID NO:304 and V_{H} comprising the amino acid sequence of SEQ ID NO:244; V_{L} comprising the amino acid sequence of SEQ ID NO:305 and V_{H} comprising the amino acid sequence of SEQ ID NO:245; V_{L} comprising the amino acid sequence of SEQ ID NO:306 and V_{H} comprising the amino acid sequence of SEQ ID NO:246; V_{L} comprising the amino acid sequence of SEQ ID NO:307 and V_{H} comprising the amino acid sequence of SEQ ID NO:247; V_{L} comprising the amino acid sequence of SEQ ID NO:308 and V_{H} comprising the amino acid sequence of SEQ ID NO:248; V_{L} comprising the amino acid sequence of SEQ ID NO:309 and V_{H} comprising the amino acid sequence of SEQ ID NO:249; V_{L} comprising the amino acid sequence of SEQ ID NO:310 and V_{H} comprising the amino acid sequence of SEQ ID NO:250; V_{L} comprising the amino acid sequence of SEQ ID NO:311 and V_{H} comprising the amino acid sequence of SEQ ID NO:251; V_{L} comprising the amino acid sequence of SEQ ID NO:312 and V_{H} comprising the amino acid sequence of SEQ ID NO:252; V_{L} comprising the amino acid sequence of SEQ ID NO:313 and V_{H} comprising the amino acid sequence of SEQ ID NO:253; V_{L} comprising the amino acid sequence of SEQ ID NO:314 and V_{H} comprising the amino acid sequence of SEQ ID NO:254; V_{L} comprising the amino acid sequence of SEQ ID NO:315 and V_{H} comprising the amino acid sequence of SEQ ID NO:255; V_{L} comprising the amino acid sequence of SEQ ID NO:316 and V_{H} comprising the amino acid sequence of SEQ ID NO:256; V_{L} comprising the amino acid sequence of SEQ ID NO:291 and V_{H} comprising the amino acid sequence of SEQ ID NO:233; and V_{L} comprising the amino acid sequence of SEQ ID NO:317 and V_{H} comprising the amino acid sequence of SEQ ID NO:256.

In some embodiments, provided herein are anti-SIRPA antibodies comprising at least one, two, three, four, five, or six HVRs selected from: (a) HVR-L1 comprising an amino acid sequence selected from the group consisting of SEQ ID NOs:9, 11, 12, and 14; (b) HVR-L2 comprising an amino acid sequence selected from the group consisting of SEQ ID NOs:32 and 33; (c) HVR-L3 comprising an amino acid sequence selected from the group consisting of SEQ ID NOs:45, 48, 53, 59, and 96; (d) HVR-H1 comprising an amino acid sequence selected from the group consisting of SEQ ID NOs:103, 318, 319, 320, 321, 322, 323, 324, 325, 326, 327, 328, 346, 347, 348, and 349; (e) HVR-H2 comprising an amino acid sequence selected from the group consisting of SEQ ID NOs:125, 329, 330, 331, 332, 333, 334, 335, 336, 337, 338, 339, 340, 341, 342, 343, 344, and 345; and (f) HVR-H3 comprising an amino acid sequence selected from the group consisting of SEQ ID NOs:154, 159, 165, 197, 350, 351, 352, 353, 354, 355, 356, 357, 358, 359, 360, 371, 372, 373, 374, 375, 376, 377, 378, 379, 380, 381, 382, 383, 384, 385, and 386.

In some embodiments, provided herein are anti-SIRPA antibodies comprising at least one, at least two, or all three V_{L} HVR sequences selected from (a) HVR-L1 comprising an amino acid sequence selected from the group consisting of SEQ ID NOs: 9, 11, 12, and 14; (b) HVR-L2 comprising an amino acid sequence selected from the group consisting of SEQ ID NOs:32 and 33; and (c) HVR-L3 comprising an amino acid sequence selected from the group consisting of SEQ ID NOs: 45, 48, 53, 59, and 96.

In some embodiments, provided herein are anti-SIRPA antibodies comprising at least one, at least two, or all three V_{H} HVR sequences selected from (a) HVR-H1 comprising an amino acid sequence selected from the group consisting of SEQ ID NOs: 103, 318, 319, 320, 321, 322, 323, 324, 325, 326, 327, 328, 346, 347, 348, and 349; (b) HVR-H2 comprising an amino acid sequence selected from the group consisting of SEQ ID NOs: 125, 329, 330, 331, 332, 333, 334, 335, 336, 337, 338, 339, 340, 341, 342, 343, 344, and 345; and (c) HVR-H3 comprising an amino acid sequence selected from the group consisting of SEQ ID NOs: 154, 159, 165, 197, 350, 351, 352, 353, 354, 355, 356, 357, 358, 359, 360, 371, 372, 373, 374, 375, 376, 377, 378, 379, 380, 381, 382, 383, 384, 385, and 386.

In some embodiments, provided herein are anti-SIRPA antibodies comprising (a) a V_{L} domain comprising at least one, at least two, or all three V_{L} HVR sequences selected from (i) HVR-L1 comprising an amino acid sequence selected from the group consisting of SEQ ID NOs:9, 11, 12, and 14, (ii) HVR-L2 comprising an amino acid sequence selected from the group consisting of SEQ ID NOs:32 and 33, and (iii) HVR-L3 comprising an amino acid sequence selected from the group consisting of SEQ ID NOs: 45, 48, 53, 59, and 96, and (b) a V_{H} domain comprising at least one, at least two, or all three V_{H} HVR sequences selected from (1) HVR-H1 comprising an amino acid sequence selected from the group consisting of SEQ ID NOs: 103, 318, 319, 320, 321, 322, 323, 324, 325, 326, 327, 328, 346, 347, 348, and 349, (ii) HVR-H2 comprising an amino acid sequence selected from the group consisting of SEQ ID NOs: 125, 329, 330, 331, 332, 333, 334, 335, 336, 337, 338, 339, 340, 341, 342, 343, 344, and 345, and (iii) HVR-H3 comprising an amino acid sequence selected from the group consisting of SEQ ID NOs: 154, 159, 165, 197, 350, 351, 352, 353, 354, 355, 356, 357, 358, 359, 360, 371, 372, 373, 374, 375, 376, 377, 378, 379, 380, 381, 382, 383, 384, 385, and 386.

In some embodiments, provided herein are anti-SIRPA antibodies comprising: (a) HVR-L1 comprising the amino acid sequence of SEQ ID NO:9; (b) HVR-L2 comprising the amino acid sequence of SEQ ID NO:32; (c) HVR-L3 comprising the amino acid sequence of SEQ ID NO:45; (d) HVR-H1 comprising the amino acid sequence of SEQ ID NO:318; (e) HVR-H2 comprising the amino acid sequence of SEQ ID NO:329; and (f) HVR-H3 comprising the amino acid sequence of SEQ ID NO:350; (a) HVR-L1 comprising the amino acid sequence of SEQ ID NO:9; (b) HVR-L2 comprising the amino acid sequence of SEQ ID NO:32; (c) HVR-L3 comprising the amino acid sequence of SEQ ID NO:45; (d) HVR-H1 comprising the amino acid sequence of SEQ ID NO:318; (e) HVR-H2 comprising the amino acid sequence of SEQ ID NO:330; and (f) HVR-H3 comprising the amino acid sequence of SEQ ID NO:351; (a) HVR-L1 comprising the amino acid sequence of SEQ ID NO:9; (b) HVR-L2 comprising the amino acid sequence of SEQ ID NO:32; (c) HVR-L3 comprising the amino acid sequence of SEQ ID NO:45; (d) HVR-H1 comprising the amino acid sequence of SEQ ID NO:319; (e) HVR-H2 comprising the amino acid sequence of SEQ ID NO:331; and (f) HVR-H3 comprising the amino acid sequence of SEQ ID NO:352; (a) HVR-L1 comprising the amino acid sequence of SEQ ID NO:9; (b) HVR-L2 comprising the amino acid sequence of SEQ ID NO:32; (c) HVR-L3 comprising the amino acid sequence of SEQ ID NO:45; (d) HVR-H1 comprising the amino acid sequence of SEQ ID NO:318; (e) HVR-H2 comprising the amino acid sequence of SEQ ID NO:125; and (f) HVR-H3 comprising the amino acid sequence of SEQ ID NO:353; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:320; (e) HVR-H2 comprising the amino acid sequence of SEQ ID NO:332; and (f) HVR-H3 comprising the amino acid sequence of SEQ ID NO:371; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:320; (e) HVR-H2 comprising the amino acid sequence of SEQ ID NO:332; and (f) HVR-H3 comprising the amino acid sequence of SEQ ID NO:154; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:103; (e) HVR-H2 comprising the amino acid sequence of SEQ ID NO:333; and (f) HVR-H3 comprising the amino acid sequence of SEQ ID NO:154; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:320; (e) HVR-H2 comprising the amino acid sequence of SEQ ID NO:334; and (f) HVR-H3 comprising the amino acid sequence of SEQ ID NO:154; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:53; (d) HVR-H1 comprising the amino acid sequence of SEQ ID NO:321; (e) HVR-H2 comprising the amino acid sequence of SEQ ID NO:125; and (f) HVR-H3 comprising the amino acid sequence of SEQ ID NO:372; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:53; (d) HVR-H1 comprising the amino acid sequence of SEQ ID NO:321; (e) HVR-H2 comprising the amino acid sequence of SEQ ID NO:335; and (f) HVR-H3 comprising the amino acid sequence of SEQ ID NO:373; (a) HVR-L1 comprising the amino acid sequence of SEQ ID NO:14; (b) HVR-L2 comprising the amino acid sequence of SEQ ID NO:32; (c) HVR-L3 comprising the amino acid sequence of SEQ ID NO:53; (d) HVR-H1 comprising the amino acid sequence of SEQ ID NO:322; (e) HVR-H2 comprising the amino acid sequence of SEQ ID NO:125; and (f) HVR-H3 comprising the amino acid sequence of SEQ ID NO:159; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:323; (e) HVR-H2 comprising the amino acid sequence of SEQ ID NO:336; and (f) HVR-H3 comprising the amino acid sequence of SEQ ID NO:354; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:323; (e) HVR-H2 comprising the amino acid sequence of SEQ ID NO:336; and (f) HVR-H3 comprising the amino acid sequence of SEQ ID NO:355; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:346; (e) HVR-H2 comprising the amino acid sequence of SEQ ID NO:336; and (f) HVR-H3 comprising the amino acid sequence of SEQ ID NO:356; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:337; and (f) HVR-H3 comprising the amino acid sequence of SEQ ID NO:357; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:337; and (f) HVR-H3 comprising the amino acid sequence of SEQ ID NO:374; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:347; (e) HVR-H2 comprising the amino acid sequence of SEQ ID NO:338; and (f) HVR-H3 comprising the amino acid sequence of SEQ ID NO:165; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:337; and (f) HVR-H3 comprising the amino acid sequence of SEQ ID NO:375; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:339; and (f) HVR-H3 comprising the amino acid sequence of SEQ ID NO:376; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:336; and (f) HVR-H3 comprising the amino acid sequence of SEQ ID NO:377; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:48; (d) HVR-H1 comprising the amino acid sequence of SEQ ID NO:348; (e) HVR-H2 comprising the amino acid sequence of SEQ ID NO:337; and (f) HVR-H3 comprising the amino acid sequence of SEQ ID NO:378; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:323; (e) HVR-H2 comprising the amino acid sequence of SEQ ID NO:336; and (f) HVR-H3 comprising the amino acid sequence of SEQ ID NO:379; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:349; (e) HVR-H2 comprising the amino acid sequence of SEQ ID NO:340; and (f) HVR-H3 comprising the amino acid sequence of SEQ ID NO:380; (a) HVR-L1 comprising the amino acid sequence of SEQ ID NO:11; (b) HVR-L2 comprising the amino acid sequence of SEQ ID NO:33; (c) HVR-L3 comprising the amino acid sequence of SEQ ID NO:59; (d) HVR-H1 comprising the amino acid sequence of SEQ ID NO:103; (e) HVR-H2 comprising the amino acid sequence of SEQ ID NO:337; and (f) HVR-H3 comprising the amino acid sequence of SEQ ID NO:381; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:324; (e) HVR-H2 comprising the amino acid sequence of SEQ ID NO:341; and (f) HVR-H3 comprising the amino acid sequence of SEQ ID NO:197; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:342; and (f) HVR-H3 comprising the amino acid sequence of SEQ ID NO:197; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:341; and (f) HVR-H3 comprising the amino acid sequence of SEQ ID NO:382; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:343; and (f) HVR-H3 comprising the amino acid sequence of SEQ ID NO:383; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:341; and (f) HVR-H3 comprising the amino acid sequence of SEQ ID NO:197; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:326; (e) HVR-H2 comprising the amino acid sequence of SEQ ID NO:341; and (f) HVR-H3 comprising the amino acid sequence of SEQ ID NO:197; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:327; (e) HVR-H2 comprising the amino acid sequence of SEQ ID NO:341; and (f) HVR-H3 comprising the amino acid sequence of SEQ ID NO:384; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:341; and (f) HVR-H3 comprising the amino acid sequence of SEQ ID NO:385; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:341; and (f) HVR-H3 comprising the amino acid sequence of SEQ ID NO:386; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:325; (e) HVR-H2 comprising the amino acid sequence of SEQ ID NO:341; and (f) HVR-H3 comprising the amino acid sequence of SEQ ID NO:358; (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:327; (e) HVR-H2 comprising the amino acid sequence of SEQ ID NO:3244; and (f) HVR-H3 comprising the amino acid sequence of SEQ ID NO:359; and (a) HVR-L1 comprising the amino acid sequence of SEQ ID NO:12; (b) HVR-L2 comprising the amino acid sequence of SEQ ID NO:32; (c) HVR-L3 comprising the amino acid sequence of SEQ ID NO:96; (d) HVR-H1 comprising the amino acid sequence of SEQ ID NO:328; (e) HVR-H2 comprising the amino acid sequence of SEQ ID NO:345; and (f) HVR-H3 comprising the amino acid sequence of SEQ ID NO:360.

In another aspect, an anti-SIRPA antibody comprises a light chain variable domain (V_{L}) sequence having at least 90%, 91%, 92%, 93%, 94%, 95%, 96%, 97%, 98%, 99%, or 100% sequence identity to an amino acid sequence selected from the group consisting of SEQ ID NOs:257-317. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs:261, 264, 269, 275, 213, and 413 contains substitutions (e.g., conservative substitutions), insertions, or deletions relative to the reference sequence, but an anti-SIRPA antibody comprising that sequence retains the ability to bind to SIRPA. In certain embodiments, a total of 1 to 10 amino acids have been substituted, inserted, and/or deleted in SEQ ID NO: 261, 264, 269, 275, 213, or 413. In certain embodiments, a total of 1 to 5 amino acids have been substituted, inserted and/or deleted in SEQ ID NO: 261, 264, 269, 275, 213, or 413. In certain embodiments, substitutions, insertions, or deletions occur in regions outside the HVRs (i.e., in the FRs). Optionally, the anti-SIRPA antibody comprises the V_{L} sequence of SEQ ID NO: 261, 264, 269, 275, 213, or 413, including post-translational modifications of that sequence. In a particular embodiment, the V_{L} comprises one, two or three HVRs selected from: (a) HVR-L1 comprising an amino acid sequence selected from the group consisting of SEQ ID NOs: 9, 11, 12, and 14, (b) HVR-L2 comprising an amino acid sequence selected from the group consisting of SEQ ID NOs: 32 and 33, and (c) HVR-L3 comprising an amino acid sequence selected from the group consisting of SEQ ID NOs: 45, 48, 53, 59, and 96.

In another aspect, an anti-SIRPA antibody is provided, wherein the antibody comprises a heavy chain variable domain (V_{H}) having at least 90%, 91%, 92%, 93%, 94%, 95%, 96%, 97%, 98%, 99%, or 100% sequence identity to an amino acid sequence selected from the group consisting of SEQ ID NOs:361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, and 412. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, and 412, and contains substitutions (e.g., conservative substitutions), insertions, or deletions relative to the reference sequence, but an anti-SIRPA antibody comprising that sequence retains the ability to bind to SIRPA. In some embodiments, a total of 1 to 10 amino acids have been substituted, inserted and/or deleted in SEQ ID NO: 361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, or 412. In certain embodiments, a total of 1 to 5 amino acids have been substituted, inserted and/or deleted in SEQ ID NO: 361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, or 412. In certain embodiments, the substitutions, insertions, or deletions occur in regions outside the HVRs (i.e., in the FRs). Optionally, the anti-SIRPA antibody comprises the V_{H} sequence of SEQ ID NO: 361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, or 412, including post-translational modifications of that sequence. In a particular embodiment, the V_{H} comprises one, two or three HVRs selected from (a) HVR-H1 comprising an amino acid sequence selected from the group consisting of SEQ ID NOs: 103, 318, 319, 320, 321, 322, 323, 324, 325, 326, 327, 328, 346, 347, 348, and 349, (b) HVR-H2 comprising an amino acid sequence selected from the group consisting of SEQ ID NOs: 125, 329, 330, 331, 332, 333, 334, 335, 336, 337, 338, 339, 340, 341, 342, 343, 344, and 345, and (c) HVR-H3 comprising an amino acid sequence selected from the group consisting of SEQ ID NOs: 154, 159, 165, 197, 350, 351, 352, 353, 354, 355, 356, 357, 358, 359, 360, 371, 372, 373, 374, 375, 376, 377, 378, 379, 380, 381, 382, 383, 384, 385, and 386.

In some embodiments, an anti-SIRPA antibody is provided, wherein the antibody comprises a V_{L} as in any of the embodiments provided above, and a V_{H} as in any of the embodiments provided above. In some embodiments, provided herein are anti-SIRPA antibodies, wherein the antibody comprises a V_{L} as in any of the embodiments provided above, and a V_{H} as in any of the embodiments provided above. In one embodiment, the antibody comprises the V_{L} and V_{H} sequences in SEQ ID NOs: 261, 264, 269, 275, 213, and 413, and SEQ ID NOs: 361, 362, 363, 364, 365, 366, 367, 368, 369, 370, 387, 388, 389, 390, 391, 392, 393, 394, 395, 396, 397, 398, 399, 400, 401, 402, 403, 404, 405, 406, 407, 408, 409, 410, 411, and 412, respectively, including post-translational modifications of those sequences.

In some embodiments, provided herein are anti-SIRPA antibodies comprising a light chain variable domain (V_{L}) and a heavy chain variable domain (V_{H}), wherein the V_{L} and V_{H} are selected from the group consisting of: V_{L} comprising the amino acid sequence of SEQ ID NO:261 and V_{H} comprising the amino acid sequence of SEQ ID NO:361; V_{L} comprising the amino acid sequence of SEQ ID NO:261 and V_{H} comprising the amino acid sequence of SEQ ID NO:362; V_{L} comprising the amino acid sequence of SEQ ID NO:261 and V_{H} comprising the amino acid sequence of SEQ ID NO:363; V_{L} comprising the amino acid sequence of SEQ ID NO:261 and V_{H} comprising the amino acid sequence of SEQ ID NO:364; V_{L} comprising the amino acid sequence of SEQ ID NO:264 and V_{H} comprising the amino acid sequence of SEQ ID NO:365; V_{L} comprising the amino acid sequence of SEQ ID NO:264 and V_{H} comprising the amino acid sequence of SEQ ID NO:366; V_{L} comprising the amino acid sequence of SEQ ID NO:264 and V_{H} comprising the amino acid sequence of SEQ ID NO:367; V_{L} comprising the amino acid sequence of SEQ ID NO:264 and V_{H} comprising the amino acid sequence of SEQ ID NO:368; V_{L} comprising the amino acid sequence of SEQ ID NO:269 and V_{H} comprising the amino acid sequence of SEQ ID NO:369; V_{L} comprising the amino acid sequence of SEQ ID NO:269 and V_{H} comprising the amino acid sequence of SEQ ID NO:370; V_{L} comprising the amino acid sequence of SEQ ID NO:269 and V_{H} comprising the amino acid sequence of SEQ ID NO:387; V_{L} comprising the amino acid sequence of SEQ ID NO:275 and V_{H} comprising the amino acid sequence of SEQ ID NO:388; V_{L} comprising the amino acid sequence of SEQ ID NO:275 and V_{H} comprising the amino acid sequence of SEQ ID NO:389; V_{L} comprising the amino acid sequence of SEQ ID NO:275 and V_{H} comprising the amino acid sequence of SEQ ID NO:390; V_{L} comprising the amino acid sequence of SEQ ID NO:275 and V_{H} comprising the amino acid sequence of SEQ ID NO:391; V_{L} comprising the amino acid sequence of SEQ ID NO:275 and V_{H} comprising the amino acid sequence of SEQ ID NO:392; V_{L} comprising the amino acid sequence of SEQ ID NO:275 and V_{H} comprising the amino acid sequence of SEQ ID NO:393; V_{L} comprising the amino acid sequence of SEQ ID NO:275 and V_{H} comprising the amino acid sequence of SEQ ID NO:394; V_{L} comprising the amino acid sequence of SEQ ID NO:275 and V_{H} comprising the amino acid sequence of SEQ ID NO:395; V_{L} comprising the amino acid sequence of SEQ ID NO:275 and V_{H} comprising the amino acid sequence of SEQ ID NO:396; V_{L} comprising the amino acid sequence of SEQ ID NO:264 and V_{H} comprising the amino acid sequence of SEQ ID NO:398; V_{L} comprising the amino acid sequence of SEQ ID NO:275 and V_{H} comprising the amino acid sequence of SEQ ID NO:399; V_{L} comprising the amino acid sequence of SEQ ID NO:275 and V_{H} comprising the amino acid sequence of SEQ ID NO:400; V_{L} comprising the amino acid sequence of SEQ ID NO:275 and V_{H} comprising the amino acid sequence of SEQ ID NO:401; V_{L} comprising the amino acid sequence of SEQ ID NO:413 and V_{H} comprising the amino acid sequence of SEQ ID NO:402; V_{L} comprising the amino acid sequence of SEQ ID NO:312 and V_{H} comprising the amino acid sequence of SEQ ID NO:403; V_{L} comprising the amino acid sequence of SEQ ID NO:312 and V_{H} comprising the amino acid sequence of SEQ ID NO:404; V_{L} comprising the amino acid sequence of SEQ ID NO:312 and V_{H} comprising the amino acid sequence of SEQ ID NO:405; V_{L} comprising the amino acid sequence of SEQ ID NO:312 and V_{H} comprising the amino acid sequence of SEQ ID NO:406; V_{L} comprising the amino acid sequence of SEQ ID NO:312 and V_{H} comprising the amino acid sequence of SEQ ID NO:407; V_{L} comprising the amino acid sequence of SEQ ID NO:312 and V_{H} comprising the amino acid sequence of SEQ ID NO:408; V_{L} comprising the amino acid sequence of SEQ ID NO:312 and V_{H} comprising the amino acid sequence of SEQ ID NO:409; V_{L} comprising the amino acid sequence of SEQ ID NO:312 and V_{H} comprising the amino acid sequence of SEQ ID NO:410; V_{L} comprising the amino acid sequence of SEQ ID NO:312 and V_{H} comprising the amino acid sequence of SEQ ID NO:411; V_{L} comprising the amino acid sequence of SEQ ID NO:312 and V_{H} comprising the amino acid sequence of SEQ ID NO:397; and V_{L} comprising the amino acid sequence of SEQ ID NO:312 and V_{H} comprising the amino acid sequence of SEQ ID NO:412.

In some embodiments, an anti-SIRPA antibody of the present disclosure competitively inhibits binding of at least one reference antibody selected from SA-1, SA-2, SA-3, SA-4, SA-5 (including SA-5-57, SA-5-58, SA-5-59, and SA-5-61), SA-6, SA-7, SA-8 (including SA-8-62, SA-8-64, SA-8-66, and SA-8-67), SA-9, SA-10, SA-11, SA-12, SA-13 (including SA-13-6, SA-13-69, and SA-13-71), SA-14, SA-15, SA-16, SA-17, SA-18, SA-19 (including SA-19-72, SA-19-73, SA-19-74, SA-19-75, SA-19-76, SA-19-77, SA-19-78, SA-19-79, SA-19-80, SA-19-81, SA-19-82, SA-19-83, and SA-19-84), SA-20, SA-21, SA-22, SA-23, SA-24, SA-25, SA-26, SA-27, SA-28, SA-29, SA-30, SA-31, SA-32, SA-33, SA-34, SA-35, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56 (including SA-56-85, SA-56-86, SA-56-87, SA-56-88, SA-56-89, SA-56, 90, SA-56, 91, SA-56-92, SA-56-93, SA-56-94, SA-56-95, and SA-56-96), SA-57, SA-58, SA-59, SA-60, SA-61, and SA-62, and any combination thereof, for binding to SIRPA.

In some embodiments, an anti-SIRPA antibody of the present disclosure binds to an epitope of human SIRPA that is the same as or overlaps with the SIRPA epitope bound by at least one reference antibody selected from SA-1, SA-2, SA-3, SA-4, SA-5 (including SA-5-57, SA-5-58, SA-5-59, and SA-5-61), SA-6, SA-7, SA-8 (including SA-8-62, SA-8-64, SA-8-66, and SA-8-67), SA-9, SA-10, SA-11, SA-12, SA-13 (including SA-13-6, SA-13-69, and SA-13-71), SA-14, SA-15, SA-16, SA-17, SA-18, SA-19 (including SA-19-72, SA-19-73, SA-19-74, SA-19-75, SA-19-76, SA-19-77, SA-19-78, SA-19-79, SA-19-80, SA-19-81, SA-19-82, SA-19-83, and SA-19-84), SA-20, SA-21, SA-22, SA-23, SA-24, SA-25, SA-26, SA-27, SA-28, SA-29, SA-30, SA-31, SA-32, SA-33, SA-34, SA-35, SA-36, SA-37, SA-38, SA-39, SA-40, SA-41, SA-42, SA-43, SA-44, SA-45, SA-46, SA-47, SA-48, SA-49, SA-50, SA-51, SA-52, SA-53, SA-54, SA-55, SA-56 (including SA-56-85, SA-56-86, SA-56-87, SA-56-88, SA-56-89, SA-56, 90, SA-56, 91, SA-56-92, SA-56-93, SA-56-94, SA-56-95, and SA-56-96), SA-57, SA-58, SA-59, SA-60, SA-61, and SA-62. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some embodiments, the anti-SIRPA antibody according to any of the above embodiments is a monoclonal antibody, including a humanized and/or human antibody. In some embodiments, the anti-SIRPA antibody is an antibody fragment, e.g., a Fv, Fab, Fab′, scFv, diabody, or F(ab′)2 fragment. In some embodiments, the anti-SIRPA antibody is a substantially full-length antibody, e.g., an IgG1 antibody, IgG2a antibody or other antibody class or isotype as defined herein.

In some embodiments, an anti-SIRPA antibody according to any of the above embodiments may incorporate any of the features, singly or in combination, as described in Sections 1-7 below:

### C. Anti-SIRPA Antibody Binding Affinity

In some embodiments of any of the anti-SIRPA antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, K_{D} is measured using a BIACORE® surface plasmon resonance assay, for example, an assay using a BIACORE®-2000 or a BIACORE®-3000 (BIAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

In some embodiments, an anti-SIRPA antibody of the present disclosure binds to human SIRPAv1 (SEQ ID NO: 1), wherein the K_{D} of binding to human SIRPAv1 is from about 0.3 nM to about 2 nM. In some embodiments, an anti-SIRPA antibody of the present disclosure binds to human SIRPAv1, wherein the K_{D} of binding to human SIRPAv1 is from about 2.8 nM to about 24 nM. In some embodiments, an anti-SIRPA antibody herein binds to human SIRPAv1, wherein the K_{D} of binding to human SIRPAv1 is from 1 nM to 50 nM, from 0.3 nM to 2 nM, or from 2 nM to 24 nM. In some embodiments, an anti-SIRPA antibody herein binds to human SIRPA with a higher affinity than that for murine SIRPA. In some embodiments, an anti-SIRPA antibody that binds specifically to human SIRPA does not bind specifically to SIRPB and/or does not bind specifically to murine SIRPA. In some embodiments, an antibody herein recognizes one or more of: D40, R54, and W68 in D1 of human SIRPA. In some embodiments, when one or more of those amino acid residues is mutated to an alanine, affinity of the antibody for human SIRPA decreases significantly.

### D. Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### E. Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### F. Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3): 185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(-2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### G. Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more SIRPA activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to SIRPA.

It may also be desirable to modify an anti-SIRPA antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-MerTK antibody of the present disclosure to modify effector function to increase finding selectivity toward the ITIM-containing FcgRIIb (CD32b) to increase clustering of SIRPA antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### H. Antibody Variants

In some embodiments of any of the antibodies provided herein, amino acid sequence variants of the antibodies are contemplated. For example, it may be desirable to improve the binding affinity and/or other biological properties of the antibody.

### 1. Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions.

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### 2. Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

### 3. Modified Constant Regions

In some embodiments of any of the antibodies provided herein, the antibody Fc is an antibody Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises N325S and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the modified Fc comprises K322A mutation according to EU numbering. In some embodiments of any of the IgG1 modified Fc, the modified Fc comprises L234A, L235A, and P331S mutations according to EU numbering. In some embodiments of any of the IgG1 modified Fc, the modified Fc comprises P331S mutation according to EU numbering. In some embodiments of any of the IgG1 modified Fc, the modified Fc comprises P331S and E430G mutations according to EU numbering. In some embodiments of any of the IgG1 modified Fc, the modified Fc comprises N325S and L328F mutations according to EU numbering. In some embodiments of any of the IgG1 modified Fc, the modified Fc comprises S267E and L328F mutations according to EU numbering.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-MerTK antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Nu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl.1):27 (2000); Armour et al. The Haematology Journal 1(Suppl.1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1 (CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y′ according to EU numbering.

### 4. Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adr. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### I. Biological Properties of the Antibodies

In some embodiments, anti-SIRPA antibodies herein act as SIRPA agonists in vitro and/or in vivo. For instance, in some embodiments, anti-SIRPA antibodies increase SIRPA activity, SIRPA signaling, CD47-induced SIRPA signaling, or any combination thereof, in macrophages, dendritic cells, and/or microglial cells. In some embodiments, anti-SIRPA antibodies decrease phagocytic activity by phagocytic cells, decrease dendritic cell cytokine release (e.g., release of TNFalpha), suppress synapse elimination in microglia-neuron co-cultures, suppress synapse elimination in mouse models, or any combination thereof.

### III. NUCLEIC ACIDS, VECTORS, AND HOST CELLS

Anti-SIRPA antibodies of the present disclosure may be produced using recombinant methods and compositions, e.g., as described in U.S. Pat. No. 4,816,567. In some embodiments, isolated nucleic acids having a nucleotide sequence encoding any of the anti-SIRPA antibodies of the present disclosure are provided. Such nucleic acids may encode an amino acid sequence comprising the V_{L} and/or an amino acid sequence comprising the V_{H} of the anti-SIRPA antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-SIRPA antibody of the present disclosure are provided. In some embodiments, the method includes culturing a host cell of the present disclosure comprising a nucleic acid encoding the anti-SIRPA antibody, under conditions suitable for expression of the antibody. In some embodiments, the antibody is subsequently recovered from the host cell (or host cell culture medium).

For recombinant production of an anti-SIRPA antibody of the present disclosure, a nucleic acid encoding the anti-SIRPA antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-SIRPA antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-SIRPA antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### IV. PHARMACEUTICAL COMPOSITIONS/FORMULATIONS

Provided herein are pharmaceutical compositions and/or pharmaceutical formulations comprising the anti-SIRPA antibodies of the present disclosure and a pharmaceutically acceptable carrier.

In some embodiments, pharmaceutically acceptable carrier preferably are nontoxic to recipients at the dosages and concentrations employed. The pharmaceutical compositions and/or pharmaceutical formulations to be used for in vivo administration can be sterile. This is readily accomplished by filtration through, e.g., sterile filtration membranes

Pharmaceutical compositions and/or pharmaceutical formulations provided herein are useful as a medicament.

### V. THERAPEUTIC USES

As disclosed herein, anti-SIRPA antibodies of the present disclosure may be used for treating diseases and disorders associated with SIRPA, including, for example, diseases or disorders associated with inflammation, transplant rejection, autoimmunity, and cognitive impairment.

In some embodiments, an anti-SIRPA antibody of the present disclosure is effective at treating inflammatory disorders. In some embodiments, an anti-SIRPA antibody of the present disclosure is effective at reducing inflammation in a subject in need thereof. In some embodiments, an anti-SIRPA antibody of the present disclosure is effective at reducing neuroinflammation in a subject in need thereof. In other embodiments, an anti-SIRPA antibody of the present disclosure is effective at reducing intestinal inflammation, such as, for example, intestinal inflammation associated with colitis.

In other embodiments, an anti-SIRPA antibody of the present disclosure is effective at treating rheumatoid arthritis. In yet other embodiments, an anti-SIRPA antibody of the present disclosure is useful for treating organ/graft transplant rejection in a subject in need thereof.

In yet other embodiments, an anti-SIRPA antibody of the present disclosure is effective at treating multiple sclerosis.

Anti-SIRPA antibodies of the present disclosure are effective at reducing synaptic pruning and reducing synaptic loss in neurons. In some embodiments, an anti-SIRPA antibody of the present disclosure is effective at reducing synaptic pruning by microglia. In other embodiments, an anti-SIRPA antibody of the present disclosure is effective at reducing microglia from eliminating synapses. Accordingly, in some embodiments, an anti-SIRPA antibody of the present disclosure reducing cognitive impairment in a subject in need thereof.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

In one aspect of the invention, an anti-SIRPA antibody is administered to treat, alleviate, and/or prevent a disease or pathology associated with SIRPA expression, activity and/or signaling in a subject. A therapeutic regimen is carried out by identifying a subject, e.g., a human patient suffering from (or at risk of developing) a disease or disorder associated with SIRPA expression, activity and/or signaling, e.g., a cancer or other neoplastic disorder, using standard methods. In some embodiments, cells having the pathology associated with SIRPA expression, activity, and/or signaling, express a SIRPA ligand, e.g., CD47. In some embodiments, cells having the pathology associated with SIRPA expression, activity, and/or signaling, express SIRPA.

As further detailed below an agent that up-regulates SIRPA, e.g., an anti-SIRPA antibody can be used in combination with an additional therapeutic agent that is used to treat the disease or pathology associated with SIRPA expression, activity, or signaling. The terms “in combination” and “in conjunction” are used interchangeably in the present disclosure. The additional therapeutic agent being administered in combination with an anti-SIRPA antibody may be administered before, after, or concurrently with the agent that down-regulates SIRPA, e.g., an anti-SIRPA antibody.

In one aspect of the present disclosure, an anti-SIRPA antibody preparation, e.g., comprising an anti-SIRPA antibody that decreases expression of SIRPA on the cell surface, but does not substantially block binding of ligand, e.g., CD47, to SIRPA, is administered to a human subject. Administration of the antibody may abrogate or inhibit or interfere with the expression, activity and/or signaling function of SIRPA that is mediated by ligand binding, e.g., CD47 binding.

In some embodiments, an agent that up-regulates SIRPA, e.g., an anti-SIRPA antibody, is administered to a patient that has a neurological disorder, or is administered to reduce risk, slow onset, or prevent a neurological disorder. In some embodiments, the neurological disorder is dementia, including frontotemporal dementia, Alzheimer's disease, or vascular dementia, mild cognitive impairment, Parkinson's disease, amyotrophic lateral sclerosis (ALS), Huntington's disease, Tauopathy diseases, schizophrenia, autism spectrum disorder (ASD), or multiple sclerosis.

In some embodiments, an agent that up-regulates SIRPA, e.g., an anti-SIRPA antibody, is administered to a patient that has Parkinson's disease, amyotrophic lateral sclerosis (ALS), Huntington's disease, Tauopathy diseases, schizophrenia, autism spectrum disorder (ASD), or multiple sclerosis. In some embodiments, the agent is administered to a patient that has Creutzfeldt-Jakob disease, normal pressure hydrocephalus, Nasu-Hakola disease, stroke, an infection, traumatic brain injury, progressive supranuclear palsy, dementia pugilistica (chronic traumatic encephalopathy), Parkinsonism linked to chromosome 17, Lytico-Bodig disease (Parkinson-dementia complex of Guam), tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), frontotemporal lobar degeneration, dementia with Lewy bodies, multiple system atrophy, Shy-Drager syndrome, progressive supranuclear palsy, or cortical basal ganglionic degeneration.

### A. Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

In some embodiments, administering an anti-SIRPA antibody of the present disclosure can prevent, reduce the risk, and/or treat dementia. In some embodiments, an anti-SIRPA antibody may modulate one or more SIRPA activities in an individual having dementia.

### 1. Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene.

In some embodiments, administering an anti-SIRPA antibody of the present disclosure, can prevent, reduce the risk, and/or treat FTD. In some embodiments, administering an anti-SIRPA antibody, may modulate one or more SIRPA activities in an individual having FTD.

### 2. Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier. Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

In some embodiments, administering an anti-SIRPA antibody of the present disclosure can prevent, reduce the risk, and/or treat Alzheimer's disease. In some embodiments, administering an anti-SIRPA antibody may modulate one or more SIRPA activities in an individual having Alzheimer's disease.

### B. Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-SIRPA antibody of the present disclosure can prevent, reduce the risk, and/or treat Parkinson's disease. In some embodiments, administering an anti-SIRPA antibody may modulate one or more SIRPA activities in an individual having Parkinson's disease.

### C. Amyotrophic Lateral Sclerosis (ALS)

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that Progranulin plays a role in ALS (Schymick, J C et al., (2007) J[0343] Neurol Neurosurg Psychiatry; 78:754-6) and protects again the damage caused by ALS causing proteins such as TDP-43 (Laird, A S et al., (2010). PLOS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp. 6226-30).

In some embodiments, administering an anti-SIRPA antibody of the present disclosure can prevent, reduce the risk, and/or treat ALS. In some embodiments, administering an anti-SIRPA antibody may modulate one or more SIRPA activities in an individual having amyotrophic lateral sclerosis.

### D. Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

In some embodiments, administering as an anti-SIRPA antibody of the present disclosure can prevent, reduce the risk, and/or treat Huntington's disease (HD). In some embodiments, administering an anti-SIRPA antibody may modulate one or more SIRPA activities in an individual having Huntington's disease.

### E. Tauopathy Disease

Tauopathy diseases, or Tauopathies, are a class of neurodegenerative disease caused by aggregation of the microtubule-associated protein tau within the brain. Alzheimer's disease (AD) is the most well-known tauopathy disease and involves an accumulation of tau protein within neurons in the form of insoluble neurofibrillary tangles (NFTs). Other tauopathy diseases and disorders include progressive supranuclear palsy, dementia pugilistica (chromic traumatic encephalopathy), frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, and frontotemporal lobar degeneration.

In some embodiments, administering an anti-SIRPA antibody of the present disclosure, can prevent, reduce the risk, and/or treat tauopathy disease. In some embodiments, administering an anti-SIRPA antibody may modulate one or more SIRPA activities in an individual having a tauopathy disease.

### F. Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-SIRPA antibody of the present disclosure can prevent, reduce the risk, and/or treat multiple sclerosis. In some embodiments, administering an anti-SIRPA antibody may modulate one or more SIRPA activities in an individual having MS.

### G. Schizophrenia

Schizophrenia is a serious mental disorder in which people interpret reality abnormally. Schizophrenia may result in some combination of hallucinations, delusions, disordered thinking, disorganized speech, and disorganized or abnormal motor behavior that impairs daily functioning, and can be disabling. Other symptoms include reduced or lack of ability to function normally, such as neglect in personal hygiene or appearing to lack emotion (i.e., doesn't make eye contact, doesn't change facial expressions, or speaks in a monotone). A person having schizophrenia may also lost interest in everyday activities, socially withdraw, or lack the ability to experience pleasure. Symptoms can vary in type and severity over time, with periods of worsening and remission of symptoms. Some symptoms may always be present.

In some embodiments, administering an anti-SIRPA antibody of the present disclosure can prevent, reduce the risk, and/or treat schizophrenia. In some embodiments, administering an anti-SIRPA antibody may modulate one or more SIRPA activities in an individual having schizophrenia.

### H. Autism Spectrum Disorder (ASD)

Autism spectrum disorder (ASD) is a condition related to brain development that impacts how a person perceives and socializes with others, causing problems in social interaction and communication. The disorder includes limited and repetitive patterns of behavior. The term “spectrum” in ASD refers to the wide range of symptoms and severity.

ASD includes conditions that were previously considered separate—autism, Asperger's syndrome, childhood disintegrative disorder, and an unspecified form of pervasive developmental disorder. The term “Asperger's syndrome” is still used and is thought to be at the mild end of ASD.

ASD begins in early childhood and eventually causes problems functioning in society-socially, in school and at work, for example. Often, children show symptoms of ASD within the first year. A small number of children appear to develop normally in the first year, and then go through a period of regression between 18 and 24 months of age when they develop ASD symptoms.

In some embodiments, administering an anti-SIRPA antibody of the present disclosure can prevent, reduce the risk, and/or treat ASD. In some embodiments, administering an anti-SIRPA antibody may modulate one or more SIRPA activities in an individual having ASD.

### VI. DIAGNOSTIC USES

In some embodiments of any of the antibodies, any of the anti-SIRPA antibodies provided herein is useful for detecting the presence of SIRPA in a sample or an individual. The term “detecting” as used herein encompasses quantitative or qualitative detection. Provided herein are methods of using the antibodies of this disclosure for diagnostic purposes, such as the detection of SIRPA in an individual or in tissue samples derived from an individual. In some embodiments, the individual is a human. In some embodiments, the tissue sample is phagocytic cells (e.g., macrophages, dendritic cells), tumor tissue, cancer cells, etc.

The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

### VII. ARTICLES OF MANUFACTURE

Provided herein are articles of manufacture (e.g., kit) comprising an anti-SIRPA antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-SIRPA antibody described herein) to treat an individual having a disease, disorder, or injury, such as for example cancer, according to any methods of this disclosure. In some embodiments, the instructions include instructions for use of the anti-SIRPA antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### VIII. EXAMPLES

The following examples are provided by way of illustration only and not by way of limitation. Those of skill in the art will readily recognize a variety of non-critical parameters that could be changed or modified to yield essentially similar results.

### Example 1: SIRPA Polypeptides

The amino acid sequence of the human SIRPA preprotein is set forth below in SEQ ID NO: 1. Human SIRPA contains a signal peptide located at amino residues 1-30 of SEQ ID NO: 1. Human SIRPA contains an extracellular immunoglobulin-like variable-type (IgV) domain located at amino residues 32-137 of SEQ ID NO: 1; additional extracellular immunoglobulin-like constant-type (IgC) domain sequences located at amino residues 148-247 and 254-348 of SEQ ID NO: 1; a transmembrane domain located at amino residues 374-394 of SEQ ID NO: 1; and an intracellular domain located at amino residues 395-504 of SEQ ID NO: 1.

The following amino acid sequences are provided below: human SIRPAv1 (SEQ ID NO:1); human SIRPAv2 (SEQ ID NO:2); mouse SIRPA (SEQ ID NO:3); human SIRPβ1 isoform 1 (SEQ ID NO:4); human SIRPβ1 isoform 3 (SEQ ID NO:5); and mouse SIRPβ1 (SEQ ID NO:6).

Human SIRPA v1
(SEQ ID NO: 1)
MEPAGPAPGR LGPLLCLLLA ASCAWSGVAG EEELQVIQPD KSVLVAAGET ATLRCTATSL
IPVGPIQWFR GAGPGRELIY NQKEGHFPRV TTVSDLTKRN NMDFSIRIGN ITPADAGTYY
CVKFRKGSPD DVEFKSGAGT ELSVRAKPSA PVVSGPAARA TPQHTVSFTC ESHGFSPRDI
TLKWFKNGNE LSDFQTNVDP VGESVSYSIH STAKVVLTRE DVHSQVICEV AHVTLQGDPL
RGTANLSETI RVPPTLEVTQ QPVRAENQVN VTCQVRKFYP QRLQLTWLEN GNVSRTETAS
TVTENKDGTY NWMSWLLVNV SAHRDDVKLT CQVEHDGQPA VSKSHDLKVS AHPKEQGSNT
AAENTGSNER NIYIVVGVVC TLLVALLMAA LYLVRIRQKK AQGSTSSTRL HEPEKNAREI
TQDTNDITYA DLNLPKGKKP APQAAEPNNH TEYASIQTSP QPASEDTLTY ADLDMVHLNR
TPKQPAPKPE PSFSEYASVQ VPRK
Human SIRPA v2
(SEQ ID NO: 2)
MEPAGPAPGR LGPLLCLLLA ASCAWSGVAG EEELQVIQPD KSVSVAAGES AILHCTVTSL
IPVGPIQWFR GAGPARELIY NQKEGHFPRV TTVSESTKRE NMDFSISISN ITPADAGTYY
CVKFRKGSPD TEFKSGAGTE LSVRAKPSAP VVSGPAARAT PQHTVSFTCE SHGFSPRDIT
LKWFKNGNEL SDFQTNVDPV GESVSYSIHS TAKVVLTRED VHSQVICEVA HVTLQGDPLR
GTANLSETIR VPPTLEVTQQ PVRAENQVNV TCQVRKFYPQ RLQLTWLENG NVSRTETAST
VTENKDGTYN WMSWLLVNVS AHRDDVKLTC QVEHDGQPAV SKSHDLKVSA HPKEQGSNTA
AENTGSNERN IYIVVGVVCT LLVALLMAAL YLVRIRQKKA QGSTSSTRLH EPEKNAREIT
QDTNDITYAD LNLPKGKKPA PQAAEPNNHT EYASIQTSPQ PASEDTLTYA DLDMVHLNRT
PKQPAPKPEP SFSEYASVQV PRK
Mouse SIRPA
(SEQ ID NO: 3)
MEPAGPAPGR LGPLLLCLLL SASCFCTGAT GKELKVTQPE KSVSVAAGDS TVLNCTLTSL
LPVGPIRWYR GVGPSRLLIY SFAGEYVPRI RNVSDTTKRN NMDFSIRISN VTPADAGIYY
CVKFQKGSSE PDTEIQSGGG TEVYVLAKPS PPEVSGPADR GIPDQKVNFT CKSHGFSPRN
ITLKWFKDGQ ELHPLETTVN PSGKNVSYNI SSTVRVVLNS MDVNSKVICE VAHITLDRSP
LRGIANLSNF IRVSPTVKVT QQSPTSMNQV NLTCRAERFY PEDLQLIWLE NGNVSRNDTP
KNLTKNTDGT YNYTSLFLVN SSAHREDVVF TCQVKHDQQP AITRNHTVLG FAHSSDQGSM
QTFPDNNATH NWNVFIGVGV ACALLVVLLM AALYLLRIKQ KKAKGSTSST RLHEPEKNAR
EITQIQDTND INDITYADLN LPKEKKPAPR APEPNNHTEY ASIETGKVPR PEDTLTYADL
DMVHLSRAQP APKPEPSFSE YASVQVQRK
Human SIRPB1 isoform 1
SEQ ID NO: 4)
MPVPASWPHL PSPFLLMTLL LGRLTGVAGE DELQVIQPEK SVSVAAGESA TLRCAMTSLI
PVGPIMWFRG AGAGRELIYN QKEGHFPRVT TVSELTKRNN LDFSISISNI TPADAGTYYC
VKFRKGSPDD VEFKSGAGTE LSVRAKPSAP VVSGPAVRAT PEHTVSFTCE SHGFSPRDIT
LKWFKNGNEL SDFQTNVDPA GDSVSYSIHS TARVVLTRGD VHSQVICEIA HITLQGDPLR
GTANLSEAIR VPPTLEVTQQ PMRAENQANV TCQVSNFYPR GLQLTWLENG NVSRTETAST
LIENKDGTYN WMSWLLVNTC AHRDDVVLTC QVEHDGQQAV SKSYALEISA HQKEHGSDIT
HEAALAPTAP LLVALLLGPK LLLVVGVSAI YICWKQKA
Human SIRPB1 isoform 3
(SEQ ID NO: 5)
MPVPASWPHL PSPFLLMTLL LGRLTGVAGE EELQVIQPDK SISVAAGESA TLHCTVTSLI
PVGPIQWFRG AGPGRELIYN QKEGHFPRVT TVSDLTKRNN MDFSIRISNI TPADAGTYYC
VKFRKGSPDH VEFKSGAGTE LSVRAKPSAP VVSGPAARAT PQHTVSFTCE SHGFSPRDIT
LKWFKNGNEL SDFQTNVDPA GDSVSYSIHS TAKVVLTRED VHSQVICEVA HVTLQGDPLR
GTANLSETIR VPPTLEVTQQ PVRAENQVNV TCQVRKFYPQ RLQLTWLENG NVSRTETAST
LTENKDGTYN WMSWLLVNVS AHRDDVKLTC QVEHDGQPAV SKSHDLKVSA HPKEQGSNTA
PGPALASAAP LLIAFLLGPK VLLVVGVSVI YVYWKQKA
Mouse SIRPB1
(SEQ ID NO: 6)
MLLLDAWTHI PHCVLLLILL LGLKGAAVRE LKVIQPVKSF FVGAGGSATL NCTVTYLLPV
GPIKWYRGVG QSRLLIYPFT GEYFPRITSV SDVKKRSNLD FSIRISNVTP ADSGTYYCVK
FQRGSSEPDI EIQSGGGTEL SVFAKPSSPM VSGPAARAVP QQTVTFTCRS HGFFPQNLTL
KWFKNGNEIS HLETSVEPEE TSVSYRVSST VQVVLEPRDV RSQIICEVDH VTLDRAPLRG
IAHISEIIQV PPTLEISQQP TMVWNVINVT CQIQKFYPRR FQVTWLENGN ISRREVPFTH
IVNKDGTYNW ISWLLVNISA LEENMVVTCQ VEHDGQAEVI ETHTVVVTEH QRVKGTSTMS
ELKTAGIAKI PVAVLLGSKI LLLIAATVIY MRKKQNA

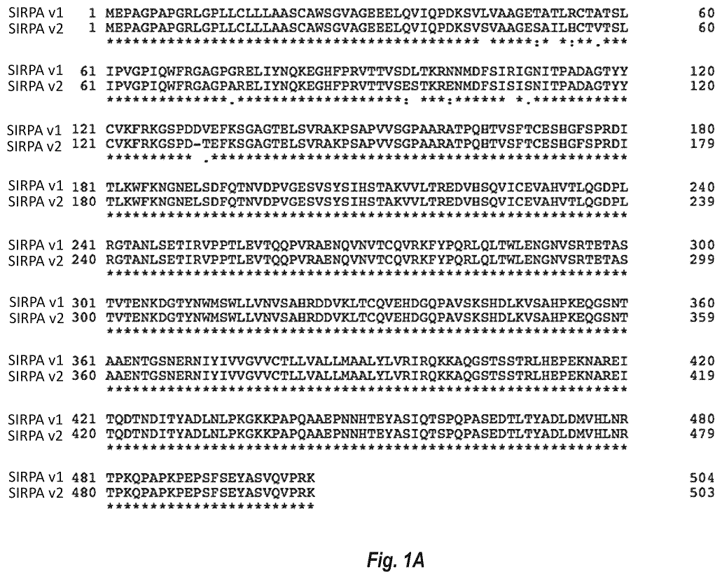

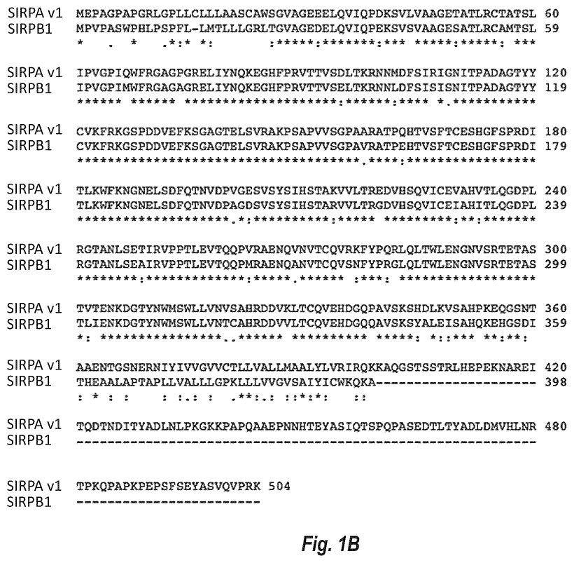

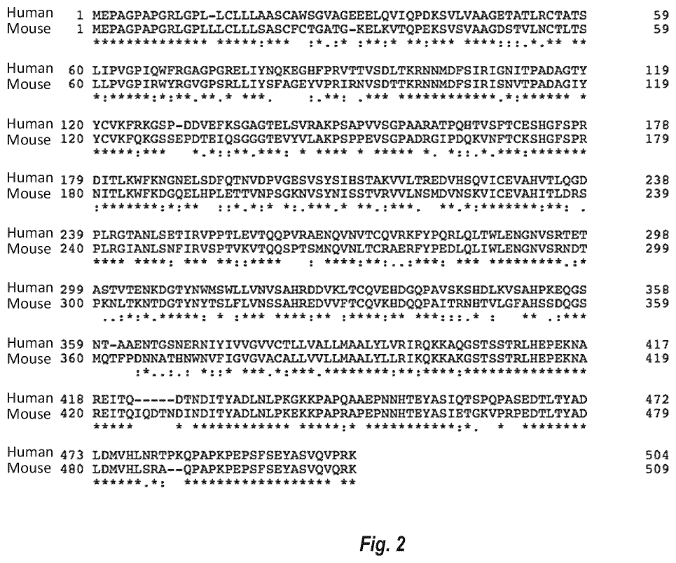

Multiple polymorphisms of SIRPA have been identified in humans. An alignment of the amino acid sequences of the two most common variants, referred to as SIRPA v1 and v2, was generated by 2-way blast (FIG. 1A). Since most variations in sequence lie beyond the ligand binding site, both SIRPA variants are reported to bind CD47 with similar affinities. Alternatively, another member of the SIRP family, SIRPβ1, shares high sequence homology with SIRPA but fails to bind CD47. An alignment of the amino acid sequences of SIRPAv1 and SIRPβ1 was generated by 2-way blast (FIG. 1B) and shows that the extracellular domain of both proteins (excluding leader sequence) shares ˜90% identity. However, a single A57M substitution is sufficient to rearrange the S59-P65 ligand-binding interface to prevent SIRPβ1 binding to CD47. Furthermore, CD47 binding is highly species-specific with human CD47 recognizing a single allelic variant of mouse SIRPA expressed only by NOD mice. An alignment of the amino acid sequences of human SIRPAv1 and C57BL6 SIRPA was generated by 2-way blast (FIG. 2) and shows that the extracellular domain of both proteins (excluding leader sequence) shares ˜60% identity.

Crystal structure analyses of SIRPA-CD47 complexes resolve the ligand binding site to the variable loops that link the β-sheet strands in the IgV domain of SIRPA. The CD47-binding interface consists of amino acid residues S59-P65, L96-F104, and K123-D130 of human SIRPA.

### Example 2: Production of Monomeric and Fc-Conjugated Human SIRPA and SIRPβ1

Mammalian expression of SIRPA and SIRPβ1 antigen was performed by cloning synthetic genes based on cDNA into mammalian expression vectors, followed by transient transfection and expression in HEK293/Tcells. Constructs included a heterologous signal peptide and human IgG1 Fc for Fc fusion constructs. Briefly, expression vectors containing the antigen of interest were transfected by complexing with a transfection reagent followed by exposure to HEK293/Tcells for one hour followed by dilution of culture media to a final density of 4 million cells per mL. The cells were then cultured for 7 days with fresh feed media every 48 hours. After 7 days, the supernatant was collected following centrifugation and purification was performed using protein Ni-sepharose and if necessary, a SEC column purification to reach >95% non-aggregated monomer content. SIRPA monomer antigens were prepared by fragmenting a SIRPA Fc fusion antigen with modified hinge region (Lynaugh et al., MAbs. 2013 October; 5(5):641-45) with FabRICATOR (IdeS) protease (Genovis, Cat #A2-FR2-1000), followed by Protein A affinity purification to remove undigested Fc fusion protein and SEC to remove aggregated monomer.

### Example 3: Library Screening for Anti-SIRPA Antibodies

Eight naive human synthetic yeast libraries each of ˜10⁹ diversity were designed, generated, and propagated as described previously (see, e.g., Xu et al, 2013; WO2009036379; WO2010105256; WO2012009568; Nu et al., Protein Eng Des Sel. 2013 October; 26(10):663-70). Ten parallel selections were performed, using the eight naive libraries for human SIRPA Fc fusion antigen selections and two pools of the eight libraries for human SIRPA monomer selections. For the first two rounds of selection, a magnetic bead sorting technique utilizing the Miltenyi MACs system was performed, essentially as described (Siegel et al., J Immunol Methods. 2004 March; 286(1-2): 141-53). Briefly, yeast cells (˜10¹⁰ cells/library) were incubated with 3 ml of 10 nM biotinylated SIRPA Fc fusion antigen or 100 nM biotinylated SIRPA monomer antigen for 15 min at room temperature in FACS wash buffer PBS with 0.1% BSA. After washing once with 50 ml ice-cold wash buffer, the cell pellet was resuspended in 40 mL wash buffer, and 500 AL Streptavidin MicroBeads (Miltenyi Biotec, Bergisch Gladbach, Germany. Cat #130-048-101) were added to the yeast and incubated for 15 min at 4° C. Next, yeast cells were pelleted, resuspended in 5 mL wash buffer, and loaded onto a MACS LS column (Miltenyi Biotec, Bergisch Gladbach, Germany. Cat. #130-042-401). After the 5 mL was loaded, the column was washed 3 times with 3 ml FACS wash buffer. The column was then removed from the magnetic field, and the yeast were eluted with 5 mL of growth media and then grown overnight. The following four rounds of sorting were performed using flow cytometry.

Approximately 1×10⁸ yeast were pelleted, washed three times with wash buffer, and incubated with 10 nM biotinylated SIRPA Fc fusion antigen or 100 nM biotinylated SIRPA monomer antigen for 10 min at room temperature. Yeast were then washed twice and stained with goat anti-human F(ab′)2 kappa-FITC diluted 1:100 (Southern Biotech, Birmingham, Alabama, Cat #2062-02) and either streptavidin-Alexa Fluor 633 (Life Technologies, Grand Island, NY, Cat #S21375) diluted 1:500, or Extravidin-phycoerthyrin (Sigma-Aldrich, St Louis, Cat #E4011) diluted 1:50, secondary reagents for 15 min at 4° C. After washing twice with ice-cold wash buffer, the cell pellets were resuspended in 0.4 mL wash buffer and transferred to strainer-capped sort tubes. Sorting was performed using a FACS ARIA sorter (BD Biosciences) and sort gates were determined to select only SIRPA binding clones for one round and the second round was a negative sort to decrease reagent binders, polyspecific binders (Xu et al., PEDS. 2013 October; 26(10):663-70), and binders to control protein human SIRPβ1 HIS tagged monomer. The third round utilized labeling with 10 nM human SIRPA Fc fusion antigens, 100 nM human SIRPA monomer antigen, and competition with CD47 using SIRPA antigens (10 nM) pre-complexed with 500 nM CD47. For yeast competitive with CD47, a final round to enrich SIRPA Fc fusion antigen binders was performed. After the final round of sorting, yeast were plated and individual colonies were picked for further characterization.

Heavy chains from the second and fourth FACS sorting selection round outputs were used to prepare light chain diversification libraries used for additional selections. For these selections, the first selection round utilized Miltenyi MACs beads and labeling with 10 nM human SIRPA Fc fusion antigen. Four rounds of FACS sorting followed. The first round used 100 nM human SIRPA monomer antigen. The second FACS round was a negative sort to decrease binding to reagent binders, polyspecific binders, and binders to control protein human SIRPβ1 HIS tagged monomer. The last two rounds utilized human SIRPA monomer titration (100 nM, 10 nM, and 1 nM) to select highest affinity binders, 100 nM human SIRPβ1 monomer, and competition with control AM4-5 antibody to assess competitor representation in the enriched population. After the final round of sorting, yeast were plated and individual colonies were picked for characterization.

### Example 4: Antibody IgG and Fab Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over CaptureSelect IgG-CH1 affinity matrix (LifeTechnologies, Cat #1943200250).

### Example 5: Characterization of Anti-SIRPA Antibodies

The affinities of the anti-SIRPA antibodies of the present disclosure were determined by measuring their K_{D} by ForteBio binding experiments. ForteBio affinity measurements were performed generally as previously described (Estep et al., MAbs. 2013 March-April; 5(2):270-8). Briefly, ForteBio affinity measurements were performed by loading IgGs on-line onto AHQ sensors. Sensors were equilibrated off-line in assay buffer for 30 min and then monitored on-line for 60 seconds for baseline establishment. For avid binding measurement, sensors with loaded IgGs were exposed to 100 nM antigen (human SIRPA or SIRPβ1 Fc fusion) for 3 min, afterwards they were transferred to assay buffer for 3 min for off-rate measurement. Additional avid binding was determined by loading biotinylated SIRPA monomer on SA sensors and exposure to 100 nM IgG in solution. Monovalent binding measurements were obtained by loading human SIRPA or SIRPβ1 Fc fusion antigens to AHQ sensor and followed by exposure to 100 nM anti-SIRPA antibody Fab. Additional monovalent measurements were made by loading biotinylated human SIRPA or SIRPβ1 monomer to SA sensor followed by exposure to 100 nM Fab in solution. Kinetics data were fit using a 1:1 binding model in the data analysis software provided by ForteBio.

Epitope binning of the anti-SIRPA antibodies of the present disclosure was performed on a ForteBio Octet Red384 system (ForteBio, Menlo Park, CA) using a standard sandwich format binning assay. Control anti-target IgG was loaded onto AHQ sensors and unoccupied Fc-binding sites on the sensor were blocked with a non-relevant human IgG1 antibody. The sensors were then exposed to 100 nM target antigen followed by a second anti-target antibody. Data was processed using ForteBio's Data Analysis Software 7.0. Additional binding by the second antibody after antigen association indicated an unoccupied epitope (non-competitor), while no binding indicated epitope blocking (competitor). This process was iterated for two reference antibodies: (i) AM4-5, which binds to the CD47 binding site on Domain 1 of SIRPA (bin 1) and (ii) SA-56, which binds to domain 1 of SIRPA without blocking CD47 binding (bin 2).

CD47 binding competition with anti-SIRPA antibodies of the present disclosure were carried out as follows. All experiments were performed on a ForteBio HTX instrument. All samples were diluted into PBSF (0.1% BSA in PBS). All dip and read ForteBio steps involved shaking at 1000 rpm.

Sensors were soaked in PBS for 10 min prior to analysis. The blank sensors were dipped into human CD47-Fc (100 nM) and were then soaked in PBSF for 10 min prior to the analysis. The CD47 loaded tips were sequentially dipped into PBSF (1 min), 100 nM human SIRPA-Fc (3 min) and finally 100 nM test antibody. The data was prepared for analysis with ForteBio Data Analysis Software version 8.1.0.36 as follows. The data was y-axis aligned and inter-step corrected to the beginning of the SIRPA capture step and then cropped to show only the SIRPA capture and antibody interaction steps. Assays in which no binding signal was observed upon addition of a given test antibody solution indicated that the anti-SIRPA antibody binding site overlaps the CD47 binding site on the surface of the receptor. Assays in which a binding signal was observed upon addition of a given test anti-SIRPA antibody solution indicated that CD47 does not block antibody binding to SIRPA.

The final set of anti-SIRPA antibodies were selected on based antigen binding affinities. Antibodies that were positive for binding to human SIRPA were tested for ability to block ligand binding and for cross-reactivity to human SIRPβ1. Anti-SIRPA antibodies were then assigned a bin based on the results of these studies.

Table 1 below shows the results from these studies characterizing various aspects of the anti-SIRPA antibodies of the present disclosure. In Table 1, “ND” refers to antibodies for which the Bin category has not been determined; “NB” refers to antibodies for which there is no binding to the indicated antigen detected; “PF” refers to antibodies for which antigen binding kinetics show poor fit to 1:1 binding model.

TABLE 1
Anti-SIRPA Antibodies.

| | | | Fab K_{D} | IgG K_{D} | IgG K_{D} | IgG K_{D} | IgG K_{D} |
|---|---|---|---|---|---|---|---|
| | Bin | | Human | Human | Mouse | Human | Mouse |
| | Code | Human | SIRPAFc | SIRPAD1 | SIRPAD1 | SIRPbD1 | SIRPbD1 |
| Clone | (Human | CD47 | (M) | Fc (M) | Fc (M) | Fc (M) | Fc (M) |
| Index | SIRPA) | Competitor | Monovalent | Avid | Avid | Avid | Avid |
| SA-1 | 1, 2 | Yes | N.B. | 2.00E−08 | N.B. | N.B. | N.B. |
| SA-2 | 1 | No | P.F. | 2.44E−09 | N.B. | N.B. | N.B. |
| SA-3 | 1 (?) | Yes | N.B. | 2.14E−08 | N.B. | N.B. | N.B. |
| SA-4 | 1, 2 | Yes | N.B. | 3.02E−08 | N.B. | N.B. | N.B. |
| SA-5 | 2 | No | N.B. | P.F. | N.B. | N.B. | N.B. |
| SA-6 | 2 | No? | N.B. | P.F. | N.B. | N.B. | N.B. |
| SA-7 | 1 (?) | Yes | N.B. | P.F. | N.B. | N.B. | N.B. |
| SA-8 | Other | No? | P.F. | P.F. | N.B. | N.B. | N.B. |
| SA-9 | 1 | No | N.B. | P.F. | N.B. | N.B. | N.B. |
| SA-10 | 2 | Yes | N.B. | P.F. | N.B. | N.B. | N.B. |
| SA-11 | 2 | No | N.B. | P.F. | N.B. | N.B. | N.B. |
| SA-12 | 1, 2 | No | N.B. | 1.30E−08 | N.B. | N.B. | N.B. |
| SA-13 | 1 | No | P.F. | P.F. | N.B. | 1.96E−08 | N.B. |
| SA-14 | 1 | Yes | N.B. | 3.10E−08 | N.B. | N.B. | N.B. |
| SA-15 | Other | Yes | N.B. | P.F. | N.B. | 2.84E−08 | N.B. |
| SA-16 | 2 | No | 2.57E−07 | 4.02E−09 | N.B. | N.B. | N.B. |
| SA-17 | 2 | No | N.B. | P.F. | N.B. | N.B. | N.B. |
| SA-18 | 2 | No? | N.B. | P.F. | N.B. | N.B. | N.B. |
| SA-19 | 2 | Yes | N.B. | P.F. | N.B. | N.B. | N.B. |
| SA-20 | Other | Yes | N.B. | P.F. | N.B. | N.B. | N.B. |
| SA-21 | 1 | No | 3.59E−07 | 6.89E−09 | N.B. | N.B. | N.B. |
| SA-22 | 1 (?) | Yes | N.B. | 2.20E−08 | N.B. | N.B. | N.B. |
| SA-23 | 1 | Yes | N.B. | 3.80E−08 | N.B. | N.B. | N.B. |
| SA-24 | 2 | No | N.B. | P.F. | N.B. | N.B. | N.B. |
| SA-25 | 2 | No | N.B. | 2.42E−08 | N.B. | N.B. | N.B. |
| SA-26 | 2 | No | N.B. | 1.38E−08 | N.B. | N.B. | N.B. |
| SA-27 | 2 | No | P.F. | 4.43E−09 | N.B. | N.B. | N.B. |
| SA-28 | 2 | No | 4.86E−07 | 6.67E−09 | N.B. | N.B. | N.B. |
| SA-29 | 2 | No | P.F. | 4.55E−09 | N.B. | N.B. | N.B. |
| SA-30 | 1 | No | N.B. | P.F. | N.B. | N.B. | N.B. |
| SA-31 | 2 | No | N.B. | 6.47E−09 | N.B. | N.B. | N.B. |
| SA-32 | 1? | Yes | N.B. | 2.44E−08 | N.B. | N.B. | N.B. |
| SA-33 | 2 | No | N.B. | P.F. | N.B. | N.B. | N.B. |
| SA-34 | Other | Yes | P.F. | 2.68E−08 | N.B. | N.B. | N.B. |
| SA-35 | 2 | No | 4.97E−07 | 1.02E−08 | N.B. | N.B. | N.B. |
| SA-36 | 2 | No | 2.07E−06 | 8.67E−09 | N.B. | N.B. | N.B. |
| SA-37 | 2 | No | N.B. | 1.02E−08 | N.B. | N.B. | N.B. |
| SA-38 | 2 | No | 2.81E−07 | 3.81E−09 | N.B. | N.B. | N.B. |
| SA-39 | 2 | No | 1.00E−07 | 3.62E−09 | N.B. | 2.23E−08 | N.B. |
| SA-40 | 2 | No | N.B. | P.F. | N.B. | N.B. | N.B. |
| SA-41 | 1? | Yes | N.B. | P.F. | N.B. | N.B. | N.B. |
| SA-42 | 1 (?) | Yes | N.B. | P.F. | N.B. | N.B. | N.B. |
| SA-43 | 2 | No | N.B. | 1.05E−08 | N.B. | N.B. | N.B. |
| SA-44 | 2 | No? | N.B. | P.F. | N.B. | N.B. | N.B. |
| SA-45 | 1 | Yes | N.B. | P.F. | N.B. | N.B. | N.B. |
| SA-46 | 1 | Yes | N.B. | P.F. | N.B. | N.B. | N.B. |
| SA-47 | 2 | No | N.B. | 1.54E−08 | N.B. | N.B. | N.B. |
| SA-48 | 1 | No? | N.B. | P.F. | N.B. | N.B. | N.B. |
| SA-49 | 1 | No? | N.B. | P.F. | N.B. | N.B. | N.B. |
| SA-50 | 1 | No | N.B. | P.F. | N.B. | N.B. | N.B. |
| SA-51 | 1 | Yes | N.B. | P.F. | N.B. | N.B. | N.B. |
| SA-52 | Other | Yes | N.B. | P.F. | N.B. | N.B. | N.B. |
| SA-53 | 2 | Yes | N.B. | P.F. | N.B. | N.B. | N.B. |
| SA-54 | 1 | No | N.B. | P.F. | N.B. | N.B. | N.B. |
| SA-55 | 1 | Yes | N.B. | P.F. | N.B. | N.B. | N.B. |
| SA-56 | 2 | No | 3.01E−07 | 2.81E−09 | N.B. | N.B. | N.B. |
| SA-57 | 2 | Yes | N.B. | P.F. | N.B. | N.B. | N.B. |
| SA-58 | 2 | Yes | P.F. | 4.31E−09 | N.B. | N.B. | N.B. |
| SA-59 | 1, 2 | Yes | N.B. | 2.36E−08 | N.B. | N.B. | N.B. |
| SA-60 | 2 | Yes | N.B. | 2.22E−08 | N.B. | N.B. | N.B. |
| SA-61 | 2 | No? | N.B. | 1.21E−08 | N.B. | N.B. | N.B. |
| SA-62 | 2 | No | N.B. | 1.65E−08 | N.B. | N.B. | N.B. |

### Example 6: Antibody Heavy Chain and Light Chain Variable Domain Sequences

Using standard techniques, the amino acid sequences encoding the light chain variable domain and the heavy chain variable domain of the generated anti-SIRPA antibodies were determined. The Kabat light chain HVR sequences of the antibodies are set forth in Table 2. The Kabat heavy chain HVR sequences of the antibodies are set forth in Table 3. Amino acid sequences of the heavy chain variable regions and light chain variable regions of the antibodies are set forth in Table 4.

TABLE 2
Kabat Light Chain HVR Sequences of the Antibodies.

| | | SEQ ID | | SEQ ID | | SEQ ID |
|---|---|---|---|---|---|---|
| Antibody | HVR-L1 | NO: | HVR-L2 | NO: | HVR-L3 | NO: |
| SA-1 | RASQSVSSYLA | 7 | DSSNRAT | 29 | QQDSDYPFT | 41 |
| SA-2 | KSSQSVLFSSN | 8 | WASTRES | 30 | QQYFLYPPT | 42 |
| | NKNYLA | | | | | |
| SA-3 | RASQSVSSYLA | 7 | DASNRAT | 31 | QQHDFWPPT | 43 |
| SA-4 | RASQSVSSYLA | 7 | DASNRAT | 31 | QQDSNFPFT | 44 |
| SA-5 | RASQDISSWLA | 9 | AASSLQS | 32 | QQGYIFPRT | 45 |
| SA-6 | RASQSVSSYLA | 7 | DSSNRAT | 29 | QQDANYPYT | 46 |
| SA-7 | KSSQSVLYSSN | 10 | WASTRES | 30 | QQYYHYPPT | 47 |
| | NKNYLA | | | | | |
| SA-8 | QASQDISNYLN | 11 | DASNLET | 33 | QQLSNYPIT | 48 |
| SA-9 | RASQGISSWLA | 12 | AASSLQS | 32 | QQAYLYPIT | 49 |
| SA-10 | RASQSISSWLA | 13 | EASSLES | 34 | QQDNSLPYT | 50 |
| SA-11 | RASQSISSWLA | 13 | DASSLES | 35 | QQYNSYSPT | 51 |
| SA-12 | RASQSVSSYLA | 7 | DASNRAT | 31 | QQRTNFPIT | 52 |
| SA-13 | RASQSISSYLN | 14 | AASSLQS | 32 | QQGYIYPPT | 53 |
| SA-14 | QASQDISNFLN | 15 | DASNLET | 33 | QQFINFPLT | 54 |
| SA-15 | RASQSVSSYLA | 7 | DASNRAT | 31 | QQPYNEPLT | 55 |
| SA-16 | RASQSVSSYLA | 7 | DASNRAT | 31 | QQFSDYPT | 56 |
| SA-17 | RASQSVSSSLA | 16 | GASTRAT | 36 | QQDYIWPLT | 57 |
| SA-18 | RASQGISSWLA | 12 | AASSLQS | 32 | QQALSFPFT | 58 |
| SA-19 | QASQDISNYLN | 11 | DASNLET | 33 | QQFTYLPPT | 59 |
| SA-20 | QASQDISNYLN | 11 | DASNLET | 33 | QQVDVLPYT | 60 |
| SA-21 | KSSQSVLFSSN | 8 | WASTRES | 30 | QQDYATPFT | 61 |
| | NKNYLA | | | | | |
| SA-22 | QASQDISNYLN | 11 | DASNLET | 33 | QQYDTLPPT | 62 |
| SA-23 | RSSQSLLHSNG | 17 | LGSNRAS | 37 | MQAQGTPYT | 63 |
| | YNYLD | | | | | |
| SA-24 | RASQSVSSYLA | 7 | DASNRAT | 31 | QQRLDWPYT | 64 |
| SA-25 | QASQDISNYLN | 11 | DASNLET | 33 | QQYSYLPT | 65 |
| SA-26 | RASQSINSYLN | 18 | AASSLQS | 32 | QQSPSDPWT | 66 |
| SA-27 | RASQSISSYLN | 14 | AASSLQS | 32 | QQSVHTPYT | 67 |
| SA-28 | RASQSVRSSYL | 19 | GASSRAT | 38 | QQAGVSPYT | 68 |
| | A | | | | | |
| SA-29 | RASQSISSYLN | 14 | AASSLQS | 32 | QQSSHTPYT | 69 |
| SA-30 | KSSQSVLYSSN | 10 | WASTRES | 30 | QQLYIYPPT | 70 |
| | NKNYLA | | | | | |
| SA-31 | RASQSVSSSYL | 20 | GASSRAT | 38 | QQFFSYPPT | 71 |
| | A | | | | | |
| SA-32 | RASQSVGSNLA | 21 | GASTRAT | 36 | QQLNVFPWT | 72 |
| SA-33 | RASQGISSWLA | 12 | AASSLQS | 32 | LQAYSFPFT | 73 |
| SA-34 | RASQSISSWLA | 13 | DASSLES | 35 | QQYELLPPT | 74 |
| SA-35 | RASQSISSWLA | 13 | KASSLES | 39 | QQYGSFPLT | 75 |
| SA-36 | QASQDISSYLA | 22 | DASNRAT | 31 | QQRTHYPWT | 76 |
| SA-37 | RASQSVSSSEL | 23 | GASSRAT | 38 | QQYDGVPIT | 77 |
| | A | | | | | |
| SA-38 | RASQSVSSSLA | 16 | GASTRAT | 36 | QQYDNYPPIT | 78 |
| SA-39 | QASQDISNSLN | 24 | DASNLET | 33 | QQVIILPLT | 79 |
| SA-40 | RASQSVSSSYL | 20 | GASSRAT | 38 | QQLAHSPFT | 80 |
| | A | | | | | |
| SA-41 | RASQSVSSSLA | 16 | GASTRAT | 36 | QQYITYPIT | 81 |
| SA-42 | RASQSVSSNLA | 25 | GASTRAT | 36 | QQYNAYPIT | 82 |
| SA-43 | RASQGISSWLA | 12 | AASSLQS | 32 | QQASAFPIT | 83 |
| SA-44 | RASQGIDSWLA | 26 | AASSLQS | 32 | QQGVSLPYT | 84 |
| SA-45 | RASQSVSSSYL | 20 | GASSRAT | 38 | QQYYDSIT | 85 |
| | A | | | | | |
| SA-46 | RASQSVSSSYL | 20 | GASSRAT | 38 | QQHFSLPPT | 86 |
| | A | | | | | |
| SA-47 | RASQGISSWLA | 12 | AASSLQS | 32 | QQISSYPIT | 87 |
| SA-48 | KSSQSVLYSSN | 10 | WASTRES | 30 | QQLYIFPPT | 88 |
| | NKNYLA | | | | | |
| SA-49 | RASQGISSWLA | 12 | AASSLQS | 32 | QQVSLYPPT | 89 |
| SA-50 | KSSQSVLYSSN | 10 | WASTRES | 30 | QQLYLYPPT | 90 |
| | NKNYLA | | | | | |
| SA-51 | RASQSVSSSYL | 20 | GASSRAT | 38 | QQYYSSYT | 91 |
| | A | | | | | |
| SA-52 | RASQSVSSSYL | 20 | GASNRAT | 40 | QQYVDLPLT | 92 |
| | A | | | | | |
| SA-53 | QASQDISNYLN | 11 | DASNLET | 33 | QQSASLPYT | 93 |
| SA-54 | RASQGISSWLA | 12 | AASSLQS | 32 | QQVYLEPIT | 94 |
| SA-55 | RASQAISSWLA | 27 | DASSLES | 35 | QQYESFPWT | 95 |
| SA-56 | RASQGISSWLA | 12 | AASSLQS | 32 | QQAIYFPIT | 96 |
| SA-57 | RASQSVSSYLA | 7 | DASNRAT | 31 | QQDSAWPFT | 97 |
| SA-58 | QASQDISNYLN | 11 | DASNLET | 33 | QQPLNLPFT | 98 |
| SA-59 | QASQDISNYLN | 11 | DASNLET | 33 | QQVIDLPYT | 99 |
| SA-60 | RASQSVSSYLA | 7 | DSSNRAT | 29 | QDRSNLPWT | 100 |
| SA-61 | RASQSISSWLA | 13 | KASSLES | 39 | QQYGSFPLT | 75 |
| SA-62 | QASQDIANYLN | 28 | DASNLET | 33 | QQYVNYWT | 101 |

TABLE 3
Kabat Light Chain HVR Sequences of the Antibodies.

| | | SEQ ID | | SEQ ID | | SEQ ID |
|---|---|---|---|---|---|---|
| Antibody | HVR-H1 | NO: | HVR-H2 | NO: | HVR-H3 | NO: |
| SA-1 | FTFGDYAMH | 102 | GISWNSGSI | 123 | AKDAPYVGLDL | 147 |
| | | | GYADSVKG | | | |
| SA-2 | FTFSSYGMH | 103 | VISYDGSNK | 124 | AKDLGGGSGGYA | 148 |
| | | | YYADSVKG | | TSDYYYGMDV | |
| SA-3 | FTFSTYAMS | 104 | AISGSGGST | 125 | AKDLIDYASGGF | 149 |
| | | | YYADSVKG | | DY | |
| SA-4 | FTFSNYAMS | 105 | AISGSGGST | 125 | AKDQGQYHWHAF | 150 |
| | | | YYADSVKG | | DI | |
| SA-5 | FTFSSYAMS | 106 | AISGSGGST | 125 | AKDTGIDFWSVT | 151 |
| | | | YYADSVKG | | PPYFDL | |
| SA-6 | FTFDDYAMH | 107 | GITWNSGSI | 126 | AKEGRYGSGITT | 152 |
| | | | GYADSVKG | | T | |
| SA-7 | FTFSSYGMH | 103 | LIWYDGSNK | 127 | AKEGSSYADGWP | 153 |
| | | | YYADSVKG | | YGMDV | |
| SA-8 | FTFSSYGMH | 103 | VISYDGSNK | 124 | AKEPRSYWHGYG | 154 |
| | | | YYADSVKG | | MDV | |
| SA-9 | FTFSSYAMS | 106 | TISGSGGST | 128 | AKGAPQTSWLYG | 155 |
| | | | YYADSVKG | | MDV | |
| SA-10 | FTFDDYAMH | 107 | GITWNSGSI | 126 | AKGKQYSANYFD | 156 |
| | | | GYADSVKG | | Y | |
| SA-11 | FTFGDYAMH | 102 | GISWNSGSI | 123 | AKTRYGYYSYDF | 157 |
| | | | GYADSVKG | | DL | |
| SA-12 | YTFTSYYIH | 108 | IINPSGGST | 129 | ARAPGSWFY | 158 |
| | | | SYAQKFQG | | | |
| SA-13 | FTFSSYAMS | 106 | AISGSGGST | 125 | ARDGRTWGPHFY | 159 |
| | | | YYADSVKG | | | |
| SA-14 | FTFSSYSMN | 109 | SISSSSSYI | 130 | ARDGSMYYGDPM | 160 |
| | | | YYADSVKG | | DV | |
| SA-15 | GTFSSYAIS | 110 | GIIPIFGTA | 131 | ARDGSYSYATY | 161 |
| | | | NYAQKFQG | | | |
| SA-16 | GSFSGYYWS | 111 | EIDHSGSTN | 132 | ARDLGPPGPHLD | 162 |
| | | | YNPSLKS | | V | |
| SA-17 | GTFSSYAIS | 110 | GIIPIFGTA | 133 | ARDLSAFYVGPF | 163 |
| | | | SYAQKFQG | | DY | |
| SA-18 | FTFSSYWMS | 112 | NIKSDGSEK | 134 | ARDLSYEPSYPF | 164 |
| | | | YYVDSVKG | | DI | |
| SA-19 | FTFSSYGMH | 103 | VISYDGSNK | 124 | ARDLTKYTLGFA | 165 |
| | | | YYADSVKG | | FDI | |
| SA-20 | FTFSSYGMH | 103 | VISYDGSNK | 124 | AKEPRSYWHGYG | 154 |
| | | | YYADSVKG | | MDV | |
| SA-21 | YTFTGSYMH | 113 | WINPNSGGT | 135 | ARDPLHYYDSSG | 166 |
| | | | NYAQKFQG | | DVGIY | |
| SA-22 | FTFSSYSMN | 109 | SISSSSSYI | 130 | ARDPTDSSSYYD | 167 |
| | | | YYADSVKG | | V | |
| SA-23 | YTFTSYGIS | 114 | WISTYNGNT | 136 | ARDQGTGTTVDL | 168 |
| | | | NYAQKLQG | | DL | |
| SA-24 | FTFSSYGMH | 103 | VIWYDGSNK | 137 | ARDRGMVSSDYF | 169 |
| | | | YYADSVKG | | DY | |
| SA-25 | GTFSSYAIS | 110 | SIIPIFGTA | 138 | ARDRGYPRYSYL | 170 |
| | | | NYAQKFQG | | DL | |
| SA-26 | GSFSGYYWS | 111 | EIDHSGSTN | 132 | ARDRLDGSLGIY | 171 |
| | | | YNPSLKS | | GMDV | |
| SA-27 | GSISSSSYY | 115 | SIYYSGSTY | 139 | ARDSDKVALDL | 172 |
| | WG | | YNPSLKS | | | |
| SA-28 | GSISSGGYY | 116 | NIYYSGSTY | 140 | ARDSDTSWGGED | 173 |
| | WS | | YNPSLKS | | Y | |
| SA-29 | GSISSGGYY | 116 | NIYYSGSTY | 140 | ARDSDTSWGGED | 173 |
| | WS | | YNPSLKS | | Y | |
| SA-30 | FTFSSYGMH | 103 | VIWYDGSNK | 137 | ARDTGSATTMWL | 174 |
| | | | YYADSVKG | | YGMDV | |
| SA-31 | GTFSSYAIS | 110 | SIIPIFGTA | 138 | ARDRGYPRYSYL | 170 |
| | | | NYAQKFQG | | DL | |
| SA-32 | FTFSNYGMH | 117 | VIWYDGSNK | 137 | ARDTMSGSSPTD | 175 |
| | | | YYADSVKG | | Y | |
| SA-33 | GTFSSYAIS | 110 | GIIPIFGTA | 133 | ARDVGMARGDPY | 176 |
| | | | SYAQKFQG | | GMDV | |
| SA-34 | YTFTNYGIS | 118 | WISAYNGNT | 141 | ARDVYSSYSY | 177 |
| | | | NYAQKLQG | | | |
| SA-35 | YTFTSYYMH | 119 | IINPSGGST | 129 | AREAGYDIKGED | 178 |
| | | | SYAQKFQG | | Y | |
| SA-36 | YTFTGSYMH | 113 | WINPNSGGT | 135 | AREGAMGYRDYY | 179 |
| | | | NYAQKFQG | | MDV | |
| SA-37 | GSISSGGYY | 116 | SIYYSGSTY | 139 | AREGAYSLYVNW | 180 |
| | WS | | YNPSLKS | | FDP | |
| SA-38 | GSISSGGYY | 116 | SIYYSGSTY | 139 | AREGAYSLYVNW | 180 |
| | WS | | YNPSLKS | | FDP | |
| SA-39 | YTFTSYYMV | 120 | IINPSGGST | 129 | AREGGYGYRAYP | 181 |
| | | | SYAQKFQG | | YGMDV | |
| SA-40 | YTFTSYYMH | 119 | IINPSGGST | 142 | AREGLASTGVSD | 182 |
| | | | TYAQKFQG | | YYYMDV | |
| SA-41 | GSISSYYWS | 121 | SIYYSGSTN | 143 | AREGVAARGYYY | 183 |
| | | | YNPSLKS | | GMDV | |
| SA-42 | GSISSYYWS | 121 | SIYYSGSTN | 143 | AREGVAARGYYY | 183 |
| | | | YNPSLKS | | GMDV | |
| SA-43 | GSISSSSYA | 122 | SIYYSGSTY | 139 | ARELETSRHDI | 184 |
| | WG | | YNPSLKS | | | |
| SA-44 | FTFSSYSMN | 109 | YISSSSSTI | 144 | AREPFYSADRED | 185 |
| | | | YYADSVKG | | Y | |
| SA-45 | YTFTSYGIS | 114 | WISAYNGNT | 141 | ARESGGTGAYGM | 186 |
| | | | NYAQKLQG | | DV | |
| SA-46 | FTFSTYAMS | 104 | AISGSGGST | 125 | ARESYDSSLHYY | 187 |
| | | | YYADSVKG | | GMDV | |
| SA-47 | YTFTSYYMH | 119 | IINPSGGST | 129 | AREVGVSGWEIT | 188 |
| | | | SYAQKFQG | | YGMDV | |
| SA-48 | FTFSSYGMH | 103 | VISYDGSNK | 124 | ARGAPIETLGVY | 189 |
| | | | YYADSVKG | | YYGMDV | |
| SA-49 | FTFSSYSMN | 109 | SISSSSSYI | 130 | ARGAPSMQYSPY | 190 |
| | | | YYADSVKG | | YYYGMDV | |
| SA-50 | FTFSSYSMN | 109 | YISSSSSTI | 144 | ARGGGGYSYSPY | 191 |
| | | | YYADSVKG | | IYGMDV | |
| SA-51 | GTFSSYAIS | 110 | SIIPIFGTA | 138 | ARGGYAAGTDYY | 192 |
| | | | NYAQKFQG | | MGV | |
| SA-52 | YTFTNYGIS | 118 | WISAYNGNT | 141 | ARGLWSEHIY | 193 |
| | | | NYAQKLQG | | | |
| SA-53 | GSISSGGYY | 116 | YIYYSGSTY | 145 | ARGPLGAAGFDY | 194 |
| | WS | | YNPSLKS | | | |
| SA-54 | GSISSSSYY | 115 | SIYYSGSTY | 139 | ARGSGIAAADTD | 195 |
| | WG | | YNPSLKS | | GLPMDV | |
| SA-55 | FTFSSYWMS | 112 | NIKQDGSEK | 146 | ARGSPYYDAYDF | 196 |
| | | | YYVDSVKG | | DL | |
| SA-56 | FTFSSYSMN | 109 | SISSSSSYI | 130 | ARGVETYSGPNW | 197 |
| | | | YYADSVKG | | FDP | |
| SA-57 | GSISSSSYA | 122 | SIYYSGSTY | 139 | ARKGMDDAGMDV | 198 |
| | WG | | YNPSLKS | | | |
| SA-58 | GSISSSSYA | 122 | SIYYSGSTY | 139 | ARLPMGCGSGMD | 199 |
| | WG | | YNPSLKS | | V | |
| SA-59 | GSISSYYWS | 121 | SIYYSGSTN | 143 | ARSGGSYSYLDV | 200 |
| | | | YNPSLKS | | | |
| SA-60 | FTFSSYSMN | 109 | YISSSSSTI | 144 | ARTHKHFDYGMD | 201 |
| | | | YYADSVKG | | V | |
| SA-61 | YTFTSYYMH | 119 | IINPSGGST | 129 | AREAGYDIKGFD | 178 |
| | | | SYAQKFQG | | Y | |
| SA-62 | FTFSSYSMN | 109 | YISSSSSTI | 144 | ARTHKHEDYGMD | 201 |
| | | | YYADSVKG | | V | |

TABLE 4
Heavy Chain Variable Regions and Light Chain Variable Regions of the Antibodies.

| | | SEQ ID | | SEQ ID |
|---|---|---|---|---|
| Antibody | Heavy Chain Variable | NO: | Light Chain Variable | NO: |
| SA-1 | EVQLVESGGGLVQPGRSLRLS | 202 | EIVLTQSPATLSLSPGERATLSC | 257 |
| | CAASGFTFGDYAMHWVRQAPG | | RASQSVSSYLAWYQQKPGQAPRL | |
| | KGLEWVSGISWNSGSIGYADS | | LIYDSSNRATGIPARFSGSGSGT | |
| | VKGRFTISRDNAKNSLYLQMN | | DFTLTISSLEPEDFAVYYCQQDS | |
| | SLRAEDTALYYCAKDAPYVGL | | DYPFTFGGGTKVEIK | |
| | DLWGRGTLVTVSS | | | |
| SA-2 | QVQLVESGGGVVQPGRSLRLS | 203 | DIVMTQSPDSLAVSLGERATINC | 258 |
| | CAASGFTFSSYGMHWVRQAPG | | KSSQSVLESSNNKNYLAWYQQKP | |
| | KGLEWVAVISYDGSNKYYADS | | GQPPKLLIYWASTRESGVPDRFS | |
| | VKGRFTISRDNSKNTLYLQMN | | GSGSGTDFTLTISSLQAEDVAVY | |
| | SLRAEDTAVYYCAKDLGGGSG | | YCQQYFLYPPTFGGGTKVEIK | |
| | GYATSDYYYGMDVWGQGTTVT | | | |
| | VSS | | | |
| SA-3 | EVQLLESGGGLVQPGGSLRLS | 204 | EIVLTQSPATLSLSPGERATLSC | 259 |
| | CAASGFTFSTYAMSWVRQAPG | | RASQSVSSYLAWYQQKPGQAPRL | |
| | KGLEWVSAISGSGGSTYYADS | | LIYDASNRATGIPARFSGSGSGT | |
| | VKGRFTISRDNSKNTLYLQMN | | DETLTISSLEPEDFAVYYCQQHD | |
| | SLRAEDTAVYYCAKDLIDYAS | | FWPPTFGGGTKVEIK | |
| | GGFDYWGQGTLVTVSS | | | |
| SA-4 | EVQLLESGGGLVQPGGSLRLS | 205 | EIVLTQSPATLSLSPGERATLSC | 260 |
| | CAASGFTFSNYAMSWVRQAPG | | RASQSVSSYLAWYQQKPGQAPRL | |
| | KGLEWVSAISGSGGSTYYADS | | LIYDASNRATGIPARESGSGSGT | |
| | VKGRFTISRDNSKNTLYLQMN | | DFTLTISSLEPEDFAVYYCQQDS | |
| | SLRAEDTAVYYCAKDQGQYHW | | NFPFTFGGGTKVEIK | |
| | HAFDIWGQGTMVTVSS | | | |
| SA-5 | EVQLLESGGGLVQPGGSLRLS | 206 | DIQLTQSPSSVSASVGDRVTITC | 261 |
| | CAASGFTFSSYAMSWVRQAPG | | RASQDISSWLAWYQQKPGKAPKL | |
| | KGLEWVSAISGSGGSTYYADS | | LIYAASSLQSGVPSRFSGSGSGT | |
| | VKGRFTISRDNSKNTLYLQMN | | DFTLTISSLQPEDFATYYCQQGY | |
| | SLRAEDTAVYYCAKDTGIDEW | | IFPRTFGGGTKVEIK | |
| | SVTPPYFDLWGRGTLVTVSS | | | |
| SA-6 | EVQLVESGGGLVQPGRSLRLS | 207 | EIVMTQSPATLSLSPGERATLSC | 262 |
| | CAASGFTFDDYAMHWVRQAPG | | RASQSVSSYLAWYQQKPGQAPRL | |
| | KGLEWVSGITWNSGSIGYADS | | LIYDSSNRATGIPARFSGSGSGT | |
| | VKGRFTISRDNAKNSLYLQMN | | DETLTISSLEPEDFAVYYCQQDA | |
| | SLRAEDTALYYCAKEGRYGSG | | NYPYTFGGGTKVEIK | |
| | ITTTWGQGTLVTVSS | | | |
| SA-7 | QVQLVESGGGVVQPGRSLRLS | 208 | DIVMTQSPDSLAVSLGERATINC | 263 |
| | CAASGFTFSSYGMHWVRQAPG | | KSSQSVLYSSNNKNYLAWYQQKP | |
| | KGLEWVALIWYDGSNKYYADS | | GQPPKLLIYWASTRESGVPDRFS | |
| | VKGRFTISRDNSKNTLYLQMN | | GSGSGTDFTLTISSLQAEDVAVY | |
| | SLRAEDTAVYYCAKEGSSYAD | | YCQQYYHYPPTFGGGTKVEIK | |
| | GWPYGMDVWGQGTTVTVSS | | | |
| SA-8 | QVQLVESGGGVVQPGRSLRLS | 209 | DIQMTQSPSSLSASVGDRVTITC | 264 |
| | CAASGFTFSSYGMHWVRQAPG | | QASQDISNYLNWYQQKPGKAPKL | |
| | KGLEWVAVISYDGSNKYYADS | | LIYDASNLETGVPSRFSGSGSGT | |
| | VKGRFTISRDNSKNTLYLQMN | | DETFTISSLQPEDIATYYCQQLS | |
| | SLRAEDTAVYYCAKEPRSYWH | | NYPITFGGGTKVEIK | |
| | GYGMDVWGQGTTVTVSS | | | |
| SA-9 | EVQLLESGGGLVQPGGSLRLS | 210 | DIQMTQSPSSVSASVGDRVTITC | 265 |
| | CAASGFTFSSYAMSWVRQAPG | | RASQGISSWLAWYQQKPGKAPKL | |
| | KGLEWVSTISGSGGSTYYADS | | LIYAASSLQSGVPSRFSGSGSGT | |
| | VKGRFTISRDNSKNTLYLQMN | | DFTLTISSLQPEDFATYYCQQAY | |
| | SLRAEDTAVYYCAKGAPQTSW | | LYPITFGGGTKVENQ | |
| | LYGMDVWGQGTTVTVSS | | | |
| SA-10 | EVQLVESGGGLVQPGRSLRLS | 211 | DIQMTQSPSTLSASVGDRVTITC | 266 |
| | CAASGFTFDDYAMHWVRQAPG | | RASQSISSWLAWYQQKPGKAPKL | |
| | KGLEWVSGITWNSGSIGYADS | | LIYEASSLESGVPSRFSGSGSGT | |
| | VKGRFTISRDNAKNSLYLQMN | | EFTLTISSLQPDDFATYYCQQDN | |
| | SLRAEDTALYYCAKGKQYSAN | | SLPYTFGGGTKVEIK | |
| | YFDYWGQGTLVTVSS | | | |
| SA-11 | EVQLVESGGGLVQPGRSLRIS | 212 | DIQMTQSPSTLSASVGDRVTITC | 267 |
| | CAASGFTFGDYAMHWVRQAPG | | RASQSISSWLAWYQQKPGKAPKL | |
| | KGLEWVSGISWNSGSIGYADS | | LIYDASSLESGVPSRFSGSGSGT | |
| | VKGRFTISRDNAKNSLYLQMN | | EFTLTISSLQPDDFATYYCQQYN | |
| | SLRAEDTALYYCAKTRYGYYS | | SYSPTFGGGTKVEIK | |
| | YDFDLWGRGTLVTVSS | | | |
| SA-12 | QVQLVQSGAEVKKPGASVKVS | 213 | EIVLTQSPATLSLSPGERATLSC | 268 |
| | CKASGYTFTSYYIHWVRQAPG | | RASQSVSSYLAWYQQKPGQAPRL | |
| | QGLEWMGIINPSGGSTSYAQK | | LIYDASNRATGIPARFSGSGSGT | |
| | FQGRVTMTRDTSTSTVYMELS | | DETLTISSLEPEDFAVYYCQQRT | |
| | SLRSEDTAVYYCARAPGSWFY | | NFPITFGGGTKVEIK | |
| | WGQGTLVTVSS | | | |
| SA-13 | EVQLLESGGGLVQPGGSLRLS | 214 | DIQMTQSPSSLSASVGDRVTITC | 269 |
| | CAASGFTFSSYAMSWVRQAPG | | RASQSISSYLNWYQQKPGKAPKL | |
| | KGLEWVSAISGSGGSTYYADS | | LIYAASSLQSGVPSRFSGSGSGT | |
| | VKGRFTISRDNSKNTLYLQMN | | DFTLTISSLQPEDFATYYCQQGY | |
| | SLRAEDTAVYYCARDGRTWGP | | IYPPTFGGGTKVEIK | |
| | HFYWGQGTLVTVSS | | | |
| SA-14 | EVQLVESGGGLVKPGGSLRLS | 215 | DIQLTQSPSSLSASVGDRVTITC | 270 |
| | CAASGFTFSSYSMNWVRQAPG | | QASQDISNFLNWYQQKPGKAPKL | |
| | KGLEWVSSISSSSSYIYYADS | | LIYDASNLETGVPSRFSGSGSGT | |
| | VKGRFTISRDNAKNSLYLQMN | | DFTFTISSLQPEDIAKYYCQQFI | |
| | SLRAEDTAVYYCARDGSMYYG | | NFPLTFGGGTKVEIK | |
| | DPMDVWGQGTTVTVSS | | | |
| SA-15 | QVQLVQSGAEVKKPGSSVKVS | 216 | EIVLTQSPATLSLSPGERATLSC | 271 |
| | CKASGGTFSSYAISWVRQAPG | | RASQSVSSYLAWYQQKPGQAPRL | |
| | QGLEWMGGIIPIFGTANYAQK | | LIYDASNRATGIPARFSGSGSGT | |
| | FQGRVTITADESTSTAYMELS | | DFTLTISSLEPEDFAVYYCQQPY | |
| | SLRSEDTAVYYCARDGSYSYA | | NFPLTFGGGTKVEIK | |
| | TYWGQGTLVTVSS | | | |
| SA-16 | QVQLQQWGAGLLKPSETLSLT | 217 | EIVLTQSPATLSLSPGERATLSC | 272 |
| | CAVYGGSFSGYYWSWIRQPPG | | RASQSVSSYLAWYQQKPGQAPRL | |
| | KGLEWIGEIDHSGSTNYNPSL | | LIYDASNRATGIPARFSGSGSGT | |
| | KSRVTISVDTSKNQFSLKLSS | | DFTLTISSLEPEDFAVYYCQQFS | |
| | VTAADTAVYYCARDLGPPGPH | | DYPTFGGGTKVEIK | |
| | LDVWGQGTMVTVSS | | | |
| SA-17 | QVQLVQSGAEVKKPGSSVKVS | 218 | EIVMTQSPATLSVSPGERATLSC | 273 |
| | CKASGGTFSSYAISWVRQAPG | | RASQSVSSSLAWYQQKPGQAPRL | |
| | QGLEWMGGIIPIFGTASYAQK | | LIYGASTRATGIPARFSGSGSGT | |
| | FQGRVTITADESTSTAYMELS | | EFTLTISSLQSEDFAVYYCQQDY | |
| | SLRSEDTAVYYCARDLSAFYV | | IWPLTFGGGTKVEIK | |
| | GPFDYWGQGTLVTVSS | | | |
| SA-18 | EVQLVESGGGLVQPGGSLRLS | 219 | DIQMTQSPSSVSASVGDRVTITC | 274 |
| | CAASGFTFSSYWMSWVRQAPG | | RASQGISSWLAWYQQKPGKAPKL | |
| | KGLEWVANIKSDGSEKYYVDS | | LIYAASSLQSGVPSRESGSGSGT | |
| | VKGRFTISRDNAKNSLYLQMN | | DFTLTISSLQPEDFATYYCQQAL | |
| | SLRAEDTAVYYCARDLSYEPS | | SFPFTFGGGTKVEIK | |
| | YPFDIWGQGTMVTVSS | | | |
| SA-19 | QVQLVESGGGVVQPGRSLRLS | 220 | DIQMTQSPSSLSASVGDRVTITC | 275 |
| | CAASGFTFSSYGMHWVRQAPG | | QASQDISNYLNWYQQKPGKAPKL | |
| | KGLEWVAVISYDGSNKYYADS | | LIYDASNLETGVPSRFSGSGSGT | |
| | VKGRFTISRDNSKNTLYLQMN | | DETFTISSLQPEDIATYYCQQFT | |
| | SLRAEDTAVYYCARDLTKYTL | | YLPPTFGGGTKVEIK | |
| | GFAFDIWGQGTMVTVSS | | | |
| SA-20 | QVQLVESGGGVVQPGRSLRLS | 209 | DIQMTQSPSSLSASVGDRVTITC | 276 |
| | CAASGFTFSSYGMHWVRQAPG | | QASQDISNYLNWYQQKPGKAPKL | |
| | KGLEWVAVISYDGSNKYYADS | | LIYDASNLETGVPSRFSGSGSGT | |
| | VKGRFTISRDNSKNTLYLQMN | | DFTFTISSLQPEDIATYYCQQVD | |
| | SLRAEDTAVYYCAKEPRSYWH | | VLPYTFGGGTKVEIK | |
| | GYGMDVWGQGTTVTVSS | | | |
| SA-21 | QVQLVQSGAEVKKPGASVKVS | 221 | DIVMTQSPDSLAVSLGERATINC | 277 |
| | CKASGYTFTGSYMHWVRQAPG | | KSSQSVLESSNNKNYLAWYQQKP | |
| | QGLEWMGWINPNSGGTNYAQK | | GQPPKLLIYWASTRESGVPDRES | |
| | FQGRVTMTRDTSISTAYMELS | | GSGSGTDFTLTISSLQAEDVAVY | |
| | RLRSDDTAVYYCARDPLHYYD | | YCQQDYATPFTFGGGTKVEIK | |
| | SSGDVGIYWGQGTLVTVSS | | | |
| SA-22 | EVQLVESGGGLVKPGGSLRLS | 222 | DIQMTQSPSSLSASVGDRVTITC | 278 |
| | CAASGFTFSSYSMNWVRQAPG | | QASQDISNYLNWYQQKPGKAPKL | |
| | KGLEWVSSISSSSSYIYYADS | | LIYDASNLETGVPSRFSGSGSGT | |
| | VKGRFTISRDNAKNSLYLQMN | | DFTFTISSLQPEDIATYYCQQYD | |
| | SLRAEDTAVYYCARDPTDSSS | | TLPPTFGGGTKVEIK | |
| | YYDVWGQGTLVTVSS | | | |
| SA-23 | QVQLVQSGAEVKKPGASVKVS | 223 | DIVMTQSPLSLPVTPGEPASISC | 279 |
| | CKASGYTFTSYGISWVRQAPG | | RSSQSLLHSNGYNYLDWYLQKPG | |
| | QGLEWMGWISTYNGNTNYAQK | | QSPQLLIYLGSNRASGVPDRFSG | |
| | LQGRVTMTTDTSTSTAYMELR | | SGSGTDFTLKISRVEAEDVGVYY | |
| | SLRSDDTAVYYCARDQGTGTT | | CMQAQGTPYTFGGGTKVEIK | |
| | VDLDLWGRGTLVTVSS | | | |
| SA-24 | QVQLVESGGGVVQPGRSLRLS | 224 | EIVLTQSPATLSLSPGERATLSC | 280 |
| | CAASGFTFSSYGMHWVRQAPG | | RASQSVSSYLAWYQQKPGQAPRL | |
| | KGLEWVAVIWYDGSNKYYADS | | LIYDASNRATGIPARFSGSGSGT | |
| | VKGRFTISRDNSKNTLYLQMN | | DFTLTISSLEPEDFAVYYCQQRL | |
| | SLRAEDTAVYYCARDRGMVSS | | DWPYTFGGGTKVEIK | |
| | DYFDYWGQGTLVTVSS | | | |
| SA-25 | QVQLVQSGAEVKKPGSSVKVS | 225 | DIQMTQSPSSLSASVGDRVTITC | 281 |
| | CKASGGTFSSYAISWVRQAPG | | QASQDISNYLNWYQQKPGKAPKL | |
| | QGLEWMGSIIPIFGTANYAQK | | LIYDASNLETGVPSRFSGSGSGT | |
| | FQGRVTITADESTSTAYMELS | | DFTFTISSLQPEDIATYYCQQYS | |
| | SLRSEDTAVYYCARDRGYPRY | | YLPTFGGGTKVEIK | |
| | SYLDLWGRGTLVTVSS | | | |
| SA-26 | QVQLQQWGAGLLKPSETLSLT | 226 | DIQMTQSPSSLSASVGDRVTITC | 282 |
| | CAVYGGSFSGYYWSWIRQPPG | | RASQSINSYLNWYQQKPGKAPKL | |
| | KGLEWIGEIDHSGSTNYNPSL | | LIYAASSLQSGVPSRFSGSGSGT | |
| | KSRVTISVDTSKNQFSLKLSS | | DFTLTISSLQPEDFATYYCQQSP | |
| | VTAADTAVYYCARDRLDGSLG | | SDPWTFGGGTKVEIK | |
| | IYGMDVWGQGTTVTVSS | | | |
| SA-27 | QLQLQESGPGLVKPSETLSLT | 227 | DIQMTQSPSSLSASVGDRVTITC | 283 |
| | CTVSGGSISSSSYYWGWIRQP | | RASQSISSYLNWYQQKPGKAPKL | |
| | PGKGLEWIGSIYYSGSTYYNP | | LIYAASSLQSGVPSRFSGSGSGT | |
| | SLKSRVTISVDTSKNQFSLKL | | DFTLTISSLQPEDFATYYCQQSV | |
| | SSVTAADTAVYYCARDSDKVA | | HTPYTFGGGTKVEIK | |
| | LDLWGRGTLVTVSS | | | |
| SA-28 | QVQLQESGPGLVKPSQTLSLT | 228 | EIVLTQSPGTLSLSPGERATLSC | 284 |
| | CTVSGGSISSGGYYWSWIRQH | | RASQSVRSSYLAWYQQKPGQAPR | |
| | PGKGLEWIGNIYYSGSTYYNP | | LLIYGASSRATGIPDRFSGSGSG | |
| | SLKSRVTISVDTSKNQFSLKL | | TDFTLTISRLEPEDFAVYYCQQA | |
| | SSVTAADTAVYYCARDSDTSW | | GVSPYTFGGGTKVEIK | |
| | GGFDYWGQGTLVTVSS | | | |
| SA-29 | QVQLQESGPGLVKPSQTLSLT | 228 | DIQMTQSPSSLSASVGDRVTITC | 285 |
| | CTVSGGSISSGGYYWSWIRQH | | RASQSISSYLNWYQQKPGKAPKL | |
| | PGKGLEWIGNIYYSGSTYYNP | | LIYAASSLQSGVPSRFSGSGSGT | |
| | SLKSRVTISVDTSKNQFSLKL | | DETLTISSLQPEDFATYYCQQSS | |
| | SSVTAADTAVYYCARDSDTSW | | HTPYTFGGGTKVEIK | |
| | GGFDYWGQGTLVTVSS | | | |
| SA-30 | QVQLVESGGGVVQPGRSLRLS | 229 | DIVMTQSPDSLAVSLGERATINC | 286 |
| | CAASGFTFSSYGMHWVRQAPG | | KSSQSVLYSSNNKNYLAWYQQKP | |
| | KGLEWVAVIWYDGSNKYYADS | | GQPPKLLIYWASTRESGVPDRES | |
| | VKGRFTISRDNSKNTLYLQMN | | GSGSGTDFTLTISSLQAEDVAVY | |
| | SLRAEDTAVYYCARDTGSATT | | YCQQLYIYPPTFGGGTKVEIK | |
| | MWLYGMDVWGKGTTVTVSS | | | |
| SA-31 | QVQLVQSGAEVKKPGSSVKVS | 225 | EIVLTQSPGTLSLSPGERATLSC | 287 |
| | CKASGGTFSSYAISWVRQAPG | | RASQSVSSSYLAWYQQKPGQAPR | |
| | QGLEWMGSIIPIFGTANYAQK | | LLIYGASSRATGIPDRFSGSGSG | |
| | FQGRVTITADESTSTAYMELS | | TDFTLTISRLEPEDFAVYYCQQF | |
| | SLRSEDTAVYYCARDRGYPRY | | FSYPPTFGGGTKVEIK | |
| | SYLDLWGRGTLVTVSS | | | |
| SA-32 | QVQLVESGGGVVQPGRSLRLS | 230 | EIVMTQSPATLSVSPGERATLSC | 288 |
| | CAASGFTFSNYGMHWVRQAPG | | RASQSVGSNLAWYQQKPGQAPRL | |
| | KGLEWVAVIWYDGSNKYYADS | | LIYGASTRATGIPARFSGSGSGT | |
| | VKGRFTISRDNSKNTLYLQMN | | EFTLTISSLQSEDFAVYYCQQLN | |
| | SLRAEDTAVYYCARDTMSGSS | | VFPWTFGGGTKVEIK | |
| | PTDYWGQGTLVTVSS | | | |
| SA-33 | QVQLVQSGAEVKKPGSSVKVS | 231 | DIQMTQSPSSVSASVGDRVTITC | 289 |
| | CKASGGTFSSYAISWVRQAPG | | RASQGISSWLAWYQQKPGKAPKL | |
| | QGLEWMGGIIPIFGTASYAQK | | LISAASSLQSGVPSRFSGSGSGT | |
| | FQGRVTITADESTSTAYMELS | | DFTLTISSLQPEDFATYYCLQAY | |
| | SLRSEDTAVYYCARDVGMARG | | SFPFTFGGGTKVEIK | |
| | DPYGMDVWGQGTTVTVSS | | | |
| SA-34 | QVQLVQSGAEVKKPGASVKVS | 232 | DIQLTQSPSTLSASVGDRVTITC | 290 |
| | CKASGYTFTNYGISWVRQAPG | | RASQSISSWLAWYQQKPGKAPKL | |
| | QGLEWMGWISAYNGNTNYAQK | | LIYDASSLESGVPSRFSGSGSGT | |
| | LQGRVTMTTDTSTSTAYMELR | | EFTLTISSLQPDDFATYYCQQYE | |
| | SLRSDDTAVYYCARDVYSSYS | | LLPPTFGGGTKVEIK | |
| | YWGQGTLVTVSS | | | |
| SA-35 | QVQLVQSGAEVKKPGASVKVS | 233 | DIQMTQSPSTLSASVGDRVTITC | 291 |
| | CKASGYTFTSYYMHWVRQAPG | | RASQSISSWLAWYQQKPGKAPKL | |
| | QGLEWMGIINPSGGSTSYAQK | | LIYKASSLESGVPSRFSGSGSGT | |
| | FQGRVTMTRDTSTSTVYMELS | | EFTLTISSLQPDDFATYYCQQYG | |
| | SLRSEDTAVYYCAREAGYDIK | | SFPLTFGGGTKVEIK | |
| | GFDYWGQGTLVTVSS | | | |
| SA-36 | QVQLVQSGAEVKKPGASVKVS | 234 | EIVLTQSPATLSLSPGERATITC | 292 |
| | CKASGYTFTGSYMHWVRQAPG | | QASQDISSYLAWYQQKPGQAPRL | |
| | QGLEWMGWINPNSGGTNYAQK | | LIYDASNRATGIPARFSGSGSGT | |
| | FQGRVTMTRDTSISTAYMELS | | DFTLTISSLEPEDFAVYYCQQRT | |
| | RLRSDDTAVYYCAREGAMGYR | | HYPWTFGGGTKVEIK | |
| | DYYMDVWGKGTTVTVSS | | | |
| SA-37 | QVQLQESGPGLVKPSQTLSLT | 235 | EIVLTQSPGTLSLSPGERATLSC | 293 |
| | CTVSGGSISSGGYYWSWIRQH | | RASQSVSSSFLAWYQQKPGQAPR | |
| | PGKGLEWIGSIYYSGSTYYNP | | LLIYGASSRATGIPDRFSGSGSG | |
| | SLKSRVTISVDTSKNQFSLKL | | TDFTLTISRLEPEDFAVYYCQQY | |
| | SSVTAADTAVYYCAREGAYSL | | DGVPITFGGGTKVEIK | |
| | YVNWFDPWGQGTLVTVSS | | | |
| SA-38 | QVQLQESGPGLVKPSQTLSLT | 235 | EIVMTQSPATLSVSPGERATLSC | 294 |
| | CTVSGGSISSGGYYWSWIRQH | | RASQSVSSSLAWYQQKPGQAPRL | |
| | PGKGLEWIGSIYYSGSTYYNP | | LIFGASTRATGIPARFSGSGSGT | |
| | SLKSRVTISVDTSKNQFSLKL | | EFTLTISSLQSEDFAVYYCQQYD | |
| | SSVTAADTAVYYCAREGAYSL | | NYPPITFGGGTKVEIK | |
| | YVNWFDPWGQGTLVTVSS | | | |
| SA-39 | QVQLVQSGAEVKKPGASVKVS | 236 | DIQMTQSPSSLSASVGDRVTITC | 295 |
| | CKASGYTFTSYYMVWVRQAPG | | QASQDISNSLNWYQQKPGKAPKL | |
| | QGLEWMGIINPSGGSTSYAQK | | LIYDASNLETGVPSRFSGSRSGT | |
| | FQGRVTMTRDTSTSTVYMELS | | DETFTISSLQPEDIATYYCQQVI | |
| | SLRSEDTAVYYCAREGGYGYR | | ILPLTFGGGTKVEIK | |
| | AYPYGMDVWGQGTTVTVSS | | | |
| SA-40 | QVQLVQSGAEVKKPGASVKVS | 237 | EIVLTQSPGTLSLSPGERATLSC | 296 |
| | CKASGYTFTSYYMHWVRQAPG | | RASQSVSSSYLAWYQQKPGQAPR | |
| | QGLEWMGIINPSGGSTTYAQK | | LLIYGASSRATGIPDRFSGSGSG | |
| | FQGRVTMTRDTSTSTVYMELS | | TDFTLTISRLEPEDFAVYYCQQL | |
| | SLRSEDTAVYYCAREGLASTG | | AHSPFTFGGGTKVEIK | |
| | VSDYYYMDVWGKGTTVTVSS | | | |
| SA-41 | QVQLQESGPGLVKPSETLSLT | 238 | EIVMTQSPATLSVSPGERATLSC | 297 |
| | CTVSGGSISSYYWSWIRQPPG | | RASQSVSSSLAWYQQKPGQAPRL | |
| | KGLEWIGSIYYSGSTNYNPSL | | LIYGASTRATGIPARFSGSGSGT | |
| | KSRVTISVDTSKNQFSLKLSS | | EFTLTISSLQSEDFAVYYCQQYI | |
| | VTAADTAVYYCAREGVAARGY | | TYPITFGGGTKVEIK | |
| | YYGMDVWGQGTTVTVSS | | | |
| SA-42 | QVQLQESGPGLVKPSETLSLT | 238 | EIVMTQSPATLSVSPGERATLSC | 298 |
| | CTVSGGSISSYYWSWIRQPPG | | RASQSVSSNLAWYQQKPGQAPRL | |
| | KGLEWIGSIYYSGSTNYNPSL | | LIFGASTRATGIPARFSGSGSGT | |
| | KSRVTISVDTSKNQFSLKLSS | | EFTLTISSLQSEDFAVYYCQQYN | |
| | VTAADTAVYYCAREGVAARGY | | AYPITFGGGTKVEIK | |
| | YYGMDVWGQGTTVTVSS | | | |
| SA-43 | QLQLQESGPGLVKPSETLSLT | 239 | DIQMTQSPSSVSASVGDRVTITC | 299 |
| | CTVSGGSISSSSYAWGWIRQP | | RASQGISSWLAWYQQKPGKAPKL | |
| | PGKGLEWIGSIYYSGSTYYNP | | LIYAASSLQSGVPSRFSGSGSGT | |
| | SLKSRVTISVDTSKNQFSLKL | | DFTLTISSLQPEDFATYYCQQAS | |
| | SSVTAADTAVYYCARELETSR | | AFPITFGGGTKVEIK | |
| | HDIWGQGTTVTVSS | | | |
| SA-44 | EVQLVESGGGLVQPGGSLRLS | 240 | DIQMTQSPSSVSASVGDRVTITC | 300 |
| | CAASGFTFSSYSMNWVRQAPG | | RASQGIDSWLAWYQQKPGKAPKL | |
| | KGLEWVSYISSSSSTIYYADS | | LIYAASSLQSGVPSRFSGSGSGT | |
| | VKGRFTISRDNAKNSLYLQMN | | DFTLTISSLQPEDFATYYCQQGV | |
| | SLRAEDTAVYYCAREPFYSAD | | SLPYTFGGGTKVEIK | |
| | RFDYWGQGTLVTVSS | | | |
| SA-45 | QVQLVQSGAEVKKPGASVKVS | 241 | DIQMTQSPSSLSASVGDRVTITC | 301 |
| | CKASGYTFTSYGISWVRQAPG | | RASQSVSSSYLAWYQQKPGQAPR | |
| | QGLEWMGWISAYNGNTNYAQK | | LLIYGASSRATGIPDRFSGSGSG | |
| | LQGRVTMTTDTSTSTAYMELR | | TDFTLTISRLEPEDFAVYYCQQY | |
| | SLRSDDTAVYYCARESGGTGA | | YDSITFGGGTKVEIK | |
| | YGMDVWGQGTTVTVSS | | | |
| SA-46 | EVQLLESGGGLVQPGGSLRLS | 242 | EIVLTQSPGTLSLSPGERATLSC | 302 |
| | CAASGFTFSTYAMSWVRQAPG | | RASQSVSSSYLAWYQQKPGQAPR | |
| | KGLEWVSAISGSGGSTYYADS | | LLIYGASSRATGIPDRFSGSGSG | |
| | VKGRFTISRDNSKNTLYLQMN | | TDFTLTISRLEPEDFAVYYCQQH | |
| | SLRAEDTAVYYCARESYDSSL | | FSLPPTFGGGTKVEIK | |
| | HYYGMDVWGQGTTVTVSS | | | |
| SA-47 | QVQLVQSGAEVKKPGASVKVS | 243 | DIQMTQSPSSVSASVGDRVTITC | 303 |
| | CKASGYTFTSYYMHWVRQAPG | | RASQGISSWLAWYQQKPGKAPKL | |
| | QGLEWMGIINPSGGSTSYAQK | | LIYAASSLQSGVPSRFSGSGSGT | |
| | FQGRVTMTRDTSTSTVYMELS | | DFTLTISSLQPEDFATYYCQQIS | |
| | SLRSEDTAVYYCAREVGVSGW | | SYPITFGGGTKVEIK | |
| | EITYGMDVWGQGTTVTVSS | | | |
| SA-48 | QVQLVESGGGVVQPGRSLRLS | 244 | DIVMTQSPDSLAVSLGERATINC | 304 |
| | CAASGFTFSSYGMHWVRQAPG | | KSSQSVLYSSNNKNYLAWYQQKP | |
| | KGLEWVAVISYDGSNKYYADS | | GQPPKLLIYWASTRESGVPDRFS | |
| | VKGRFTISRDNSKNTLYLQMN | | GSGSGTDFTLTISSLQAEDVAVY | |
| | SLRAEDTAVYYCARGAPIETL | | YCQQLYIFPPTFGGGTKVEIK | |
| | GVYYYGMDVWGQGTTVTVSS | | | |
| SA-49 | EVQLVESGGGLVKPGGSLRLS | 245 | DIQMTQSPSSVSASVGDRVTITC | 305 |
| | CAASGFTFSSYSMNWVRQAPG | | RASQGISSWLAWYQQKPGKAPKL | |
| | KGLEWVSSISSSSSYIYYADS | | LIYAASSLQSGVPSRFSGSGSGT | |
| | VKGRFTISRDNAKNSLYLQMN | | DFTLTISSLQPEDFATYYCQQVS | |
| | SLRAEDTAVYYCARGAPSMQY | | LYPPTFGGGTKVEIK | |
| | SPYYYYGMDVWGQGTTVTVSS | | | |
| SA-50 | EVQLVESGGGLVQPGGSLRLS | 246 | DIVMTQSPDSLAVSLGERATINC | 306 |
| | CAASGFTFSSYSMNWVRQAPG | | KSSQSVLYSSNNKNYLAWYQQKP | |
| | KGLEWVSYISSSSSTIYYADS | | GQPPKLLIYWASTRESGVPDRFS | |
| | VKGRFTISRDNAKNSLYLQMN | | GSGSGTDFTLTISSLQAEDVAVY | |
| | SLRAEDTAVYYCARGGGGYSY | | YCQQLYLYPPTFGGGTKVEIK | |
| | SPYIYGMDVWGQGTTVTVSS | | | |
| SA-51 | QVQLVQSGAEVKKPGSSVKVS | 247 | EIVLTQSPGTLSLSPGERATLSC | 307 |
| | CKASGGTFSSYAISWVRQAPG | | RASQSVSSSYLAWYQQKPGQAPR | |
| | QGLEWMGSIIPIFGTANYAQK | | LLIYGASSRATGIPDRFSGSGSG | |
| | FQGRVTITADESTSTAYMELS | | TDFTLTISRLEPEDFAVYYCQQY | |
| | SLRSEDTAVYYCARGGYAAGT | | YSSYTFGGGTKVEIK | |
| | DYYMGVWGKGTTVTVSS | | | |
| SA-52 | QVQLVQSGAEVKKPGASVKVS | 248 | EIVMTQSPGTLSLSPGERATLSC | 308 |
| | CKASGYTFTNYGISWVRQAPG | | RASQSVSSSYLAWYQQKPGQAPR | |
| | QGLEWMGWISAYNGNTNYAQK | | LLIYGASNRATGIPDRFSGSGSG | |
| | LQGRVTMTTDTSTSTAYMELR | | TDFTLTISRLEPEDFAVYYCQQY | |
| | SLRSDDTAVYYCARGLWSEHI | | VDLPLTFGGGTKVEIK | |
| | YWGQGTLVTVSS | | | |
| SA-53 | QVQLQESGPGLVKPSQTLSLT | 249 | DIQMTQSPSSLSASVGDRVTITC | 309 |
| | CTVSGGSISSGGYYWSWIRQH | | QASQDISNYLNWYQQKPGKAPKL | |
| | PGKGLEWIGYIYYSGSTYYNP | | LIYDASNLETGVPSRFSGSGSGT | |
| | SLKSRVTISVDTSKNQFSLKL | | DFTFTISSLQPEDIATYYCQQSA | |
| | SSVTAADTAVYYCARGPLGAA | | SLPYTFGGGTKVEIK | |
| | GFDYWGQGTLVTVSS | | | |
| SA-54 | QLQLQESGPGLVKPSETLSLT | 250 | DIQMTQSPSSVSASVGDRVTITC | 310 |
| | CTVSGGSISSSSYYWGWIRQP | | RASQGISSWLAWYQQKPGKAPKL | |
| | PGKGLEWIGSIYYSGSTYYNP | | LIYAASSLQSGVPSRFSGSGSGT | |
| | SLKSRVTISVDTSKNQFSLKL | | DETLTISSLQPEDFATYYCQQVY | |
| | SSVTAADTAVYYCARGSGIAA | | LFPITFGGGTKVEIK | |
| | ADTDGLPMDVWGQGTTVTVSS | | | |
| SA-55 | EVQLVESGGGLVQPGGSLRLS | 251 | DIQLTQSPSTLSASVGDRVTITC | 311 |
| | CAASGFTFSSYWMSWVRQAPG | | RASQAISSWLAWYQQKPGKAPKL | |
| | KGLEWVANIKQDGSEKYYVDS | | LIYDASSLESGVPSRFSGSGSGT | |
| | VKGRFTISRDNAKNSLYLQMN | | EFTLTISSLQPDDFATYYCQQYE | |
| | SLRAEDTAVYYCARGSPYYDA | | SFPWTFGGGTKVEIK | |
| | YDFDLWGRGTLVTVSS | | | |
| SA-56 | EVQLVESGGGLVKPGGSLRLS | 252 | DIQMTQSPSSVSASVGDRVTITC | 312 |
| | CAASGFTFSSYSMNWVRQAPG | | RASQGISSWLAWYQQKPGKAPKL | |
| | KGLEWVSSISSSSSYIYYADS | | LIYAASSLQSGVPSRFSGSGSGT | |
| | VKGRFTISRDNAKNSLYLQMN | | DFTLTISSLQPEDFATYYCQQAI | |
| | SLRAEDTAVYYCARGVETYSG | | YFPITFGGGTKVEIK | |
| | PNWEDPWGQGTLVTVSS | | | |
| SA-57 | QLQLQESGPGLVKPSETLSLT | 253 | EIVLTQSPATLSLSPGERATLSC | 313 |
| | CTVSGGSISSSSYAWGWIRQP | | RASQSVSSYLAWYQQKPGQAPRL | |
| | PGKGLEWIGSIYYSGSTYYNP | | LIYDASNRATGIPARFSGSGSGT | |
| | SLKSRVTISVDTSKNQFSLKL | | DETLTISSLEPEDFAVYYCQQDS | |
| | SSVTAADTAVYYCARKGMDDA | | AWPFTFGGGTKVEIK | |
| | GMDVWGQGTTVTVSS | | | |
| SA-58 | QLQLQESGPGLVKPSETLSLT | 254 | DIQLTQSPSSLSASVGDRVTITC | 314 |
| | CTVSGGSISSSSYAWGWIRQP | | QASQDISNYLNWYQQKPGKAPKL | |
| | PGKGLEWIGSIYYSGSTYYNP | | LIYDASNLETGVPSRFSGSGSGT | |
| | SLKSRVTISVDTSKNQFSLKL | | DFTFTISSLQPEDIATYYCQQPL | |
| | SSVTAADTAVYYCARLPMGCG | | NLPFTFGGGTKVEIK | |
| | SGMDVWGQGTTVTVSS | | | |
| SA-59 | QVQLQESGPGLVKPSETLSLT | 255 | DIQMTQSPSSLSASVGDRVTITC | 315 |
| | CTVSGGSISSYYWSWIRQPPG | | QASQDISNYLNWYQQKPGKAPKL | |
| | KGLEWIGSIYYSGSTNYNPSL | | LIYDASNLETGVPSRFSGSGSGT | |
| | KSRVTISVDTSKNQFSLKLSS | | DFTFTISSLQPEDIATYYCQQVI | |
| | VTAADTAVYYCARSGGSYSYL | | DLPYTFGGGTKVEIK | |
| | DVWGQGTMVTVSS | | | |
| SA-60 | EVQLVESGGGLVQPGGSLRLS | 256 | EIVLTQSPATLSLSPGERATLSC | 316 |
| | CAASGFTFSSYSMNWVRQAPG | | RASQSVSSYLAWYQQKPGQAPRL | |
| | KGLEWVSYISSSSSTIYYADS | | LIYDSSNRATGIPARFSGSGSGT | |
| | VKGRFTISRDNAKNSLYLQMN | | DFTLTISSLEPEDFAVYYCQDRS | |
| | SLRAEDTAVYYCARTHKHEDY | | NLPWTFGGGTKVEIK | |
| | GMDVWGQGTTVTVSS | | | |
| SA-61 | QVQLVQSGAEVKKPGASVKVS | 233 | DIQMTQSPSTLSASVGDRVTITC | 291 |
| | CKASGYTFTSYYMHWVRQAPG | | RASQSISSWLAWYQQKPGKAPKL | |
| | QGLEWMGIINPSGGSTSYAQK | | LIYKASSLESGVPSRFSGSGSGT | |
| | FQGRVTMTRDTSTSTVYMELS | | EFTLTISSLQPDDFATYYCQQYG | |
| | SLRSEDTAVYYCAREAGYDIK | | SFPLTFGGGTKVEIK | |
| | GFDYWGQGTLVTVSS | | | |
| SA-62 | EVQLVESGGGLVQPGGSLRLS | 256 | DIQMTQSPSSLSASVGDRVTITC | 317 |
| | CAASGFTFSSYSMNWVRQAPG | | QASQDIANYLNWYQQKPGKAPKL | |
| | KGLEWVSYISSSSSTIYYADS | | LIYDASNLETGVPSRFSGSGSGT | |
| | VKGRFTISRDNAKNSLYLQMN | | DETFTISSLQPEDIATYYCQQYV | |
| | SLRAEDTAVYYCARTHKHEDY | | NYWTFGGGTKVEIK | |
| | GMDVWGQGTTVTVSS | | | |

### Example 7: Characterization of Anti-SIRPA Antibody Binding

Initial characterization of anti-SIRPA antibodies of the present disclosure involved determining their ability to bind cell lines overexpressing human or mouse SIRPA. Cells were harvested, plated at 10⁶/ml in a 96 well plate, washed, and incubated in 100 ul PBS containing 1 ug/ml Mab for 1 hour in ice. Cells were then washed twice and incubated in 100 ul PBS containing 5 ug/ml PE-conjugated secondary antibody for 30 minutes in ice. Cells were washed twice in cold PBS and acquired on a BD FACS Canto. Data analysis and calculation of mean fluorescence intensity (MFI) values or % positive cells was performed with FlowJo® (TreeStar) software version 10.0.7.

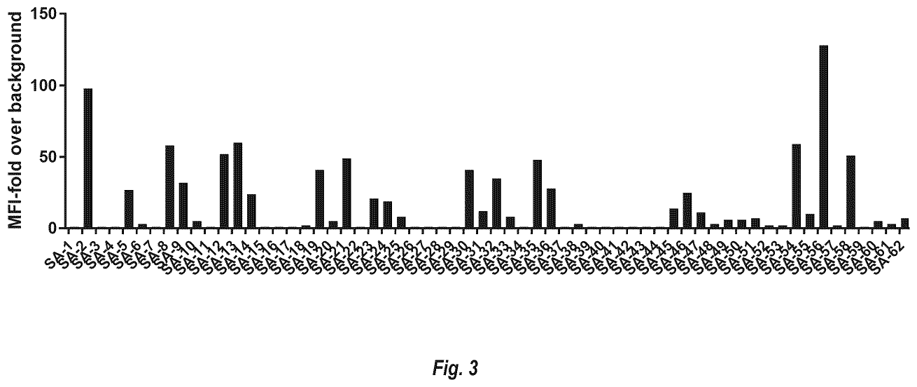

FIG. 3 shows the mean fluorescent intensity (MFI) values of anti-SIRPA antibodies binding to the Chinese hamster ovary (CHO) cell line overexpressing human SIRPA. The human IgG1 isotype control established the background fluorescent signal set to 1 on the y-axis. Of the 62 anti-SIRPA antibody clones tested, 23 clones bound to cells with an MFI ≥10-fold over background. As a negative control, the anti-SIRPA antibodies were also screened for surface binding to CHO cells overexpressing mouse SIRPA. As expected, none of the test antibodies bound to mouse SIRPA since clones were originally selected for binding the human antigen.

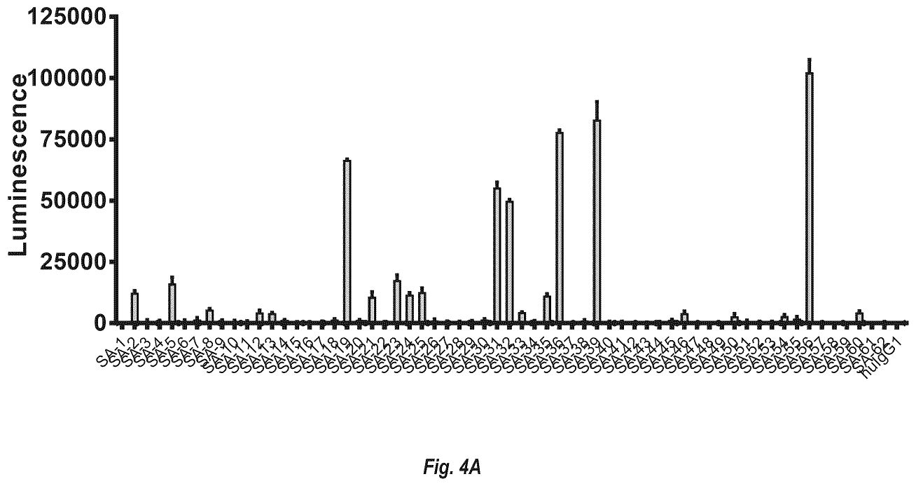

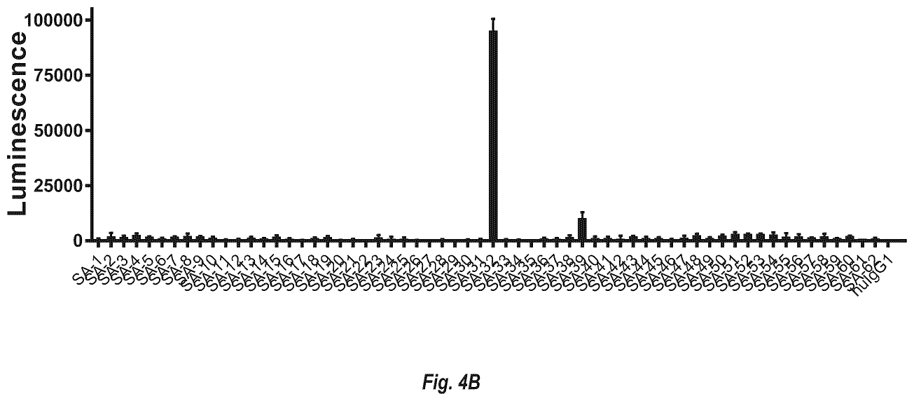

Given the high sequence similarity between receptors of the SIRP family, anti-SIRPA antibodies of the present disclosure were also screened for cross-reactivity to human SIRPβ1 by using a reporter cell line expressing the luciferase gene under the control of an NFAT (nuclear factor of activated T-cells) promoter. The cell line BW5147.G.1.4 (ATCC® TIB48™), derived from mouse thymus lymphoma T lymphocytes, was infected with Cignal Lenti NFAT-luciferase virus (Qiagen). Subsequently, cells were transduced with either a lentivirus expressing human SIRPA-DAP12 chimera, in which the intracellular ITIM motif of SIRPA was substituted with the intracellular ITAM motif of DAP12, or with two lentiviruses expressing human SIRPβ1 and human DAP12. Test antibodies, as well as the human IgG1 isotype control, were adsorbed onto a 96-well plate at 10 ug/mL. After washing, 10⁵ NFAT-luciferase reporter cells expressing the huSIRPA/DAP12 chimera (BWZ-huSIRPA) or co-expressing huSIRPβ1 and DAP12 (BWZ-huSIRPβ1) were seeded onto plates and incubated overnight at 37 C. Luciferase activity was measured by adding OneGlo Reagent (Promega) to each well and incubating samples for 3 min at room temperature on a plate shaker. The luminescence signal was quantified using a BioTek Synergy™ Microplate Reader using GEN5™ 2.04 software. As shown in FIG. 4A, only certain anti-SIRPA antibodies (13 of 62 clones) retained the ability to induce luciferase expression in the BWZ-huSIRPA reporter cells. In contrast, only one clone (SA-32) significantly induced luciferase expression when BWZ-huSIRPβ1 reporter cells were added onto antibody-coated wells. These results indicated that most anti-SIRPA antibodies capable of binding membrane-bound antigen demonstrate specificity towards the target antigen without cross-reacting to other SIRP receptors. The MFI values for cell lines expressing human SIRPA or mouse SIRPA bound by anti-SIRPA antibodies and luminescence values measured by plate-bound antibodies on reporter cells are listed in Table 5.

TABLE 5
MFI Values.

| Clone | CHO | CHO- | CHO- |
|---|---|---|---|
| Index | parent | huSIRPA | muSIRPA |
| SA-1 | 0.443103 | 1.46831 | 0.544643 |
| SA-2 | 0.487931 | 98.33099 | 0.526786 |
| SA-3 | 0.456897 | 0.59507 | 0.517857 |
| SA-4 | 0.427586 | 0.869718 | 0.491071 |
| SA-5 | 0.536207 | 27.4507 | 0.532738 |
| SA-6 | 0.518966 | 3.130282 | 0.544643 |
| SA-7 | 0.482759 | 0.707746 | 0.514881 |
| SA-8 | 0.453448 | 57.76761 | 0.5 |
| SA-9 | 0.643966 | 32.48944 | 0.645833 |
| SA-10 | 0.562069 | 4.623239 | 0.583333 |
| SA-11 | 0.806034 | 0.971831 | 0.85119 |
| SA-12 | 0.546552 | 52.00704 | 0.547619 |
| SA-13 | 0.596552 | 59.89437 | 0.571429 |
| SA-14 | 0.948276 | 24.41901 | 0.732143 |
| SA-15 | 0.474138 | 0.767606 | 0.511905 |
| SA-16 | 0.525 | 0.591549 | 0.53869 |
| SA-17 | 0.463793 | 0.989437 | 0.47619 |
| SA-18 | 0.539655 | 2.126761 | 0.559524 |
| SA-19 | 0.612931 | 41.20775 | 0.60119 |
| SA-20 | 0.965517 | 5.190141 | 0.785714 |
| SA-21 | 0.531897 | 49.21127 | 0.541667 |
| SA-22 | 0.676724 | 1.390845 | 0.619048 |
| SA-23 | 0.532759 | 21.09507 | 0.532738 |
| SA-24 | 0.587931 | 19.3838 | 0.541667 |
| SA-25 | 0.597414 | 8.267606 | 0.58631 |
| SA-26 | 0.600862 | 1.098592 | 0.574405 |
| SA-27 | 0.637931 | 0.721831 | 0.619048 |
| SA-28 | 0.45 | 0.774648 | 0.467262 |
| SA-29 | 0.467241 | 0.59507 | 0.479167 |
| SA-30 | 0.609483 | 41.23592 | 0.595238 |
| SA-31 | 0.52069 | 12.3662 | 0.508929 |
| SA-32 | 0.698276 | 35.37324 | 0.684524 |
| SA-33 | 0.735345 | 8.34507 | 0.708333 |
| SA-34 | 0.708621 | 0.806338 | 0.613095 |
| SA-35 | 0.680172 | 48.20775 | 0.491071 |
| SA-36 | 0.759483 | 27.86972 | 0.574405 |
| SA-37 | 0.478448 | 1.271127 | 0.464286 |
| SA-38 | 0.469828 | 2.957746 | 0.458333 |
| SA-39 | 0.626724 | 1.471831 | 0.571429 |
| SA-40 | 0.477586 | 0.542254 | 0.446429 |
| SA-41 | 0.511207 | 0.989437 | 0.470238 |
| SA-42 | 0.518966 | 1.285211 | 0.443452 |
| SA-43 | 0.510345 | 0.559859 | 0.479167 |
| SA-44 | 0.656034 | 1.204225 | 0.559524 |
| SA-45 | 0.567241 | 14.10211 | 0.511905 |
| SA-46 | 0.584483 | 25.44014 | 0.47619 |
| SA-47 | 0.576724 | 11.26408 | 0.479167 |
| SA-48 | 0.716379 | 3.271127 | 0.544643 |
| SA-49 | 0.607759 | 5.651408 | 0.529762 |
| SA-50 | 0.593103 | 5.556338 | 0.517857 |
| SA-51 | 0.57069 | 6.964789 | 0.508929 |
| SA-52 | 0.685345 | 1.901408 | 0.556548 |
| SA-53 | 0.82931 | 2.133803 | 0.72619 |
| SA-54 | 0.575862 | 59.48592 | 0.46131 |
| SA-55 | 0.558621 | 10.27113 | 0.464286 |
| SA-56 | 0.696552 | 128.1127 | 0.583333 |
| SA-57 | 0.57931 | 1.894366 | 0.464286 |
| SA-58 | 1 | 50.78521 | 0.738095 |
| SA-59 | 0.716379 | 0.630282 | 0.529762 |
| SA-60 | 1 | 4.848592 | 0.53869 |
| SA-61 | 0.566379 | 2.911972 | 0.46131 |
| SA-62 | 0.788793 | 7.088028 | 0.556548 |

### Example 8: Identifying CD47-Blocking and Non-Blocking Anti-SIRPA Antibodies

Given the role of the SIRPA-CD47 axis pathway in suppressing phagocytic cell effector functions, antagonistic agents described to date rely on competitive inhibition to block receptor-ligand interaction. Similarly, SIRPA antibodies of the present disclosure were screened for their ability to block CD47 binding to BWZ-huSIRPA. Cells were harvested, plated at 10⁵ cells/well in a 96-well plate, washed, and incubated in 100 μl FACS buffer containing 10 μg/ml of indicated monoclonal antibody or isotype control. Cells were then washed and incubated in FACS buffer containing 250 nM His-tagged, soluble human CD47 for 30 minutes on ice. Cells were washed again and stained with PE-conjugated anti-His tag monoclonal antibody to detect surface bound CD47. Data analysis and calculation of MFI values or % positive cells was performed with FlowJo (TreeStar) software version 10.0.7.

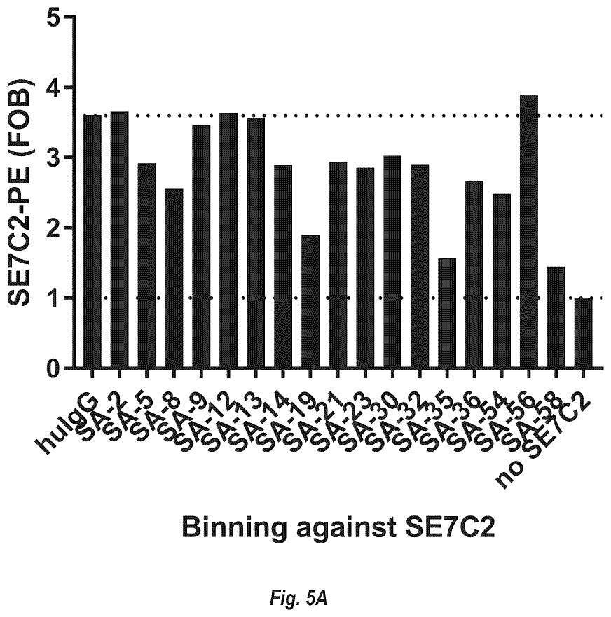

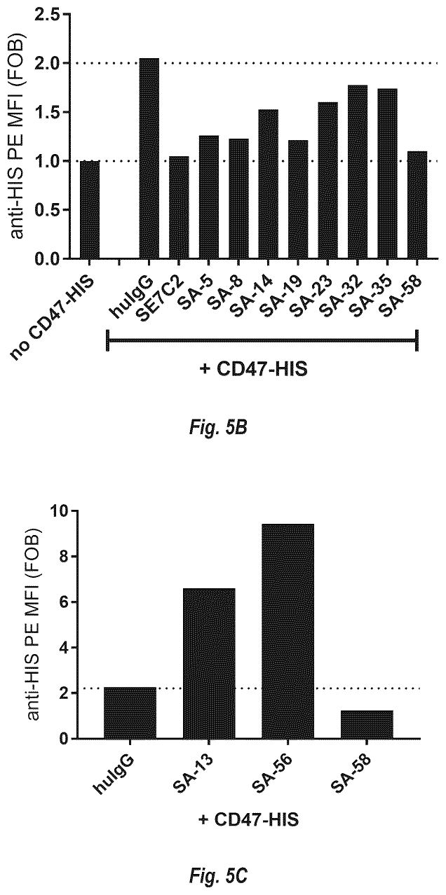

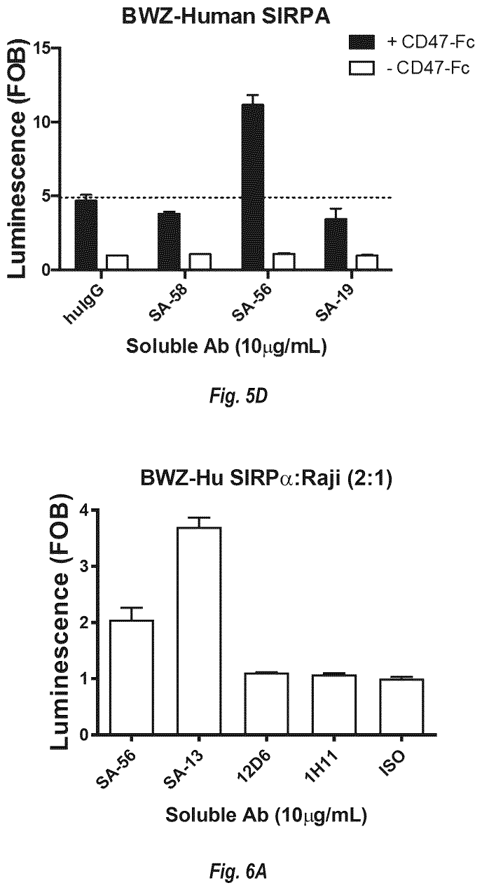

As an initial screen for CD47 ligand blocking antibodies, test antibodies were first binned against a commercial anti-SIRPA antibody (clone SE7C2, Santa Cruz Bio.) previously shown to block CD47 interaction. BWZ-huSIRPA cells were first incubated with indicated test antibodies or isotype control and subsequently stained with PE-conjugated anti-SIRPA antibody SE7C2. As shown in FIG. 5A, certain anti-SIRPA antibodies of the present disclosure (e.g., SA-5, SA-8, SA-14, SA-19, SA-35, SA-36, SA-54, and SA-58), reduced fluorescence signal close to background level, suggesting that these anti-SIRPA antibodies belong to the same or overlapping epitope bin as the anti-CD47-blocking reference antibody (Bin 1). Other anti-SIRPA antibodies of the present disclosure, such as SA-2, SA-13, and SA-56, exhibited no effect on anti-SIRPA antibody SE7C2 binding to cells, placing these antibodies on a distinct epitope bin (Bin 2). Certain anti-SIRPA antibodies of the present disclosure (SA-5, SA-8, SA-14, SA-21, SA-23, SA-30, SA-32, SA-36, SA-54) demonstrated partial interference with anti-SIRPA antibody SE7C2 binding, suggesting that their epitopes overlap with the reference antibody. These epitope bin designations differ from those listed on Table 1 as a result of utilizing a different reference antibody for the FACS-based assay.

Next, anti-SIRPA antibodies of the present disclosure that effectively or partially impeded anti-SIRPA antibody SE7C2 binding (bin 1) were assessed for their CD47 ligand blocking activity. As shown in FIG. 5B, some bin 1 anti-SIRPA antibodies, such as SA-5, SA-8, SA-19, and SA-58, effectively blocked soluble CD47 binding to cells. Remaining anti-SIRPA antibodies demonstrated partial interference. In contrast, bin 2 anti-SIRPA antibodies, specifically anti-SIRPA antibodies SA-13 and SA-56, enhanced (i.e., increased) soluble CD47 binding to SIRPA when compared to that observed in isotype control treated cells (FIG. 5C).

To ascertain if increased CD47 binding to SIRPA leads to greater SIRPA signaling, BWZ-huSIRPA reporter cells were treated with anti-SIRPA antibodies in the presence or absence of plate-bound CD47 ligand. As shown in FIG. 5D, BWZ-huSIRPA reporter cells fail to express luciferase in the absence of CD47, regardless of antibody treatment. In the presence of CD47, reporter cells treated with isotype control antibody emit a luminescence signal ˜5-fold over background levels. This signal is partially inhibited in the presence of CD47-blocking, bin 1 anti-SIRPA antibodies SA-19 and SA-58. However, stimulating BWZ-huSIRPA reporter cells with anti-SIRPA antibody SA-56 (bin 2) and plate-bound CD47 augmented the luminescence signal ˜10-fold over background, consistent with the observation that bin 2 anti-SIRPA antibodies increase CD47 binding. These results also showed that anti-SIRPA antibodies of the present disclosure are effective at increasing SIRPA signaling.

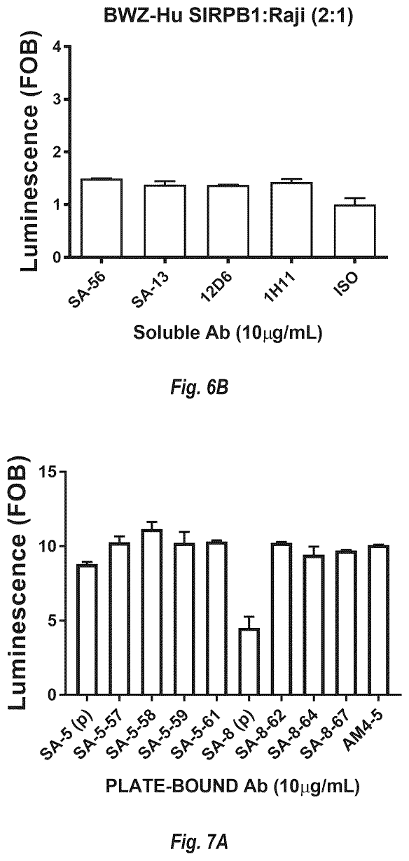

To verify that bin 2 anti-SIRPA antibodies can increase SIRPA binding to cell surface expressed CD47, BW Z-huSIRPA reporter cells were co-incubated with the Raji Burkitt's lymphoma-derived cell line, which are immortalized human B cells previously shown to highly express CD47. As shown in FIG. 6, mixing reporter cells and Raji cells overnight at a 2:1 ratio did not induce luciferase expression in the presence of isotype control antibody. Adding either soluble anti-SIRPA antibody SA-56 or SA-13 to the cell mixture, however, stimulated luciferase expression in BWZ-huSIRPA cells. Importantly, this effect was specific to bin 2 anti-SIRPA antibodies, specifically anti-SIRPA antibodies SA-13 and SA-56, since bin 1 anti-SIRPA antibodies (antibody 12D6 and antibody 1H11) did not increase luminescence in this assay. To verify that induction of gene expression in reporter cells was dependent on ligand binding to SIRPA, Raji cells were mixed with BWZ-huSIRPβ1 reporter cells, which do not bind CD47. As shown in FIG. 6B, co-incubation of BW Z-huSIRPβ1 cells with Raji cells failed to induce luciferase expression regardless of antibody treatment. Thus, whereas certain anti-SIRPA antibodies of the present disclosure that belong to the same epitope bin as commercially-available anti-SIRPA antibody SE7C2 potentially block ligand binding, anti-SIRPA antibodies that fall outside of this bin, specifically anti-SIRPA antibody SA-13 and SA-56, increased CD47 binding and increased CD47-induced SIRPA signaling in cells. Taken together, these results indicated that anti-SIRPA antibodies of the present disclosure are effective at increasing SIRPA signaling in cells.

### Example 9: Affinity Maturation of Anti-SIRPA Antibodies

Five anti-SIRPA antibodies of the present disclosure (anti-SIRPA antibodies SA-5, SA-8, SA-13, SA-19, and SA-56 (herein termed “parent” antibodies), with various physical and functional attributes, were affinity-matured as follows. Briefly, diversified antibody libraries were created in yeast for each of the starting parent anti-SIRPA antibodies. Diversity was created by utilizing standard molecular cloning techniques to combine the parental heavy chain CDR-H3 and light chain (LC) with pre-existing genetic diversity in the CDR-H1 and CDR-H2 regions of the heavy chain (HC) (termed “H1/H2” optimization). This resulted in six libraries of roughly 10⁵ in size that were ready for selection to enrich for anti-SIRPA antibodies having improved affinity. Selection pressures used for screening the libraries included human SIRPA and SIRPβ1 antigen equilibrium titration, parental antibody Fab competition kinetics, and the use of polyspecificity reagent deselection (as described, for example, in WO 2014/179363; Nu et al., Protein Eng Des Sel, Vol. 26(10), pp. 663-670). FACS flow cytometry was then employed to visualize and select antibodies, using standard techniques (see, e.g., Chao et al. Nature Protocols, 2006). The desired population was then carried forward into additional selection rounds. After 6 rounds of enrichment, yeast were plated out in order to obtain single antibody isolates, which were then produced and characterized as described in Example 1. Forty-one affinity-improved antibodies from each of the five starting parental antibodies were thus obtained.

a. Antibody IgG and Fab Production and Purification

Yeast clones were grown to saturation and then induced for 48 h at 30° C. with shaking. After induction, yeast cells were pelleted and the supernatants were harvested for purification.

Immunoglobulins were purified using a Protein A column and eluted with acetic acid, pH 2.0. Fab fragments were generated by papain digestion and purified over CaptureSelect IgG-CH1 affinity matrix (LifeTechnologies).

b. Affinity Determination

The affinities of the anti-SIRPA antibodies were determined by measuring K_{D} values by ForteBio and MSD. ForteBio affinity measurements were performed, at room temperature, generally as previously described (Estep et al, MAbs. 2013 March-April; 5(2):270-8). Briefly, ForteBio affinity measurements were performed by loading immunoglobulins (IgGs) on-line onto AHQ sensors. Sensors were equilibrated off-line in assay buffer for 30 min and then monitored on-line for 60 seconds for baseline establishment. For avid binding measurement, sensors with loaded IgGs were exposed to 100 nM antigen (human SIRPA or SIRPβ1 Fc fusion) for 3 min, afterwards they were transferred to assay buffer for 3 min for off-rate measurement. Additional avid binding was determined by loading biotinylated SIRPA monomer on SA sensors and exposure to 100 nM IgG in solution. Monovalent binding measurements were obtained by loading human SIRPA Fc fusion antigens to AHQ sensor and followed by exposure to 100 nM anti-SIRPA antibody Fab.

Additional monovalent measurements were made by loading biotinylated human SIRPA monomer to SA sensor followed by exposure to 100 nM Fab in solution. Kinetics data were fit using a 1:1 binding model in the data analysis software provided by ForteBio.

For MSD-SET KD measurements, solution equilibrium titrations (SET) were performed in PBS+0.1% IgG-Free BSA (PBSF) with recombinant human SIRPA, held constant at 100 pM and incubated with 3- to 5-fold serial dilutions of antibody starting at around 50 nM. Antibodies (20 nM in PBS) were coated onto standard bind MSD-ECL plates overnight at 4° C. or at room temperature for 30 min. Plates were then blocked with 1% BSA for 30 min with shaking at 700 rpm, followed by three washes with wash buffer (PBSF+0.05% Tween 20). SET samples were applied and incubated on the plates for 150s with shaking at 700 rpm followed by one wash. Antigen captured on a plate was detected with 250 ng/ml sulfotag-labeled streptavidin in PBSF by incubation on the plate for 3 min. The plates were washed three times with wash buffer and then read on the MSD Sector Imager 2400 instrument using 1× Read Buffer T with surfactant. The percent free antigen was plotted as a function of titrated antibody in Prism and fit to a quadratic equation to extract the KD. To improve throughput, liquid handling robots were used throughout MSD-SET experiments, including SET sample preparation.

Cell binding affinity measurements were performed at 4° C. using BWZ reporter cells either expressing either human SIRPA or SIRPβ1. Briefly, cells were harvested, washed in PBS and incubated with increasing concentration of anti-SIRPA antibodies or isotype control. Antibodies were diluted in FACS buffer (PBS+2% FBS). After incubation on ice for 30 min, cells were washed two times in FACS buffer and incubated with anti-human PE conjugated secondary antibody (BD Biosciences) for 30 min on ice. Then cells were washed twice in 200 ul FACS buffer, and subsequently analyzed on a FACS Canto screening instrument (BD). Apparent K_{D} values were determined by non-linear curve fitting (modified OneSiteTotal, Graph Pad Prism).

c. Anti-SIRPA Antibody Selection

Affinity-matured anti-SIRPA antibody clones (generated as described above), which showed improved affinity compared to the respective parental antibody, were characterized further. After initial screening of all affinity-matured antibody clones, clones for each parental antibody were selected for further analysis.

d. Antibody Heavy Chain and Light Chain Variable Domain Sequences

Using standard techniques, the amino acid sequences encoding the light chain variable and the heavy chain variable domains of the generated anti-SIRPA antibodies were determined. The Kabat light chain HVR sequences of the affinity matured antibodies are set forth in Table 6. The Kabat heavy chain HVR sequences of the antibodies are set forth in Table 7. The heavy chain variable region and light chain variable region amino acid sequences are shown in Table 8.

TABLE 6
Kabat Light Chain HVR Sequences.

| | | SEQ ID | | SEQ ID | | SEQ ID |
|---|---|---|---|---|---|---|
| Antibody | HVR-L1 | NO: | HVR-L2 | NO: | HVR-L3 | NO: |
| SA-5 | RASQDISSWLA | 9 | AASSLQS | 32 | QQGYIFPRT | 45 |
| (parental) | | | | | | |
| SA-5-57 | RASQDISSWLA | 9 | AASSLQS | 32 | QQGYIFPRT | 45 |
| SA-5-58 | RASQDISSWLA | 9 | AASSLQS | 32 | QQGYIFPRT | 45 |
| SA-5-59 | RASQDISSWLA | 9 | AASSLQS | 32 | QQGYIFPRT | 45 |
| SA-5-61 | RASQDISSWLA | 9 | AASSLQS | 32 | QQGYIFPRT | 45 |
| SA-8 | QASQDISNYLN | 11 | DASNLET | 33 | QQLSNYPIT | 48 |
| (parental) | | | | | | |
| SA-8-62 | QASQDISNYLN | 11 | DASNLET | 33 | QQLSNYPIT | 48 |
| SA-8-64 | QASQDISNYLN | 11 | DASNLET | 33 | QQLSNYPIT | 48 |
| SA-8-66 | QASQDISNYLN | 11 | DASNLET | 33 | QQLSNYPIT | 48 |
| SA-8-67 | QASQDISNYLN | 11 | DASNLET | 33 | QQLSNYPIT | 48 |
| SA-13 | RASQSISSYLN | 14 | AASSLQS | 32 | QQGYIYPPT | 53 |
| (parental) | | | | | | |
| SA-13-68 | RASQSISSYLN | 14 | AASSLQS | 32 | QQGYIYPPT | 53 |
| SA-13-69 | RASQSISSYLN | 14 | AASSLQS | 32 | QQGYIYPPT | 53 |
| SA-13-71 | RASQSISSYLN | 14 | AASSLQS | 32 | QQGYIYPPT | 53 |
| SA-19 | QASQDISNYLN | 11 | DASNLET | 33 | QQFTYLPPT | 59 |
| (parental) | | | | | | |
| SA-19-72 | QASQDISNYLN | 11 | DASNLET | 33 | QQFTYLPPT | 59 |
| SA-19-73 | QASQDISNYLN | 11 | DASNLET | 33 | QQFTYLPPT | 59 |
| SA-19-74 | QASQDISNYLN | 11 | DASNLET | 33 | QQFTYLPPT | 59 |
| SA-19-75 | QASQDISNYLN | 11 | DASNLET | 33 | QQFTYLPPT | 59 |
| SA-19-76 | QASQDISNYLN | 11 | DASNLET | 33 | QQFTYLPPT | 59 |
| SA-19-77 | QASQDISNYLN | 11 | DASNLET | 33 | QQFTYLPPT | 59 |
| SA-19-78 | QASQDISNYLN | 11 | DASNLET | 33 | QQFTYLPPT | 59 |
| SA-19-79 | QASQDISNYLN | 11 | DASNLET | 33 | QQFTYLPPT | 59 |
| SA-19-80 | QASQDISNYLN | 11 | DASNLET | 33 | QQFTYLPPT | 59 |
| SA-19-81 | QASQDISNYLN | 11 | DASNLET | 33 | QQLSNYPIT | 48 |
| SA-19-82 | QASQDISNYLN | 11 | DASNLET | 33 | QQFTYLPPT | 59 |
| SA-19-83 | QASQDISNYLN | 11 | DASNLET | 33 | QQFTYLPPT | 59 |
| SA-19-84 | QASQDISNYLN | 11 | DASNLET | 33 | QQFTYLPPT | 59 |
| SA-56 | RASQGISSWLA | 12 | AASSLQS | 32 | QQAIYFPIT | 96 |
| (parental) | | | | | | |
| SA-56-85 | RASQGISSWLA | 12 | AASSLQS | 32 | QQAIYFPIT | 96 |
| SA-56-86 | RASQGISSWLA | 12 | AASSLQS | 32 | QQAIYFPIT | 96 |
| SA-56-87 | RASQGISSWLA | 12 | AASSLQS | 32 | QQAIYFPIT | 96 |
| SA-56-88 | RASQGISSWLA | 12 | AASSLQS | 32 | QQAIYFPIT | 96 |
| SA-56-89 | RASQGISSWLA | 12 | AASSLQS | 32 | QQAIYFPIT | 96 |
| SA-56-90 | RASQGISSWLA | 12 | AASSLQS | 32 | QQAIYFPIT | 96 |
| SA-56-91 | RASQGISSWLA | 12 | AASSLQS | 32 | QQAIYFPIT | 96 |
| SA-56-92 | RASQGISSWLA | 12 | AASSLQS | 32 | QQAIYFPIT | 96 |
| SA-56-93 | RASQGISSWLA | 12 | AASSLQS | 32 | QQAIYFPIT | 96 |
| SA-56-94 | RASQGISSWLA | 12 | AASSLQS | 32 | QQAIYFPIT | 96 |
| SA-56-95 | RASQGISSWLA | 12 | AASSLQS | 32 | QQAIYFPIT | 96 |
| SA-56-96 | RASQGISSWLA | 12 | AASSLQS | 32 | QQAIYFPIT | 96 |

TABLE 7
Kabat Light Chain HVR Sequences.

| | | SEQ | | SEQ | | SEQ |
|---|---|---|---|---|---|---|
| | | ID | | ID | | ID |
| Antibody | HVR-H1 | NO: | HVR-H2 | NO: | HVR-H3 | NO: |
| SA-5 | FTFSSYAMS | 106 | AISGSGGSTYYA | 125 | AKDTGIDFWSVTPP | 151 |
| (parental) | | | DSVKG | | YFDL | |
| SA-5-57 | FTFDSYAMS | 318 | ATSGGGGSTYHA | 329 | AKDITDDFWSVTPP | 350 |
| | | | DPVKG | | YFDL | |
| SA-5-58 | FTFDSYAMS | 318 | AISGSGGSTYHA | 330 | AKDTGISLWSVTPP | 351 |
| | | | DSVKG | | YFDL | |
| SA-5-59 | FTFDNYAMS | 319 | AVSGSGGSTYYA | 33 | AKDTGIDEWARTPP | 352 |
| | | | DSVRG | | YFDL | |
| SA-5-61 | FTFDSYAMS | 318 | AISGSGGSTYYA | 125 | AKDTGIDFWGQYPP | 353 |
| | | | DSVKG | | YFDL | |
| SA-8 | FTFSSYGMH | 103 | VISYDGSNKYYA | 124 | AKEPRSYWHGYGMD | 154 |
| (parental) | | | DSVKG | | V | |
| SA-8-62 | FTFSSYSMH | 320 | SISSSDSWIYYA | 332 | AKERVDYWHGYGMD | 371 |
| | | | DSVKG | | V | |
| SA-8-64 | FTFSSYSMH | 320 | SISSSDSWIYYA | 332 | AKEPRSYWHGYGMD | 154 |
| | | | DSVKG | | V | |
| SA-8-66 | FTFSSYGMH | 103 | VINGDGSNKYYA | 333 | AKEPRSYWHGYGMD | 154 |
| | | | DSVKG | | V | |
| SA-8-67 | FTFSSYSMH | 320 | SISSSPSHIYYA | 334 | AKEPRSYWHGYGMD | 154 |
| | | | DSVKG | | V | |
| SA-13 | FTFSSYAMS | 106 | AISGSGGSTYYA | 125 | ARDGRTWGPHFY | 159 |
| (parental) | | | DSVKG | | | |
| SA-13-68 | FTFTEYAMS | 321 | AISGSGGSTYYA | 125 | ARDNIQWGPHFY | 372 |
| | | | DSVKG | | | |
| SA-13-69 | FTFTEYAMS | 321 | AISGSGGSTYYA | 335 | ARDGTQWGPHFY | 373 |
| | | | DSVKA | | | |
| SA-13-71 | FTFSEWAMS | 322 | AISGSGGSTYYA | 125 | ARDGRTWGPHFY | 159 |
| | | | DSVKG | | | |
| SA-19 | FTFSSYGMH | 103 | VISYDGSNKYYA | 124 | ARDLTKYTLGFAFD | 165 |
| (parental) | | | DSVKG | | I | |
| SA-19-72 | FTFSSYAMH | 323 | GILYDGSNKYYA | 336 | ARSNEKYTLGFAFD | 354 |
| | | | DSVKG | | I | |
| SA-19-73 | FTFSSYAMH | 323 | GILYDGSNKYYA | 330 | ANSDTKYTLGFAFD | 355 |
| | | | DSVKG | | I | |
| SA-19-74 | FAFSSYAMH | 346 | GILYDGSNKYYA | 336 | ARHSQKYTLGFAFD | 356 |
| | | | DSVKG | | I | |
| SA-19-75 | FTESSYGMH | 103 | VIAYDGSNKLYA | 337 | ALMRTKYTLGFAFD | 357 |
| | | | DSVKG | | I | |
| SA-19-76 | FTFSSYGMH | 103 | VIAYDGSNKLYA | 337 | ATMRTKYTLGFAFD | 374 |
| | | | DSVKG | | I | |
| SA-19-77 | FTFSSYGMA | 347 | QILYDGSNEYYA | 338 | ARDLTKYTLGFAFD | 165 |
| | | | DSVKG | | I | |
| SA-19-78 | FTFSSYGMH | 103 | VIAYDGSNKLYA | 337 | ATSDTKYTLGFAFD | 375 |
| | | | DSVKG | | I | |
| SA-19-79 | FTFSSYGMH | 103 | VIAYDGSNKIYA | 339 | ATTDTKYTLGFAFD | 376 |
| | | | DSVKG | | I | |
| SA-19-80 | FTFSSYGMH | 103 | GILYDGSNKYYA | 336 | ARHSVKYTLGFAFD | 377 |
| | | | DSVKG | | I | |
| SA-19-81 | FTSSSYGMH | 348 | VIAYDGSNKLYA | 337 | ARTPTKYTLGFAFD | 378 |
| | | | DSVKG | | I | |
| SA-19-82 | FTFSSYAMH | 323 | GILYDGSNKYYA | 336 | ARHVLKYTLGFAFD | 379 |
| | | | DSVKG | | I | |
| SA-19-83 | FTFDSYLMH | 349 | SIIYDGSNKYYA | 340 | ASVRTKYTLGFAFD | 380 |
| | | | DSVKG | | I | |
| SA-19-84 | FTFSSYGMH | 103 | VIAYDGSNKLYA | 337 | ARTQLKYTLGFAFD | 381 |
| | | | DSVKG | | I | |
| SA-56 | FTFSSYSMN | 109 | SISSSSSYIYYA | 130 | ARGVETYSGPNWFD | 197 |
| (parental) | | | DSVKG | | P | |
| SA-56-85 | STFSYYGMS | 324 | SIWVDGSNKYYA | 341 | ARGVETYSGPNWFD | 197 |
| | | | DSVKG | | P | |
| SA-56-86 | FTFSYYGMS | 325 | SIWVDGSSKYYA | 342 | ARGVETYSGPNWFD | 197 |
| | | | DSVKG | | P | |
| SA-56-87 | FTFSYYGMS | 325 | SIWVDGSNKYYA | 341 | AFGLETYSGPNWFD | 382 |
| | | | DSVKG | | P | |
| SA-56-88 | FTFSYYGMS | 325 | SIWVDGSNKYYA | 343 | AEGKETYSGPNWFD | 383 |
| | | | DSAKG | | P | |
| SA-56-89 | FTFSYYGMS | 325 | SIWVDGSNKYYA | 341 | ARGVETYSGPNWFD | 197 |
| | | | DSVKG | | P | |
| SA-56-90 | STFSHYGMS | 326 | SIWVDGSNKYYA | 341 | ARGVETYSGPNWFD | 197 |
| | | | DSVKG | | P | |
| SA-56-91 | FTFAHYGMA | 327 | SIWVDGSNKYYA | 341 | ATAVETYSGPNWFD | 384 |
| | | | DSVKG | | P | |
| SA-56-92 | FTFSYYGMS | 325 | SIWVDGSNKYYA | 341 | ATGVETYSGPNWFD | 385 |
| | | | DSVKG | | P | |
| SA-56-93 | FTFSYYGMS | 325 | SIWVDGSNKYYA | 341 | AVSRETYSGPNWFD | 386 |
| | | | DSVKG | | P | |
| SA-56-94 | FTFSYYGMS | 325 | SIWVDGSNKYYA | 341 | AVGTETYSGPNWFD | 358 |
| | | | DSVKG | | P | |
| SA-56-95 | FTFAHYGMA | 327 | VIWVDGSPKYYA | 344 | AVARETYSGPNWFD | 359 |
| | | | DSVKG | | P | |
| SA-56-96 | FTFSSYAMG | 328 | VIWVDGSLKYYA | 345 | ATGRETYSGPNWFD | 360 |
| | | | DSVKG | | P | |

TABLE 8
Heavy Chain Variable Region and Light Chain Variable Region.

| | | SEQ | | SEQ |
|---|---|---|---|---|
| | | ID | | ID |
| Antibody | Heavy Chain Variable | NO: | Light Chain Variable | NO: |
| SA-5-57 | EVQLLESGGGLVQPGGSLRLSC | 361 | DIQLTQSPSSVSASVGDRVTI | 261 |
| | AASGFTFDSYAMSWVRQAPGKG | | TCRASQDISSWLAWYQQKPGK | |
| | LEWVSATSGGGGSTYHADPVKG | | APKLLIYAASSLQSGVPSRFS | |
| | RFTISRDNSKNTLYLQMNSLRA | | GSGSGTDFTLTISSLQPEDFA | |
| | EDTAVYYCAKDITDDFWSVTPP | | TYYCQQGYIFPRTFGGGTKVE | |
| | YFDLWGRGTLVTVSS | | IK | |
| SA-5-58 | EVQLLESGGGLVQPGGSLRLSC | 362 | DIQLTQSPSSVSASVGDRVTI | 261 |
| | AASGFTFDSYAMSWVRQAPGKG | | TCRASQDISSWLAWYQQKPGK | |
| | LEWVSAISGSGGSTYHADSVKG | | APKLLIYAASSLQSGVPSRFS | |
| | RFTISRDNSKNTLYLQMNSLRA | | GSGSGTDFTLTISSLQPEDFA | |
| | EDTAVYYCAKDTGISLWSVTPP | | TYYCQQGYIFPRTFGGGTKVE | |
| | YFDLWGRGTLVTVSS | | IK | |
| SA-5-59 | EVQLLESGGGLVQPGGSLRLSC | 363 | DIQLTQSPSSVSASVGDRVTI | 261 |
| | AASGFTFDNYAMSWVRQAPGEG | | TCRASQDISSWLAWYQQKPGK | |
| | LEWVSAVSGSGGSTYYADSVRG | | APKLLIYAASSLQSGVPSRFS | |
| | RFTISRDNSKNTLYLQMDSLRA | | GSGSGTDFTLTISSLQPEDFA | |
| | EDTAVYYCAKDTGIDEWARTPP | | TYYCQQGYIFPRTFGGGTKVE | |
| | YFDLWGRGTLVTVSS | | IK | |
| SA-5-61 | EVQLLESGGGLVQPGGSLKLSC | 364 | DIQLTQSPSSVSASVGDRVTI | 261 |
| | AASGFTFDSYAMSWVRQAPGKG | | TCRASQDISSWLAWYQQKPGK | |
| | LEWVSAISGSGGSTYYADSVKG | | APKLLIYAASSLQSGVPSRFS | |
| | RFTISRDNSKNTLYLQMNSLRA | | GSGSGTDFTLTISSLQPEDFA | |
| | EDTAVYYCAKDTGIDFWGQYPP | | TYYCQQGYIFPRTFGGGTKVE | |
| | YFDLWGRGTLVTVSS | | IK | |
| SA-8-62 | QVQLVESGGGLVKPGGSLRLSC | 365 | DIQMTQSPSSLSASVGDRVTI | 264 |
| | AASGFTFSSYSMHWVRQAPGKG | | TCQASQDISNYLNWYQQKPGK | |
| | LEWVSSISSSDSWIYYADSVKG | | APKLLIYDASNLETGVPSRFS | |
| | RFTISRDNAKNSLYLQMDSLRA | | GSGSGTDFTFTISSLQPEDIA | |
| | EDTAVYYCAKERVDYWHGYGMD | | TYYCQQLSNYPITFGGGTKVE | |
| | VWGQGTTVTVSS | | IK | |
| SA-8-64 | QVQLVESGGGLVKPGGSLRLSC | 366 | DIQMTQSPSSLSASVGDRVTI | 264 |
| | AASGFTFSSYSMHWVRQAPGKG | | TCQASQDISNYLNWYQQKPGK | |
| | LEWVSSISSSDSWIYYADSVKG | | APKLLIYDASNLETGVPSRFS | |
| | RFTISRDNAKNSLYLQMNSLRA | | GSGSGTDFTFTISSLQPEDIA | |
| | EDTAVYYCAKEPRSYWHGYGMD | | TYYCQQLSNYPITFGGGTKVE | |
| | VWGQGTTVTVSS | | IK | |
| SA-8-66 | QVQLVESGGGVVQPGRSLRLSC | 367 | DIQMTQSPSSLSASVGDRVTI | 264 |
| | AASGFTFSSYGMHWVRQAPGKG | | TCQASQDISNYLNWYQQKPGK | |
| | LEWVAVINGDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCAKEPRSYWHGYGMD | | TYYCQQLSNYPITFGGGTKVE | |
| | VWGQGTTVTVSS | | IK | |
| SA-8-67 | EVQLVESGGGLVKPGGSLRLSC | 368 | DIQMTQSPSSLSASVGDRVTI | 264 |
| | AASGFTFSSYSMHWVRQAPGKG | | TCQASQDISNYLNWYQQKPGK | |
| | LEWVSSISSSPSHIYYADSVKG | | APKLLIYDASNLETGVPSRFS | |
| | RFTISRDNAKNSLYLQMNSLRA | | GSGSGTDFTFTISSLQPEDIA | |
| | EDTAVYYCAKEPRSYWHGYGMD | | TYYCQQLSNYPITFGGGTKVE | |
| | VWGQGTTVTVSS | | IK | |
| SA-13-68 | EVQLLESGGGLVQPGGSLRLSC | 369 | DIQMTQSPSSLSASVGDRVTI | 269 |
| | AASGFTFTEYAMSWVRQAPGKR | | TCRASQSISSYLNWYQQKPGK | |
| | LEWVSAISGSGGSTYYADSVKG | | APKLLIYAASSLQSGVPSRFS | |
| | RFTISRDNSKNTLYLQMSSLRA | | GSGSGTDFTLTISSLQPEDFA | |
| | EDTAVYYCARDNIQWGPHFYWG | | TYYCQQGYIYPPTFGGGTKVE | |
| | QGTLVTVSS | | IK | |
| SA-13-69 | EVQLLESGGGLVQPGGSLRLSC | 370 | DIQMTQSPSSLSASVGDRVTI | 269 |
| | AASGFTFTEYAMSWVRQAPGKG | | TCRASQSISSYLNWYQQKPGK | |
| | LEWVSAISGSGGSTYYADSVKA | | APKLLIYAASSLQSGVPSRFS | |
| | RFTISRDNSKNTLYLQMNSLRA | | GSGSGTDFTLTISSLQPEDFA | |
| | EDTAVYYCARDGTQWGPHFYWG | | TYYCQQGYIYPPTFGGGTKVE | |
| | QGTLVTVSS | | IK | |
| SA-13-71 | EVQLLESGGGLVQPGGSLRLSC | 387 | DIQMTQSPSSLSASVGDRVTI | 269 |
| | AASGFTFSEWAMSWVRQAPGKG | | TCRASQSISSYLNWYQQKPGK | |
| | LEWVSAISGSGGSTYYADSVKG | | APKLLIYAASSLQSGVPSRFS | |
| | RFTISRDNSKNTLYLQMNSLRA | | GSGSGTDFTLTISSLQPEDFA | |
| | EDTAVYYCARDGRTWGPHFYWG | | TYYCQQGYIYPPTFGGGTKVE | |
| | QGTLVTVSS | | IK | |
| SA-19-72 | QVQLVESGGGVVQPGRSLGLSC | 388 | DIQMTQSPSSLSASVGDRVTI | 275 |
| | AASGFTFSSYAMHWVRQAPGKG | | TCQASQDISNYLNWYQQKPGK | |
| | LEWVAGILYDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCARSNEKYTLGFAFD | | TYYCQQFTYLPPTFGGGTKVE | |
| | IWGQGTMVTVSS | | IK | |
| SA-19-73 | QVQLVESGGGLVQPGGSLRLSC | 389 | DIQMTQSPSSLSASVGDRVTI | 275 |
| | AASGFTFSSYAMHWVRQAPGKG | | TCQASQDISNYLNWYQQKPGK | |
| | LEWVAGILYDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCANSDTKYTLGFAFD | | TYYCQQFTYLPPTFGGGTKVE | |
| | IWGQGTMVTVSS | | IK | |
| SA-19-74 | QVQLVESGGGVVQPGRSLRLSC | 390 | DIQMTQSPSSLSASVGDRVTI | 275 |
| | AASGFAFSSYAMHWVRQAPGKG | | TCQASQDISNYLNWYQQKPGK | |
| | LEWVAGILYDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCARHSQKYTLGFAFD | | TYYCQQFTYLPPTFGGGTKVE | |
| | IWGQGTMVTVSS | | IK | |
| SA-19-75 | QVQLVESGGGVVQPGRSLRLSC | 391 | DIQMTQSPSSLSASVGDRVTI | 275 |
| | AASGFTFSSYGMHWVRQAPGKG | | TCQASQDISNYLNWYQQKPGK | |
| | LEWVAVIAYDGSNKLYADSVKG | | APKLLIYDASNLETGVPSRFS | |
| | RFTISRDNSKNTLYLQMNSLRA | | GSGSGTDFTFTISSLQPEDIA | |
| | EDTAVYYCALMRTKYTLGFAFD | | TYYCQQFTYLPPTFGGGTKVE | |
| | IWGQGTMVTVSS | | IK | |
| SA-19-76 | QVQLVESGGGVVQPGRSLRLSC | 392 | DIQMTQSPSSLSASVGDRVTI | 275 |
| | AASGFTFSSYGMHWVRQAPGKG | | TCQASQDISNYLNWYQQKPGK | |
| | LEWVAVIAYDGSNKLYADSVKG | | APKLLIYDASNLETGVPSRFS | |
| | RFTISRDNAKNSLYLQMNSPRA | | GSGSGTDFTFTISSLQPEDIA | |
| | EDTAVYYCATMRTKYTLGFAFD | | TYYCQQFTYLPPTFGGGTKVE | |
| | IWGQGTMVTVSS | | IK | |
| SA-19-77 | QVQLVESGGGVVQPGRSLRLSC | 393 | DIQMTQSPSSLSASVGDRVTI | 275 |
| | AASGFTFSSYGMAWVRQAPGKG | | TCQASQDISNYLNWYQQKPGK | |
| | LEWVAQILYDGSNEYYADSVKG | | APKLLIYDASNLETGVPSRFS | |
| | RFTISRDNSKNTLYLQMNSLRA | | GSGSGTDFTFTISSLQPEDIA | |
| | EDTAVYYCARDLTKYTLGFAFD | | TYYCQQFTYLPPTFGGGTKVE | |
| | IWGQGTMVTVSS | | IK | |
| SA-19-78 | QVQLVESGGGVVQPGRSLRLSC | 394 | DIQMTQSPSSLSASVGDRVTI | 275 |
| | AASGFTFSSYGMHWVRQAPGKG | | TCQASQDISNYLNWYQQKPGK | |
| | LEWVAVIAYDGSNKLYADSVKG | | APKLLIYDASNLETGVPSRFS | |
| | RFTISRDNSKNTLYLQMDSLRA | | GSGSGTDFTFTISSLQPEDIA | |
| | EDTAVYYCATSDTKYTLGFAFD | | TYYCQQFTYLPPTFGGGTKVE | |
| | IWGQGTMVTVSS | | IK | |
| SA-19-79 | QVQLVESGGGVVQPGRSLRLSC | 395 | DIQMTQSPSSLSASVGDRVTI | 275 |
| | AASGFTFSSYGMHWVRQAPGKG | | TCQASQDISNYLNWYQQKPGK | |
| | LEWVAVIAYDGSNKIYADSVKG | | APKLLIYDASNLETGVPSRFS | |
| | RFTISRDNSKNTLYLQMNSLRA | | GSGSGTDFTFTISSLQPEDIA | |
| | EDTAVYYCATTDTKYTLGFAFD | | TYYCQQFTYLPPTFGGGTKVE | |
| | IWGQGTMVTVSS | | IK | |
| SA-19-80 | EVQLLESGGGVVQPGRSLRLSC | 396 | DIQMTQSPSSLSASVGDRVTI | 275 |
| | AASGFTFSSYGMHWVRQAPGKG | | TCQASQDISNYLNWYQQKPGK | |
| | LEWVAGILYDGSNKYYADSVKG | | APKLLIYDASNLETGVPSRFS | |
| | RFTISRDNSKNTLYLQMDGLRA | | GSGSGTDFTFTISSLQPEDIA | |
| | EDTAVYYCARHSVKYTLGFAFD | | TYYCQQFTYLPPTFGGGTKVE | |
| | IWGQGTMVTVSS | | IK | |
| SA-19-81 | QVQLVESGGGVVQPGRSLRLSC | 398 | DIQMTQSPSSLSASVGDRVTI | 264 |
| | AASGFTSSSYGMHWVRQAPGKG | | TCQASQDISNYLNWYQQKPGK | |
| | LEWVAVIAYDGSNKLYADSVKG | | APKLLIYDASNLETGVPSRFS | |
| | RFTISRDNSKNTLYLQMNSLRA | | GSGSGTDFTFTISSLQPEDIA | |
| | EDTAVYYCARTPTKYTLGFAFD | | TYYCQQLSNYPITFGGGTKVE | |
| | IWGQGTMVTVSS | | IK | |
| SA-19-82 | QVQLVESGGGVVQPGRSLRLSC | 399 | DIQMTQSPSSLSASVGDRVTI | 275 |
| | AASGFTFSSYAMHWVRQAPGKG | | TCQASQDISNYLNWYQQKPGK | |
| | LEWVAGILYDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCARHVLKYTLGFAFD | | TYYCQQFTYLPPTFGGGTKVE | |
| | IWGQGTMVTVSS | | IK | |
| SA-19-83 | QVQLVESGGGVVQPGRSLRLSC | 400 | DIQMTQSPSSLSASVGDRVTI | 275 |
| | AASGFTFDSYLMHWVRQAPGKG | | TCQASQDISNYLNWYQQKPGK | |
| | LEWVASIIYDGSNKYYADSVKG | | APKLLIYDASNLETGVPSRFS | |
| | RFTISRDNSKNTLYLQMNSLRA | | GSGSGTDFTFTISSLQPEDIA | |
| | EDTAVYYCASVRTKYTLGFAFD | | TYYCQQFTYLPPTFGGGTKVE | |
| | IWGQGTMVTVSS | | IK | |
| SA-19-84 | QVQLVESGGGVVQPGRSLRLSC | 401 | DIQMTQSPSSLSASVGDRVTI | 275 |
| | AASGFTFSSYGMHWVRQAPGKG | | TCQASQDISNYLNWYQQKPGK | |
| | LEWVAVIAYDGSNKLYADSVKG | | APKLLIYDASNLETGVPSRFS | |
| | RFTISRDNSKNTLYLQMNSLRA | | GSGSGTDFTFTISSLQPEDIA | |
| | EDTAVYYCARTQLKYTLGFAFD | | TYYCQQFTYLPPTFGGGTKVE | |
| | IWGQGTMVTVSS | | IK | |
| SA-56-85 | EVQLLESGGGVVQPGRSLGLSC | 402 | DIQMTQSPSSVSASVGDRVTI | 413 |
| | AASGSTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKSTLYLQMNSLRA | | GSGSGTDETLTISSLQPEDFA | |
| | EDTAVYYCARGVETYSGPNWED | | TYYCQQAIYFPITFGGGTKVE | |
| | PWGQGTLVTVSS | | NQ | |
| SA-56-86 | QVQLVESGGGVVQPGGSLRLSC | 403 | DIQMTQSPSSVSASVGDRVTI | 312 |
| | AASGFTFSYYGMSWVRQAPGKG | | TCRASQGISSWLAWYQQKPGK | |
| | LEWVASIWVDGSSKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCARGVETYSGPNWED | | TYYCQQAIYFPITFGGGTKVE | |
| | PWGQGTLVTVSS | | IK | |
| SA-56-87 | EVQLVESGGGVVQPGRSLRLSC | 404 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCAFGLETYSGPNWED | | TYYCQQAIYFPITFGGGTKVE | |
| | PWGQGTLVTVSS | | IK | |
| SA-56-88 | EVQLVESGGGVVQPGRSLRLSC | 405 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSAKG | | APKLLIYAASSLQSGVPSRFS | |
| | RFTISRDNSKNTLYLQMNSLRA | | GSGSGTDFTLTISSLQPEDFA | |
| | EDTAVYYCAEGKETYSGPNWED | | TYYCQQAIYFPITFGGGTKVE | |
| | PWGQGTLVTVSS | | IK | |
| SA-56-89 | EVQLVESGGGVVQPGGSLRLSC | 406 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTVSRDNSKNTLYLQMDSLRA | | GSGSGTDFTLTISSLQPEDFA | |
| | EDTAVYYCARGVETYSGPNWED | | TYYCQQAIYFPITFGGGTKVE | |
| | PWGQGTLVTVSS | | IK | |
| SA-56-90 | QVQLVESGGGVVQPGRSLRLSC | 407 | DIQMTQSPSSVSASVGDRVTI | 312 |
| | AASGSTFSHYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNTLYLQMDSLRA | | GSGSGTDFTLTISSLQPEDFA | |
| | EDTAVYYCARGVETYSGPNWED | | TYYCQQAIYFPITFGGGTKVE | |
| | PWGQGTLVTVSS | | IK | |
| SA-56-91 | EVQLVESGGGVVQPGRSLRLSC | 408 | DIQMTQSPSSVSASVGDRVTI | 312 |
| | AASGFTFAHYGMAWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCATAVETYSGPNWED | | TYYCQQAIYFPITFGGGTKVE | |
| | PWGQGTLVTVSS | | IK | |
| SA-56-92 | EVQLVESGGGVVQPGRSLRLSC | 409 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNTLYLQMDSLRA | | GSGSGTDFTLTISSLQPEDFA | |
| | EDTAVYYCATGVETYSGPNWED | | TYYCQQAIYFPITFGGGTKVE | |
| | PWGQGTLVTVSS | | IK | |
| SA-56-93 | EVQLLESGGGVVQPGRSLRLSC | 410 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNALYLQMNSLRA | | GSGSGTDFTLTISSLQPEDFA | |
| | EDTAVYYCAVSRETYSGPNWED | | TYYCQQAIYFPITFGGGTKVE | |
| | PWGQGTLVTVSS | | IK | |
| SA-56-94 | EVQLVESGGGVVQPGRSLRLSC | 411 | DIQMTQSPSSVSASVGDRVTI | 312 |
| | AASGFTFSYYGMSWVRQAPGKG | | TCRASQGISSWLAWYQQKPGK | |
| | LEWVASIWVDGSNKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCAVGTETYSGPNWED | | TYYCQQAIYFPITFGGGTKVE | |
| | PWGQGTLVTVSS | | IK | |
| SA-56-95 | EVQLLESGGGVVQPGRSLRLSC | 397 | DIQMTQSPSSVSASVGDRVTI | 312 |
| | AASGFTFAHYGMAWVRQAPGKG | | TCRASQGISSWLAWYQQKPGK | |
| | LEWVAVIWVDGSPKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCAVARETYSGPNWED | | TYYCQQAIYFPITFGGGTKVE | |
| | PWGQGTLVTVSS | | IK | |
| SA-56-96 | EVQLLESGGGVVQPGRSLRLSC | 412 | DIQMTQSPSSVSASVGDRVTI | 312 |
| | AASGFTFSSYAMGWVRQAPGKG | | TCRASQGISSWLAWYQQKPGK | |
| | LEWVAVIWVDGSLKYYADSVKG | | APKLLIYAASSLQSGVPSRFS | |
| | RFTISRDNSKNTLYLQMNSLRA | | GSGSGTDFTLTISSLQPEDFA | |
| | EDTAVYYCATGRETYSGPNWED | | TYYCQQAIYFPITFGGGTKVE | |
| | PWGQGTLVTVSS | | IK | |

### Example 10: Characterization of Affinity-Matured Anti-SIRPA Antibody Binding

The affinity matured anti-SIRPA antibodies of the present disclosure were selected based on antigen binding affinities. Antibodies that were positive for binding to human SIRPA were tested for ability to block ligand binding and for cross-reactivity to human SIRPβ1. The biochemical characteristics of each antibody are listed in Table 9. In Table 9, “NB” refers to antibodies for which there is no binding to indicated antigen; “PF” refers to antibodies for which antigen binding kinetics show poor fit to 1:1 binding model.

TABLE 9
Biochemical Characteristics of the Antibodies.

| | | MSD Fab | | | | |
|---|---|---|---|---|---|---|
| | | K_{D} | | | | |
| | | Biotinylated | | | ForteBio | ForteBio |
| | | Human | ForteBio | ForteBio | IgG K_{D} | IgG K_{D} |
| | | SIRPA (M) | Fab K_{D} | Fab K_{D} | Human | Human |
| | | Monovalent | Human | Human | SIRPb | SIRPA |
| | Ligand | Equilibrium | SIRPA (M) | SIRPb (M) | (M) | (M) |
| Antibody | Competitor | K_{D} | Monovalent | Monovalent | Avid | Avid |
| SA-5 | No | N.M. | N.B. | N.B. | N.B. | P.F. |
| (parental) | | | | | | |
| SA-5-57 | No | N.M. | 2.33E−08 | N.B. | N.B. | 2.61E−09 |
| SA-5-58 | No | N.M. | P.F. | N.B. | N.B. | 2.78E−09 |
| SA-5-59 | No | N.M. | P.F. | N.B. | N.B. | 3.03E−09 |
| SA-5-61 | No | N.M. | P.F. | N.B. | 9.31E−09 | 2.30E−09 |
| SA-8 | Yes | N.M. | P.F. | N.B. | 7.56E−09 | P.F. |
| (parental) | | | | | | |
| SA-8-62 | Yes | N.M. | 8.92E−09 | N.B. | 4.67E−09 | 3.71E−10 |
| SA-8-64 | Yes | N.M. | 2.77E−08 | N.B. | 8.36E−09 | 9.52E−10 |
| SA-8-66 | Yes | N.M. | P.F. | N.B. | 1.31E−08 | 2.14E−09 |
| SA-8-67 | Yes | N.M. | 1.31E−07 | N.B. | 1.42E−08 | 1.84E−09 |
| SA-13 | No | N.M. | N.B. | N.B. | 1.23E−08 | P.F. |
| (parental) | | | | | | |
| SA-13-68 | No | N.M. | 8.15E−08 | N.B. | 3.45E−09 | 1.92E−09 |
| SA-13-69 | No | N.M. | P.F. | N.B. | 3.99E−09 | P.F. |
| SA-13-71 | No | N.M. | P.F. | N.B. | 6.56E−09 | P.F. |
| SA-19 | Yes | N.D. | N.B. | N.B. | 7.39E−09 | P.F. |
| (parental) | | | | | | |
| SA-19-72 | Yes | 8.8E−10 | 4.95E−09 | N.B. | 4.75E−09 | 1.10E−09 |
| SA-19-73 | Yes | 1.8E−10 | 1.62E−09 | N.B. | 2.95E−09 | 7.10E−10 |
| SA-19-74 | Yes | 1.1E−09 | 3.16E−09 | N.B. | 4.08E−09 | 6.76E−10 |
| SA-19-75 | Yes | 4.2E−10 | 1.67E−09 | N.B. | 3.13E−09 | 5.37E−10 |
| SA-19-76 | Yes | 4.2E−10 | 1.64E−09 | N.B. | 2.05E−09 | 5.04E−10 |
| SA-19-77 | Yes | 1.2E−09 | 3.61E−09 | N.B. | 3.94E−09 | 4.41E−10 |
| SA-19-78 | Yes | 8.4E−11 | 1.53E−09 | N.B. | 2.08E−09 | 1.06E−09 |
| SA-19-79 | Yes | 1.4E−10 | 6.89E−10 | N.B. | 1.36E−09 | 3.10E−10 |
| SA-19-80 | Yes | 2.2E−10 | 2.26E−09 | N.B. | 4.25E−09 | 1.41E−09 |
| SA-19-81 | Yes | 3.7E−10 | 1.53E−09 | N.B. | 2.59E−09 | 7.11E−10 |
| SA-19-82 | Yes | 2.7E−10 | 1.44E−09 | N.B. | 3.45E−09 | 7.42E−10 |
| SA-19-83 | Yes | 3.1E−10 | 1.44E−09 | N.B. | 2.82E−09 | 5.05E−10 |
| SA-19-84 | Yes | 6.4E−10 | 2.02E−09 | N.B. | 2.60E−09 | 5.73E−10 |
| SA-56 | No | N.D. | 2.06E−07 | N.B. | N.B. | 2.04E−09 |
| (parental) | | | | | | |
| SA-56-85 | No | 1.2E−10 | 7.72E−10 | N.B. | N.B. | 3.41E−10 |
| SA-56-86 | No | 1.8E−10 | 1.02E−09 | N.B. | 1.24E−08 | 3.39E−10 |
| SA-56-87 | No | 1.7E−10 | 1.10E−09 | N.B. | N.B. | 3.96E−10 |
| SA-56-88 | No | 1.6E−10 | 8.36E−10 | N.B. | N.B. | 3.79E−10 |
| SA-56-89 | No | 2.5E−10 | 1.03E−09 | N.B. | N.B. | 4.15E−10 |
| SA-56-90 | No | 1.7E−10 | 1.11E−09 | N.B. | N.B. | 3.78E−10 |
| SA-56-91 | No | 5.1E−10 | 1.64E−09 | N.B. | N.B. | 4.12E−10 |
| SA-56-92 | No | 3.5E−10 | 1.29E−09 | N.B. | N.B. | 3.65E−10 |
| SA-56-93 | No | 3.6E−10 | 2.34E−09 | N.B. | N.B. | 8.62E−10 |
| SA-56-94 | No | 8.8E−10 | 3.43E−09 | N.B. | N.B. | 5.66E−10 |
| SA-56-95 | No | 5.2E−10 | 2.98E−09 | N.B. | N.B. | 1.06E−09 |
| SA-56-96 | No | 1.6E−09 | 5.35E−09 | N.B. | N.B. | 1.04E−09 |

Though the original five parental anti-SIRPA antibodies of the present disclosure did not demonstrate SIRPβ1 cross-reactivity, ForteBio analysis revealed that progeny anti-SIRPA antibodies from 3 of the 5 parental antibodies acquired avid binding to SIRPβ1 despite the application of negative selection pressure during library screening.

To determine if avid binding triggers SIRPβ1-dependent signaling, anti-SIRPA antibodies of the present disclosure were assessed for their ability to induce gene expression in human SIRPA and SIRPβ1 reporter cells. As described previously, test antibodies or the positive control anti-SIRPA/B1 antibody (clone AM4-5) were adsorbed onto 96-well plates at 10 μg/mL. After washing, 10⁵ BWZ-huSIRPA or BWZ-huSIRPβ1 NFAT-luciferase reporter cells were seeded onto wells and incubated overnight at 37 C. Luciferase activity was quantified by adding OneGlo reagent (Promega) to each well and incubating samples at room temperature for 3 min on a plate shaker. The luminescence signal was quantified using a BioTek Synergy™ Microplate Reader using GEN5™ 2.04 software.

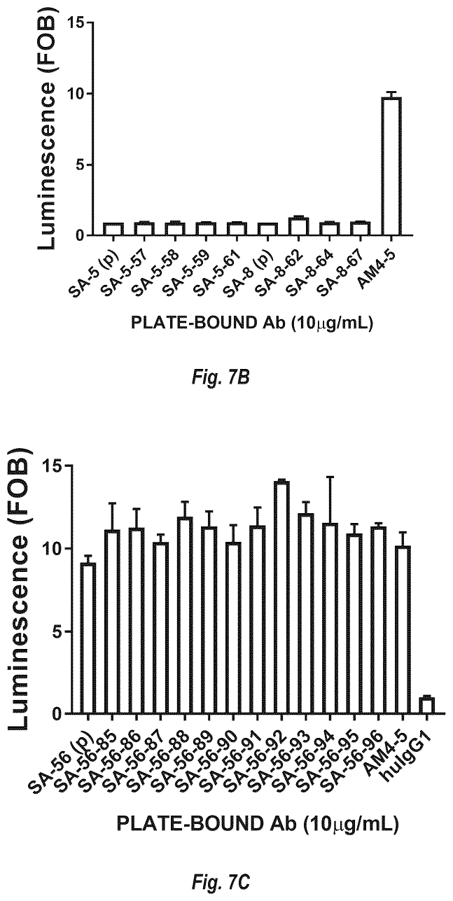

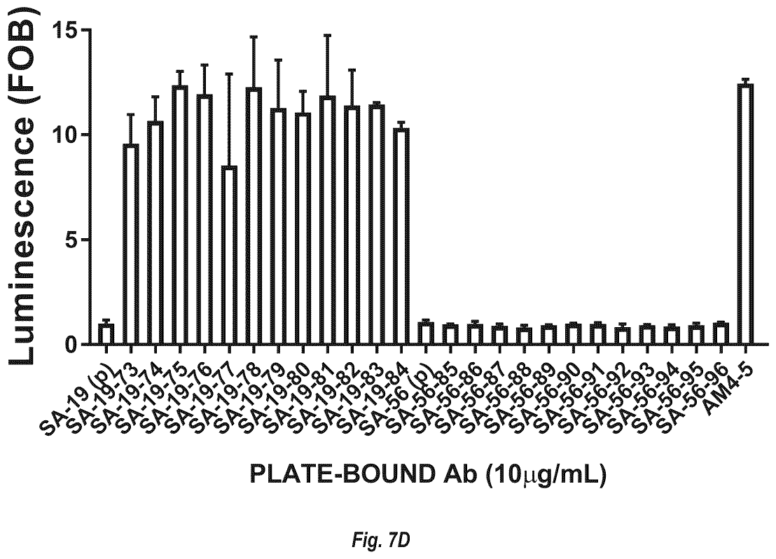

As shown in FIG. 7A and FIG. 7C, both the parental (p) anti-SIRPA antibodies and their affinity-matured progeny retained the ability to induce luciferase expression in the BWZ-huSIRPA reporter cells. In contrast, only the affinity matured clones derived from one parental clone (SA-19) significantly induced luciferase expression when BWZ-huSIRPβ1 reporter cells were added onto antibody-coated wells (FIG. 7B and FIG. 7D). As a positive control, the anti-SIRPA/β1 antibody, clone AM4-5, induced luciferase expression in both BWZ-huSIRPA and BWZ-huSIRPβ1 reporter cells (FIG. 7A-7D). These results suggested that avid binding to soluble SIRPβ1 does not translate to functional engagement of membrane-bound SIRPβ1. Thus, most anti-SIRPA antibodies of the present disclosure selected demonstrated functional specificity towards membrane-bound human SIRPA.

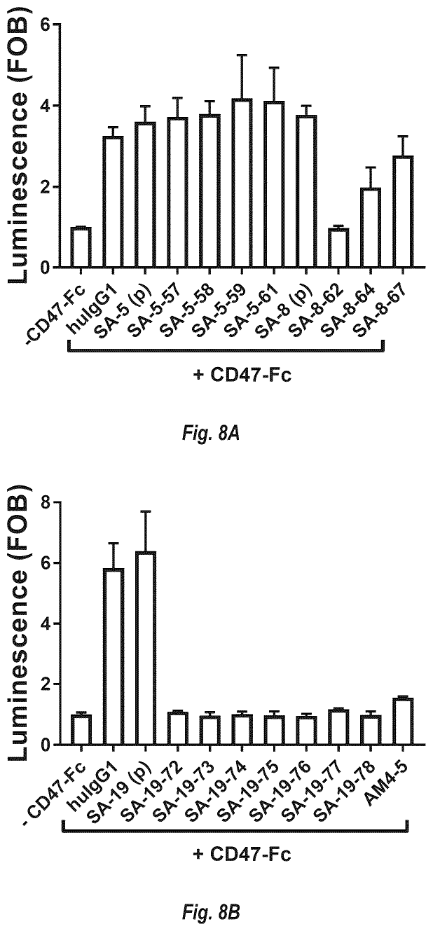

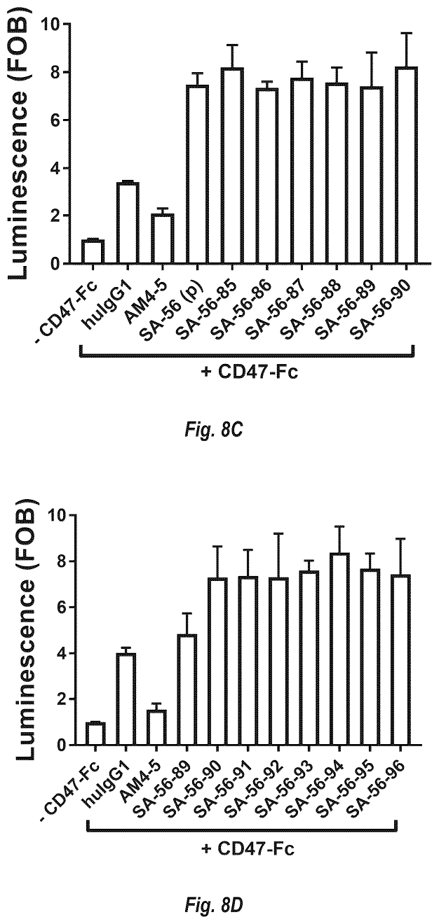

Next, affinity matured anti-SIRPA antibodies of the present disclosure were assessed for either ligand blocking or ligand enhancing properties. As described previously, soluble CD47-Fc was adsorbed onto 96-well plates at 37 C for several hours. After washing, 10⁵ BWZ-huSIRPA reporter cells were added to wells in the presence of 2 μg/mL of test antibody or isotype control and incubated overnight at 37 C. Luciferase activity was quantified by adding OneGlo reagent (Promega) and capturing the luminescence signal with a BioTek Synergy™ Microplate Reader using GEN5™ 2.04 software. As shown in FIG. 8A and FIG. 8B, anti-SIRPA antibodies derived from the parental anti-SIRPA antibodies SA-8 and SA-19 demonstrated ligand blocking activity consistent with the ForteBio competition assay described above. Antibodies derived from parental anti-SIRPA antibody SA-5 failed to show any significant effect in CD47-induced gene expression in reporter cells (FIG. 8A).

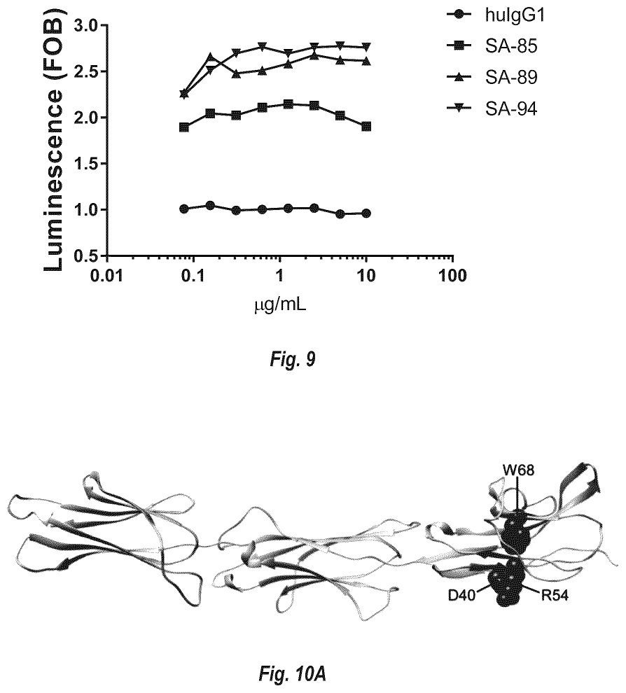

Among the anti-SIRPA antibody SA-8 derived antibodies, anti-SIRPA antibody SA-8-62 exhibited the greatest ligand blocking activity at the concentration tested, which correlated with this clone attaining the highest affinity towards the antigen. Among the anti-SIRPA antibody SA-19 derived antibodies, all progeny antibodies tested at the indicated concentration effectively blocked CD47-induced luciferase expression in reporter cells comparable to the positive control anti-SIRPA/β1 antibody, AM4-5. In contrast to ligand blocking antibodies, FIG. 8C and FIG. 8D show that antibodies derived from the anti-SIRPA antibody SA-56 parental clone augmented CD47-induced gene expression in reporter cells. Furthermore, to verify that anti-SIRPA antibody SA-56 derived antibodies also enhanced ligand binding from cell surface expressed CD47, BWZ-huSIRPA reporter cells were mixed with Raji B cells in the presence of test antibodies (SA-85, SA-89, and SA-94) or isotype control and incubated overnight at 37 C. As shown in FIG. 9, the addition of anti-SIRPA antibodies stimulated gene expression in reporter cells when combined with CD47-expressing Raji cells. The isotype control showed no stimulatory effect on SIRPA signaling. These results suggested that the affinity matured anti-SIRPA antibodies derived from parental anti-SIRPA antibody SA-56 retained the epitope recognized by the parental clone allowing enhanced ligand binding to the receptor.

### Example 11: Epitope Mapping of Anti-SIRPA Antibodies

Epitope mapping of anti-SIRPA antibodies was performed using an alanine-scanning library created by shotgun mutagenesis of the human SIRPA cDNA sequence. A SIRPA expression construct encoding a C-terminal V5 epitope tag was subjected to high-throughput alanine scanning mutagenesis (outlined in Davidson and Doranz, 2014 Immunology 143, 13-20) to generate a comprehensive mutation library. Each of the residues representing the SIRPA extracellular domain (amino acids 31-374) was mutated, most to alanine, while alanine codons were mutated to serine.

The SIRPA mutant library clones, arrayed in a 384-well microplate, were transfected individually into HEK-293T cells and allowed to express for 22 hours. Antibodies were digested to generate Fabs, after which cells were incubated with Fabs diluted in 10% normal goat serum (NGS) (Sigma-Aldrich, St. Louis, MO). Prior to library screening, primary Fab concentrations were determined using an independent immunofluorescence titration curve against cells expressing wild type SIRPA to ensure that signals were within the linear range of detection. Fabs were detected using 7.5 μg/ml AlexaFluor488-conjugated secondary antibody (Jackson ImmunoResearch Laboratories, Westgrove, PA) in 10% NGS. Cells were washed twice with PBS and resuspended in Cellstripper (Cellgro, Manassas, VA) with 0.1% BSA (Sigma-Aldrich, St. Louis, MO). In some cases, higher stringency conditions were used, including increased pH, increased temperature, and increased dissociation time. Mean cellular fluorescence was detected using the Intellicyt high throughput flow cytometer (HTFC, Intellicyt, Albuquerque, NM). Fab reactivities against each mutant clone were calculated relative to wild-type SIRPA protein reactivity by subtracting the signal from mock-transfected controls, and normalizing to the signal from wild-type SIRPA transfected controls.

Mutated residues within library clones were identified as critical to the Fab epitope if they did not support reactivity of the test Fab but did support reactivity of commercially available reference antibody, MAB4546 (R&D Systems), or additional anti-SIRPA Fabs. This counter-screen strategy facilitated the exclusion of SIRPA mutants that were locally misfolded or that had an expression defect.

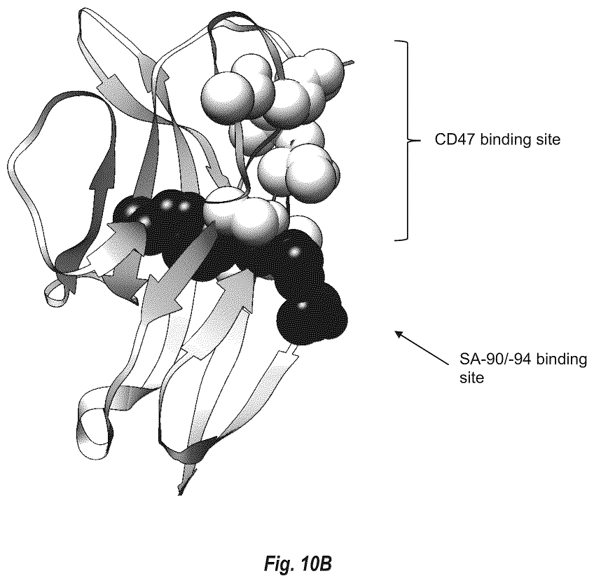

Table 10 depicts the mean binding reactivities and ranges for all critical residues identified in these screens. Primary critical residues were defined as residues where mutations were negative for test antibody binding (<30% of binding to WT) but positive for the control antibody (>80% WT). FIG. 10A and FIG. 10B depict crystal structure models of SIRPA (PDB ID 2WNG; Hatherley et al., 2009) highlighting the critical residues for binding anti-SIRPA antibodies SA-90 and SA-94 as black spheres. The amino acid residues critical for antibody binding are listed in Table 11.

TABLE 10
Binding Reactivity (% WT).

| Mutation | SA-56-90 Fab | SA-56-94 Fab |
|---|---|---|
| D40A | 0.4 (2) | 0.2 (1) |
| R54A | 1.7 (0) | 2.9 (3) |
| W68A | 19.3 (6) | 25.6 (11) |

As indicated in Table 11, the critical SIRPA residues involved in binding by anti-SIRPA antibodies SA-56-90 and SA-56-94 corresponded to amino acid residues D40, R54, and W68 of SEQ ID NO:1 [human SIRPAv1 sequence]. These residues lie within the membrane-distal IgV domain of SIRPA, referred to in the literature as the D1 domain, which correspond to amino acids 32-137 of SEQ ID NO:1. Multiple published reports demonstrate that the D1 domain of human SIRPA binds to CD47. The critical residues for anti-SIRPA antibodies SA-56-90 and SA-56-94 appear to border the CD47 binding site (FIG. 10B, white spheres), which may potentially stabilize the interaction between ligand and receptor, leading to increased CD47 binding to SIRPA.

TABLE 11
SIRPA Amino Acids for Antibody Binding

| Antibody | Critical SIRPA Residues |
|---|---|
| SA-56-90 | D40, R54, W68 |
| SA-56-94 | D40, R54, W68 |

### Example 12: Agonistic Anti-SIRPA Antibodies Decrease Phagocytic Activity by Phagocytic Cells

The SIRPA-CD47 axis provides an inhibitory signal to modulate myeloid cell activity. Tumor cells exploit this pathway by upregulating expression of CD47 to evade macrophage-mediated engulfment. Therapeutic agents developed for oncology indications targeting SIRPA and/or CD47 aim to block receptor-ligand interaction to antagonize the inhibitory signal from SIRPA. In contrast, the anti-SIRPA antibodies disclosed herein promote SIRPA-CD47 interaction with the purpose of inhibiting myeloid cell activation during, for example, inflammation. To ascertain if such agonistic anti-SIRPA antibodies suppress macrophage cellular functions, anti-SIRPA antibodies of the present disclosure were assessed in a tumor cell phagocytosis assay. Briefly, Human primary monocytes were isolated from heparinized human blood (Blood Centers of the Pacific) using RosetteSep Human Monocyte Enrichment Cocktail (STEMCELL Technologies), according to the manufacturer's protocol. Monocytes were seeded in RPMI (Invitrogen) containing 10% Fetal Calf Serum (Hyclone) and 50 μg/ml M-CSF (Peprotech) to induce differentiation to macrophages After 5-6 days, macrophages were harvested by scraping cells attached to plastic.

Red Avidin (Invitrogen) is a streptavidin molecule conjugated with pHrodo red dye, a fluorogenic marker that acquires fluorescence in acidic environments, such as the phagosome. For target tumor cell labeling, 500 nM Red Avidin was mixed with 15 nM biotinylated Lens Culinaris Agglutinin (LCA; Vector Labs). Red Avidin-LCA complexes were then mixed in a 1:1 volumetric ratio with ≥250,000 Raji cells in serum-free RPMI media on ice. The sugar-binding properties of LCA links Red Avidin to carbohydrate structures on the tumor cell surface. After brief washing steps, Red Avidin-LCA-labeled Raji cells were mixed with monocyte-derived human macrophages in serum-free RPMI media and incubated at 37 C for 2 hours. Macrophages were then collected and stained on ice with anti-CD14 APC in FACS buffer containing FcγR-blocking antibodies. Phagocytic activity was measured by counting percent of APC/pHrodo-double positive macrophages. As a control, unlabeled Raji cells were mixed with macrophages to establish background fluorescence.

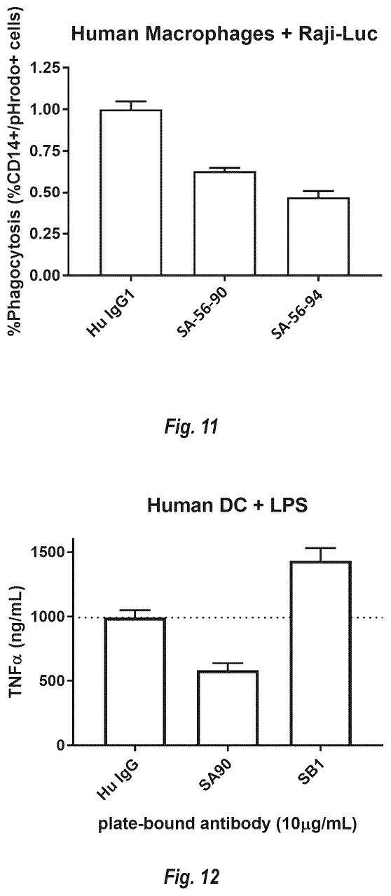

To test anti-SIRPA antibodies, macrophages were incubated overnight with the indicated candidate antibody or isotype control. The following day, pHrodo-labeled Raji cells were mixed with treated macrophages followed by quantification of phagocytic activity. As shown in FIG. 11, both ligand enhancing anti-SIRPA antibodies SA-56-90 and SA-56-94 suppressed tumor cell phagocytosis by macrophages compared to isotype control. These results suggested that the ligand enhancing properties of anti-SIRPA antibodies of the present disclosure may function, at least in part, by decreasing phagocytosis by a phagocytic cell.

### Example 13: Agonistic Anti-SIRPA Antibodies Decrease Dendritic Cell Cytokine Release

As noted previously, several reports implicate SIRPA+ dendritic cell subsets in the initiation and maintenance of airway and intestinal inflammation. For example, CD103− SIRPA+ dendritic cell isolated from the small intestine demonstrate an ability to drive Th17 polarization and to secrete high levels of pro-inflammatory cytokines upon stimulation. Thus, agonistic anti-SIRPA antibodies of the present disclosure were also assessed for suppression of human dendritic cells. Briefly, human primary monocytes were isolated from heparinized human blood (Blood Centers of the Pacific) using RosetteSep Human Monocyte Enrichment Cocktail (STEMCELL Technologies), according to the manufacturer's protocol. Monocytes were seeded in RPMI (Invitrogen) containing 10% Fetal Calf Serum (Hyclone) and 100 μg/ml IL-4+100 μg/ml GM-CSF (Peprotech) to induce differentiation to dendritic cells. After 5 days, immature dendritic cells were harvested by collecting cells in suspension.

Antibodies were adsorbed onto 96-well plates at 10 μg/mL. After washing, 10⁵ monocyte-derived dendritic cells were seeded onto wells in the presence of 0.5 ng/mL LPS and incubated overnight at 37 C. The following day, cells were pelleted, and supernatant fraction collected for human TNFα quantification by ELISA according to the manufacturer's instructions (ThermoFisher Scientific). Dendritic cells were seeded onto wells coated with human IgG1 isotype control or agonistic anti-SIRPA antibody SA-56-90. As a further comparison, dendritic cells were also seeded onto wells coated with an agonistic anti-SIRPβ1 antibody, which activates dendritic cells by signaling through the ITAM-bearing DAP12 adaptor protein. As shown in FIG. 12, LPS potently induced TNFα release from dendritic cells cultured on isotype control antibody (Hu IgG). However, dendritic cells cultured in the presence of anti-SIRPA antibody SA-56-90 released ˜42% less TNFα upon exposure to LPS compared to that observed with isotype control antibody. In contrast, dendritic cells cultured in the presence of an anti-SIRPβ1 antibody released 44% more TNFα upon exposure to LPS. These results indicated that the effect of anti-SIRPA antibodies of the present disclosure on dendritic cells corresponded to the intracellular signaling motif associated with the specific receptor targeted. By identifying SIRPA-specific antibodies that increase CD47 binding to SIRPA, agonistic anti-SIRPA antibodies are effective at delivering an inhibitory signal to suppress myeloid cells.

### Example 14: Anti-SIRPA Antibodies Suppress Synapse Elimination in Microglia-Neuron Co-Cultures

In vitro model systems have been developed to measure microglial elimination of neuronal synapses. These model systems demonstrate that microglia in contact with neurons reduce spine density and mEPSC frequency, which is a measure of neuronal communication through functional synapses. To determine the role of SIRPA in synaptic pruning, microglia-neuronal co-cultures are established from dissociated brain harvested from huSIRPA×huCD47 knock-in mice. Briefly, cerebral cortices are harvested from neonatal mice before digestion and centrifugation to obtain a pellet for cell seeding. Primary cortical microglia are harvested from the astrocyte layer by shaking the flasks at 200 rpm for 1-2 h at 37° C., isolated microglia are added into DIV 14 neurons at a 1:3 microglia to neuron ratio for 3 days co-culture. Cells are then treated with anti-SIRPA antibodies, CD47 blocking anti-SIRPA antibodies, or isotype control antibody. Co-cultures are terminated by fixing cells with 4% PFA for analysis. Neuron-microglia and synaptic density is quantified by immunofluorescence staining of cells with antibodies labeling Iba-1 (microglia), MAP2 (neurons), Synapsin I (marker of pre-synapses), and PSD-95 (postsynaptic density protein 95, a marker of post-synapses). Images are captured by confocal microscopy. To quantify the density of Synapsin or PSD-95 puncta, concentric circles are drawn around the microglia. The puncta number within each concentric circle are counted. Synaptic density is calculated by puncta number/neurites length in the given area. All images are processed using ImageJ.

Since microglia engulf synapses through the recognition of pro-phagocytic “eat-me” signals bound to synaptic membranes, such as phosphatidylserine and complement factors, antibodies that agonize the SIRPA-CD47 pathway suppress these clearance mechanisms. Microglia-neuronal co-cultures treated with anti-SIRPA antibodies of the present disclosure demonstrate increased staining for synapsin and PSD-95 on neurons near microglia when compared to isotype control-treated co-cultures. Microglia-neuronal co-cultures treated with CD47-blocking anti-SIRPA or anti-CD47 antibodies exacerbate synaptic pruning and result in significantly reduced staining for synapsin and PSD-95. Oligomeric forms of Aβ₄₂ peptides have been shown to enhance synaptic pruning by downregulating SIRPA expression on microglia in neuronal-glia co-culture system. Treating microglia with anti-SIRPA antibodies of the present disclosure in the presence of Aβ₄₂ oligomers protect synapses from elimination when compared to co-cultures treated with Aβ₄₂ oligomers alone.

### Example 15: Anti-SIRPA Antibodies Suppress Synapse Elimination in Mouse Models of Neurodegeneration

Cognitive decline in neurodegeneration is attributed to the significant loss of neuronal synapses in the diseased brain. To evaluate the role of SIRPA in protecting synapses in neurodegeneration, huSIRPA×huCD47 knock-in mice are crossed to various mouse models of neurodegeneration, such as 5×FAD mouse model. 5×FAD mice overexpress mutant human APP (695) with the Swedish (K670N, M671L), Florida (1716V), and London (V7171) familial Alzheimer's disease (FAD) mutations, along with human PS1 harboring two FAD mutations, M146L and L286V. Both transgenes are regulated by the mouse Thy1 promoter to drive over expression on the brain and recapitulate major features of AD. Mice are treated weekly with 50 mg/kg anti-SIRPA antibody or with isotype control antibody mIgG1 (clone MOPC-21, Bioxcell) starting from 14 weeks of age. Mice are tested for the number of microglia in the brain, and for reduction in cognitive deficit using Morris Water maze, a spatial learning and memory task, Radial Arm Water Maze, a spatial learning and memory task, Y Maze (quantifies spontaneous alternation as a measure of spatial cognition), novelty preference in in an open field, operant learning to assess learning and memory, and fear conditioning. To measure synaptic density, 20 μm mice brain sections stained with pre-/post-synaptic markers (Homer1/Vglut1, Homer1/Vglut2, Synapsin1/PSD95) are captured by confocal microscope or laser scanning microscope through ×63 objective lens. Captured images are used to quantify the number of colocalized pre and postsynaptic puncta by ImageJ software, and single-channel images are used to quantify single synaptic marker density by ImageJ. Synaptic density is determined as puncta number/given area.

Mice treated with agonistic anti-SIRPA antibodies of the present disclosure significantly increase synaptic density in the brains of 5×FAD mice as measured by staining and co-localization of pre- and post-synaptic markers when compared to mice treated with isotype control antibody. Increased synaptic density in brains of mice treated with anti-SIRPA antibodies of the present disclosure correlates with improved cognitive and behavioral scores when compared to that observed in mice treated with isotype control antibody.

1. An isolated antibody that specifically binds to human SIRPA, wherein the antibody comprises a heavy chain variable region (VH) and a light chain variable region (VL), wherein the heavy chain variable region (VH) comprises:
a. an HVR-H1 comprising an amino acid sequence chosen from any one of SEQ ID NOs: 102-122, 318-328, and 346-349;
b. an HVR-H2 comprising an amino acid sequence chosen from any one of SEQ ID NOs: 123-146 and 329-345; and
c. an HVR-H3 comprising an amino acid sequence chosen from any one of SEQ ID NOs: 147-201, 350-360, and 371-386.

2. The antibody of claim 1, wherein the light chain variable region (VL) comprises:
a. an HVR-L1 comprising an amino acid sequence chosen from any one of SEQ ID NOs: 7-28;
b. an HVR-L2 comprising an amino acid sequence chosen from any one of SEQ ID NOs: 29-40; and
c. an HVR-L3 comprising an amino acid sequence chosen from any one of SEQ ID NOs: 41-101.

3. The antibody of claim 1, wherein the antibody comprises a VH that is at least 90%, at least 95%, at least 97%, or at least 99% identical to an amino acid sequence chosen from any one of SEQ ID NOs: 202-256, 361-370, and 387-412.

4. The antibody of claim 1, wherein the antibody comprises a VL that is at least 90%, at least 95%, at least 97%, or at least 99% identical to an amino acid sequence chosen from any one of SEQ ID NOs: 257-317, 261, 264, 269, 275, 312, and 413.

5. The antibody of claim 1, wherein the VH comprises 1, 2, 3, 4, 5, 6, 7, 8, 9, or 10 amino acid substitutions, insertions, and/or deletions compared to an amino acid sequence chosen from any one of SEQ ID NOs: 202-256, 361-370, and 387-412.

6. The antibody of claim 1, wherein the VL comprises 1, 2, 3, 4, 5, 6, 7, 8, 9, or 10 amino acid substitutions, insertions, and/or deletions compared to an amino acid sequence chosen from any one of SEQ ID NOs: 257-317, 261, 264, 269, 275, 312, and 413.

7. The antibody of claim 1, wherein the antibody comprises a VH comprising an amino acid sequence chosen from any one of SEQ ID NOs: 202-256, 361-370, and 387-412.

8. The antibody of claim 1, wherein the antibody comprises a VL comprising an amino acid sequence chosen from any one of SEQ ID NOs: 257-317, 261, 264, 269, 275, 312, and 413.

9. (canceled)

10. (canceled)

11. (canceled)

12. An isolated antibody that specifically binds to human SIRPA, wherein the antibody comprises:
a. a VH comprising an HVR-H1 comprising the amino acid sequence of SEQ ID NO: 102, an HVR-H2 comprising the amino acid sequence of SEQ ID NO: 123, and an HVR-H3 comprising the amino acid sequence of SEQ ID NO: 147; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 41;
b. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 148; and a VL comprising an HVR-L1 comprising the amino acid sequence of SEQ ID NO: 8, an HVR-L2 comprising the amino acid sequence of SEQ ID NO: 30, and an HVR-L3 comprising the amino acid sequence of SEQ ID NO: 42;
c. a VH comprising an HVR-H1 comprising the amino acid sequence of SEQ ID NO: 104, an HVR-H2 comprising the amino acid sequence of SEQ ID NO: 125, and an HVR-H3 comprising the amino acid sequence of SEQ ID NO: 149; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 43
d. a VH comprising an HVR-H1 comprising the amino acid sequence of SEQ ID NO: 105, an HVR-H2 comprising the amino acid sequence of SEQ ID NO: 125, and an HVR-H3 comprising the amino acid sequence of SEQ ID NO: 150; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 44;
e. a VH comprising an HVR-H1 comprising the amino acid sequence of SEQ ID NO: 106, an HVR-H2 comprising the amino acid sequence of SEQ ID NO: 125, and an HVR-H3 comprising the amino acid sequence of SEQ ID NO: 151; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
f. a VH comprising an HVR-H1 comprising the amino acid sequence of SEQ ID NO: 107, an HVR-H2 comprising the amino acid sequence of SEQ ID NO: 126, and an HVR-H3 comprising the amino acid sequence of SEQ ID NO: 152; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 46;
g. a VH comprising an HVR-H1 comprising the amino acid sequence of SEQ ID NO: 103, an HVR-H2 comprising the amino acid sequence of SEQ ID NO: 127, and an HVR-H3 comprising the amino acid sequence of SEQ ID NO: 153; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 47;
h. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
i. a VH comprising an HVR-H1 comprising the amino acid sequence of SEQ ID NO: 106, an HVR-H2 comprising the amino acid sequence of SEQ ID NO: 128, and an HVR-H3 comprising the amino acid sequence of SEQ ID NO: 155; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 49;
j. a VH comprising an HVR-H1 comprising the amino acid sequence of SEQ ID NO: 107, an HVR-H2 comprising the amino acid sequence of SEQ ID NO: 126, and an HVR-H3 comprising the amino acid sequence of SEQ ID NO: 156; and a VL comprising an HVR-L1 comprising the amino acid sequence of SEQ ID NO: 13, an HVR-L2 comprising the amino acid sequence of SEQ ID NO: 34, and an HVR-L3 comprising the amino acid sequence of SEQ ID NO: 50;
k. a VH comprising an HVR-H1 comprising the amino acid sequence of SEQ ID NO: 102, an HVR-H2 comprising the amino acid sequence of SEQ ID NO: 123, and an HVR-H3 comprising the amino acid sequence of SEQ ID NO: 157; and a VL comprising an HVR-L1 comprising the amino acid sequence of SEQ ID NO: 13, an HVR-L2 comprising the amino acid sequence of SEQ ID NO: 35, and an HVR-L3 comprising the amino acid sequence of SEQ ID NO: 51;
l. a VH comprising an HVR-H1 comprising the amino acid sequence of SEQ ID NO: 108, an HVR-H2 comprising the amino acid sequence of SEQ ID NO: 129, and an HVR-H3 comprising the amino acid sequence of SEQ ID NO: 158; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 52;
m. a VH comprising an HVR-H1 comprising the amino acid sequence of SEQ ID NO: 106, an HVR-H2 comprising the amino acid sequence of SEQ ID NO: 125, and an HVR-H3 comprising the amino acid sequence of SEQ ID NO: 159; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
n. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 160; and a VL comprising an HVR-L1 comprising the amino acid sequence of SEQ ID NO: 15, an HVR-L2 comprising the amino acid sequence of SEQ ID NO: 33, and an HVR-L3 comprising the amino acid sequence of SEQ ID NO: 54;
o. a VH comprising an HVR-H1 comprising the amino acid sequence of SEQ ID NO: 110, an HVR-H2 comprising the amino acid sequence of SEQ ID NO: 131, and an HVR-H3 comprising the amino acid sequence of SEQ ID NO: 161; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 55;
p. a VH comprising an HVR-H1 comprising the amino acid sequence of SEQ ID NO: 111, an HVR-H2 comprising the amino acid sequence of SEQ ID NO: 132, and an HVR-H3 comprising the amino acid sequence of SEQ ID NO: 162; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 56;
q. a VH comprising an HVR-H1 comprising the amino acid sequence of SEQ ID NO: 110, an HVR-H2 comprising the amino acid sequence of SEQ ID NO: 133, and an HVR-H3 comprising the amino acid sequence of SEQ ID NO: 163; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 57;
r. a VH comprising an HVR-H1 comprising the amino acid sequence of SEQ ID NO: 112, an HVR-H2 comprising the amino acid sequence of SEQ ID NO: 134, and an HVR-H3 comprising the amino acid sequence of SEQ ID NO: 164; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 58;
s. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 165; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
t. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 60;
u. a VH comprising an HVR-H1 comprising the amino acid sequence of SEQ ID NO: 113, an HVR-H2 comprising the amino acid sequence of SEQ ID NO: 135, and an HVR-H3 comprising the amino acid sequence of SEQ ID NO: 166; and a VL comprising an HVR-L1 comprising the amino acid sequence of SEQ ID NO: 8, an HVR-L2 comprising the amino acid sequence of SEQ ID NO: 30, and an HVR-L3 comprising the amino acid sequence of SEQ ID NO: 61;
v. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 167; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 62;
w. a VH comprising an HVR-H1 comprising the amino acid sequence of SEQ ID NO: 114, an HVR-H2 comprising the amino acid sequence of SEQ ID NO: 136, and an HVR-H3 comprising the amino acid sequence of SEQ ID NO: 168; and a VL comprising an HVR-L1 comprising the amino acid sequence of SEQ ID NO: 17, an HVR-L2 comprising the amino acid sequence of SEQ ID NO: 37, and an HVR-L3 comprising the amino acid sequence of SEQ ID NO: 63;
x. a VH comprising an HVR-H1 comprising the amino acid sequence of SEQ ID NO: 103, an HVR-H2 comprising the amino acid sequence of SEQ ID NO: 137, and an HVR-H3 comprising the amino acid sequence of SEQ ID NO: 169; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 64;
y. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 170; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 65;
z. a VH comprising an HVR-H1 comprising the amino acid sequence of SEQ ID NO: 111, an HVR-H2 comprising the amino acid sequence of SEQ ID NO: 132, and an HVR-H3 comprising the amino acid sequence of SEQ ID NO: 171; and a VL comprising an HVR-L1 comprising the amino acid sequence of SEQ ID NO: 18, an HVR-L2 comprising the amino acid sequence of SEQ ID NO: 32, and an HVR-L3 comprising the amino acid sequence of SEQ ID NO: 66;
aa. a VH comprising an HVR-H1 comprising the amino acid sequence of SEQ ID NO: 115, an HVR-H2 comprising the amino acid sequence of SEQ ID NO: 139, and an HVR-H3 comprising the amino acid sequence of SEQ ID NO: 172; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 67;
bb. a VH comprising an HVR-H1 comprising the amino acid sequence of SEQ ID NO: 116, an HVR-H2 comprising the amino acid sequence of SEQ ID NO: 140, and an HVR-H3 comprising the amino acid sequence of SEQ ID NO: 173; and a VL comprising an HVR-L1 comprising the amino acid sequence of SEQ ID NO: 19, an HVR-L2 comprising the amino acid sequence of SEQ ID NO: 38, and an HVR-L3 comprising the amino acid sequence of SEQ ID NO: 68;
cc. a VH comprising an HVR-H1 comprising the amino acid sequence of SEQ ID NO: 116, an HVR-H2 comprising the amino acid sequence of SEQ ID NO: 140, and an HVR-H3 comprising the amino acid sequence of SEQ ID NO: 173; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 69;
dd. a VH comprising an HVR-H1 comprising the amino acid sequence of SEQ ID NO: 103, an HVR-H2 comprising the amino acid sequence of SEQ ID NO: 137, and an HVR-H3 comprising the amino acid sequence of SEQ ID NO: 174; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 70;
ee. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 170; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 71;
ff. a VH comprising an HVR-H1 comprising the amino acid sequence of SEQ ID NO: 117, an HVR-H2 comprising the amino acid sequence of SEQ ID NO: 137, and an HVR-H3 comprising the amino acid sequence of SEQ ID NO: 175; and a VL comprising an HVR-L1 comprising the amino acid sequence of SEQ ID NO: 21, an HVR-L2 comprising the amino acid sequence of SEQ ID NO: 36, and an HVR-L3 comprising the amino acid sequence of SEQ ID NO: 72;
gg. a VH comprising an HVR-H1 comprising the amino acid sequence of SEQ ID NO: 110, an HVR-H2 comprising the amino acid sequence of SEQ ID NO: 133, and an HVR-H3 comprising the amino acid sequence of SEQ ID NO: 176; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 73;
hh. a VH comprising an HVR-H1 comprising the amino acid sequence of SEQ ID NO: 118, an HVR-H2 comprising the amino acid sequence of SEQ ID NO: 141, and an HVR-H3 comprising the amino acid sequence of SEQ ID NO: 177; and a VL comprising an HVR-L1 comprising the amino acid sequence of SEQ ID NO: 13, an HVR-L2 comprising the amino acid sequence of SEQ ID NO: 35, and an HVR-L3 comprising the amino acid sequence of SEQ ID NO: 74;
ii. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 178; and a VL comprising an HVR-L1 comprising the amino acid sequence of SEQ ID NO: 13, an HVR-L2 comprising the amino acid sequence of SEQ ID NO: 39, and an HVR-L3 comprising the amino acid sequence of SEQ ID NO: 75;
jj. a VH comprising an HVR-H1 comprising the amino acid sequence of SEQ ID NO: 113, an HVR-H2 comprising the amino acid sequence of SEQ ID NO: 135, and an HVR-H3 comprising the amino acid sequence of SEQ ID NO: 179; and a VL comprising an HVR-L1 comprising the amino acid sequence of SEQ ID NO: 22, an HVR-L2 comprising the amino acid sequence of SEQ ID NO: 31, and an HVR-L3 comprising the amino acid sequence of SEQ ID NO: 76;
kk. a VH comprising an HVR-H1 comprising the amino acid sequence of SEQ ID NO: 116, an HVR-H2 comprising the amino acid sequence of SEQ ID NO: 139, and an HVR-H3 comprising the amino acid sequence of SEQ ID NO: 180; and a VL comprising an HVR-L1 comprising the amino acid sequence of SEQ ID NO: 23, an HVR-L2 comprising the amino acid sequence of SEQ ID NO: 38, and an HVR-L3 comprising the amino acid sequence of SEQ ID NO: 77;
ll. a VH comprising an HVR-H1 comprising the amino acid sequence of SEQ ID NO: 116, an HVR-H2 comprising the amino acid sequence of SEQ ID NO: 139, and an HVR-H3 comprising the amino acid sequence of SEQ ID NO: 180; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 78;
mm. a VH comprising an HVR-H1 comprising the amino acid sequence of SEQ ID NO: 120, an HVR-H2 comprising the amino acid sequence of SEQ ID NO: 129, and an HVR-H3 comprising the amino acid sequence of SEQ ID NO: 181; and a VL comprising an HVR-L1 comprising the amino acid sequence of SEQ ID NO: 24, an HVR-L2 comprising the amino acid sequence of SEQ ID NO: 33, and an HVR-L3 comprising the amino acid sequence of SEQ ID NO: 79;
nn. a VH comprising an HVR-H1 comprising the amino acid sequence of SEQ ID NO: 119, an HVR-H2 comprising the amino acid sequence of SEQ ID NO: 142, and an HVR-H3 comprising the amino acid sequence of SEQ ID NO: 182; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 80;
oo. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 183; and a VL comprising an HVR-L1 comprising the amino acid sequence of SEQ ID NO: 16, an HVR-L2 comprising the amino acid sequence of SEQ ID NO: 36, and an HVR-L3 comprising the amino acid sequence of SEQ ID NO: 81;
pp. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 183; and a VL comprising an HVR-L1 comprising the amino acid sequence of SEQ ID NO: 25, an HVR-L2 comprising the amino acid sequence of SEQ ID NO: 36, and an HVR-L3 comprising the amino acid sequence of SEQ ID NO: 82;
qq. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 184; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 83;
rr. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 185; and a VL comprising an HVR-L1 comprising the amino acid sequence of SEQ ID NO: 26 an HVR-L2 comprising the amino acid sequence of SEQ ID NO: 32, and an HVR-L3 comprising the amino acid sequence of SEQ ID NO: 84;
ss. a VH comprising an HVR-H1 comprising the amino acid sequence of SEQ ID NO: 114, an HVR-H2 comprising the amino acid sequence of SEQ ID NO: 141, and an HVR-H3 comprising the amino acid sequence of SEQ ID NO: 186; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 85;
tt. a VH comprising an HVR-H1 comprising the amino acid sequence of SEQ ID NO: 104, an HVR-H2 comprising the amino acid sequence of SEQ ID NO: 125, and an HVR-H3 comprising the amino acid sequence of SEQ ID NO: 187; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 86;
uu. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 188; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 87;
vv. a VH comprising an HVR-H1 comprising the amino acid sequence of SEQ ID NO: 103, an HVR-H2 comprising the amino acid sequence of SEQ ID NO: 124, and an HVR-H3 comprising the amino acid sequence of SEQ ID NO: 189; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 88;
ww. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 190; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 89;
xx. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 191; and a VL comprising an HVR-L1 comprising the amino acid sequence of SEQ ID NO: 10, an HVR-L2 comprising the amino acid sequence of SEQ ID NO: 30, and an HVR-L3 comprising the amino acid sequence of SEQ ID NO: 90;
yy. a VH comprising an HVR-H1 comprising the amino acid sequence of SEQ ID NO: 110, an HVR-H2 comprising the amino acid sequence of SEQ ID NO: 138, and an HVR-H3 comprising the amino acid sequence of SEQ ID NO: 192; and a VL comprising an HVR-L1 comprising the amino acid sequence of SEQ ID NO: 20, an HVR-L2 comprising the amino acid sequence of SEQ ID NO: 38, and an HVR-L3 comprising the amino acid sequence of SEQ ID NO: 91;
zz. a VH comprising an HVR-H1 comprising the amino acid sequence of SEQ ID NO: 118, an HVR-H2 comprising the amino acid sequence of SEQ ID NO: 141, and an HVR-H3 comprising the amino acid sequence of SEQ ID NO: 193; and a VL comprising an HVR-L1 comprising the amino acid sequence of SEQ ID NO: 20, an HVR-L2 comprising the amino acid sequence of SEQ ID NO: 40, and an HVR-L3 comprising the amino acid sequence of SEQ ID NO: 92;
aaa. a VH comprising an HVR-H1 comprising the amino acid sequence of SEQ ID NO: 116, an HVR-H2 comprising the amino acid sequence of SEQ ID NO: 145, and an HVR-H3 comprising the amino acid sequence of SEQ ID NO: 194; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 93;
bbb. a VH comprising an HVR-H1 comprising the amino acid sequence of SEQ ID NO: 115, an HVR-H2 comprising the amino acid sequence of SEQ ID NO: 139, and an HVR-H3 comprising the amino acid sequence of SEQ ID NO: 195; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 94;
ccc. a VH comprising an HVR-H1 comprising the amino acid sequence of SEQ ID NO: 112, an HVR-H2 comprising the amino acid sequence of SEQ ID NO: 146, and an HVR-H3 comprising the amino acid sequence of SEQ ID NO: 196; and a VL comprising an HVR-L1 comprising the amino acid sequence of SEQ ID NO: 27, an HVR-L2 comprising the amino acid sequence of SEQ ID NO: 35, and an HVR-L3 comprising the amino acid sequence of SEQ ID NO: 95;
ddd. a VH comprising an HVR-H1 comprising the amino acid sequence of SEQ ID NO: 109, an HVR-H2 comprising the amino acid sequence of SEQ ID NO: 130, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
eee. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 198; and a VL comprising an HVR-L1 comprising the amino acid sequence of SEQ ID NO: 7, an HVR-L2 comprising the amino acid sequence of SEQ ID NO: 31, and an HVR-L3 comprising the amino acid sequence of SEQ ID NO: 97;
fff. a VH comprising an HVR-H1 comprising the amino acid sequence of SEQ ID NO: 122, an HVR-H2 comprising the amino acid sequence of SEQ ID NO: 139, and an HVR-H3 comprising the amino acid sequence of SEQ ID NO: 199; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 98;
ggg. a VH comprising an HVR-H1 comprising the amino acid sequence of SEQ ID NO: 121, an HVR-H2 comprising the amino acid sequence of SEQ ID NO: 143, and an HVR-H3 comprising the amino acid sequence of SEQ ID NO: 200; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 99;
hhh. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 201; and a VL comprising an HVR-L1 comprising the amino acid sequence of SEQ ID NO: 7, an HVR-L2 comprising the amino acid sequence of SEQ ID NO: 29, and an HVR-L3 comprising the amino acid sequence of SEQ ID NO: 100;
iii. a VH comprising an HVR-H1 comprising the amino acid sequence of SEQ ID NO: 119, an HVR-H2 comprising the amino acid sequence of SEQ ID NO: 129, and an HVR-H3 comprising the amino acid sequence of SEQ ID NO: 178; and a VL comprising an HVR-L1 comprising the amino acid sequence of SEQ ID NO: 13, an HVR-L2 comprising the amino acid sequence of SEQ ID NO: 39, and an HVR-L3 comprising the amino acid sequence of SEQ ID NO: 75;
jjj. a VH comprising an HVR-H1 comprising the amino acid sequence of SEQ ID NO: 109, an HVR-H2 comprising the amino acid sequence of SEQ ID NO: 144, and an HVR-H3 comprising the amino acid sequence of SEQ ID NO: 201; and a VL comprising an HVR-L1 comprising the amino acid sequence of SEQ ID NO: 28, an HVR-L2 comprising the amino acid sequence of SEQ ID NO: 33, and an HVR-L3 comprising the amino acid sequence of SEQ ID NO: 101;
kkk. a VH comprising an HVR-H1 comprising the amino acid sequence of SEQ ID NO: 318, an HVR-H2 comprising the amino acid sequence of SEQ ID NO: 329, and an HVR-H3 comprising the amino acid sequence of SEQ ID NO: 350; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
lll. a VH comprising an HVR-H1 comprising the amino acid sequence of SEQ ID NO: 318, an HVR-H2 comprising the amino acid sequence of SEQ ID NO: 330, and an HVR-H3 comprising the amino acid sequence of SEQ ID NO: 351; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO:45;
mmm. a VH comprising an HVR-H1 comprising the amino acid sequence of SEQ ID NO: 319, an HVR-H2 comprising the amino acid sequence of SEQ ID NO: 331, and an HVR-H3 comprising the amino acid sequence of SEQ ID NO: 352; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
nnn. a VH comprising an HVR-H1 comprising the amino acid sequence of SEQ ID NO: 318, an HVR-H2 comprising the amino acid sequence of SEQ ID NO: 125, and an HVR-H3 comprising the amino acid sequence of SEQ ID NO: 353; and a VL comprising an HVR-L1 comprising the amino acid sequence of SEQ ID NO: 9, an HVR-L2 comprising the amino acid sequence of SEQ ID NO: 32, and an HVR-L3 comprising the amino acid sequence of SEQ ID NO: 45;
ooo. a VH comprising an HVR-H1 comprising the amino acid sequence of SEQ ID NO: 320, an HVR-H2 comprising the amino acid sequence of SEQ ID NO: 332, and an HVR-H3 comprising the amino acid sequence of SEQ ID NO: 371; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
ppp. a VH comprising an HVR-H1 comprising the amino acid sequence of SEQ ID NO: 320, an HVR-H2 comprising the amino acid sequence of SEQ ID NO: 332, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
qqq. a VH comprising an HVR-H1 comprising the amino acid sequence of SEQ ID NO: 103, an HVR-H2 comprising the amino acid sequence of SEQ ID NO: 333, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 45;
rrr. a VH comprising an HVR-H1 comprising the amino acid sequence of SEQ ID NO: 320, an HVR-H2 comprising the amino acid sequence of SEQ ID NO: 334, and an HVR-H3 comprising the amino acid sequence of SEQ ID NO: 154; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 48;
sss. a VH comprising an HVR-H1 comprising the amino acid sequence of SEQ ID NO: 321, an HVR-H2 comprising the amino acid sequence of SEQ ID NO: 125, and an HVR-H3 comprising the amino acid sequence of SEQ ID NO: 372; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
ttt. a VH comprising an HVR-H1 comprising the amino acid sequence of SEQ ID NO: 321, an HVR-H2 comprising the amino acid sequence of SEQ ID NO: 335, and an HVR-H3 comprising the amino acid sequence of SEQ ID NO: 373; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
uuu. a VH comprising an HVR-H1 comprising the amino acid sequence of SEQ ID NO: 322, an HVR-H2 comprising the amino acid sequence of SEQ ID NO: 125, and an HVR-H3 comprising the amino acid sequence of SEQ ID NO: 159; and a VL comprising an HVR-L1 comprising the amino acid sequence of SEQ ID NO: 14, an HVR-L2 comprising the amino acid sequence of SEQ ID NO: 32, and an HVR-L3 comprising the amino acid sequence of SEQ ID NO: 53;
vvv. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 354; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
www. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 355; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
xxx. a VH comprising an HVR-H1 comprising the amino acid sequence of SEQ ID NO: 346, an HVR-H2 comprising the amino acid sequence of SEQ ID NO: 336, and an HVR-H3 comprising the amino acid sequence of SEQ ID NO: 356; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
yyy. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 357; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
zzz. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 374; and a VL comprising an HVR-L1 comprising the amino acid sequence of SEQ ID NO: 33, an HVR-L2 comprising the amino acid sequence of SEQ ID NO: and an HVR-L3 comprising the amino acid sequence of SEQ ID NO: 59;
aaaa. a VH comprising an HVR-H1 comprising the amino acid sequence of SEQ ID NO: 347, an HVR-H2 comprising the amino acid sequence of SEQ ID NO: 338, and an HVR-H3 comprising the amino acid sequence of SEQ ID NO: 165; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
bbbb. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 375; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
cccc. a VH comprising an HVR-H1 comprising the amino acid sequence of SEQ ID NO: 103, an HVR-H2 comprising the amino acid sequence of SEQ ID NO: 339, and an HVR-H3 comprising the amino acid sequence of SEQ ID NO: 376; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
dddd. a VH comprising an HVR-H1 comprising the amino acid sequence of SEQ ID NO: 103, an HVR-H2 comprising the amino acid sequence of SEQ ID NO: 336, and an HVR-H3 comprising the amino acid sequence of SEQ ID NO: 377; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
eeee. a VH comprising an HVR-H1 comprising the amino acid sequence of SEQ ID NO: 348, an HVR-H2 comprising the amino acid sequence of SEQ ID NO: 337, and an HVR-H3 comprising the amino acid sequence of SEQ ID NO: 378; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
ffff. a VH comprising an HVR-H1 comprising the amino acid sequence of SEQ ID NO: 323, an HVR-H2 comprising the amino acid sequence of SEQ ID NO: 336, and an HVR-H3 comprising the amino acid sequence of SEQ ID NO: 379; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
gggg. a VH comprising an HVR-H1 comprising the amino acid sequence of SEQ ID NO: 349, an HVR-H2 comprising the amino acid sequence of SEQ ID NO: 340, and an HVR-H3 comprising the amino acid sequence of SEQ ID NO: 380; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
hhhh. a VH comprising an HVR-H1 comprising the amino acid sequence of SEQ ID NO: 103, an HVR-H2 comprising the amino acid sequence of SEQ ID NO: 337, and an HVR-H3 comprising the amino acid sequence of SEQ ID NO: 381; and a VL comprising an HVR-L1 comprising the amino acid sequence of SEQ ID NO: 11, an HVR-L2 comprising the amino acid sequence of SEQ ID NO: 33, and an HVR-L3 comprising the amino acid sequence of SEQ ID NO: 59;
iiii. a VH comprising an HVR-H1 comprising the amino acid sequence of SEQ ID NO: 324, an HVR-H2 comprising the amino acid sequence of SEQ ID NO: 341, and an HVR-H3 comprising the amino acid sequence of SEQ ID NO: 197; and a VL comprising comprising the amino acid sequence of SEQ ID NO: 32, and an HVR-L3 comprising the amino acid sequence of SEQ ID NO: 96;
jjjj. a VH comprising an HVR-H1 comprising the amino acid sequence of SEQ ID NO: 325, an HVR-H2 comprising the amino acid sequence of SEQ ID NO: 342, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
kkkk. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 382; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
llll. a VH comprising an HVR-H1 comprising the amino acid sequence of SEQ ID NO: 325, an HVR-H2 comprising the amino acid sequence of SEQ ID NO: 343, and an HVR-H3 comprising the amino acid sequence of SEQ ID NO: 383; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
mmmm. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 197; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
nnnn. a VH comprising an HVR-H1 comprising the amino acid sequence of SEQ ID NO: 326, an HVR-H2 comprising the amino acid sequence of SEQ ID NO: 341, and an HVR-H3 comprising the amino acid sequence of SEQ ID NO: 197; and a VL comprising comprising the amino acid sequence of SEQ ID NO: 32, and an HVR-L3 comprising the amino acid sequence of SEQ ID NO: 96;
oooo. a VH comprising an HVR-H1 comprising the amino acid sequence of SEQ ID NO: 327, an HVR-H2 comprising the amino acid sequence of SEQ ID NO: 341, and an HVR-H3 comprising the amino acid sequence of SEQ ID NO: 384; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
pppp. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 385; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
qqqq. a VH comprising an HVR-H1 comprising the amino acid sequence of SEQ ID NO: 325, an HVR-H2 comprising the amino acid sequence of SEQ ID NO: 341, and an HVR-H3 comprising the amino acid sequence of SEQ ID NO: 386; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
rrrr. a VH comprising an HVR-H1 comprising the amino acid sequence of SEQ ID NO: 325, an HVR-H2 comprising the amino acid sequence of SEQ ID NO: 341 and an HVR-H3 comprising the amino acid sequence of SEQ ID NO: 358; and a VL comprising an HVR-L1 comprising the amino acid sequence of SEQ ID NO: 12, an HVR-L2 comprising the amino acid sequence of SEQ ID NO: 32, and an HVR-L3 comprising the amino acid sequence of SEQ ID NO: 96;
ssss. a VH comprising an HVR-H1 comprising the amino acid sequence of SEQ ID NO: 327, an HVR-H2 comprising the amino acid sequence of SEQ ID NO: 344, and an HVR-H3 comprising the amino acid sequence of SEQ ID NO: 359; and a VL comprising comprising the amino acid sequence of SEQ ID NO: 32, and an HVR-L3 comprising the amino acid sequence of SEQ ID NO: 96; or
tttt. a VH comprising an HVR-H1 comprising the amino acid sequence of SEQ ID NO: 328, an HVR-H2 comprising the amino acid sequence of SEQ ID NO: 345, and an HVR-H3 comprising the amino acid sequence of SEQ ID NO: 360; and a VL comprising an HVR-L1 comprising the amino acid sequence of SEQ ID NO:12, an HVR-L2 comprising the amino acid sequence of SEQ ID NO: 32, and an HVR-L3 comprising the amino acid sequence of SEQ ID NO: 96.

13. The antibody of claim 12, wherein the antibody comprises:
a. the HVRs of claim 12.a. and further comprises a VH that is at least 90%, at least 95%, at least 97%, or at least 99% identical to the amino acid sequence of SEQ ID NO: 202 and a VL that is at least 90%, at least 95%, at least 97%, or at least 99% identical to the amino acid sequence of SEQ ID NO: 257;
b. the HVRs of claim 12.b. and further comprises a VH that is at least 90%, at least 95%, at least 97%, or at least 99% identical to the amino acid sequence of SEQ ID NO: 203 and a VL that is at least 90%, at least 95%, at least 97%, or at least 99% identical to the amino acid sequence of SEQ ID NO: 258;
c. the HVRs of claim 12.c. and further comprises a VH that is at least 90%, at least 95%, at least 97%, or at least 99% identical to the amino acid sequence of SEQ ID NO: 204 and a VL that is at least 90%, at least 95%, at least 97%, or at least 99% identical to the amino acid sequence of SEQ ID NO: 259;
d. the HVRs of claim 12.d. and further comprises a VH that is at least 90%, at least 95%, at least 97%, or at least 99% identical to the amino acid sequence of SEQ ID NO: 205 and a VL that is at least 90%, at least 95%, at least 97%, or at least 99% identical to the amino acid sequence of SEQ ID NO: 260;
e. the HVRs of claim 12.e. and further comprises a VH that is at least 90%, at least 95%, at least 97%, or at least 99% identical to the amino acid sequence of SEQ ID NO: 206 and a VL that is at least 90%, at least 95%, at least 97%, or at least 99% identical to the amino acid sequence of SEQ ID NO: 261;
f. the HVRs of claim 12.f. and further comprises a VH that is at least 90%, at least 95%, at least 97%, or at least 99% identical to the amino acid sequence of SEQ ID NO: 207 and a VL that is at least 90%, at least 95%, at least 97%, or at least 99% identical to the amino acid sequence of SEQ ID NO: 262;
g. the HVRs of claim 12.g. and further comprises a VH that is at least 90%, at least 95%, at least 97%, or at least 99% identical to the amino acid sequence of SEQ ID NO: 208 and a VL that is at least 90%, at least 95%, at least 97%, or at least 99% identical to the amino acid sequence of SEQ ID NO: 263;
h. the HVRs of claim 12.h. and further comprises a VH that is at least 90%, at least 95%, at least 97%, or at least 99% identical to the amino acid sequence of SEQ ID NO: 209 and a VL that is at least 90%, at least 95%, at least 97%, or at least 99% identical to the amino acid sequence of SEQ ID NO: 264;
1. the HVRs of claim 12.i. and further comprises a VH that is at least 90%, at least 95%, at least 97%, or at least 99% identical to the amino acid sequence of SEQ ID NO: 210 and a VL that is at least 90%, at least 95%, at least 97%, or at least 99% identical to the amino acid sequence of SEQ ID NO: 265;
j. the HVRs of claim 12.j. and further comprises a VH that is at least 90%, at least 95%, at least 97%, or at least 99% identical to the amino acid sequence of SEQ ID NO: 211 and a VL that is at least 90%, at least 95%, at least 97%, or at least 99% identical to the amino acid sequence of SEQ ID NO: 266;
k. the HVRs of claim 12.k. and further comprises a VH that is at least 90%, at least 95%, at least 97%, or at least 99% identical to the amino acid sequence of SEQ ID NO: 212 and a VL that is at least 90%, at least 95%, at least 97%, or at least 99% identical to the amino acid sequence of SEQ ID NO: 267;
l. the HVRs of claim 12.l. and further comprises a VH that is at least 90%, at least 95%, at least 97%, or at least 99% identical to the amino acid sequence of SEQ ID NO: 213 and a VL that is at least 90%, at least 95%, at least 97%, or at least 99% identical to the amino acid sequence of SEQ ID NO: 268;
m. the HVRs of claim 12.m. and further comprises a VH that is at least 90%, at least 95%, at least 97%, or at least 99% identical to the amino acid sequence of SEQ ID NO: 214 and a VL that is at least 90%, at least 95%, at least 97%, or at least 99% identical to the amino acid sequence of SEQ ID NO: 269;
n. the HVRs of claim 12.n. and further comprises a VH that is at least 90%, at least 95%, at least 97%, or at least 99% identical to the amino acid sequence of SEQ ID NO: 215 and a VL that is at least 90%, at least 95%, at least 97%, or at least 99% identical to the amino acid sequence of SEQ ID NO: 270;
o. the HVRs of claim 12.o. and further comprises a VH that is at least 90%, at least 95%, at least 97%, or at least 99% identical to the amino acid sequence of SEQ ID NO: 216 and a VL that is at least 90%, at least 95%, at least 97%, or at least 99% identical to the amino acid sequence of SEQ ID NO: 271;
p. the HVRs of claim 12.p. and further comprises a VH that is at least 90%, at least 95%, at least 97%, or at least 99% identical to the amino acid sequence of SEQ ID NO: 217 and a VL that is at least 90%, at least 95%, at least 97%, or at least 99% identical to the amino acid sequence of SEQ ID NO: 272;
q. the HVRs of claim 12.q. and further comprises a VH that is at least 90%, at least 95%, at least 97%, or at least 99% identical to the amino acid sequence of SEQ ID NO: 218 and a VL that is at least 90%, at least 95%, at least 97%, or at least 99% identical to the amino acid sequence of SEQ ID NO: 273;
r. the HVRs of claim 12.r. and further comprises a VH that is at least 90%, at least 95%, at least 97%, or at least 99% identical to the amino acid sequence of SEQ ID NO: 219 and a VL that is at least 90%, at least 95%, at least 97%, or at least 99% identical to the amino acid sequence of SEQ ID NO: 274;
s. the HVRs of claim 12.s. and further comprises a VH that is at least 90%, at least 95%, at least 97%, or at least 99% identical to the amino acid sequence of SEQ ID NO: 220 and a VL that is at least 90%, at least 95%, at least 97%, or at least 99% identical to the amino acid sequence of SEQ ID NO: 275;
t. the HVRs of claim 12.t. and further comprises a VH that is at least 90%, at least 95%, at least 97%, or at least 99% identical to the amino acid sequence of SEQ ID NO: 209 and a VL that is at least 90%, at least 95%, at least 97%, or at least 99% identical to the amino acid sequence of SEQ ID NO: 276;
u. the HVRs of claim 12.u. and further comprises a VH that is at least 90%, at least 95%, at least 97%, or at least 99% identical to the amino acid sequence of SEQ ID NO: 221 and a VL that is at least 90%, at least 95%, at least 97%, or at least 99% identical to the amino acid sequence of SEQ ID NO: 277;
v. the HVRs of claim 12.v. and further comprises a VH that is at least 90%, at least 95%, at least 97%, or at least 99% identical to the amino acid sequence of SEQ ID NO: 222 and a VL that is at least 90%, at least 95%, at least 97%, or at least 99% identical to the amino acid sequence of SEQ ID NO: 278;
w. the HVRs of claim 12.w. and further comprises a VH that is at least 90%, at least 95%, at least 97%, or at least 99% identical to the amino acid sequence of SEQ ID NO: 223 and a VL that is at least 90%, at least 95%, at least 97%, or at least 99% identical to the amino acid sequence of SEQ ID NO: 279;
x. the HVRs of claim 12.x. and further comprises a VH that is at least 90%, at least 95%, at least 97%, or at least 99% identical to the amino acid sequence of SEQ ID NO: 224 and a VL that is at least 90%, at least 95%, at least 97%, or at least 99% identical to the amino acid sequence of SEQ ID NO: 280;
y. the HVRs of claim 12.y. and further comprises a VH that is at least 90%, at least 95%, at least 97%, or at least 99% identical to the amino acid sequence of SEQ ID NO: 225 and a VL that is at least 90%, at least 95%, at least 97%, or at least 99% identical to the amino acid sequence of SEQ ID NO: 281;
z. the HVRs of claim 12.z. and further comprises a VH that is at least 90%, at least 95%, at least 97%, or at least 99% identical to the amino acid sequence of SEQ ID NO: 226 and a VL that is at least 90%, at least 95%, at least 97%, or at least 99% identical to the amino acid sequence of SEQ ID NO: 282;
aa. the HVRs of claim 12.aa. and further comprises a VH that is at least 90%, at least 95%, at least 97%, or at least 99% identical to the amino acid sequence of SEQ ID NO: 227 and a VL that is at least 90%, at least 95%, at least 97%, or at least 99% identical to the amino acid sequence of SEQ ID NO: 283;
bb. the HVRs of claim 12.bb. and further comprises a VH that is at least 90%, at least 95%, at least 97%, or at least 99% identical to the amino acid sequence of SEQ ID NO: 228 and a VL that is at least 90%, at least 95%, at least 97%, or at least 99% identical to the amino acid sequence of SEQ ID NO: 284;
cc. the HVRs of claim 12.cc. and further comprises a VH that is at least 90%, at least 95%, at least 97%, or at least 99% identical to the amino acid sequence of SEQ ID NO: 228 and a VL that is at least 90%, at least 95%, at least 97%, or at least 99% identical to the amino acid sequence of SEQ ID NO: 285;
dd. the HVRs of claim 12.dd. and further comprises a VH that is at least 90%, at least 95%, at least 97%, or at least 99% identical to the amino acid sequence of SEQ ID NO: 229 and a VL that is at least 90%, at least 95%, at least 97%, or at least 99% identical to the amino acid sequence of SEQ ID NO: 286;
ee. the HVRs of claim 12.ee. and further comprises a VH that is at least 90%, at least 95%, at least 97%, or at least 99% identical to the amino acid sequence of SEQ ID NO: 225 and a VL that is at least 90%, at least 95%, at least 97%, or at least 99% identical to the amino acid sequence of SEQ ID NO: 287;
ff. the HVRs of claim 12.ff. and further comprises a VH that is at least 90%, at least 95%, at least 97%, or at least 99% identical to the amino acid sequence of SEQ ID NO: 230 and a VL that is at least 90%, at least 95%, at least 97%, or at least 99% identical to the amino acid sequence of SEQ ID NO: 288;
gg. the HVRs of claim 12.gg. and further comprises a VH that is at least 90%, at least 95%, at least 97%, or at least 99% identical to the amino acid sequence of SEQ ID NO: 231 and a VL that is at least 90%, at least 95%, at least 97%, or at least 99% identical to the amino acid sequence of SEQ ID NO: 289;
hh. the HVRs of claim 12.hh. and further comprises a VH that is at least 90%, at least 95%, at least 97%, or at least 99% identical to the amino acid sequence of SEQ ID NO: 232 and a VL that is at least 90%, at least 95%, at least 97%, or at least 99% identical to the amino acid sequence of SEQ ID NO: 290;
ii. the HVRs of claim 12.ii. and further comprises a VH that is at least 90%, at least 95%, at least 97%, or at least 99% identical to the amino acid sequence of SEQ ID NO: 233 and a VL that is at least 90%, at least 95%, at least 97%, or at least 99% identical to the amino acid sequence of SEQ ID NO: 291;
jj. the HVRs of claim 12.jj. and further comprises a VH that is at least 90%, at least 95%, at least 97%, or at least 99% identical to the amino acid sequence of SEQ ID NO: 234 and a VL that is at least 90%, at least 95%, at least 97%, or at least 99% identical to the amino acid sequence of SEQ ID NO: 292;
kk. the HVRs of claim 12.kk. and further comprises a VH that is at least 90%, at least 95%, at least 97%, or at least 99% identical to the amino acid sequence of SEQ ID NO: 235 and a VL that is at least 90%, at least 95%, at least 97%, or at least 99% identical to the amino acid sequence of SEQ ID NO: 293;
ll. the HVRs of claim 12.ll. and further comprises a VH that is at least 90%, at least 95%, at least 97%, or at least 99% identical to the amino acid sequence of SEQ ID NO: 235 and a VL that is at least 90%, at least 95%, at least 97%, or at least 99% identical to the amino acid sequence of SEQ ID NO: 294;
mm. the HVRs of claim 12.mm. and further comprises a VH that is at least 90%, at least 95%, at least 97%, or at least 99% identical to the amino acid sequence of SEQ ID NO: 236 and a VL that is at least 90%, at least 95%, at least 97%, or at least 99% identical to the amino acid sequence of SEQ ID NO: 295;
nn. the HVRs of claim 12.nn. and further comprises a VH that is at least 90%, at least 95%, at least 97%, or at least 99% identical to the amino acid sequence of SEQ ID NO: 237 and a VL that is at least 90%, at least 95%, at least 97%, or at least 99% identical to the amino acid sequence of SEQ ID NO: 296;
oo. the HVRs of claim 12.oo. and further comprises a VH that is at least 90%, at least 95%, at least 97%, or at least 99% identical to the amino acid sequence of SEQ ID NO: 238 and a VL that is at least 90%, at least 95%, at least 97%, or at least 99% identical to the amino acid sequence of SEQ ID NO: 297;
pp. the HVRs of claim 12.pp. and further comprises a VH that is at least 90%, at least 95%, at least 97%, or at least 99% identical to the amino acid sequence of SEQ ID NO: 238 and a VL that is at least 90%, at least 95%, at least 97%, or at least 99% identical to the amino acid sequence of SEQ ID NO: 298;
qq. the HVRs of claim 12.qq. and further comprises a VH that is at least 90%, at least 95%, at least 97%, or at least 99% identical to the amino acid sequence of SEQ ID NO: 239 and a VL that is at least 90%, at least 95%, at least 97%, or at least 99% identical to the amino acid sequence of SEQ ID NO: 299;
rr. the HVRs of claim 12.rr. and further comprises a VH that is at least 90%, at least 95%, at least 97%, or at least 99% identical to the amino acid sequence of SEQ ID NO: 240 and a VL that is at least 90%, at least 95%, at least 97%, or at least 99% identical to the amino acid sequence of SEQ ID NO: 300;
ss. the HVRs of claim 12.ss. and further comprises a VH that is at least 90%, at least 95%, at least 97%, or at least 99% identical to the amino acid sequence of SEQ ID NO: 241 and a VL that is at least 90%, at least 95%, at least 97%, or at least 99% identical to the amino acid sequence of SEQ ID NO: 301;
tt. the HVRs of claim 12.tt. and further comprises a VH that is at least 90%, at least 95%, at least 97%, or at least 99% identical to the amino acid sequence of SEQ ID NO: 242 and a VL that is at least 90%, at least 95%, at least 97%, or at least 99% identical to the amino acid sequence of SEQ ID NO: 302;
uu. the HVRs of claim 12.uu. and further comprises a VH that is at least 90%, at least 95%, at least 97%, or at least 99% identical to the amino acid sequence of SEQ ID NO: 243 and a VL that is at least 90%, at least 95%, at least 97%, or at least 99% identical to the amino acid sequence of SEQ ID NO: 303;
vv. the HVRs of claim 12.vv. and further comprises a VH that is at least 90%, at least 95%, at least 97%, or at least 99% identical to the amino acid sequence of SEQ ID NO: 244 and a VL that is at least 90%, at least 95%, at least 97%, or at least 99% identical to the amino acid sequence of SEQ ID NO: 304;
ww. the HVRs of claim 12.ww. and further comprises a VH that is at least 90%, at least 95%, at least 97%, or at least 99% identical to the amino acid sequence of SEQ ID NO: 245 and a VL that is at least 90%, at least 95%, at least 97%, or at least 99% identical to the amino acid sequence of SEQ ID NO: 305;
xx. the HVRs of claim 12.xx. and further comprises a VH that is at least 90%, at least 95%, at least 97%, or at least 99% identical to the amino acid sequence of SEQ ID NO: 246 and a VL that is at least 90%, at least 95%, at least 97%, or at least 99% identical to the amino acid sequence of SEQ ID NO: 306;
yy. the HVRs of claim 12.yy. and further comprises a VH that is at least 90%, at least 95%, at least 97%, or at least 99% identical to the amino acid sequence of SEQ ID NO: 247 and a VL that is at least 90%, at least 95%, at least 97%, or at least 99% identical to the amino acid sequence of SEQ ID NO: 307;
zz. the HVRs of claim 12.zz. and further comprises a VH that is at least 90%, at least 95%, at least 97%, or at least 99% identical to the amino acid sequence of SEQ ID NO: 248 and a VL that is at least 90%, at least 95%, at least 97%, or at least 99% identical to the amino acid sequence of SEQ ID NO: 308;
aaa. the HVRs of claim 12.aaa. and further comprises a VH that is at least 90%, at least 95%, at least 97%, or at least 99% identical to the amino acid sequence of SEQ ID NO: 249 and a VL that is at least 90%, at least 95%, at least 97%, or at least 99% identical to the amino acid sequence of SEQ ID NO: 309;
bbb. the HVRs of claim 12.bbb. and further comprises a VH that is at least 90%, at least 95%, at least 97%, or at least 99% identical to the amino acid sequence of SEQ ID NO: 250 and a VL that is at least 90%, at least 95%, at least 97%, or at least 99% identical to the amino acid sequence of SEQ ID NO: 310;
ccc. the HVRs of claim 12.ccc. and further comprises a VH that is at least 90%, at least 95%, at least 97%, or at least 99% identical to the amino acid sequence of SEQ ID NO: 251 and a VL that is at least 90%, at least 95%, at least 97%, or at least 99% identical to the amino acid sequence of SEQ ID NO: 311;
ddd. the HVRs of claim 12.ddd. and further comprises a VH that is at least 90%, at least 95%, at least 97%, or at least 99% identical to the amino acid sequence of SEQ ID NO: 252 and a VL that is at least 90%, at least 95%, at least 97%, or at least 99% identical to the amino acid sequence of SEQ ID NO: 312;
eee. the HVRs of claim 12.eee. and further comprises a VH that is at least 90%, at least 95%, at least 97%, or at least 99% identical to the amino acid sequence of SEQ ID NO: 253 and a VL that is at least 90%, at least 95%, at least 97%, or at least 99% identical to the amino acid sequence of SEQ ID NO: 313;
fff. the HVRs of claim 12.fff. and further comprises a VH that is at least 90%, at least 95%, at least 97%, or at least 99% identical to the amino acid sequence of SEQ ID NO: 254 and a VL that is at least 90%, at least 95%, at least 97%, or at least 99% identical to the amino acid sequence of SEQ ID NO: 314;
ggg. the HVRs of claim 12.ggg. and further comprises a VH that is at least 90%, at least 95%, at least 97%, or at least 99% identical to the amino acid sequence of SEQ ID NO: 255 and a VL that is at least 90%, at least 95%, at least 97%, or at least 99% identical to the amino acid sequence of SEQ ID NO: 315;
hhh. the HVRs of claim 12.hhh. and further comprises a VH that is at least 90%, at least 95%, at least 97%, or at least 99% identical to the amino acid sequence of SEQ ID NO: 256 and a VL that is at least 90%, at least 95%, at least 97%, or at least 99% identical to the amino acid sequence of SEQ ID NO: 316;
iii. the HVRs of claim 12.iii. and further comprises a VH that is at least 90%, at least 95%, at least 97%, or at least 99% identical to the amino acid sequence of SEQ ID NO: 233 and a VL that is at least 90%, at least 95%, at least 97%, or at least 99% identical to the amino acid sequence of SEQ ID NO: 291;
jjj. the HVRs of claim 12.jjj. and further comprises a VH that is at least 90%, at least 95%, at least 97%, or at least 99% identical to the amino acid sequence of SEQ ID NO: 256 and a VL that is at least 90%, at least 95%, at least 97%, or at least 99% identical to the amino acid sequence of SEQ ID NO: 317;
kkk. the HVRs of claim 12.kkk. and further comprises a VH that is at least 90%, at least 95%, at least 97%, or at least 99% identical to the amino acid sequence of SEQ ID NO: 361 and a VL that is at least 90%, at least 95%, at least 97%, or at least 99% identical to the amino acid sequence of SEQ ID NO: 261;
lll. the HVRs of claim 12.lll. and further comprises a VH that is at least 90%, at least 95%, at least 97%, or at least 99% identical to the amino acid sequence of SEQ ID NO: 362 and a VL that is at least 90%, at least 95%, at least 97%, or at least 99% identical to the amino acid sequence of SEQ ID NO: 261;
mmm. the HVRs of claim 12.mmm. and further comprises a VH that is at least 90%, at least 95%, at least 97%, or at least 99% identical to the amino acid sequence of SEQ ID NO: 363 and a VL that is at least 90%, at least 95%, at least 97%, or at least 99% identical to the amino acid sequence of SEQ ID NO: 261;
nnn. the HVRs of claim 12.nnn. and further comprises a VH that is at least 90%, at least 95%, at least 97%, or at least 99% identical to the amino acid sequence of SEQ ID NO: 364 and a VL that is at least 90%, at least 95%, at least 97%, or at least 99% identical to the amino acid sequence of SEQ ID NO: 261;
ooo. the HVRs of claim 12.ooo. and further comprises a VH that is at least 90%, at least 95%, at least 97%, or at least 99% identical to the amino acid sequence of SEQ ID NO: 365 and a VL that is at least 90%, at least 95%, at least 97%, or at least 99% identical to the amino acid sequence of SEQ ID NO: 264;
ppp. the HVRs of claim 12.ppp. and further comprises a VH that is at least 90%, at least 95%, at least 97%, or at least 99% identical to the amino acid sequence of SEQ ID NO: 366 and a VL that is at least 90%, at least 95%, at least 97%, or at least 99% identical to the amino acid sequence of SEQ ID NO: 264;
qqq. the HVRs of claim 12.qqq. and further comprises a VH that is at least 90%, at least 95%, at least 97%, or at least 99% identical to the amino acid sequence of SEQ ID NO: 367 and a VL that is at least 90%, at least 95%, at least 97%, or at least 99% identical to the amino acid sequence of SEQ ID NO: 264;
rrr. the HVRs of claim 12.rrr. and further comprises a VH that is at least 90%, at least 95%, at least 97%, or at least 99% identical to the amino acid sequence of SEQ ID NO: 368 and a VL that is at least 90%, at least 95%, at least 97%, or at least 99% identical to the amino acid sequence of SEQ ID NO: 264;
sss. the HVRs of claim 12.sss. and further comprises a VH that is at least 90%, at least 95%, at least 97%, or at least 99% identical to the amino acid sequence of SEQ ID NO: 369 and a VL that is at least 90%, at least 95%, at least 97%, or at least 99% identical to the amino acid sequence of SEQ ID NO: 269;
ttt. the HVRs of claim 12.ttt. and further comprises a VH that is at least 90%, at least 95%, at least 97%, or at least 99% identical to the amino acid sequence of SEQ ID NO: 370 and a VL that is at least 90%, at least 95%, at least 97%, or at least 99% identical to the amino acid sequence of SEQ ID NO: 269;
uuu. the HVRs of claim 12.uuu. and further comprises a VH that is at least 90%, at least 95%, at least 97%, or at least 99% identical to the amino acid sequence of SEQ ID NO: 387 and a VL that is at least 90%, at least 95%, at least 97%, or at least 99% identical to the amino acid sequence of SEQ ID NO: 269;
vvv. the HVRs of claim 12.vvv. and further comprises a VH that is at least 90%, at least 95%, at least 97%, or at least 99% identical to the amino acid sequence of SEQ ID NO: 388 and a VL that is at least 90%, at least 95%, at least 97%, or at least 99% identical to the amino acid sequence of SEQ ID NO: 275;
www. the HVRs of claim 12.www. and further comprises a VH that is at least 90%, at least 95%, at least 97%, or at least 99% identical to the amino acid sequence of SEQ ID NO: 389 and a VL that is at least 90%, at least 95%, at least 97%, or at least 99% identical to the amino acid sequence of SEQ ID NO: 275;
xxx. the HVRs of claim 12.xxx. and further comprises a VH that is at least 90%, at least 95%, at least 97%, or at least 99% identical to the amino acid sequence of SEQ ID NO: 390 and a VL that is at least 90%, at least 95%, at least 97%, or at least 99% identical to the amino acid sequence of SEQ ID NO: 275;
yyy. the HVRs of claim 12.yyy. and further comprises a VH that is at least 90%, at least 95%, at least 97%, or at least 99% identical to the amino acid sequence of SEQ ID NO: 391 and a VL that is at least 90%, at least 95%, at least 97%, or at least 99% identical to the amino acid sequence of SEQ ID NO: 275;
zzz. the HVRs of claim 12.zzz. and further comprises a VH that is at least 90%, at least 95%, at least 97%, or at least 99% identical to the amino acid sequence of SEQ ID NO: 392 and a VL that is at least 90%, at least 95%, at least 97%, or at least 99% identical to the amino acid sequence of SEQ ID NO: 275;
aaaa. the HVRs of claim 12.aaaa. and further comprises a VH that is at least 90%, at least 95%, at least 97%, or at least 99% identical to the amino acid sequence of SEQ ID NO: 393 and a VL that is at least 90%, at least 95%, at least 97%, or at least 99% identical to the amino acid sequence of SEQ ID NO: 275;
bbbb. the HVRs of claim 12.bbbb. and further comprises a VH that is at least 90%, at least 95%, at least 97%, or at least 99% identical to the amino acid sequence of SEQ ID NO: 394 and a VL that is at least 90%, at least 95%, at least 97%, or at least 99% identical to the amino acid sequence of SEQ ID NO: 275;
cccc. the HVRs of claim 12.cccc. and further comprises a VH that is at least 90%, at least 95%, at least 97%, or at least 99% identical to the amino acid sequence of SEQ ID NO: 395 and a VL that is at least 90%, at least 95%, at least 97%, or at least 99% identical to the amino acid sequence of SEQ ID NO: 275;
dddd. the HVRs of claim 12.dddd. and further comprises a VH that is at least 90%, at least 95%, at least 97%, or at least 99% identical to the amino acid sequence of SEQ ID NO: 396 and a VL that is at least 90%, at least 95%, at least 97%, or at least 99% identical to the amino acid sequence of SEQ ID NO: 275;
eeee. the HVRs of claim 12.eeee. and further comprises a VH that is at least 90%, at least 95%, at least 97%, or at least 99% identical to the amino acid sequence of SEQ ID NO: 398 and a VL that is at least 90%, at least 95%, at least 97%, or at least 99% identical to the amino acid sequence of SEQ ID NO: 264;
ffff. the HVRs of claim 12.ffff. and further comprises a VH that is at least 90%, at least 95%, at least 97%, or at least 99% identical to the amino acid sequence of SEQ ID NO: 399 and a VL that is at least 90%, at least 95%, at least 97%, or at least 99% identical to the amino acid sequence of SEQ ID NO: 275;
gggg. the HVRs of claim 12.gggg. and further comprises a VH that is at least 90%, at least 95%, at least 97%, or at least 99% identical to the amino acid sequence of SEQ ID NO: 400 and a VL that is at least 90%, at least 95%, at least 97%, or at least 99% identical to the amino acid sequence of SEQ ID NO: 275;
hhhh. the HVRs of claim 9.hhhh. and further comprises a VH that is at least 90%, at least 95%, at least 97%, or at least 99% identical to the amino acid sequence of SEQ ID NO: 401 and a VL that is at least 90%, at least 95%, at least 97%, or at least 99% identical to the amino acid sequence of SEQ ID NO: 275;
iiii. the HVRs of claim 12.iiii. and further comprises a VH that is at least 90%, at least 95%, at least 97%, or at least 99% identical to the amino acid sequence of SEQ ID NO: 402 and a VL that is at least 90%, at least 95%, at least 97%, or at least 99% identical to the amino acid sequence of SEQ ID NO: 416;
jjjj. the HVRs of claim 12.jjjj. and further comprises a VH that is at least 90%, at least 95%, at least 97%, or at least 99% identical to the amino acid sequence of SEQ ID NO: 403 and a VL that is at least 90%, at least 95%, at least 97%, or at least 99% identical to the amino acid sequence of SEQ ID NO: 312;
kkkk. the HVRs of claim 12.kkkk. and further comprises a VH that is at least 90%, at least 95%, at least 97%, or at least 99% identical to the amino acid sequence of SEQ ID NO: 404 and a VL that is at least 90%, at least 95%, at least 97%, or at least 99% identical to the amino acid sequence of SEQ ID NO: 312;
llll. the HVRs of claim 12.llll. and further comprises a VH that is at least 90%, at least 95%, at least 97%, or at least 99% identical to the amino acid sequence of SEQ ID NO: 405 and a VL that is at least 90%, at least 95%, at least 97%, or at least 99% identical to the amino acid sequence of SEQ ID NO: 312;
mmmm. the HVRs of claim 12.mmmm. and further comprises a VH that is at least 90%, at least 95%, at least 97%, or at least 99% identical to the amino acid sequence of SEQ ID NO: 406 and a VL that is at least 90%, at least 95%, at least 97%, or at least 99% identical to the amino acid sequence of SEQ ID NO: 312;
nnnn. the HVRs of claim 12.nnnn. and further comprises a VH that is at least 90%, at least 95%, at least 97%, or at least 99% identical to the amino acid sequence of SEQ ID NO: 407 and a VL that is at least 90%, at least 95%, at least 97%, or at least 99% identical to the amino acid sequence of SEQ ID NO: 312;
oooo. the HVRs of claim 12.oooo. and further comprises a VH that is at least 90%, at least 95%, at least 97%, or at least 99% identical to the amino acid sequence of SEQ ID NO: 408 and a VL that is at least 90%, at least 95%, at least 97%, or at least 99% identical to the amino acid sequence of SEQ ID NO: 312;
pppp. the HVRs of claim 12.pppp. and further comprises a VH that is at least 90%, at least 95%, at least 97%, or at least 99% identical to the amino acid sequence of SEQ ID NO: 409 and a VL that is at least 90%, at least 95%, at least 97%, or at least 99% identical to the amino acid sequence of SEQ ID NO: 312;
qqqq. the HVRs of claim 12.qqqq. and further comprises a VH that is at least 90%, at least 95%, at least 97%, or at least 99% identical to the amino acid sequence of SEQ ID NO: 410 and a VL that is at least 90%, at least 95%, at least 97%, or at least 99% identical to the amino acid sequence of SEQ ID NO: 312;
rrrr. the HVRs of claim 12.rrrr. and further comprises a VH that is at least 90%, at least 95%, at least 97%, or at least 99% identical to the amino acid sequence of SEQ ID NO: 411 and a VL that is at least 90%, at least 95%, at least 97%, or at least 99% identical to the amino acid sequence of SEQ ID NO: 312;
ssss. the HVRs of claim 12.ssss. and further comprises a VH that is at least 90%, at least 95%, at least 97%, or at least 99% identical to the amino acid sequence of SEQ ID NO: 397 and a VL that is at least 90%, at least 95%, at least 97%, or at least 99% identical to the amino acid sequence of SEQ ID NO: 312; or
tttt. the HVRs of claim 12.tttt. and further comprises a VH that is at least 90%, at least 95%, at least 97%, or at least 99% identical to the amino acid sequence of SEQ ID NO: 412 and a VL that is at least 90%, at least 95%, at least 97%, or at least 99% identical to the amino acid sequence of SEQ ID NO: 312.

14. (canceled)

15. (canceled)

16. (canceled)

17. The antibody of claim 12, wherein the antibody comprises:
a. a VH comprising the amino acid sequence of SEQ ID NO: 202 and a VL comprising the amino acid sequence of SEQ ID NO: 257;
b. a VH comprising the amino acid sequence of SEQ ID NO: 203 and a VL comprising the amino acid sequence of SEQ ID NO: 258;
c. a VH comprising the amino acid sequence of SEQ ID NO: 204 and a VL comprising the amino acid sequence of SEQ ID NO: 259;
d. a VH comprising the amino acid sequence of SEQ ID NO: 205 and a VL comprising the amino acid sequence of SEQ ID NO: 260;
e. a VH comprising the amino acid sequence of SEQ ID NO: 206 and a VL comprising the amino acid sequence of SEQ ID NO: 261;
f. a VH comprising the amino acid sequence of SEQ ID NO: 207 and a VL comprising the amino acid sequence of SEQ ID NO: 262;
g. a VH comprising the amino acid sequence of SEQ ID NO: 208 and a VL comprising the amino acid sequence of SEQ ID NO: 263;
h. a VH comprising the amino acid sequence of SEQ ID NO: 209 and a VL comprising the amino acid sequence of SEQ ID NO: 264;
i. a VH comprising the amino acid sequence of SEQ ID NO: 210 and a VL comprising the amino acid sequence of SEQ ID NO: 265;
j. a VH comprising the amino acid sequence of SEQ ID NO: 211 and a VL comprising the amino acid sequence of SEQ ID NO: 266;
k. a VH comprising the amino acid sequence of SEQ ID NO: 212 and a VL comprising the amino acid sequence of SEQ ID NO: 267;
l. a VH comprising the amino acid sequence of SEQ ID NO: 213 and a VL comprising the amino acid sequence of SEQ ID NO: 268;
m. a VH comprising the amino acid sequence of SEQ ID NO: 214 and a VL comprising the amino acid sequence of SEQ ID NO: 269;
n. a VH comprising the amino acid sequence of SEQ ID NO: 215 and a VL comprising the amino acid sequence of SEQ ID NO: 270;
o. a VH comprising the amino acid sequence of SEQ ID NO: 216 and a VL comprising the amino acid sequence of SEQ ID NO: 271;
p. a VH comprising the amino acid sequence of SEQ ID NO: 217 and a VL comprising the amino acid sequence of SEQ ID NO: 272;
q. a VH comprising the amino acid sequence of SEQ ID NO: 218 and a VL comprising the amino acid sequence of SEQ ID NO: 273;
r. a VH comprising the amino acid sequence of SEQ ID NO: 219 and a VL comprising the amino acid sequence of SEQ ID NO: 274;
s. a VH comprising the amino acid sequence of SEQ ID NO: 220 and a VL comprising the amino acid sequence of SEQ ID NO: 275;
t. a VH comprising the amino acid sequence of SEQ ID NO: 209 and a VL comprising the amino acid sequence of SEQ ID NO: 276;
u. a VH comprising the amino acid sequence of SEQ ID NO: 221 and a VL comprising the amino acid sequence of SEQ ID NO: 277;
v. a VH comprising the amino acid sequence of SEQ ID NO: 222 and a VL comprising the amino acid sequence of SEQ ID NO: 278;
w. a VH comprising the amino acid sequence of SEQ ID NO: 223 and a VL comprising the amino acid sequence of SEQ ID NO: 279;
x. a VH comprising the amino acid sequence of SEQ ID NO: 224 and a VL comprising the amino acid sequence of SEQ ID NO: 280;
y. a VH comprising the amino acid sequence of SEQ ID NO: 225 and a VL comprising the amino acid sequence of SEQ ID NO: 281;
z. a VH comprising the amino acid sequence of SEQ ID NO: 226 and a VL comprising the amino acid sequence of SEQ ID NO: 282;
aa. a VH comprising the amino acid sequence of SEQ ID NO: 227 and a VL comprising the amino acid sequence of SEQ ID NO: 283;
bb. a VH comprising the amino acid sequence of SEQ ID NO: 228 and a VL comprising the amino acid sequence of SEQ ID NO: 284;
cc. a VH comprising the amino acid sequence of SEQ ID NO: 228 and a VL comprising the amino acid sequence of SEQ ID NO: 285;
dd. a VH comprising the amino acid sequence of SEQ ID NO: 229 and a VL comprising the amino acid sequence of SEQ ID NO: 286;
ee. a VH comprising the amino acid sequence of SEQ ID NO: 225 and a VL comprising the amino acid sequence of SEQ ID NO: 287;
ff. a VH comprising the amino acid sequence of SEQ ID NO: 230 and a VL comprising the amino acid sequence of SEQ ID NO: 288;
gg. a VH comprising the amino acid sequence of SEQ ID NO: 231 and a VL comprising the amino acid sequence of SEQ ID NO: 289;
hh. a VH comprising the amino acid sequence of SEQ ID NO: 232 and a VL comprising the amino acid sequence of SEQ ID NO: 290;
ii. a VH comprising the amino acid sequence of SEQ ID NO: 233 and a VL comprising the amino acid sequence of SEQ ID NO: 291;
jj. a VH comprising the amino acid sequence of SEQ ID NO: 234 and a VL comprising the amino acid sequence of SEQ ID NO: 292;
kk. a VH comprising the amino acid sequence of SEQ ID NO: 235 and a VL comprising the amino acid sequence of SEQ ID NO: 293;
ll. a VH comprising the amino acid sequence of SEQ ID NO: 235 and a VL comprising the amino acid sequence of SEQ ID NO: 294;
mm. a VH comprising the amino acid sequence of SEQ ID NO: 236 and a VL comprising the amino acid sequence of SEQ ID NO: 295;
nn. a VH comprising the amino acid sequence of SEQ ID NO: 237 and a VL comprising the amino acid sequence of SEQ ID NO: 296;
oo. a VH comprising the amino acid sequence of SEQ ID NO: 238 and a VL comprising the amino acid sequence of SEQ ID NO: 297;
pp. a VH comprising the amino acid sequence of SEQ ID NO: 238 and a VL comprising the amino acid sequence of SEQ ID NO: 298;
qq. a VH comprising the amino acid sequence of SEQ ID NO: 239 and a VL comprising the amino acid sequence of SEQ ID NO: 299;
rr. a VH comprising the amino acid sequence of SEQ ID NO: 240 and a VL comprising the amino acid sequence of SEQ ID NO: 300;
ss. a VH comprising the amino acid sequence of SEQ ID NO: 241 and a VL comprising the amino acid sequence of SEQ ID NO: 301;
tt. a VH comprising the amino acid sequence of SEQ ID NO: 242 and a VL comprising the amino acid sequence of SEQ ID NO: 302;
uu. a VH comprising the amino acid sequence of SEQ ID NO: 243 and a VL comprising the amino acid sequence of SEQ ID NO: 303;
vv. a VH comprising the amino acid sequence of SEQ ID NO: 244 and a VL comprising the amino acid sequence of SEQ ID NO: 304;
ww. a VH comprising the amino acid sequence of SEQ ID NO: 245 and a VL comprising the amino acid sequence of SEQ ID NO: 305;
xx. a VH comprising the amino acid sequence of SEQ ID NO: 246 and a VL comprising the amino acid sequence of SEQ ID NO: 306;
yy. a VH comprising the amino acid sequence of SEQ ID NO: 247 and a VL comprising the amino acid sequence of SEQ ID NO: 307;
zz. a VH comprising the amino acid sequence of SEQ ID NO: 248 and a VL comprising the amino acid sequence of SEQ ID NO: 308;
aaa. a VH comprising the amino acid sequence of SEQ ID NO: 249 and a VL comprising the amino acid sequence of SEQ ID NO: 309;
bbb. a VH comprising the amino acid sequence of SEQ ID NO: 250 and a VL comprising the amino acid sequence of SEQ ID NO: 310;
ccc. a VH comprising the amino acid sequence of SEQ ID NO: 251 and a VL comprising the amino acid sequence of SEQ ID NO: 311;
ddd. a VH comprising the amino acid sequence of SEQ ID NO: 252 and a VL comprising the amino acid sequence of SEQ ID NO: 312;
eee. a VH comprising the amino acid sequence of SEQ ID NO: 253 and a VL comprising the amino acid sequence of SEQ ID NO: 313;
fff. a VH comprising the amino acid sequence of SEQ ID NO: 254 and a VL comprising the amino acid sequence of SEQ ID NO: 314;
ggg. a VH comprising the amino acid sequence of SEQ ID NO: 255 and a VL comprising the amino acid sequence of SEQ ID NO: 315;
hhh. a VH comprising the amino acid sequence of SEQ ID NO: 256 and a VL comprising the amino acid sequence of SEQ ID NO: 316;
iii. a VH comprising the amino acid sequence of SEQ ID NO: 233 and a VL comprising the amino acid sequence of SEQ ID NO: 291;
jjj. a VH comprising the amino acid sequence of SEQ ID NO: 256 and a VL comprising the amino acid sequence of SEQ ID NO: 317;
kkk. a VH comprising the amino acid sequence of SEQ ID NO: 361 and a VL comprising the amino acid sequence of SEQ ID NO: 261;
lll. a VH comprising the amino acid sequence of SEQ ID NO: 362 and a VL comprising the amino acid sequence of SEQ ID NO: 261;
mmm. a VH comprising the amino acid sequence of SEQ ID NO: 363 and a VL comprising the amino acid sequence of SEQ ID NO: 261;
nnn. a VH comprising the amino acid sequence of SEQ ID NO: 364 and a VL comprising the amino acid sequence of SEQ ID NO: 261;
ooo. a VH comprising the amino acid sequence of SEQ ID NO: 365 and a VL comprising the amino acid sequence of SEQ ID NO: 264;
ppp. a VH comprising the amino acid sequence of SEQ ID NO: 366 and a VL comprising the amino acid sequence of SEQ ID NO: 264;
qqq. a VH comprising the amino acid sequence of SEQ ID NO: 367 and a VL comprising the amino acid sequence of SEQ ID NO: 264;
rrr. a VH comprising the amino acid sequence of SEQ ID NO: 368 and a VL comprising the amino acid sequence of SEQ ID NO: 264;
sss. a VH comprising the amino acid sequence of SEQ ID NO: 369 and a VL comprising the amino acid sequence of SEQ ID NO: 269;
ttt. a VH comprising the amino acid sequence of SEQ ID NO: 370 and a VL comprising the amino acid sequence of SEQ ID NO: 269;
uuu. a VH comprising the amino acid sequence of SEQ ID NO: 387 and a VL comprising the amino acid sequence of SEQ ID NO: 269;
vvv. a VH comprising the amino acid sequence of SEQ ID NO: 388 and a VL comprising the amino acid sequence of SEQ ID NO: 275;
www. a VH comprising the amino acid sequence of SEQ ID NO: 389 and a VL comprising the amino acid sequence of SEQ ID NO: 275;
xxx. a VH comprising the amino acid sequence of SEQ ID NO: 390 and a VL comprising the amino acid sequence of SEQ ID NO: 275;
yyy. a VH comprising the amino acid sequence of SEQ ID NO: 391 and a VL comprising the amino acid sequence of SEQ ID NO: 275;
zzz. a VH comprising the amino acid sequence of SEQ ID NO: 392 and a VL comprising the amino acid sequence of SEQ ID NO: 275;
aaaa. a VH comprising the amino acid sequence of SEQ ID NO: 393 and a VL comprising the amino acid sequence of SEQ ID NO: 275;
bbbb. a VH comprising the amino acid sequence of SEQ ID NO: 394 and a VL comprising the amino acid sequence of SEQ ID NO: 275;
cccc. a VH comprising the amino acid sequence of SEQ ID NO: 395 and a VL comprising the amino acid sequence of SEQ ID NO: 275;
dddd. a VH comprising the amino acid sequence of SEQ ID NO: 396 and a VL comprising the amino acid sequence of SEQ ID NO: 275;
eeee. a VH comprising the amino acid sequence of SEQ ID NO: 398 and a VL comprising the amino acid sequence of SEQ ID NO: 264;
ffff. a VH comprising the amino acid sequence of SEQ ID NO: 399 and a VL comprising the amino acid sequence of SEQ ID NO: 275;
gggg. a VH comprising the amino acid sequence of SEQ ID NO: 400 and a VL comprising the amino acid sequence of SEQ ID NO: 275;
hhhh. a VH comprising the amino acid sequence of SEQ ID NO: 401 and a VL comprising the amino acid sequence of SEQ ID NO: 275;
iiii. a VH comprising the amino acid sequence of SEQ ID NO: 402 and a VL comprising the amino acid sequence of SEQ ID NO: 413;
jjjj. a VH comprising the amino acid sequence of SEQ ID NO: 403 and a VL comprising the amino acid sequence of SEQ ID NO: 312;
kkkk. a VH comprising the amino acid sequence of SEQ ID NO: 404 and a VL comprising the amino acid sequence of SEQ ID NO: 312;
llll. a VH comprising the amino acid sequence of SEQ ID NO: 405 and a VL comprising the amino acid sequence of SEQ ID NO: 312;
mmmm. a VH comprising the amino acid sequence of SEQ ID NO: 406 and a VL comprising the amino acid sequence of SEQ ID NO: 312;
nnnn. a VH comprising the amino acid sequence of SEQ ID NO: 407 and a VL comprising the amino acid sequence of SEQ ID NO: 312;
oooo. a VH comprising the amino acid sequence of SEQ ID NO: 408 and a VL comprising the amino acid sequence of SEQ ID NO: 312;
pppp. a VH comprising the amino acid sequence of SEQ ID NO: 409 and a VL comprising the amino acid sequence of SEQ ID NO: 312;
qqqq. a VH comprising the amino acid sequence of SEQ ID NO: 410 and a VL comprising the amino acid sequence of SEQ ID NO: 312;
rrrr. a VH comprising the amino acid sequence of SEQ ID NO: 411 and a VL comprising the amino acid sequence of SEQ ID NO: 312;
ssss. a VH comprising the amino acid sequence of SEQ ID NO: 397 and a VL comprising the amino acid sequence of SEQ ID NO: 312;
tttt. a VH comprising the amino acid sequence of SEQ ID NO: 412 and a VL comprising the amino acid sequence of SEQ ID NO: 312.

18. (canceled)

19. The antibody of claim 12, wherein the antibody has one or more of the following properties:
a. the antibody increases SIRPA activity, SIRPA signaling, CD47-induced SIRPA signaling, or any combination thereof, in macrophages, dendritic cells, and/or microglial cells;
b. the antibody decreases phagocytic activity by phagocytic cells, decreases dendritic cell cytokine release (e.g., release of TNFalpha), suppresses synapse elimination in microglia-neuron co-cultures, suppresses synapse elimination in mouse models, or any combination thereof;
c. the antibody has an affinity (KD) to human SIRPA of less than 1 μM, less than 100 nM, less than 10 nM, less than 1 nM, less than 0.1 nM, less than 0.01 nM, or less than 0.001 nM;
d. the antibody has an affinity (KD) to human SIRPAv1 (SEQ ID NO: 1) of 1 nM to 50 nM, 0.3 nM to 2 nM, or 2 nM to 24 nM;
e. the antibody has higher affinity to human SIRPA than to mouse SIRPA;
f the antibody does not bind to mouse SIRPA;
g. the antibody has higher affinity to human SIRPA than to human SIRPB;
h. the antibody does not bind to human SIRPB;
i. the antibody binds to one or more amino acids in human SIRPA v1 (SEQ ID NO: 1) chosen from D40, R54, and W68;
j. the antibody has reduced binding affinity to a human SIRPAv1 (SEQ ID NO: 1) in comprising one or more of the following substitutions: D40A, R54A, W68A, compared to wild-type SIRPAv1 consisting of SEQ ID NO: 1.

20. (canceled)

21. The antibody of claim 12, wherein the antibody is a humanized antibody.

22. The antibody of claim 12, wherein the antibody is an antigen binding fragment, such as an Fab, Fab′, Fab′-SH, F(ab′)₂, Fv, or scFv fragment.

23. The antibody of claim 12, wherein the antibody is a bispecific or multispecific antibody.

24. The antibody of claim 12, wherein the antibody is of the IgG class, the IgM class, or the IgA class.

25. The antibody of claim 24, wherein the antibody is of the IgG class and is of a human IgG1, IgG2, IgG3, or IgG4 isotype or of a mouse IgG1 or IgG2 isotype.

26. The antibody of claim 12, wherein the antibody binds to an inhibitory Fc receptor.

27. The antibody of claim 26, wherein the inhibitory Fc receptor is inhibitory Fc-gamma receptor IIB (FcgRIIB).

28. The antibody of claim 27, wherein the antibody decreases cellular levels of FcgRIIB.

29. The antibody of claim 12, wherein the anti-SIRPA antibody has a human or mouse IgG1 isotype and comprises one or more amino acid substitutions in the Fc region at an amino acid residue selected from the group consisting of: N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, N325S, L328F, T394D, and any combination thereof, wherein the numbering of the residues is according to EU numbering, or comprises an amino acid deletion in the Fc region at a position corresponding to glycine 236.

30. The antibody of claim 12, wherein the antibody comprises one or more amino acid substitutions in the Fc region at a residue position selected from the group consisting of: C127S, L234A, L234F, L235A, L235E, S267E, K322A, N325S, L328F, A330S, P331S, E345R, E430G, S440Y, and any combination thereof, wherein the numbering of the amino acid residues is according to EU or Kabat numbering.

31. A pharmaceutical composition comprising the anti-SIRPA antibody of claim 12 and a pharmaceutically acceptable carrier.

32. An isolated nucleic acid comprising a nucleic acid sequence encoding the anti-SIRPA antibody of claim 12.

33. An isolated vector comprising the nucleic acid of claim 32.

34. An isolated host cell comprising the nucleic acid of claim 32.

35. A method of producing an antibody that binds to human SIRPA, comprising culturing the cell of claim 34 so that the antibody is produced.

36. The method of claim 35, further comprising recovering the antibody produced by the cell.

37. A method of treating a disease or disorder associated with inflammation, transplant rejection, autoimmunity, or cognitive impairment, the method comprising administering to an individual in need thereof a therapeutically effective amount of an anti-SIRPA antibody of claim 12, thereby treating the disease or disorder.

38. The method of claim 37, wherein the disease or disorder is chosen from inflammation, intestinal inflammation, intestinal inflammation associated with colitis, rheumatoid arthritis, organ/graft transplant rejection, multiple sclerosis, synaptic pruning in neurons, synaptic loss in neurons, synaptic pruning by microglia, and cognitive impairment.

39. A method of detecting the presence of SIRPA in a sample or an individual, the method comprising an anti-SIRPA antibody of claim 12.

40. The method of claim 39, further comprising quantification of antigen-bound anti-SIRPA antibody.


SEQUENCE LISTING (413 sequences)

SEQ ID NO: 1 - protein, 504 aa, Homo sapiens
  REGION 1..504: note = misc_feature - Human SIRPA v1
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 503 aa, Homo sapiens
  REGION 1..503: note = misc_feature - Human SIRPA v2
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 3 - protein, 509 aa, Mus musculus
  REGION 1..509: note = misc_feature - Mouse SIRPA
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 4 - protein, 398 aa, Homo sapiens
  REGION 1..398: note = misc_feature - Human SIRPB1 isoform 1
MPVPASWPHLPSPFLLMTLLLGRLTGVAGEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAALAPTAPLLVALLLGPKLLLVVGVSAIYICWKQKA

SEQ ID NO: 5 - protein, 398 aa, Homo sapiens
  REGION 1..398: note = misc_feature - Human SIRPB1 isoform 3
MPVPASWPHLPSPFLLMTLLLGRLTGVAGEEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPSAPVVSGPAARATPQHTVSFTCESHGFSPRDITLKWFKNGNELSDFQTNVDPAGDSVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAPGPALASAAPLLIAFLLGPKVLLVVGVSVIYVYWKQKA

SEQ ID NO: 6 - protein, 397 aa, Mus musculus
  REGION 1..397: note = misc_feature - Mouse SIRPB1
MLLLDAWTHIPHCVLLLILLLGLKGAAVRELKVIQPVKSFFVGAGGSATLNCTVTYLLPVGPIKWYRGVGQSRLLIYPFTGEYFPRITSVSDVKKRSNLDFSIRISNVTPADSGTYYCVKFQRGSSEPDIEIQSGGGTELSVFAKPSSPMVSGPAARAVPQQTVTFTCRSHGFFPQNLTLKWFKNGNEISHLETSVEPEETSVSYRVSSTVQVVLEPRDVRSQIICEVDHVTLDRAPLRGIAHISEIIQVPPTLEISQQPTMVWNVINVTCQIQKFYPRRFQVTWLENGNISRREVPFTHIVNKDGTYNWISWLLVNISALEENMVVTCQVEHDGQAEVIETHTVVVTEHQRVKGTSTMSELKTAGIAKIPVAVLLGSKILLLIAATVIYMRKKQNA

SEQ ID NO: 7 - protein, 11 aa, synthetic construct
  REGION 1..11: note = Synthetic: HVR-L1
RASQSVSSYLA

SEQ ID NO: 8 - protein, 17 aa, synthetic construct
  REGION 1..17: note = Synthetic: HVR-L1
KSSQSVLFSSNNKNYLA

SEQ ID NO: 9 - protein, 11 aa, synthetic construct
  REGION 1..11: note = Synthetic: HVR-L1
RASQDISSWLA

SEQ ID NO: 10 - protein, 17 aa, synthetic construct
  REGION 1..17: note = Synthetic: HVR-L1
KSSQSVLYSSNNKNYLA

SEQ ID NO: 11 - protein, 11 aa, synthetic construct
  REGION 1..11: note = Synthetic: HVR-L1
QASQDISNYLN

SEQ ID NO: 12 - protein, 11 aa, synthetic construct
  REGION 1..11: note = Synthetic: HVR-L1
RASQGISSWLA

SEQ ID NO: 13 - protein, 11 aa, synthetic construct
  REGION 1..11: note = Synthetic: HVR-L1
RASQSISSWLA

SEQ ID NO: 14 - protein, 11 aa, synthetic construct
  REGION 1..11: note = Synthetic: HVR-L1
RASQSISSYLN

SEQ ID NO: 15 - protein, 11 aa, synthetic construct
  REGION 1..11: note = Synthetic: HVR-L1
QASQDISNFLN

SEQ ID NO: 16 - protein, 11 aa, synthetic construct
  REGION 1..11: note = Synthetic: HVR-L1
RASQSVSSSLA

SEQ ID NO: 17 - protein, 16 aa, synthetic construct
  REGION 1..16: note = Synthetic: HVR-L1
RSSQSLLHSNGYNYLD

SEQ ID NO: 18 - protein, 11 aa, synthetic construct
  REGION 1..11: note = Synthetic: HVR-L1
RASQSINSYLN

SEQ ID NO: 19 - protein, 12 aa, synthetic construct
  REGION 1..12: note = Synthetic: HVR-L1
RASQSVRSSYLA

SEQ ID NO: 20 - protein, 12 aa, synthetic construct
  REGION 1..12: note = Synthetic: HVR-L1
RASQSVSSSYLA

SEQ ID NO: 21 - protein, 11 aa, synthetic construct
  REGION 1..11: note = Synthetic: HVR-L1
RASQSVGSNLA

SEQ ID NO: 22 - protein, 11 aa, synthetic construct
  REGION 1..11: note = Synthetic: HVR-L1
QASQDISSYLA

SEQ ID NO: 23 - protein, 12 aa, synthetic construct
  REGION 1..12: note = Synthetic: HVR-L1
RASQSVSSSFLA

SEQ ID NO: 24 - protein, 11 aa, synthetic construct
  REGION 1..11: note = Synthetic: HVR-L1
QASQDISNSLN

SEQ ID NO: 25 - protein, 11 aa, synthetic construct
  REGION 1..11: note = Synthetic: HVR-L1
RASQSVSSNLA

SEQ ID NO: 26 - protein, 11 aa, synthetic construct
  REGION 1..11: note = Synthetic: HVR-L1
RASQGIDSWLA

SEQ ID NO: 27 - protein, 11 aa, synthetic construct
  REGION 1..11: note = Synthetic: HVR-L1
RASQAISSWLA

SEQ ID NO: 28 - protein, 11 aa, synthetic construct
  REGION 1..11: note = Synthetic: HVR-L1
QASQDIANYLN

SEQ ID NO: 29 - protein, 7 aa, synthetic construct
  REGION 1..7: note = Synthetic: HVR-L2
DSSNRAT

SEQ ID NO: 30 - protein, 7 aa, synthetic construct
  REGION 1..7: note = Synthetic: HVR-L2
WASTRES

SEQ ID NO: 31 - protein, 7 aa, synthetic construct
  REGION 1..7: note = Synthetic: HVR-L2
DASNRAT

SEQ ID NO: 32 - protein, 7 aa, synthetic construct
  REGION 1..7: note = Synthetic: HVR-L2
AASSLQS

SEQ ID NO: 33 - protein, 7 aa, synthetic construct
  REGION 1..7: note = Synthetic: HVR-L2
DASNLET

SEQ ID NO: 34 - protein, 7 aa, synthetic construct
  REGION 1..7: note = Synthetic: HVR-L2
EASSLES

SEQ ID NO: 35 - protein, 7 aa, synthetic construct
  REGION 1..7: note = Synthetic: HVR-L2
DASSLES

SEQ ID NO: 36 - protein, 7 aa, synthetic construct
  REGION 1..7: note = Synthetic: HVR-L2
GASTRAT

SEQ ID NO: 37 - protein, 7 aa, synthetic construct
  REGION 1..7: note = Synthetic: HVR-L2
LGSNRAS

SEQ ID NO: 38 - protein, 7 aa, synthetic construct
  REGION 1..7: note = Synthetic: HVR-L2
GASSRAT

SEQ ID NO: 39 - protein, 7 aa, synthetic construct
  REGION 1..7: note = Synthetic: HVR-L2
KASSLES

SEQ ID NO: 40 - protein, 7 aa, synthetic construct
  REGION 1..7: note = Synthetic: HVR-L2
GASNRAT

SEQ ID NO: 41 - protein, 9 aa, synthetic construct
  REGION 1..9: note = Synthetic: HVR-L3
QQDSDYPFT

SEQ ID NO: 42 - protein, 9 aa, synthetic construct
  REGION 1..9: note = Synthetic: HVR-L3
QQYFLYPPT

SEQ ID NO: 43 - protein, 9 aa, synthetic construct
  REGION 1..9: note = Synthetic: HVR-L3
QQHDFWPPT

SEQ ID NO: 44 - protein, 9 aa, synthetic construct
  REGION 1..9: note = Synthetic: HVR-L3
QQDSNFPFT

SEQ ID NO: 45 - protein, 9 aa, synthetic construct
  REGION 1..9: note = Synthetic: HVR-L3
QQGYIFPRT

SEQ ID NO: 46 - protein, 9 aa, synthetic construct
  REGION 1..9: note = Synthetic: HVR-L3
QQDANYPYT

SEQ ID NO: 47 - protein, 9 aa, synthetic construct
  REGION 1..9: note = Synthetic: HVR-L3
QQYYHYPPT

SEQ ID NO: 48 - protein, 9 aa, synthetic construct
  REGION 1..9: note = Synthetic: HVR-L3
QQLSNYPIT

SEQ ID NO: 49 - protein, 9 aa, synthetic construct
  REGION 1..9: note = Synthetic: HVR-L3
QQAYLYPIT

SEQ ID NO: 50 - protein, 9 aa, synthetic construct
  REGION 1..9: note = Synthetic: HVR-L3
QQDNSLPYT

SEQ ID NO: 51 - protein, 9 aa, synthetic construct
  REGION 1..9: note = Synthetic: HVR-L3
QQYNSYSPT

SEQ ID NO: 52 - protein, 9 aa, synthetic construct
  REGION 1..9: note = Synthetic: HVR-L3
QQRTNFPIT

SEQ ID NO: 53 - protein, 9 aa, synthetic construct
  REGION 1..9: note = Synthetic: HVR-L3
QQGYIYPPT

SEQ ID NO: 54 - protein, 9 aa, synthetic construct
  REGION 1..9: note = Synthetic: HVR-L3
QQFINFPLT

SEQ ID NO: 55 - protein, 9 aa, synthetic construct
  REGION 1..9: note = Synthetic: HVR-L3
QQPYNFPLT

SEQ ID NO: 56 - protein, 8 aa, synthetic construct
  REGION 1..8: note = Synthetic: HVR-L3
QQFSDYPT

SEQ ID NO: 57 - protein, 9 aa, synthetic construct
  REGION 1..9: note = Synthetic: HVR-L3
QQDYIWPLT

SEQ ID NO: 58 - protein, 9 aa, synthetic construct
  REGION 1..9: note = Synthetic: HVR-L3
QQALSFPFT

SEQ ID NO: 59 - protein, 9 aa, synthetic construct
  REGION 1..9: note = Synthetic: HVR-L3
QQFTYLPPT

SEQ ID NO: 60 - protein, 9 aa, synthetic construct
  REGION 1..9: note = Synthetic: HVR-L3
QQVDVLPYT

SEQ ID NO: 61 - protein, 9 aa, synthetic construct
  REGION 1..9: note = Synthetic: HVR-L3
QQDYATPFT

SEQ ID NO: 62 - protein, 9 aa, synthetic construct
  REGION 1..9: note = Synthetic: HVR-L3
QQYDTLPPT

SEQ ID NO: 63 - protein, 9 aa, synthetic construct
  REGION 1..9: note = Synthetic: HVR-L3
MQAQGTPYT

SEQ ID NO: 64 - protein, 9 aa, synthetic construct
  REGION 1..9: note = Synthetic: HVR-L3
QQRLDWPYT

SEQ ID NO: 65 - protein, 8 aa, synthetic construct
  REGION 1..8: note = Synthetic: HVR-L3
QQYSYLPT

SEQ ID NO: 66 - protein, 9 aa, synthetic construct
  REGION 1..9: note = Synthetic: HVR-L3
QQSPSDPWT

SEQ ID NO: 67 - protein, 9 aa, synthetic construct
  REGION 1..9: note = Synthetic: HVR-L3
QQSVHTPYT

SEQ ID NO: 68 - protein, 9 aa, synthetic construct
  REGION 1..9: note = Synthetic: HVR-L3
QQAGVSPYT

SEQ ID NO: 69 - protein, 9 aa, synthetic construct
  REGION 1..9: note = Synthetic: HVR-L3
QQSSHTPYT

SEQ ID NO: 70 - protein, 9 aa, synthetic construct
  REGION 1..9: note = Synthetic: HVR-L3
QQLYIYPPT

SEQ ID NO: 71 - protein, 9 aa, synthetic construct
  REGION 1..9: note = Synthetic: HVR-L3
QQFFSYPPT

SEQ ID NO: 72 - protein, 9 aa, synthetic construct
  REGION 1..9: note = Synthetic: HVR-L3
QQLNVFPWT

SEQ ID NO: 73 - protein, 9 aa, synthetic construct
  REGION 1..9: note = Synthetic: HVR-L3
LQAYSFPFT

SEQ ID NO: 74 - protein, 9 aa, synthetic construct
  REGION 1..9: note = Synthetic: HVR-L3
QQYELLPPT

SEQ ID NO: 75 - protein, 9 aa, synthetic construct
  REGION 1..9: note = Synthetic: HVR-L3
QQYGSFPLT

SEQ ID NO: 76 - protein, 9 aa, synthetic construct
  REGION 1..9: note = Synthetic: HVR-L3
QQRTHYPWT

SEQ ID NO: 77 - protein, 9 aa, synthetic construct
  REGION 1..9: note = Synthetic: HVR-L3
QQYDGVPIT

SEQ ID NO: 78 - protein, 10 aa, synthetic construct
  REGION 1..10: note = Synthetic: HVR-L3
QQYDNYPPIT

SEQ ID NO: 79 - protein, 9 aa, synthetic construct
  REGION 1..9: note = Synthetic: HVR-L3
QQVIILPLT

SEQ ID NO: 80 - protein, 9 aa, synthetic construct
  REGION 1..9: note = Synthetic: HVR-L3
QQLAHSPFT

SEQ ID NO: 81 - protein, 9 aa, synthetic construct
  REGION 1..9: note = Synthetic: HVR-L3
QQYITYPIT

SEQ ID NO: 82 - protein, 9 aa, synthetic construct
  REGION 1..9: note = Synthetic: HVR-L3
QQYNAYPIT

SEQ ID NO: 83 - protein, 9 aa, synthetic construct
  REGION 1..9: note = Synthetic: HVR-L3
QQASAFPIT

SEQ ID NO: 84 - protein, 9 aa, synthetic construct
  REGION 1..9: note = Synthetic: HVR-L3
QQGVSLPYT

SEQ ID NO: 85 - protein, 8 aa, synthetic construct
  REGION 1..8: note = Synthetic: HVR-L3
QQYYDSIT

SEQ ID NO: 86 - protein, 9 aa, synthetic construct
  REGION 1..9: note = Synthetic: HVR-L3
QQHFSLPPT

SEQ ID NO: 87 - protein, 9 aa, synthetic construct
  REGION 1..9: note = Synthetic: HVR-L3
QQISSYPIT

SEQ ID NO: 88 - protein, 9 aa, synthetic construct
  REGION 1..9: note = Synthetic: HVR-L3
QQLYIFPPT

SEQ ID NO: 89 - protein, 9 aa, synthetic construct
  REGION 1..9: note = Synthetic: HVR-L3
QQVSLYPPT

SEQ ID NO: 90 - protein, 9 aa, synthetic construct
  REGION 1..9: note = Synthetic: HVR-L3
QQLYLYPPT

SEQ ID NO: 91 - protein, 8 aa, synthetic construct
  REGION 1..8: note = Synthetic: HVR-L3
QQYYSSYT

SEQ ID NO: 92 - protein, 9 aa, synthetic construct
  REGION 1..9: note = Synthetic: HVR-L3
QQYVDLPLT

SEQ ID NO: 93 - protein, 9 aa, synthetic construct
  REGION 1..9: note = Synthetic: HVR-L3
QQSASLPYT

SEQ ID NO: 94 - protein, 9 aa, synthetic construct
  REGION 1..9: note = Synthetic: HVR-L3
QQVYLFPIT

SEQ ID NO: 95 - protein, 9 aa, synthetic construct
  REGION 1..9: note = Synthetic: HVR-L3
QQYESFPWT

SEQ ID NO: 96 - protein, 9 aa, synthetic construct
  REGION 1..9: note = Synthetic: HVR-L3
QQAIYFPIT

SEQ ID NO: 97 - protein, 9 aa, synthetic construct
  REGION 1..9: note = Synthetic: HVR-L3
QQDSAWPFT

SEQ ID NO: 98 - protein, 9 aa, synthetic construct
  REGION 1..9: note = Synthetic: HVR-L3
QQPLNLPFT

SEQ ID NO: 99 - protein, 9 aa, synthetic construct
  REGION 1..9: note = Synthetic: HVR-L3
QQVIDLPYT

SEQ ID NO: 100 - protein, 9 aa, synthetic construct
  REGION 1..9: note = Synthetic: HVR-L3
QDRSNLPWT

SEQ ID NO: 101 - protein, 8 aa, synthetic construct
  REGION 1..8: note = Synthetic: HVR-L3
QQYVNYWT

SEQ ID NO: 102 - protein, 9 aa, synthetic construct
  REGION 1..9: note = Synthetic: HVR-H1
FTFGDYAMH

SEQ ID NO: 103 - protein, 9 aa, synthetic construct
  REGION 1..9: note = Synthetic: HVR-H1
FTFSSYGMH

SEQ ID NO: 104 - protein, 9 aa, synthetic construct
  REGION 1..9: note = Synthetic: HVR-H1
FTFSTYAMS

SEQ ID NO: 105 - protein, 9 aa, synthetic construct
  REGION 1..9: note = Synthetic: HVR-H1
FTFSNYAMS

SEQ ID NO: 106 - protein, 9 aa, synthetic construct
  REGION 1..9: note = Synthetic: HVR-H1
FTFSSYAMS

SEQ ID NO: 107 - protein, 9 aa, synthetic construct
  REGION 1..9: note = Synthetic: HVR-H1
FTFDDYAMH

SEQ ID NO: 108 - protein, 9 aa, synthetic construct
  REGION 1..9: note = Synthetic: HVR-H1
YTFTSYYIH

SEQ ID NO: 109 - protein, 9 aa, synthetic construct
  REGION 1..9: note = Synthetic: HVR-H1
FTFSSYSMN

SEQ ID NO: 110 - protein, 9 aa, synthetic construct
  REGION 1..9: note = Synthetic: HVR-H1
GTFSSYAIS

SEQ ID NO: 111 - protein, 9 aa, synthetic construct
  REGION 1..9: note = Synthetic: HVR-H1
GSFSGYYWS

SEQ ID NO: 112 - protein, 9 aa, synthetic construct
  REGION 1..9: note = Synthetic: HVR-H1
FTFSSYWMS

SEQ ID NO: 113 - protein, 9 aa, synthetic construct
  REGION 1..9: note = Synthetic: HVR-H1
YTFTGSYMH

SEQ ID NO: 114 - protein, 9 aa, synthetic construct
  REGION 1..9: note = Synthetic: HVR-H1
YTFTSYGIS

SEQ ID NO: 115 - protein, 11 aa, synthetic construct
  REGION 1..11: note = Synthetic: HVR-H1
GSISSSSYYWG

SEQ ID NO: 116 - protein, 11 aa, synthetic construct
  REGION 1..11: note = Synthetic: HVR-H1
GSISSGGYYWS

SEQ ID NO: 117 - protein, 9 aa, synthetic construct
  REGION 1..9: note = Synthetic: HVR-H1
FTFSNYGMH

SEQ ID NO: 118 - protein, 9 aa, synthetic construct
  REGION 1..9: note = Synthetic: HVR-H1
YTFTNYGIS

SEQ ID NO: 119 - protein, 9 aa, synthetic construct
  REGION 1..9: note = Synthetic: HVR-H1
YTFTSYYMH

SEQ ID NO: 120 - protein, 9 aa, synthetic construct
  REGION 1..9: note = Synthetic: HVR-H1
YTFTSYYMV

SEQ ID NO: 121 - protein, 9 aa, synthetic construct
  REGION 1..9: note = Synthetic: HVR-H1
GSISSYYWS

SEQ ID NO: 122 - protein, 11 aa, synthetic construct
  REGION 1..11: note = Synthetic: HVR-H1
GSISSSSYAWG

SEQ ID NO: 123 - protein, 17 aa, synthetic construct
  REGION 1..17: note = Synthetic: HVR-H2
GISWNSGSIGYADSVKG

SEQ ID NO: 124 - protein, 17 aa, synthetic construct
  REGION 1..17: note = Synthetic: HVR-H2
VISYDGSNKYYADSVKG

SEQ ID NO: 125 - protein, 17 aa, synthetic construct
  REGION 1..17: note = Synthetic: HVR-H2
AISGSGGSTYYADSVKG

SEQ ID NO: 126 - protein, 17 aa, synthetic construct
  REGION 1..17: note = Synthetic: HVR-H2
GITWNSGSIGYADSVKG

SEQ ID NO: 127 - protein, 17 aa, synthetic construct
  REGION 1..17: note = Synthetic: HVR-H2
LIWYDGSNKYYADSVKG

SEQ ID NO: 128 - protein, 17 aa, synthetic construct
  REGION 1..17: note = Synthetic: HVR-H2
TISGSGGSTYYADSVKG

SEQ ID NO: 129 - protein, 17 aa, synthetic construct
  REGION 1..17: note = Synthetic: HVR-H2
IINPSGGSTSYAQKFQG

SEQ ID NO: 130 - protein, 17 aa, synthetic construct
  REGION 1..17: note = Synthetic: HVR-H2
SISSSSSYIYYADSVKG

SEQ ID NO: 131 - protein, 17 aa, synthetic construct
  REGION 1..17: note = Synthetic: HVR-H2
GIIPIFGTANYAQKFQG

SEQ ID NO: 132 - protein, 16 aa, synthetic construct
  REGION 1..16: note = Synthetic: HVR-H2
EIDHSGSTNYNPSLKS

SEQ ID NO: 133 - protein, 17 aa, synthetic construct
  REGION 1..17: note = Synthetic: HVR-H2
GIIPIFGTASYAQKFQG

SEQ ID NO: 134 - protein, 17 aa, synthetic construct
  REGION 1..17: note = Synthetic: HVR-H2
NIKSDGSEKYYVDSVKG

SEQ ID NO: 135 - protein, 17 aa, synthetic construct
  REGION 1..17: note = Synthetic: HVR-H2
WINPNSGGTNYAQKFQG

SEQ ID NO: 136 - protein, 17 aa, synthetic construct
  REGION 1..17: note = Synthetic: HVR-H2
WISTYNGNTNYAQKLQG

SEQ ID NO: 137 - protein, 17 aa, synthetic construct
  REGION 1..17: note = Synthetic: HVR-H2
VIWYDGSNKYYADSVKG

SEQ ID NO: 138 - protein, 17 aa, synthetic construct
  REGION 1..17: note = Synthetic: HVR-H2
SIIPIFGTANYAQKFQG

SEQ ID NO: 139 - protein, 16 aa, synthetic construct
  REGION 1..16: note = Synthetic: HVR-H2
SIYYSGSTYYNPSLKS

SEQ ID NO: 140 - protein, 16 aa, synthetic construct
  REGION 1..16: note = Synthetic: HVR-H2
NIYYSGSTYYNPSLKS

SEQ ID NO: 141 - protein, 17 aa, synthetic construct
  REGION 1..17: note = Synthetic: HVR-H2
WISAYNGNTNYAQKLQG

SEQ ID NO: 142 - protein, 17 aa, synthetic construct
  REGION 1..17: note = Synthetic: HVR-H2
IINPSGGSTTYAQKFQG

SEQ ID NO: 143 - protein, 16 aa, synthetic construct
  REGION 1..16: note = Synthetic: HVR-H2
SIYYSGSTNYNPSLKS

SEQ ID NO: 144 - protein, 17 aa, synthetic construct
  REGION 1..17: note = Synthetic: HVR-H2
YISSSSSTIYYADSVKG

SEQ ID NO: 145 - protein, 16 aa, synthetic construct
  REGION 1..16: note = Synthetic: HVR-H2
YIYYSGSTYYNPSLKS

SEQ ID NO: 146 - protein, 17 aa, synthetic construct
  REGION 1..17: note = Synthetic: HVR-H2
NIKQDGSEKYYVDSVKG

SEQ ID NO: 147 - protein, 11 aa, synthetic construct
  REGION 1..11: note = Synthetic: HVR-H3
AKDAPYVGLDL

SEQ ID NO: 148 - protein, 22 aa, synthetic construct
  REGION 1..22: note = Synthetic: HVR-H3
AKDLGGGSGGYATSDYYYGMDV

SEQ ID NO: 149 - protein, 14 aa, synthetic construct
  REGION 1..14: note = Synthetic: HVR-H3
AKDLIDYASGGFDY

SEQ ID NO: 150 - protein, 14 aa, synthetic construct
  REGION 1..14: note = Synthetic: HVR-H3
AKDQGQYHWHAFDI

SEQ ID NO: 151 - protein, 18 aa, synthetic construct
  REGION 1..18: note = Synthetic: HVR-H3
AKDTGIDFWSVTPPYFDL

SEQ ID NO: 152 - protein, 13 aa, synthetic construct
  REGION 1..13: note = Synthetic: HVR-H3
AKEGRYGSGITTT

SEQ ID NO: 153 - protein, 17 aa, synthetic construct
  REGION 1..17: note = Synthetic: HVR-H3
AKEGSSYADGWPYGMDV

SEQ ID NO: 154 - protein, 15 aa, synthetic construct
  REGION 1..15: note = Synthetic: HVR-H3
AKEPRSYWHGYGMDV

SEQ ID NO: 155 - protein, 15 aa, synthetic construct
  REGION 1..15: note = Synthetic: HVR-H3
AKGAPQTSWLYGMDV

SEQ ID NO: 156 - protein, 13 aa, synthetic construct
  REGION 1..13: note = Synthetic: HVR-H3
AKGKQYSANYFDY

SEQ ID NO: 157 - protein, 14 aa, synthetic construct
  REGION 1..14: note = Synthetic: HVR-H3
AKTRYGYYSYDFDL

SEQ ID NO: 158 - protein, 9 aa, synthetic construct
  REGION 1..9: note = Synthetic: HVR-H3
ARAPGSWFY

SEQ ID NO: 159 - protein, 12 aa, synthetic construct
  REGION 1..12: note = Synthetic: HVR-H3
ARDGRTWGPHFY

SEQ ID NO: 160 - protein, 14 aa, synthetic construct
  REGION 1..14: note = Synthetic: HVR-H3
ARDGSMYYGDPMDV

SEQ ID NO: 161 - protein, 11 aa, synthetic construct
  REGION 1..11: note = Synthetic: HVR-H3
ARDGSYSYATY

SEQ ID NO: 162 - protein, 13 aa, synthetic construct
  REGION 1..13: note = Synthetic: HVR-H3
ARDLGPPGPHLDV

SEQ ID NO: 163 - protein, 14 aa, synthetic construct
  REGION 1..14: note = Synthetic: HVR-H3
ARDLSAFYVGPFDY

SEQ ID NO: 164 - protein, 14 aa, synthetic construct
  REGION 1..14: note = Synthetic: HVR-H3
ARDLSYEPSYPFDI

SEQ ID NO: 165 - protein, 15 aa, synthetic construct
  REGION 1..15: note = Synthetic: HVR-H3
ARDLTKYTLGFAFDI

SEQ ID NO: 166 - protein, 17 aa, synthetic construct
  REGION 1..17: note = Synthetic: HVR-H3
ARDPLHYYDSSGDVGIY

SEQ ID NO: 167 - protein, 13 aa, synthetic construct
  REGION 1..13: note = Synthetic: HVR-H3
ARDPTDSSSYYDV

SEQ ID NO: 168 - protein, 14 aa, synthetic construct
  REGION 1..14: note = Synthetic: HVR-H3
ARDQGTGTTVDLDL

SEQ ID NO: 169 - protein, 14 aa, synthetic construct
  REGION 1..14: note = Synthetic: HVR-H3
ARDRGMVSSDYFDY

SEQ ID NO: 170 - protein, 14 aa, synthetic construct
  REGION 1..14: note = Synthetic: HVR-H3
ARDRGYPRYSYLDL

SEQ ID NO: 171 - protein, 16 aa, synthetic construct
  REGION 1..16: note = Synthetic: HVR-H3
ARDRLDGSLGIYGMDV

SEQ ID NO: 172 - protein, 11 aa, synthetic construct
  REGION 1..11: note = Synthetic: HVR-H3
ARDSDKVALDL

SEQ ID NO: 173 - protein, 13 aa, synthetic construct
  REGION 1..13: note = Synthetic: HVR-H3
ARDSDTSWGGFDY

SEQ ID NO: 174 - protein, 17 aa, synthetic construct
  REGION 1..17: note = Synthetic: HVR-H3
ARDTGSATTMWLYGMDV

SEQ ID NO: 175 - protein, 13 aa, synthetic construct
  REGION 1..13: note = Synthetic: HVR-H3
ARDTMSGSSPTDY

SEQ ID NO: 176 - protein, 16 aa, synthetic construct
  REGION 1..16: note = Synthetic: HVR-H3
ARDVGMARGDPYGMDV

SEQ ID NO: 177 - protein, 10 aa, synthetic construct
  REGION 1..10: note = Synthetic: HVR-H3
ARDVYSSYSY

SEQ ID NO: 178 - protein, 13 aa, synthetic construct
  REGION 1..13: note = Synthetic: HVR-H3
AREAGYDIKGFDY

SEQ ID NO: 179 - protein, 15 aa, synthetic construct
  REGION 1..15: note = Synthetic: HVR-H3
AREGAMGYRDYYMDV

SEQ ID NO: 180 - protein, 15 aa, synthetic construct
  REGION 1..15: note = Synthetic: HVR-H3
AREGAYSLYVNWFDP

SEQ ID NO: 181 - protein, 17 aa, synthetic construct
  REGION 1..17: note = Synthetic: HVR-H3
AREGGYGYRAYPYGMDV

SEQ ID NO: 182 - protein, 18 aa, synthetic construct
  REGION 1..18: note = Synthetic: HVR-H3
AREGLASTGVSDYYYMDV

SEQ ID NO: 183 - protein, 16 aa, synthetic construct
  REGION 1..16: note = Synthetic: HVR-H3
AREGVAARGYYYGMDV

SEQ ID NO: 184 - protein, 11 aa, synthetic construct
  REGION 1..11: note = Synthetic: HVR-H3
ARELETSRHDI

SEQ ID NO: 185 - protein, 13 aa, synthetic construct
  REGION 1..13: note = Synthetic: HVR-H3
AREPFYSADRFDY

SEQ ID NO: 186 - protein, 14 aa, synthetic construct
  REGION 1..14: note = Synthetic: HVR-H3
ARESGGTGAYGMDV

SEQ ID NO: 187 - protein, 16 aa, synthetic construct
  REGION 1..16: note = Synthetic: HVR-H3
ARESYDSSLHYYGMDV

SEQ ID NO: 188 - protein, 17 aa, synthetic construct
  REGION 1..17: note = Synthetic: HVR-H3
AREVGVSGWEITYGMDV

SEQ ID NO: 189 - protein, 18 aa, synthetic construct
  REGION 1..18: note = Synthetic: HVR-H3
ARGAPIETLGVYYYGMDV

SEQ ID NO: 190 - protein, 19 aa, synthetic construct
  REGION 1..19: note = Synthetic: HVR-H3
ARGAPSMQYSPYYYYGMDV

SEQ ID NO: 191 - protein, 18 aa, synthetic construct
  REGION 1..18: note = Synthetic: HVR-H3
ARGGGGYSYSPYIYGMDV

SEQ ID NO: 192 - protein, 15 aa, synthetic construct
  REGION 1..15: note = Synthetic: HVR-H3
ARGGYAAGTDYYMGV

SEQ ID NO: 193 - protein, 10 aa, synthetic construct
  REGION 1..10: note = Synthetic: HVR-H3
ARGLWSEHIY

SEQ ID NO: 194 - protein, 12 aa, synthetic construct
  REGION 1..12: note = Synthetic: HVR-H3
ARGPLGAAGFDY

SEQ ID NO: 195 - protein, 18 aa, synthetic construct
  REGION 1..18: note = Synthetic: HVR-H3
ARGSGIAAADTDGLPMDV

SEQ ID NO: 196 - protein, 14 aa, synthetic construct
  REGION 1..14: note = Synthetic: HVR-H3
ARGSPYYDAYDFDL

SEQ ID NO: 197 - protein, 15 aa, synthetic construct
  REGION 1..15: note = Synthetic: HVR-H3
ARGVETYSGPNWFDP

SEQ ID NO: 198 - protein, 12 aa, synthetic construct
  REGION 1..12: note = Synthetic: HVR-H3
ARKGMDDAGMDV

SEQ ID NO: 199 - protein, 13 aa, synthetic construct
  REGION 1..13: note = Synthetic: HVR-H3
ARLPMGCGSGMDV

SEQ ID NO: 200 - protein, 12 aa, synthetic construct
  REGION 1..12: note = Synthetic: HVR-H3
ARSGGSYSYLDV

SEQ ID NO: 201 - protein, 13 aa, synthetic construct
  REGION 1..13: note = Synthetic: HVR-H3
ARTHKHFDYGMDV

SEQ ID NO: 202 - protein, 118 aa, synthetic construct
  REGION 1..118: note = Synthetic: Heavy Chain Variable
EVQLVESGGGLVQPGRSLRLSCAASGFTFGDYAMHWVRQAPGKGLEWVSGISWNSGSIGYADSVKGRFTISRDNAKNSLYLQMNSLRAEDTALYYCAKDAPYVGLDLWGRGTLVTVSS

SEQ ID NO: 203 - protein, 129 aa, synthetic construct
  REGION 1..129: note = Synthetic: Heavy Chain Variable
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLGGGSGGYATSDYYYGMDVWGQGTTVTVSS

SEQ ID NO: 204 - protein, 121 aa, synthetic construct
  REGION 1..121: note = Synthetic: Heavy Chain Variable
EVQLLESGGGLVQPGGSLRLSCAASGFTFSTYAMSWVRQAPGKGLEWVSAISGSGGSTYYADSVKGRFTISRDNSKNTLYLQMNSLRAEDTAVYYCAKDLIDYASGGFDYWGQGTLVTVSS

SEQ ID NO: 205 - protein, 121 aa, synthetic construct
  REGION 1..121: note = Synthetic: Heavy Chain Variable
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKDQGQYHWHAFDIWGQGTMVTVSS

SEQ ID NO: 206 - protein, 125 aa, synthetic construct
  REGION 1..125: note = Synthetic: Heavy Chain Variable
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDTGIDFWSVTPPYFDLWGRGTLVTVSS

SEQ ID NO: 207 - protein, 120 aa, synthetic construct
  REGION 1..120: note = Synthetic: Heavy Chain Variable
EVQLVESGGGLVQPGRSLRLSCAASGFTFDDYAMHWVRQAPGKGLEWVSGITWNSGSIGYADSVKGRFTISRDNAKNSLYLQMNSLRAEDTALYYCAKEGRYGSGITTTWGQGTLVTVSS

SEQ ID NO: 208 - protein, 124 aa, synthetic construct
  REGION 1..124: note = Synthetic: Heavy Chain Variable
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCAKEGSSYADGWPYGMDVWGQGTTVTVSS

SEQ ID NO: 209 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKEPRSYWHGYGMDVWGQGTTVTVSS

SEQ ID NO: 210 - protein, 122 aa, synthetic construct
  REGION 1..122: note = Synthetic: Heavy Chain Variable
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCAKGAPQTSWLYGMDVWGQGTTVTVSS

SEQ ID NO: 211 - protein, 120 aa, synthetic construct
  REGION 1..120: note = Synthetic: Heavy Chain Variable
EVQLVESGGGLVQPGRSLRLSCAASGFTFDDYAMHWVRQAPGKGLEWVSGITWNSGSIGYADSVKGRFTISRDNAKNSLYLQMNSLRAEDTALYYCAKGKQYSANYFDYWGQGTLVTVSS

SEQ ID NO: 212 - protein, 121 aa, synthetic construct
  REGION 1..121: note = Synthetic: Heavy Chain Variable
EVQLVESGGGLVQPGRSLRISCAASGFTFGDYAMHWVRQAPGKGLEWVSGISWNSGSIGYADSVKGRFTISRDNAKNSLYLQMNSLRAEDTALYYCAKTRYGYYSYDFDLWGRGTLVTVSS

SEQ ID NO: 213 - protein, 116 aa, synthetic construct
  REGION 1..116: note = Synthetic: Heavy Chain Variable
QVQLVQSGAEVKKPGASVKVSCKASGYTFTSYYIHWVRQAPGQGLEWMGIINPSGGSTSYAQKFQGRVTMTRDTSTSTVYMELSSLRSEDTAVYYCARAPGSWFYWGQGTLVTVSS

SEQ ID NO: 214 - protein, 119 aa, synthetic construct
  REGION 1..119: note = Synthetic: Heavy Chain Variable
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGRTWGPHFYWGQGTLVTVSS

SEQ ID NO: 215 - protein, 121 aa, synthetic construct
  REGION 1..121: note = Synthetic: Heavy Chain Variable
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGSMYYGDPMDVWGQGTTVTVSS

SEQ ID NO: 216 - protein, 118 aa, synthetic construct
  REGION 1..118: note = Synthetic: Heavy Chain Variable
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGSYSYATYWGQGTLVTVSS

SEQ ID NO: 217 - protein, 119 aa, synthetic construct
  REGION 1..119: note = Synthetic: Heavy Chain Variable
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARDLGPPGPHLDVWGQGTMVTVSS

SEQ ID NO: 218 - protein, 121 aa, synthetic construct
  REGION 1..121: note = Synthetic: Heavy Chain Variable
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARDLSAFYVGPFDYWGQGTLVTVSS

SEQ ID NO: 219 - protein, 121 aa, synthetic construct
  REGION 1..121: note = Synthetic: Heavy Chain Variable
EVQLVESGGGLVQPGGSLRLSCAASGFTFSSYWMSWVRQAPGKGLEWVANIKSDGSEKYYVDSVKGRFTISRDNAKNSLYLQMNSLRAEDTAVYYCARDLSYEPSYPFDIWGQGTMVTVSS

SEQ ID NO: 220 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDLTKYTLGFAFDIWGQGTMVTVSS

SEQ ID NO: 221 - protein, 124 aa, synthetic construct
  REGION 1..124: note = Synthetic: Heavy Chain Variable
QVQLVQSGAEVKKPGASVKVSCKASGYTFTGSYMHWVRQAPGQGLEWMGWINPNSGGTNYAQKFQGRVTMTRDTSISTAYMELSRLRSDDTAVYYCARDPLHYYDSSGDVGIYWGQGTLVTVSS

SEQ ID NO: 222 - protein, 120 aa, synthetic construct
  REGION 1..120: note = Synthetic: Heavy Chain Variable
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDPTDSSSYYDVWGQGTLVTVSS

SEQ ID NO: 223 - protein, 121 aa, synthetic construct
  REGION 1..121: note = Synthetic: Heavy Chain Variable
QVQLVQSGAEVKKPGASVKVSCKASGYTFTSYGISWVRQAPGQGLEWMGWISTYNGNTNYAQKLQGRVTMTTDTSTSTAYMELRSLRSDDTAVYYCARDQGTGTTVDLDLWGRGTLVTVSS

SEQ ID NO: 224 - protein, 121 aa, synthetic construct
  REGION 1..121: note = Synthetic: Heavy Chain Variable
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDRGMVSSDYFDYWGQGTLVTVSS

SEQ ID NO: 225 - protein, 121 aa, synthetic construct
  REGION 1..121: note = Synthetic: Heavy Chain Variable
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARDRGYPRYSYLDLWGRGTLVTVSS

SEQ ID NO: 226 - protein, 122 aa, synthetic construct
  REGION 1..122: note = Synthetic: Heavy Chain Variable
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARDRLDGSLGIYGMDVWGQGTTVTVSS

SEQ ID NO: 227 - protein, 119 aa, synthetic construct
  REGION 1..119: note = Synthetic: Heavy Chain Variable
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDSDKVALDLWGRGTLVTVSS

SEQ ID NO: 228 - protein, 121 aa, synthetic construct
  REGION 1..121: note = Synthetic: Heavy Chain Variable
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARDSDTSWGGFDYWGQGTLVTVSS

SEQ ID NO: 229 - protein, 124 aa, synthetic construct
  REGION 1..124: note = Synthetic: Heavy Chain Variable
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDTGSATTMWLYGMDVWGKGTTVTVSS

SEQ ID NO: 230 - protein, 120 aa, synthetic construct
  REGION 1..120: note = Synthetic: Heavy Chain Variable
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCARDTMSGSSPTDYWGQGTLVTVSS

SEQ ID NO: 231 - protein, 123 aa, synthetic construct
  REGION 1..123: note = Synthetic: Heavy Chain Variable
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARDVGMARGDPYGMDVWGQGTTVTVSS

SEQ ID NO: 232 - protein, 117 aa, synthetic construct
  REGION 1..117: note = Synthetic: Heavy Chain Variable
QVQLVQSGAEVKKPGASVKVSCKASGYTFTNYGISWVRQAPGQGLEWMGWISAYNGNTNYAQKLQGRVTMTTDTSTSTAYMELRSLRSDDTAVYYCARDVYSSYSYWGQGTLVTVSS

SEQ ID NO: 233 - protein, 120 aa, synthetic construct
  REGION 1..120: note = Synthetic: Heavy Chain Variable
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREAGYDIKGFDYWGQGTLVTVSS

SEQ ID NO: 234 - protein, 122 aa, synthetic construct
  REGION 1..122: note = Synthetic: Heavy Chain Variable
QVQLVQSGAEVKKPGASVKVSCKASGYTFTGSYMHWVRQAPGQGLEWMGWINPNSGGTNYAQKFQGRVTMTRDTSISTAYMELSRLRSDDTAVYYCAREGAMGYRDYYMDVWGKGTTVTVSS

SEQ ID NO: 235 - protein, 123 aa, synthetic construct
  REGION 1..123: note = Synthetic: Heavy Chain Variable
QVQLQESGPGLVKPSQTLSLTCTVSGGSISSGGYYWSWIRQHPGKGLEWIGSIYYSGSTYYNPSLKSRVTISVDTSKNQFSLKLSSVTAADTAVYYCAREGAYSLYVNWFDPWGQGTLVTVSS

SEQ ID NO: 236 - protein, 124 aa, synthetic construct
  REGION 1..124: note = Synthetic: Heavy Chain Variable
QVQLVQSGAEVKKPGASVKVSCKASGYTFTSYYMVWVRQAPGQGLEWMGIINPSGGSTSYAQKFQGRVTMTRDTSTSTVYMELSSLRSEDTAVYYCAREGGYGYRAYPYGMDVWGQGTTVTVSS

SEQ ID NO: 237 - protein, 125 aa, synthetic construct
  REGION 1..125: note = Synthetic: Heavy Chain Variable
QVQLVQSGAEVKKPGASVKVSCKASGYTFTSYYMHWVRQAPGQGLEWMGIINPSGGSTTYAQKFQGRVTMTRDTSTSTVYMELSSLRSEDTAVYYCAREGLASTGVSDYYYMDVWGKGTTVTVSS

SEQ ID NO: 238 - protein, 122 aa, synthetic construct
  REGION 1..122: note = Synthetic: Heavy Chain Variable
QVQLQESGPGLVKPSETLSLTCTVSGGSISSYYWSWIRQPPGKGLEWIGSIYYSGSTNYNPSLKSRVTISVDTSKNQFSLKLSSVTAADTAVYYCAREGVAARGYYYGMDVWGQGTTVTVSS

SEQ ID NO: 239 - protein, 119 aa, synthetic construct
  REGION 1..119: note = Synthetic: Heavy Chain Variable
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARELETSRHDIWGQGTTVTVSS

SEQ ID NO: 240 - protein, 120 aa, synthetic construct
  REGION 1..120: note = Synthetic: Heavy Chain Variable
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAREPFYSADRFDYWGQGTLVTVSS

SEQ ID NO: 241 - protein, 121 aa, synthetic construct
  REGION 1..121: note = Synthetic: Heavy Chain Variable
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESGGTGAYGMDVWGQGTTVTVSS

SEQ ID NO: 242 - protein, 123 aa, synthetic construct
  REGION 1..123: note = Synthetic: Heavy Chain Variable
EVQLLESGGGLVQPGGSLRLSCAASGFTFSTYAMSWVRQAPGKGLEWVSAISGSGGSTYYADSVKGRFTISRDNSKNTLYLQMNSLRAEDTAVYYCARESYDSSLHYYGMDVWGQGTTVTVSS

SEQ ID NO: 243 - protein, 124 aa, synthetic construct
  REGION 1..124: note = Synthetic: Heavy Chain Variable
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREVGVSGWEITYGMDVWGQGTTVTVSS

SEQ ID NO: 244 - protein, 125 aa, synthetic construct
  REGION 1..125: note = Synthetic: Heavy Chain Variable
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGAPIETLGVYYYGMDVWGQGTTVTVSS

SEQ ID NO: 245 - protein, 126 aa, synthetic construct
  REGION 1..126: note = Synthetic: Heavy Chain Variable
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPSMQYSPYYYYGMDVWGQGTTVTVSS

SEQ ID NO: 246 - protein, 125 aa, synthetic construct
  REGION 1..125: note = Synthetic: Heavy Chain Variable
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGGGYSYSPYIYGMDVWGQGTTVTVSS

SEQ ID NO: 247 - protein, 122 aa, synthetic construct
  REGION 1..122: note = Synthetic: Heavy Chain Variable
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGGYAAGTDYYMGVWGKGTTVTVSS

SEQ ID NO: 248 - protein, 117 aa, synthetic construct
  REGION 1..117: note = Synthetic: Heavy Chain Variable
QVQLVQSGAEVKKPGASVKVSCKASGYTFTNYGISWVRQAPGQGLEWMGWISAYNGNTNYAQKLQGRVTMTTDTSTSTAYMELRSLRSDDTAVYYCARGLWSEHIYWGQGTLVTVSS

SEQ ID NO: 249 - protein, 120 aa, synthetic construct
  REGION 1..120: note = Synthetic: Heavy Chain Variable
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARGPLGAAGFDYWGQGTLVTVSS

SEQ ID NO: 250 - protein, 126 aa, synthetic construct
  REGION 1..126: note = Synthetic: Heavy Chain Variable
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSGIAAADTDGLPMDVWGQGTTVTVSS

SEQ ID NO: 251 - protein, 121 aa, synthetic construct
  REGION 1..121: note = Synthetic: Heavy Chain Variable
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGSPYYDAYDFDLWGRGTLVTVSS

SEQ ID NO: 252 - protein, 122 aa, synthetic construct
  REGION 1..122: note = Synthetic: Heavy Chain Variable
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGVETYSGPNWFDPWGQGTLVTVSS

SEQ ID NO: 253 - protein, 120 aa, synthetic construct
  REGION 1..120: note = Synthetic: Heavy Chain Variable
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARKGMDDAGMDVWGQGTTVTVSS

SEQ ID NO: 254 - protein, 121 aa, synthetic construct
  REGION 1..121: note = Synthetic: Heavy Chain Variable
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARLPMGCGSGMDVWGQGTTVTVSS

SEQ ID NO: 255 - protein, 118 aa, synthetic construct
  REGION 1..118: note = Synthetic: Heavy Chain Variable
QVQLQESGPGLVKPSETLSLTCTVSGGSISSYYWSWIRQPPGKGLEWIGSIYYSGSTNYNPSLKSRVTISVDTSKNQFSLKLSSVTAADTAVYYCARSGGSYSYLDVWGQGTMVTVSS

SEQ ID NO: 256 - protein, 120 aa, synthetic construct
  REGION 1..120: note = Synthetic: Heavy Chain Variable
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARTHKHFDYGMDVWGQGTTVTVSS

SEQ ID NO: 257 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDSSNRATGIPARFSGSGSGTDFTLTISSLEPEDFAVYYCQQDSDYPFTFGGGTKVEIK

SEQ ID NO: 258 - protein, 113 aa, synthetic construct
  REGION 1..113: note = Synthetic: Light Chain Variable
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYFLYPPTFGGGTKVEIK

SEQ ID NO: 259 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQHDFWPPTFGGGTKVEIK

SEQ ID NO: 260 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQDSNFPFTFGGGTKVEIK

SEQ ID NO: 261 - protein, 107 aa, synthetic construct
  REGION 1..107: note = Synthetic: Light Chain Variable
DIQLTQSPSSVSASVGDRVTITCRASQDISSWLAWYQQKPGKAPKLLIYAASSLQSGVPSRFSGSGSGTDFTLTISSLQPEDFATYYCQQGYIFPRTFGGGTKVEIK

SEQ ID NO: 262 - protein, 107 aa, synthetic construct
  REGION 1..107: note = Synthetic: Light Chain Variable
EIVMTQSPATLSLSPGERATLSCRASQSVSSYLAWYQQKPGQAPRLLIYDSSNRATGIPARFSGSGSGTDFTLTISSLEPEDFAVYYCQQDANYPYTFGGGTKVEIK

SEQ ID NO: 263 - protein, 113 aa, synthetic construct
  REGION 1..113: note = Synthetic: Light Chain Variable
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYHYPPTFGGGTKVEIK

SEQ ID NO: 264 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQLSNYPITFGGGTKVEIK

SEQ ID NO: 265 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQAYLYPITFGGGTKVENQ

SEQ ID NO: 266 - protein, 107 aa, synthetic construct
  REGION 1..107: note = Synthetic: Light Chain Variable
DIQMTQSPSTLSASVGDRVTITCRASQSISSWLAWYQQKPGKAPKLLIYEASSLESGVPSRFSGSGSGTEFTLTISSLQPDDFATYYCQQDNSLPYTFGGGTKVEIK

SEQ ID NO: 267 - protein, 107 aa, synthetic construct
  REGION 1..107: note = Synthetic: Light Chain Variable
DIQMTQSPSTLSASVGDRVTITCRASQSISSWLAWYQQKPGKAPKLLIYDASSLESGVPSRFSGSGSGTEFTLTISSLQPDDFATYYCQQYNSYSPTFGGGTKVEIK

SEQ ID NO: 268 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQRTNFPITFGGGTKVEIK

SEQ ID NO: 269 - protein, 107 aa, synthetic construct
  REGION 1..107: note = Synthetic: Light Chain Variable
DIQMTQSPSSLSASVGDRVTITCRASQSISSYLNWYQQKPGKAPKLLIYAASSLQSGVPSRFSGSGSGTDFTLTISSLQPEDFATYYCQQGYIYPPTFGGGTKVEIK

SEQ ID NO: 270 - protein, 107 aa, synthetic construct
  REGION 1..107: note = Synthetic: Light Chain Variable
DIQLTQSPSSLSASVGDRVTITCQASQDISNFLNWYQQKPGKAPKLLIYDASNLETGVPSRFSGSGSGTDFTFTISSLQPEDIAKYYCQQFINFPLTFGGGTKVEIK

SEQ ID NO: 271 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQPYNFPLTFGGGTKVEIK

SEQ ID NO: 272 - protein, 106 aa, synthetic construct
  REGION 1..106: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQFSDYPTFGGGTKVEIK

SEQ ID NO: 273 - protein, 107 aa, synthetic construct
  REGION 1..107: note = Synthetic: Light Chain Variable
EIVMTQSPATLSVSPGERATLSCRASQSVSSSLAWYQQKPGQAPRLLIYGASTRATGIPARFSGSGSGTEFTLTISSLQSEDFAVYYCQQDYIWPLTFGGGTKVEIK

SEQ ID NO: 274 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQALSFPFTFGGGTKVEIK

SEQ ID NO: 275 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQFTYLPPTFGGGTKVEIK

SEQ ID NO: 276 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQVDVLPYTFGGGTKVEIK

SEQ ID NO: 277 - protein, 113 aa, synthetic construct
  REGION 1..113: note = Synthetic: Light Chain Variable
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQDYATPFTFGGGTKVEIK

SEQ ID NO: 278 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQYDTLPPTFGGGTKVEIK

SEQ ID NO: 279 - protein, 112 aa, synthetic construct
  REGION 1..112: note = Synthetic: Light Chain Variable
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAQGTPYTFGGGTKVEIK

SEQ ID NO: 280 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQRLDWPYTFGGGTKVEIK

SEQ ID NO: 281 - protein, 106 aa, synthetic construct
  REGION 1..106: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQYSYLPTFGGGTKVEIK

SEQ ID NO: 282 - protein, 107 aa, synthetic construct
  REGION 1..107: note = Synthetic: Light Chain Variable
DIQMTQSPSSLSASVGDRVTITCRASQSINSYLNWYQQKPGKAPKLLIYAASSLQSGVPSRFSGSGSGTDFTLTISSLQPEDFATYYCQQSPSDPWTFGGGTKVEIK

SEQ ID NO: 283 - protein, 107 aa, synthetic construct
  REGION 1..107: note = Synthetic: Light Chain Variable
DIQMTQSPSSLSASVGDRVTITCRASQSISSYLNWYQQKPGKAPKLLIYAASSLQSGVPSRFSGSGSGTDFTLTISSLQPEDFATYYCQQSVHTPYTFGGGTKVEIK

SEQ ID NO: 284 - protein, 108 aa, synthetic construct
  REGION 1..108: note = Synthetic: Light Chain Variable
EIVLTQSPGTLSLSPGERATLSCRASQSVRSSYLAWYQQKPGQAPRLLIYGASSRATGIPDRFSGSGSGTDFTLTISRLEPEDFAVYYCQQAGVSPYTFGGGTKVEIK

SEQ ID NO: 285 - protein, 107 aa, synthetic construct
  REGION 1..107: note = Synthetic: Light Chain Variable
DIQMTQSPSSLSASVGDRVTITCRASQSISSYLNWYQQKPGKAPKLLIYAASSLQSGVPSRFSGSGSGTDFTLTISSLQPEDFATYYCQQSSHTPYTFGGGTKVEIK

SEQ ID NO: 286 - protein, 113 aa, synthetic construct
  REGION 1..113: note = Synthetic: Light Chain Variable
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYIYPPTFGGGTKVEIK

SEQ ID NO: 287 - protein, 108 aa, synthetic construct
  REGION 1..108: note = Synthetic: Light Chain Variable
EIVLTQSPGTLSLSPGERATLSCRASQSVSSSYLAWYQQKPGQAPRLLIYGASSRATGIPDRFSGSGSGTDFTLTISRLEPEDFAVYYCQQFFSYPPTFGGGTKVEIK

SEQ ID NO: 288 - protein, 107 aa, synthetic construct
  REGION 1..107: note = Synthetic: Light Chain Variable
EIVMTQSPATLSVSPGERATLSCRASQSVGSNLAWYQQKPGQAPRLLIYGASTRATGIPARFSGSGSGTEFTLTISSLQSEDFAVYYCQQLNVFPWTFGGGTKVEIK

SEQ ID NO: 289 - protein, 107 aa, synthetic construct
  REGION 1..107: note = Synthetic: Light Chain Variable
DIQMTQSPSSVSASVGDRVTITCRASQGISSWLAWYQQKPGKAPKLLISAASSLQSGVPSRFSGSGSGTDFTLTISSLQPEDFATYYCLQAYSFPFTFGGGTKVEIK

SEQ ID NO: 290 - protein, 107 aa, synthetic construct
  REGION 1..107: note = Synthetic: Light Chain Variable
DIQLTQSPSTLSASVGDRVTITCRASQSISSWLAWYQQKPGKAPKLLIYDASSLESGVPSRFSGSGSGTEFTLTISSLQPDDFATYYCQQYELLPPTFGGGTKVEIK

SEQ ID NO: 291 - protein, 107 aa, synthetic construct
  REGION 1..107: note = Synthetic: Light Chain Variable
DIQMTQSPSTLSASVGDRVTITCRASQSISSWLAWYQQKPGKAPKLLIYKASSLESGVPSRFSGSGSGTEFTLTISSLQPDDFATYYCQQYGSFPLTFGGGTKVEIK

SEQ ID NO: 292 - protein, 107 aa, synthetic construct
  REGION 1..107: note = Synthetic: Light Chain Variable
EIVLTQSPATLSLSPGERATITCQASQDISSYLAWYQQKPGQAPRLLIYDASNRATGIPARFSGSGSGTDFTLTISSLEPEDFAVYYCQQRTHYPWTFGGGTKVEIK

SEQ ID NO: 293 - protein, 108 aa, synthetic construct
  REGION 1..108: note = Synthetic: Light Chain Variable
EIVLTQSPGTLSLSPGERATLSCRASQSVSSSFLAWYQQKPGQAPRLLIYGASSRATGIPDRFSGSGSGTDFTLTISRLEPEDFAVYYCQQYDGVPITFGGGTKVEIK

SEQ ID NO: 294 - protein, 108 aa, synthetic construct
  REGION 1..108: note = Synthetic: Light Chain Variable
EIVMTQSPATLSVSPGERATLSCRASQSVSSSLAWYQQKPGQAPRLLIFGASTRATGIPARFSGSGSGTEFTLTISSLQSEDFAVYYCQQYDNYPPITFGGGTKVEIK

SEQ ID NO: 295 - protein, 107 aa, synthetic construct
  REGION 1..107: note = Synthetic: Light Chain Variable
DIQMTQSPSSLSASVGDRVTITCQASQDISNSLNWYQQKPGKAPKLLIYDASNLETGVPSRFSGSRSGTDFTFTISSLQPEDIATYYCQQVIILPLTFGGGTKVEIK

SEQ ID NO: 296 - protein, 108 aa, synthetic construct
  REGION 1..108: note = Synthetic: Light Chain Variable
EIVLTQSPGTLSLSPGERATLSCRASQSVSSSYLAWYQQKPGQAPRLLIYGASSRATGIPDRFSGSGSGTDFTLTISRLEPEDFAVYYCQQLAHSPFTFGGGTKVEIK

SEQ ID NO: 297 - protein, 107 aa, synthetic construct
  REGION 1..107: note = Synthetic: Light Chain Variable
EIVMTQSPATLSVSPGERATLSCRASQSVSSSLAWYQQKPGQAPRLLIYGASTRATGIPARFSGSGSGTEFTLTISSLQSEDFAVYYCQQYITYPITFGGGTKVEIK

SEQ ID NO: 298 - protein, 107 aa, synthetic construct
  REGION 1..107: note = Synthetic: Light Chain Variable
EIVMTQSPATLSVSPGERATLSCRASQSVSSNLAWYQQKPGQAPRLLIFGASTRATGIPARFSGSGSGTEFTLTISSLQSEDFAVYYCQQYNAYPITFGGGTKVEIK

SEQ ID NO: 299 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQASAFPITFGGGTKVEIK

SEQ ID NO: 300 - protein, 107 aa, synthetic construct
  REGION 1..107: note = Synthetic: Light Chain Variable
DIQMTQSPSSVSASVGDRVTITCRASQGIDSWLAWYQQKPGKAPKLLIYAASSLQSGVPSRFSGSGSGTDFTLTISSLQPEDFATYYCQQGVSLPYTFGGGTKVEIK

SEQ ID NO: 301 - protein, 107 aa, synthetic construct
  REGION 1..107: note = Synthetic: Light Chain Variable
DIQMTQSPSSLSASVGDRVTITCRASQSVSSSYLAWYQQKPGQAPRLLIYGASSRATGIPDRFSGSGSGTDFTLTISRLEPEDFAVYYCQQYYDSITFGGGTKVEIK

SEQ ID NO: 302 - protein, 108 aa, synthetic construct
  REGION 1..108: note = Synthetic: Light Chain Variable
EIVLTQSPGTLSLSPGERATLSCRASQSVSSSYLAWYQQKPGQAPRLLIYGASSRATGIPDRFSGSGSGTDFTLTISRLEPEDFAVYYCQQHFSLPPTFGGGTKVEIK

SEQ ID NO: 303 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQISSYPITFGGGTKVEIK

SEQ ID NO: 304 - protein, 113 aa, synthetic construct
  REGION 1..113: note = Synthetic: Light Chain Variable
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYIFPPTFGGGTKVEIK

SEQ ID NO: 305 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQVSLYPPTFGGGTKVEIK

SEQ ID NO: 306 - protein, 113 aa, synthetic construct
  REGION 1..113: note = Synthetic: Light Chain Variable
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYLYPPTFGGGTKVEIK

SEQ ID NO: 307 - protein, 107 aa, synthetic construct
  REGION 1..107: note = Synthetic: Light Chain Variable
EIVLTQSPGTLSLSPGERATLSCRASQSVSSSYLAWYQQKPGQAPRLLIYGASSRATGIPDRFSGSGSGTDFTLTISRLEPEDFAVYYCQQYYSSYTFGGGTKVEIK

SEQ ID NO: 308 - protein, 108 aa, synthetic construct
  REGION 1..108: note = Synthetic: Light Chain Variable
EIVMTQSPGTLSLSPGERATLSCRASQSVSSSYLAWYQQKPGQAPRLLIYGASNRATGIPDRFSGSGSGTDFTLTISRLEPEDFAVYYCQQYVDLPLTFGGGTKVEIK

SEQ ID NO: 309 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQSASLPYTFGGGTKVEIK

SEQ ID NO: 310 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQVYLFPITFGGGTKVEIK

SEQ ID NO: 311 - protein, 107 aa, synthetic construct
  REGION 1..107: note = Synthetic: Light Chain Variable
DIQLTQSPSTLSASVGDRVTITCRASQAISSWLAWYQQKPGKAPKLLIYDASSLESGVPSRFSGSGSGTEFTLTISSLQPDDFATYYCQQYESFPWTFGGGTKVEIK

SEQ ID NO: 312 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQAIYFPITFGGGTKVEIK

SEQ ID NO: 313 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDASNRATGIPARFSGSGSGTDFTLTISSLEPEDFAVYYCQQDSAWPFTFGGGTKVEIK

SEQ ID NO: 314 - protein, 107 aa, synthetic construct
  REGION 1..107: note = Synthetic: Light Chain Variable
DIQLTQSPSSLSASVGDRVTITCQASQDISNYLNWYQQKPGKAPKLLIYDASNLETGVPSRFSGSGSGTDFTFTISSLQPEDIATYYCQQPLNLPFTFGGGTKVEIK

SEQ ID NO: 315 - protein, 107 aa, synthetic construct
  REGION 1..107: note = Synthetic: Light Chain Variable
DIQMTQSPSSLSASVGDRVTITCQASQDISNYLNWYQQKPGKAPKLLIYDASNLETGVPSRFSGSGSGTDFTFTISSLQPEDIATYYCQQVIDLPYTFGGGTKVEIK

SEQ ID NO: 316 - protein, 107 aa, synthetic construct
  REGION 1..107: note = Synthetic: Light Chain Variable
EIVLTQSPATLSLSPGERATLSCRASQSVSSYLAWYQQKPGQAPRLLIYDSSNRATGIPARFSGSGSGTDFTLTISSLEPEDFAVYYCQDRSNLPWTFGGGTKVEIK

SEQ ID NO: 317 - protein, 106 aa, synthetic construct
  REGION 1..106: note = Synthetic: Light Chain Variable
DIQMTQSPSSLSASVGDRVTITCQASQDIANYLNWYQQKPGKAPKLLIYDASNLETGVPSRFSGSGSGTDFTFTISSLQPEDIATYYCQQYVNYWTFGGGTKVEIK

SEQ ID NO: 318 - protein, 9 aa, synthetic construct
  REGION 1..9: note = Synthetic: HVR-H1
FTFDSYAMS

SEQ ID NO: 319 - protein, 9 aa, synthetic construct
  REGION 1..9: note = Synthetic: HVR-H1
FTFDNYAMS

SEQ ID NO: 320 - protein, 9 aa, synthetic construct
  REGION 1..9: note = Synthetic: HVR-H1
FTFSSYSMH

SEQ ID NO: 321 - protein, 9 aa, synthetic construct
  REGION 1..9: note = Synthetic: HVR-H1
FTFTEYAMS

SEQ ID NO: 322 - protein, 9 aa, synthetic construct
  REGION 1..9: note = Synthetic: HVR-H1
FTFSEWAMS

SEQ ID NO: 323 - protein, 9 aa, synthetic construct
  REGION 1..9: note = Synthetic: HVR-H1
FTFSSYAMH

SEQ ID NO: 324 - protein, 9 aa, synthetic construct
  REGION 1..9: note = Synthetic: HVR-H1
STFSYYGMS

SEQ ID NO: 325 - protein, 9 aa, synthetic construct
  REGION 1..9: note = Synthetic: HVR-H1
FTFSYYGMS

SEQ ID NO: 326 - protein, 9 aa, synthetic construct
  REGION 1..9: note = Synthetic: HVR-H1
STFSHYGMS

SEQ ID NO: 327 - protein, 9 aa, synthetic construct
  REGION 1..9: note = Synthetic: HVR-H1
FTFAHYGMA

SEQ ID NO: 328 - protein, 9 aa, synthetic construct
  REGION 1..9: note = Synthetic: HVR-H1
FTFSSYAMG

SEQ ID NO: 329 - protein, 17 aa, synthetic construct
  REGION 1..17: note = Synthetic: HVR-H2
ATSGGGGSTYHADPVKG

SEQ ID NO: 330 - protein, 17 aa, synthetic construct
  REGION 1..17: note = Synthetic: HVR-H2
AISGSGGSTYHADSVKG

SEQ ID NO: 331 - protein, 17 aa, synthetic construct
  REGION 1..17: note = Synthetic: HVR-H2
AVSGSGGSTYYADSVRG

SEQ ID NO: 332 - protein, 17 aa, synthetic construct
  REGION 1..17: note = Synthetic: HVR-H2
SISSSDSWIYYADSVKG

SEQ ID NO: 333 - protein, 17 aa, synthetic construct
  REGION 1..17: note = Synthetic: HVR-H2
VINGDGSNKYYADSVKG

SEQ ID NO: 334 - protein, 17 aa, synthetic construct
  REGION 1..17: note = Synthetic: HVR-H2
SISSSPSHIYYADSVKG

SEQ ID NO: 335 - protein, 17 aa, synthetic construct
  REGION 1..17: note = Synthetic: HVR-H2
AISGSGGSTYYADSVKA

SEQ ID NO: 336 - protein, 17 aa, synthetic construct
  REGION 1..17: note = Synthetic: HVR-H2
GILYDGSNKYYADSVKG

SEQ ID NO: 337 - protein, 17 aa, synthetic construct
  REGION 1..17: note = Synthetic: HVR-H2
VIAYDGSNKLYADSVKG

SEQ ID NO: 338 - protein, 17 aa, synthetic construct
  REGION 1..17: note = Synthetic: HVR-H2
QILYDGSNEYYADSVKG

SEQ ID NO: 339 - protein, 17 aa, synthetic construct
  REGION 1..17: note = Synthetic: HVR-H2
VIAYDGSNKIYADSVKG

SEQ ID NO: 340 - protein, 17 aa, synthetic construct
  REGION 1..17: note = Synthetic: HVR-H2
SIIYDGSNKYYADSVKG

SEQ ID NO: 341 - protein, 17 aa, synthetic construct
  REGION 1..17: note = Synthetic: HVR-H2
SIWVDGSNKYYADSVKG

SEQ ID NO: 342 - protein, 17 aa, synthetic construct
  REGION 1..17: note = Synthetic: HVR-H2
SIWVDGSSKYYADSVKG

SEQ ID NO: 343 - protein, 17 aa, synthetic construct
  REGION 1..17: note = Synthetic: HVR-H2
SIWVDGSNKYYADSAKG

SEQ ID NO: 344 - protein, 17 aa, synthetic construct
  REGION 1..17: note = Synthetic: HVR-H2
VIWVDGSPKYYADSVKG

SEQ ID NO: 345 - protein, 17 aa, synthetic construct
  REGION 1..17: note = Synthetic: HVR-H2
VIWVDGSLKYYADSVKG

SEQ ID NO: 346 - protein, 9 aa, synthetic construct
  REGION 1..9: note = Synthetic: HVR-H1
FAFSSYAMH

SEQ ID NO: 347 - protein, 9 aa, synthetic construct
  REGION 1..9: note = Synthetic: HVR-H1
FTFSSYGMA

SEQ ID NO: 348 - protein, 9 aa, synthetic construct
  REGION 1..9: note = Synthetic: HVR-H1
FTSSSYGMH

SEQ ID NO: 349 - protein, 9 aa, synthetic construct
  REGION 1..9: note = Synthetic: HVR-H1
FTFDSYLMH

SEQ ID NO: 350 - protein, 18 aa, synthetic construct
  REGION 1..18: note = Synthetic: HVR-H3
AKDITDDFWSVTPPYFDL

SEQ ID NO: 351 - protein, 18 aa, synthetic construct
  REGION 1..18: note = Synthetic: HVR-H3
AKDTGISLWSVTPPYFDL

SEQ ID NO: 352 - protein, 18 aa, synthetic construct
  REGION 1..18: note = Synthetic: HVR-H3
AKDTGIDFWARTPPYFDL

SEQ ID NO: 353 - protein, 18 aa, synthetic construct
  REGION 1..18: note = Synthetic: HVR-H3
AKDTGIDFWGQYPPYFDL

SEQ ID NO: 354 - protein, 15 aa, synthetic construct
  REGION 1..15: note = Synthetic: HVR-H3
ARSNEKYTLGFAFDI

SEQ ID NO: 355 - protein, 15 aa, synthetic construct
  REGION 1..15: note = Synthetic: HVR-H3
ANSDTKYTLGFAFDI

SEQ ID NO: 356 - protein, 15 aa, synthetic construct
  REGION 1..15: note = Synthetic: HVR-H3
ARHSQKYTLGFAFDI

SEQ ID NO: 357 - protein, 15 aa, synthetic construct
  REGION 1..15: note = Synthetic: HVR-H3
ALMRTKYTLGFAFDI

SEQ ID NO: 358 - protein, 15 aa, synthetic construct
  REGION 1..15: note = Synthetic: HVR-H3
AVGTETYSGPNWFDP

SEQ ID NO: 359 - protein, 15 aa, synthetic construct
  REGION 1..15: note = Synthetic: HVR-H3
AVARETYSGPNWFDP

SEQ ID NO: 360 - protein, 15 aa, synthetic construct
  REGION 1..15: note = Synthetic: HVR-H3
ATGRETYSGPNWFDP

SEQ ID NO: 361 - protein, 125 aa, synthetic construct
  REGION 1..125: note = Synthetic: Heavy Chain Variable
EVQLLESGGGLVQPGGSLRLSCAASGFTFDSYAMSWVRQAPGKGLEWVSATSGGGGSTYHADPVKGRFTISRDNSKNTLYLQMNSLRAEDTAVYYCAKDITDDFWSVTPPYFDLWGRGTLVTVSS

SEQ ID NO: 362 - protein, 125 aa, synthetic construct
  REGION 1..125: note = Synthetic: Heavy Chain Variable
EVQLLESGGGLVQPGGSLRLSCAASGFTFDSYAMSWVRQAPGKGLEWVSAISGSGGSTYHADSVKGRFTISRDNSKNTLYLQMNSLRAEDTAVYYCAKDTGISLWSVTPPYFDLWGRGTLVTVSS

SEQ ID NO: 363 - protein, 125 aa, synthetic construct
  REGION 1..125: note = Synthetic: Heavy Chain Variable
EVQLLESGGGLVQPGGSLRLSCAASGFTFDNYAMSWVRQAPGEGLEWVSAVSGSGGSTYYADSVRGRFTISRDNSKNTLYLQMDSLRAEDTAVYYCAKDTGIDFWARTPPYFDLWGRGTLVTVSS

SEQ ID NO: 364 - protein, 125 aa, synthetic construct
  REGION 1..125: note = Synthetic: Heavy Chain Variable
EVQLLESGGGLVQPGGSLKLSCAASGFTFDSYAMSWVRQAPGKGLEWVSAISGSGGSTYYADSVKGRFTISRDNSKNTLYLQMNSLRAEDTAVYYCAKDTGIDFWGQYPPYFDLWGRGTLVTVSS

SEQ ID NO: 365 - protein, 122 aa, synthetic construct
  REGION 1..122: note = Synthetic: Heavy Chain Variable
QVQLVESGGGLVKPGGSLRLSCAASGFTFSSYSMHWVRQAPGKGLEWVSSISSSDSWIYYADSVKGRFTISRDNAKNSLYLQMDSLRAEDTAVYYCAKERVDYWHGYGMDVWGQGTTVTVSS

SEQ ID NO: 366 - protein, 122 aa, synthetic construct
  REGION 1..122: note = Synthetic: Heavy Chain Variable
QVQLVESGGGLVKPGGSLRLSCAASGFTFSSYSMHWVRQAPGKGLEWVSSISSSDSWIYYADSVKGRFTISRDNAKNSLYLQMNSLRAEDTAVYYCAKEPRSYWHGYGMDVWGQGTTVTVSS

SEQ ID NO: 367 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVINGDGSNKYYADSVKGRFTISRDNSKNTLYLQMNSLRAEDTAVYYCAKEPRSYWHGYGMDVWGQGTTVTVSS

SEQ ID NO: 368 - protein, 122 aa, synthetic construct
  REGION 1..122: note = Synthetic: Heavy Chain Variable
EVQLVESGGGLVKPGGSLRLSCAASGFTFSSYSMHWVRQAPGKGLEWVSSISSSPSHIYYADSVKGRFTISRDNAKNSLYLQMNSLRAEDTAVYYCAKEPRSYWHGYGMDVWGQGTTVTVSS

SEQ ID NO: 369 - protein, 119 aa, synthetic construct
  REGION 1..119: note = Synthetic: Heavy Chain Variable
EVQLLESGGGLVQPGGSLRLSCAASGFTFTEYAMSWVRQAPGKRLEWVSAISGSGGSTYYADSVKGRFTISRDNSKNTLYLQMSSLRAEDTAVYYCARDNIQWGPHFYWGQGTLVTVSS

SEQ ID NO: 370 - protein, 119 aa, synthetic construct
  REGION 1..119: note = Synthetic: Heavy Chain Variable
EVQLLESGGGLVQPGGSLRLSCAASGFTFTEYAMSWVRQAPGKGLEWVSAISGSGGSTYYADSVKARFTISRDNSKNTLYLQMNSLRAEDTAVYYCARDGTQWGPHFYWGQGTLVTVSS

SEQ ID NO: 371 - protein, 15 aa, synthetic construct
  REGION 1..15: note = Synthetic: HVR-H3
AKERVDYWHGYGMDV

SEQ ID NO: 372 - protein, 12 aa, synthetic construct
  REGION 1..12: note = Synthetic: HVR-H3
ARDNIQWGPHFY

SEQ ID NO: 373 - protein, 12 aa, synthetic construct
  REGION 1..12: note = Synthetic: HVR-H3
ARDGTQWGPHFY

SEQ ID NO: 374 - protein, 15 aa, synthetic construct
  REGION 1..15: note = Synthetic: HVR-H3
ATMRTKYTLGFAFDI

SEQ ID NO: 375 - protein, 15 aa, synthetic construct
  REGION 1..15: note = Synthetic: HVR-H3
ATSDTKYTLGFAFDI

SEQ ID NO: 376 - protein, 15 aa, synthetic construct
  REGION 1..15: note = Synthetic: HVR-H3
ATTDTKYTLGFAFDI

SEQ ID NO: 377 - protein, 15 aa, synthetic construct
  REGION 1..15: note = Synthetic: HVR-H3
ARHSVKYTLGFAFDI

SEQ ID NO: 378 - protein, 15 aa, synthetic construct
  REGION 1..15: note = Synthetic: HVR-H3
ARTPTKYTLGFAFDI

SEQ ID NO: 379 - protein, 15 aa, synthetic construct
  REGION 1..15: note = Synthetic: HVR-H3
ARHVLKYTLGFAFDI

SEQ ID NO: 380 - protein, 15 aa, synthetic construct
  REGION 1..15: note = Synthetic: HVR-H3
ASVRTKYTLGFAFDI

SEQ ID NO: 381 - protein, 15 aa, synthetic construct
  REGION 1..15: note = Synthetic: HVR-H3
ARTQLKYTLGFAFDI

SEQ ID NO: 382 - protein, 15 aa, synthetic construct
  REGION 1..15: note = Synthetic: HVR-H3
AFGLETYSGPNWFDP

SEQ ID NO: 383 - protein, 15 aa, synthetic construct
  REGION 1..15: note = Synthetic: HVR-H3
AEGKETYSGPNWFDP

SEQ ID NO: 384 - protein, 15 aa, synthetic construct
  REGION 1..15: note = Synthetic: HVR-H3
ATAVETYSGPNWFDP

SEQ ID NO: 385 - protein, 15 aa, synthetic construct
  REGION 1..15: note = Synthetic: HVR-H3
ATGVETYSGPNWFDP

SEQ ID NO: 386 - protein, 15 aa, synthetic construct
  REGION 1..15: note = Synthetic: HVR-H3
AVSRETYSGPNWFDP

SEQ ID NO: 387 - protein, 119 aa, synthetic construct
  REGION 1..119: note = Synthetic: Heavy Chain Variable
EVQLLESGGGLVQPGGSLRLSCAASGFTFSEWAMSWVRQAPGKGLEWVSAISGSGGSTYYADSVKGRFTISRDNSKNTLYLQMNSLRAEDTAVYYCARDGRTWGPHFYWGQGTLVTVSS

SEQ ID NO: 388 - protein, 122 aa, synthetic construct
  REGION 1..122: note = Synthetic: Heavy Chain Variable
QVQLVESGGGVVQPGRSLGLSCAASGFTFSSYAMHWVRQAPGKGLEWVAGILYDGSNKYYADSVKGRFTISRDNSKNTLYLQMNSLRAEDTAVYYCARSNEKYTLGFAFDIWGQGTMVTVSS

SEQ ID NO: 389 - protein, 122 aa, synthetic construct
  REGION 1..122: note = Synthetic: Heavy Chain Variable
QVQLVESGGGLVQPGGSLRLSCAASGFTFSSYAMHWVRQAPGKGLEWVAGILYDGSNKYYADSVKGRFTISRDNSKNTLYLQMNSLRAEDTAVYYCANSDTKYTLGFAFDIWGQGTMVTVSS

SEQ ID NO: 390 - protein, 122 aa, synthetic construct
  REGION 1..122: note = Synthetic: Heavy Chain Variable
QVQLVESGGGVVQPGRSLRLSCAASGFAFSSYAMHWVRQAPGKGLEWVAGILYDGSNKYYADSVKGRFTISRDNSKNTLYLQMNSLRAEDTAVYYCARHSQKYTLGFAFDIWGQGTMVTVSS

SEQ ID NO: 391 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVIAYDGSNKLYADSVKGRFTISRDNSKNTLYLQMNSLRAEDTAVYYCALMRTKYTLGFAFDIWGQGTMVTVSS

SEQ ID NO: 392 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVIAYDGSNKLYADSVKGRFTISRDNAKNSLYLQMNSPRAEDTAVYYCATMRTKYTLGFAFDIWGQGTMVTVSS

SEQ ID NO: 393 - protein, 122 aa, synthetic construct
  REGION 1..122: note = Synthetic: Heavy Chain Variable
QVQLVESGGGVVQPGRSLRLSCAASGFTFSSYGMAWVRQAPGKGLEWVAQILYDGSNEYYADSVKGRFTISRDNSKNTLYLQMNSLRAEDTAVYYCARDLTKYTLGFAFDIWGQGTMVTVSS

SEQ ID NO: 394 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVIAYDGSNKLYADSVKGRFTISRDNSKNTLYLQMDSLRAEDTAVYYCATSDTKYTLGFAFDIWGQGTMVTVSS

SEQ ID NO: 395 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVIAYDGSNKIYADSVKGRFTISRDNSKNTLYLQMNSLRAEDTAVYYCATTDTKYTLGFAFDIWGQGTMVTVSS

SEQ ID NO: 396 - protein, 122 aa, synthetic construct
  REGION 1..122: note = Synthetic: Heavy Chain Variable
EVQLLESGGGVVQPGRSLRLSCAASGFTFSSYGMHWVRQAPGKGLEWVAGILYDGSNKYYADSVKGRFTISRDNSKNTLYLQMDGLRAEDTAVYYCARHSVKYTLGFAFDIWGQGTMVTVSS

SEQ ID NO: 397 - protein, 122 aa, synthetic construct
  REGION 1..122: note = Synthetic: Heavy Chain Variable
EVQLLESGGGVVQPGRSLRLSCAASGFTFAHYGMAWVRQAPGKGLEWVAVIWVDGSPKYYADSVKGRFTISRDNSKNTLYLQMNSLRAEDTAVYYCAVARETYSGPNWFDPWGQGTLVTVSS

SEQ ID NO: 398 - protein, 122 aa, synthetic construct
  REGION 1..122: note = Synthetic: Heavy Chain Variable
QVQLVESGGGVVQPGRSLRLSCAASGFTSSSYGMHWVRQAPGKGLEWVAVIAYDGSNKLYADSVKGRFTISRDNSKNTLYLQMNSLRAEDTAVYYCARTPTKYTLGFAFDIWGQGTMVTVSS

SEQ ID NO: 399 - protein, 122 aa, synthetic construct
  REGION 1..122: note = Synthetic: Heavy Chain Variable
QVQLVESGGGVVQPGRSLRLSCAASGFTFSSYAMHWVRQAPGKGLEWVAGILYDGSNKYYADSVKGRFTISRDNSKNTLYLQMNSLRAEDTAVYYCARHVLKYTLGFAFDIWGQGTMVTVSS

SEQ ID NO: 400 - protein, 122 aa, synthetic construct
  REGION 1..122: note = Synthetic: Heavy Chain Variable
QVQLVESGGGVVQPGRSLRLSCAASGFTFDSYLMHWVRQAPGKGLEWVASIIYDGSNKYYADSVKGRFTISRDNSKNTLYLQMNSLRAEDTAVYYCASVRTKYTLGFAFDIWGQGTMVTVSS

SEQ ID NO: 401 - protein, 122 aa, synthetic construct
  REGION 1..122: note = Synthetic: Heavy Chain Variable
QVQLVESGGGVVQPGRSLRLSCAASGFTFSSYGMHWVRQAPGKGLEWVAVIAYDGSNKLYADSVKGRFTISRDNSKNTLYLQMNSLRAEDTAVYYCARTQLKYTLGFAFDIWGQGTMVTVSS

SEQ ID NO: 402 - protein, 122 aa, synthetic construct
  REGION 1..122: note = Synthetic: Heavy Chain Variable
EVQLLESGGGVVQPGRSLGLSCAASGSTFSYYGMSWVRQAPGKGLEWVASIWVDGSNKYYADSVKGRFTISRDNSKSTLYLQMNSLRAEDTAVYYCARGVETYSGPNWFDPWGQGTLVTVSS

SEQ ID NO: 403 - protein, 122 aa, synthetic construct
  REGION 1..122: note = Synthetic: Heavy Chain Variable
QVQLVESGGGVVQPGGSLRLSCAASGFTFSYYGMSWVRQAPGKGLEWVASIWVDGSSKYYADSVKGRFTISRDNSKNTLYLQMNSLRAEDTAVYYCARGVETYSGPNWFDPWGQGTLVTVSS

SEQ ID NO: 404 - protein, 122 aa, synthetic construct
  REGION 1..122: note = Synthetic: Heavy Chain Variable
EVQLVESGGGVVQPGRSLRLSCAASGFTFSYYGMSWVRQAPGKGLEWVASIWVDGSNKYYADSVKGRFTISRDNSKNTLYLQMNSLRAEDTAVYYCAFGLETYSGPNWFDPWGQGTLVTVSS

SEQ ID NO: 405 - protein, 122 aa, synthetic construct
  REGION 1..122: note = Synthetic: Heavy Chain Variable
EVQLVESGGGVVQPGRSLRLSCAASGFTFSYYGMSWVRQAPGKGLEWVASIWVDGSNKYYADSAKGRFTISRDNSKNTLYLQMNSLRAEDTAVYYCAEGKETYSGPNWFDPWGQGTLVTVSS

SEQ ID NO: 406 - protein, 122 aa, synthetic construct
  REGION 1..122: note = Synthetic: Heavy Chain Variable
EVQLVESGGGVVQPGGSLRLSCAASGFTFSYYGMSWVRQAPGKGLEWVASIWVDGSNKYYADSVKGRFTVSRDNSKNTLYLQMDSLRAEDTAVYYCARGVETYSGPNWFDPWGQGTLVTVSS

SEQ ID NO: 407 - protein, 122 aa, synthetic construct
  REGION 1..122: note = Synthetic: Heavy Chain Variable
QVQLVESGGGVVQPGRSLRLSCAASGSTFSHYGMSWVRQAPGKGLEWVASIWVDGSNKYYADSVKGRFTISRDNSKNTLYLQMDSLRAEDTAVYYCARGVETYSGPNWFDPWGQGTLVTVSS

SEQ ID NO: 408 - protein, 122 aa, synthetic construct
  REGION 1..122: note = Synthetic: Heavy Chain Variable
EVQLVESGGGVVQPGRSLRLSCAASGFTFAHYGMAWVRQAPGKGLEWVASIWVDGSNKYYADSVKGRFTISRDNSKNTLYLQMNSLRAEDTAVYYCATAVETYSGPNWFDPWGQGTLVTVSS

SEQ ID NO: 409 - protein, 122 aa, synthetic construct
  REGION 1..122: note = Synthetic: Heavy Chain Variable
EVQLVESGGGVVQPGRSLRLSCAASGFTFSYYGMSWVRQAPGKGLEWVASIWVDGSNKYYADSVKGRFTISRDNSKNTLYLQMDSLRAEDTAVYYCATGVETYSGPNWFDPWGQGTLVTVSS

SEQ ID NO: 410 - protein, 122 aa, synthetic construct
  REGION 1..122: note = Synthetic: Heavy Chain Variable
EVQLLESGGGVVQPGRSLRLSCAASGFTFSYYGMSWVRQAPGKGLEWVASIWVDGSNKYYADSVKGRFTISRDNSKNALYLQMNSLRAEDTAVYYCAVSRETYSGPNWFDPWGQGTLVTVSS

SEQ ID NO: 411 - protein, 122 aa, synthetic construct
  REGION 1..122: note = Synthetic: Heavy Chain Variable
EVQLVESGGGVVQPGRSLRLSCAASGFTFSYYGMSWVRQAPGKGLEWVASIWVDGSNKYYADSVKGRFTISRDNSKNTLYLQMNSLRAEDTAVYYCAVGTETYSGPNWFDPWGQGTLVTVSS

SEQ ID NO: 412 - protein, 122 aa, synthetic construct
  REGION 1..122: note = Synthetic: Heavy Chain Variable
EVQLLESGGGVVQPGRSLRLSCAASGFTFSSYAMGWVRQAPGKGLEWVAVIWVDGSLKYYADSVKGRFTISRDNSKNTLYLQMNSLRAEDTAVYYCATGRETYSGPNWFDPWGQGTLVTVSS

SEQ ID NO: 413 - protein, 107 aa, synthetic construct
  REGION 1..107: note = Synthetic: Light Chain Variable
DIQMTQSPSSVSASVGDRVTITCRASQGISSWLAWYQQKPGKAPKLLIYAASSLQSGVPSRFSGSGSGTDFTLTISSLQPEDFATYYCQQAIYFPITFGGGTKVENQ